(19) United States
(12) Patent Application Publication
(10) Pub. No.: US 2024/0261292 A1
(43) Pub. Date: Aug. 8, 2024

(54) COMBINATION THERAPIES

(71) Applicant: Janssen Pharmaceutica NV, Beerse (BE)

(72) Inventors: Nikki Daskalakis, Colts Neck, NJ (US); Christina Diane Guttke, Sellersville, PA (US); Min Chul Kwon, Mol (BE); Lucille Angela Ferrante, Winston-Salem, NC (US); Kathryn Elizabeth Packman, Cambridge, MA (US); Eva Christine Pietsch, Spring House, PA (US); Ulrike Philippar, Antwerpen (BE); Tinne Ann J. Verhulst, Balen (BE); Sumia Ali-Ahmed, Beerse (BE); Balpreet Bhogal, Plainsboro, NJ (US); Yu Sun, Belle Mead, NJ (US); Wei Cai, Shanghai (CN); Xuedong Dai, Shanghai (CN); Olivier Alexis Georges Querolle, Saint Vigor (FR); Johannes Wilhelmus J. Thuring, Antwerpen (BE); Yingtao Liu, Shanghai (CN); Lianzhu Liu, Shanghai (CN); Yanping Xu, Noblesville, IN (US); Liqiang Fu, Shanghai (CN); Ming Li, Shanghai (CN); Lichao Fang, Shanghai (CN); Xiangjun Deng, Shanghai (CN); Alicia Tee Fuay Ng, Shanghai (CN); Nicolas Freddy J. Darville, Linkhout (BE); Vineet Pande, Vosselaar (BE)

(21) Appl. No.: 18/560,233

(22) PCT Filed: May 9, 2022
(86) PCT No.: PCT/CN2022/091678

§ 371 Date: Nov. 10, 2023

(30) Foreign Application Priority Data
May 11, 2021 (WO) PCT/CN2021/093036
Jun. 17, 2021 (WO) PCT/CN2021/100522
Jun. 17, 2021 (WO) PCT/CN2021/100523
Apr. 11, 2022 (WO) PCT/CN2022/086003
Apr. 11, 2022 (WO) PCT/CN2022/086004

(51) Int. Cl. A61K 31/53; A61K 31/4725; A61K 31/553; A61K 31/704; A61K 31/706; A61P 35/00

(52) U.S. Cl. CPC A61K 31/53; A61K 31/4725; A61K 31/553; A61K 31/704; A61K 31/706; A61P 35/00

(57) ABSTRACT

Disclosed are combinations comprising a therapeutically effective amount of a menin-MLL inhibitor of Formula (I) or a pharmaceutically acceptable salt or a solvate thereof; and a therapeutically effective amount of at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor. Also disclosed are methods for treating a subject who has been diagnosed with cancer using such combinations. Compounds are represented by Formula (I) as follows:

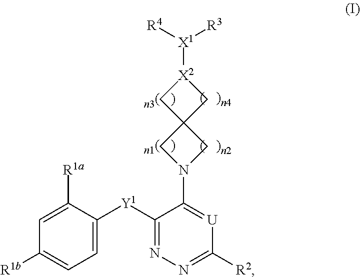

wherein R^{1a}, R^{1b}, R², R³, R⁴, U, Y¹, X¹, X², n1, n2, n3 and n4 are defined herein.

### FIELD OF THE INVENTION

The present invention relates to novel combinations comprising a therapeutically effective amount of a menin-mixed-lineage leukemia 1 (menin-MLL) inhibitor of Formula (I), or a pharmaceutically acceptable salt or a solvate thereof; and a therapeutically effective amount of at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor; as well as to methods for treating a subject diagnosed with cancer using such combinations.

### BACKGROUND OF THE INVENTION

Cancer is the leading cause of death worldwide. Of the 10 million cancer deaths recorded by GLOBOCAN in 2020, 7.1% are attributed to hematopoietic disorders. Accordingly, new treatment modalities are urgently needed for hematopoietic disorders, including acute myeloid leukemia (AML), myelodysplastic syndrome (MDS) and acute lymphoblastic leukemia (ALL) as further detailed below.

AML is a common hematological malignancy whose incidence rises from 3:100,000 in young adults to greater than 20:100,000 in older adults. For patients <60 years of age, overall survival (OS) is 40 to 50%, but is only 5% for patients >60 years of age. The majority of newly diagnosed patients with AML are over the age of 60. In this patient population, standard induction chemotherapy is often not an option due to increased treatment-related mortality as a result of age and co-morbidities. Standard of care for AML patients unfit for combination chemotherapy is treatment with hypomethylating agents (azacitidine or decitabine) or low dose cytarabine. Relapsed/refractory AML with a FMS-like tyrosine kinase 3 (FLT3) mutation is treated with a FLT3 kinase inhibitor (e.g., gilteritinib, midostaurin). Despite these frontline treatments, median OS is only about 10 months. In all types of AML, disease relapse is common despite an initial therapeutic response and is the most common reason for death. Standard chemotherapy and allogeneic stem cell transplant (when used) often fail to eradicate all tumor-propagating cells and select for chemotherapy-resistant leukemia-propagating subclones. Patients refractory to salvage therapy are treated palliatively, as current treatment options are extremely limited. These patients have a median survival of 2 months. In addition, patients with newly diagnosed intermediate or higher-risk MDS and those who relapse after standard care have a poor prognosis and high risk of progression to AML. Therefore, there is an urgent need for new treatment modalities for relapsed/refractory (R/R) AML and MDS patients, newly diagnosed AML patients ineligible for induction chemotherapy based on age and co-morbidities, and newly diagnosed intermediate/high/very high risk MDS patients.

ALL is a hematologic malignancy propagated by impaired differentiation, proliferation, and accumulation of lymphoid progenitor cells in the bone marrow and/or extramedullary sites. ALL represents 12% of all leukemia cases and is the most common childhood acute leukemia, with a worldwide incidence projected to be 1 to 4.75 per 100,000 people. ALL represents about 20% of adult leukemias. Despite high rates of complete remission (CR) (80% to 90%) with current therapies, the majority of adult patients with ALL relapse. The 5-year overall survival rate is approximately 30 to 40% in adults and elderly patients. Therefore, there is an urgent need for new treatment modalities for the treatment of cancer, in particular relapsed/refractory ALL, more particularly in adult and especially elderly patients.

### SUMMARY OF THE INVENTION

Embodiments of the present invention relate to novel combinations of a menin-MLL inhibitor of Formula (I), or a pharmaceutically acceptable salt or a solvate thereof; and at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

Embodiments of the present invention relate to uses of such combinations for treating a subject who has been diagnosed with a hematopoietic disorder, including but not limited to, blood cancers, using a menin-MLL inhibitor described herein in combination with at least one other therapeutic agent.

Embodiments of the present invention relate to novel methods for treating a subject who has been diagnosed with a hematopoietic disorder using such combinations. Embodiments of the novel methods comprise administering to the subject a therapeutically effective amount of a menin-MLL inhibitor as described herein; and a therapeutically effective amount of at least one other therapeutic agent; wherein the menin-MLL inhibitor is a compound of Formula (I), or a pharmaceutically acceptable salt or a solvate thereof.

Embodiments of the present invention relate to novel methods for treating a subject who has been diagnosed with a hematopoietic disorder using such combinations. Embodiments of the novel methods comprise administering to the subject a therapeutically effective amount of a menin-MLL inhibitor as described herein; and a therapeutically effective amount of at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor; wherein the menin-MLL inhibitor is a compound of Formula (I), or a pharmaceutically acceptable salt or a solvate thereof.

In some embodiments, the present invention is directed to methods for treating a subject who has been diagnosed with a hematopoietic disorder, the methods comprising administering to the subject a therapeutically effective amount of a menin-MLL inhibitor of Formula (I), or a pharmaceutically acceptable salt or a solvate thereof; and a therapeutically effective amount of azacitidine or a pharmaceutically acceptable salt or solvate thereof.

In some embodiments, the present invention is directed to methods for treating a subject who has been diagnosed with a hematopoietic disorder, the methods comprising administering to the subject a therapeutically effective amount of a menin-MLL inhibitor of Formula (I), or a pharmaceutically acceptable salt or a solvate thereof; and a therapeutically effective amount of azacitidine or a pharmaceutically acceptable salt or solvate thereof; wherein the azacitidine, or a pharmaceutically acceptable salt or solvate thereof, is administered to the subject prior to, simultaneous with, or after the administration of the menin-MLL inhibitor.

In embodiments, the menin-MLL inhibitor of Formula (I) is:

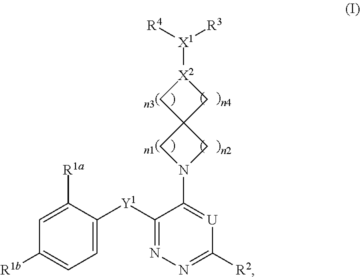

and the tautomers and the stereoisomeric forms thereof, wherein
R^{1a} represents —C(═O)—NR^{xa}R^{xb}; Het; or

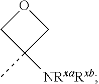

Het represents a 5- or 6-membered monocyclic aromatic ring containing one, two or three nitrogen atoms and optionally a carbonyl moiety;
wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}, —O— or —NR^{5c}—;
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}, —C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl; wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl; and
R^{9a}, R^{9b}, R^{10a}, R^{10b}, R^{10c}, R¹¹, R^{12a}, and R^{12b} are each independently selected from the group consisting of hydrogen and C₁₋₆alkyl;
and the pharmaceutically acceptable salts and the solvates thereof.

In particular embodiments, the menin-MLL inhibitor of Formula (I) is (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt (benzenesulfonate salt):

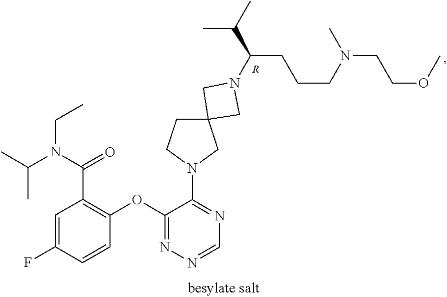

and solvates thereof.

A skilled person will understand that the ‘and solvates thereof’ refer to the besylate salt of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide.

In particular embodiments, the menin-MLL inhibitor of Formula (I) is (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt or hydrates thereof.

In particular embodiments, the menin-MLL inhibitor of Formula (I) is (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or solvates thereof.

In particular embodiments, the menin-MLL inhibitor of Formula (I) is (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or hydrates thereof.

In particular the present invention is directed to (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt 0.5-2.0 equivalents hydrate.

In particular the present invention is directed to (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt 2.0 equivalents hydrate.

In particular embodiments, the menin-MLL inhibitor of Formula (I) is a crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate.

In particular embodiments, the menin-MLL inhibitor of Formula (I) is a crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt 0.5-2.0 equivalents hydrate.

More in particular the present invention is directed to a crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt 2.0 equivalents hydrate.

Additional embodiments, features, and advantages of the invention will be apparent from the following detailed description and through practice of the invention.

### BRIEF DESCRIPTION OF THE DRAWINGS

FIG. 1 is an X-ray powder diffraction (XRPD) pattern of Compound A4: a crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate.

FIG. 2 depicts a comparison of tumor volumes as a function of time for the control group and for the treatment groups treated with a regimen comprising various amounts of Compound A3.

FIG. 3 depicts a comparison of tumor percent survival as a function of time (e.g., Kaplan-Meier survival curves) for the control group and for the treatment groups treated with a regimen comprising various amounts of Compound A3.

FIG. 4A depicts a comparison of percent survival as a function of time of mice bearing established OCI-AML3 tumors following treatment with vehicle, monotherapy with either azacitidine or Compound A1, or the doublet combination of Compound A1 and azacitidine.

FIG. 4B depicts a comparison of percent survival as a function of time of mice bearing established MOLM-13 tumors following treatment with vehicle, monotherapy with either azacitidine or Compound A1, or the doublet combination of Compound A1 and azacitidine.

FIG. 5 depicts a comparison of percent survival as a function of time of mice bearing established MOLM-13 tumors following treatment with vehicle, monotherapy with either gilteritinib or Compound A1, or the doublet combination of Compound A1 and gilteritinib.

FIG. 6A is a contour plot for maxR which illustrates the effect of Compound A4 in combination with gilteritinib on proliferation of MOLM-13 cells in vitro.

FIG. 6B is a contour plot for maxR which illustrates the effect of Compound A4 in combination with gilteritinib on proliferation of MV4-11 cells in vitro.

FIG. 7A is a contour plot for maxR which illustrates the effect of Compound A4 in combination with midostaurin on proliferation of MOLM-13 cells in vitro.

FIG. 7B is a contour plot for maxR which illustrates the effect of Compound A4 in combination with midostaurin on proliferation of MV4-11 cells in vitro.

FIG. 8A is a contour plot for maxR which illustrates the effect of Compound A4 in combination with idarubicin on proliferation of MOLM-13 cells in vitro.

FIG. 8B is a contour plot for maxR which illustrates the effect of Compound A4 in combination with idarubicin on proliferation of OCI-AML3 cells in vitro.

FIG. 9A is a contour plot for maxR which illustrates the effect of Compound A4 in combination with decitabine on proliferation of MOLM-13 cells in vitro.

FIG. 9B is a contour plot for maxR which illustrates the effect of Compound A4 in combination with decitabine on proliferation of OCI-AML3 cells in vitro.

FIG. 10A is a contour plot for maxR which illustrates the effect of Compound A3 in combination with DHODH inhibitor Compound 22 on proliferation of MOLM-13 cells in vitro.

FIG. 10B is a contour plot for maxR which illustrates the effect of Compound A3 in combination with DHODH inhibitor Compound 22 on proliferation of OCI-AML3 cells in vitro.

FIG. 11A depicts a comparison of percent survival as a function of time of mice bearing established MOLM-13 tumors following treatment with vehicle, monotherapy with either menin-MLL inhibitor Compound A1 or DHODH inhibitor Compound 22, or the doublet combination of Compound A1 and Compound 22.

FIG. 11B depicts a comparison of percent survival as a function of time of mice bearing established OCI-AML3 tumors following treatment with vehicle, monotherapy with either menin-MLL inhibitor Compound A1 or DHODH inhibitor Compound 22, or the doublet combination of Compound A1 and Compound 22.

### DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain saturated hydrocarbon radical having from 1 to 4 carbon atoms, such as methyl, ethyl, n-propyl, isopropyl, n-butyl, s-butyl, t-butyl and the like.

Similar, the term ‘C₁₋₆alkyl’ as used herein as a group or part of a group represents a straight or branched chain saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₃₋₆cycloalkyl’ as used herein as a group or part of a group defines a saturated, cyclic hydrocarbon radical having from 3 to 6 carbon atoms, such as cyclopropyl, cyclobutyl, cyclopentyl and cyclohexyl.

It will be clear for the skilled person that S(═O)₂ or SO₂ represents a sulfonyl moiety.

It will be clear for the skilled person that CO or C(═O) represents a carbonyl moiety.

It will be clear for the skilled person that a group such as —CRR— represents

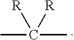

An example of such a group is —CR^{5a}R^{5b}—.

It will be clear for the skilled person that a group such as —NR— represents

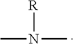

An example of such a group is —NR^{5c}—.

Non-limiting examples of ‘monocyclic 5- or 6-membered aromatic rings containing one, two or three nitrogen atoms and optionally a carbonyl moiety’, include, but are not limited to pyrazolyl, imidazolyl, pyridinyl, pyridazinyl, pyrimidinyl, pyrazinyl, triazinyl or 1,2-dihydro-2-oxo-4-pyridinyl.

The skilled person will understand that a 5- or 6-membered monocyclic aromatic ring containing one, two or three nitrogen atoms and a carbonyl moiety includes, but is not limited to

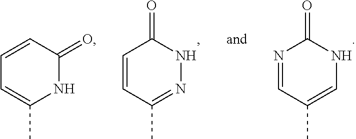

When any variable occurs more than one time in any constituent, each definition is independent.

When any variable occurs more than one time in any formula (e.g., Formula (I)), each definition is independent.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e., a compound that is sufficiently robust to survive isolation to a useful degree of purity from a reaction mixture (isolation after a reaction e.g., purification by silica gel chromatography). In a particular embodiment, when the number of substituents is not explicitly specified, the number of substituents is one.

Combinations of substituents and/or variables are permissible only if such combinations result in chemically stable compounds. ‘Stable compound’ is in this context meant to indicate a compound that is sufficiently robust to survive isolation to a useful degree of purity from a reaction mixture (isolation after a reaction e.g., purification by silica gel chromatography).

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

Within the context of this invention ‘saturated’ means ‘fully saturated’, if not otherwise specified.

Unless otherwise specified or clear from the context, aromatic rings groups, can be attached to the remainder of the molecule of Formula (I) through any available ring carbon atom (C-linked) or nitrogen atom (N-linked).

Unless otherwise specified or clear from the context, aromatic rings groups, may optionally be substituted, where possible, on carbon and/or nitrogen atoms according to the embodiments.

The term “comprising” as used herein, encompasses the terms “consisting of” and “consisting essentially of.” All embodiments described herein using the term “comprising” are also applicable for embodiments of the invention wherein the term “comprising” is limited to “consisting of.” Likewise, all embodiments described herein using the term“comprising” are also applicable for embodiments of the invention wherein the term “comprising” is limited to “consisting essentially of.”

The term “subject” as used herein, refers to an animal, preferably a mammal (e.g., cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medicinal doctor or other clinician, which includes alleviation or reversal of the symptoms of the disease or disorder being treated.

The term “composition” is intended to encompass a product including the specified ingredients in the specified amounts, as well as any product which results, directly or indirectly, from combinations of the specified ingredients in the specified amounts.

The terms “treatment” and “treating,” as used herein, are intended to refer to all processes wherein there may be a slowing, interrupting, arresting or stopping of the progression of a disorder, or amelioration of one or more symptoms thereof, but does not necessarily indicate a total elimination of all symptoms.

As used herein, any chemical formula with bonds shown only as solid lines and not as solid wedged or hashed wedged bonds, or otherwise indicated as having a particular configuration (e.g., R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

Hereinbefore and hereinafter, the term “compound(s) of Formula (Z)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropisomers) are stereoisomers which have a particular spatial configuration, resulting from a restricted rotation about a single bond, due to large steric hindrance. All atropisomeric forms of the compounds of Formula (I) are intended to be included within the scope of the present invention.

Diastereomers (or diastereoisomers) are stereoisomers that are not enantiomers, i.e., they are not related as mirror images. If a compound contains a double bond, the substituents may be in the E or the Z configuration.

Substituents on bivalent cyclic saturated or partially saturated radicals may have either the cis- or trans-configuration; for example, if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e., enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light.

When a specific stereoisomer is identified, this means that said stereoisomer is substantially free, i.e., associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

Some of the compounds according to Formula (I) may also exist in their tautomeric form. Such forms in so far as they may exist, although not explicitly indicated in the above Formula (I) are intended to be included within the scope of the present invention. It follows that a single compound may exist in both stereoisomeric and tautomeric form.

Pharmaceutically acceptable salts include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g., in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I) and solvates thereof, are able to form.

Appropriate acids comprise, for example, inorganic acids such as hydrohalic acids, e.g., hydrochloric or hydrobromic acid, sulfuric, nitric, phosphoric and the like acids; or organic acids such as, for example, acetic, propanoic, hydroxyacetic, lactic, pyruvic, oxalic (i.e., ethanedioic), malonic, succinic (i.e., butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g., the lithium, sodium, potassium, cesium, magnesium, calcium salts and the like, salts with organic bases, e.g., primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term “prodrug” includes any compound that, following oral or parenteral administration, in particular oral administration, is metabolised in vivo to a (more) active form in an experimentally-detectable amount, and within a predetermined time (e.g., within a dosing interval of between 0.5 and 24 hours, or e.g., within a dosing interval of between 6 and 24 hours (i.e., once to four times daily)). For the avoidance of doubt, the term “parenteral” administration includes all forms of administration other than oral administration, in particular intravenous (IV), intramuscular (IM), and subcutaneous (SC) injection.

Prodrugs may be prepared by modifying functional groups present on a compound in such a way that the modifications are cleaved in vivo when such prodrug is administered to a mammalian subject. The modifications typically are achieved by synthesizing the parent compound with a prodrug substituent. In general, prodrugs include compounds wherein a hydroxyl, amino, sulfhydryl, carboxy or carbonyl group is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g., in Bundegaard, H. “Design of Prodrugs” p. 1-92, Elesevier, New York-Oxford (1985).

The term solvate comprises the solvent addition forms as well as the salts thereof, which the compounds of Formula (I) are able to form. Examples of such solvent addition forms are e.g., hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C, ¹³C and ¹⁸F. Preferably, the isotope is selected from the group of ²H, ³H, ¹¹C and ¹⁸F. More preferably, the isotope is ²H, ³H or ¹³C. More preferably, the isotope is ²H or ¹³C. More preferably, the isotope is ²H. In particular, deuterated compounds and ¹³C-enriched compounds are intended to be included within the scope of the present invention. In particular, deuterated compounds are intended to be included within the scope of the present invention.

Certain isotopically-labeled compounds (e.g., those labeled with ³H and ¹⁴C) may be useful for example in substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumors, stage the disease and determine suitable treatment. Human cancer cells overexpress many receptors or proteins that are potential disease-specific molecular targets. Radiolabelled tracers that bind with high affinity and specificity to such receptors or proteins on tumor cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

Solid oral dosage forms such as, tablets or capsules, containing one or more compounds described herein may be administered in at least one dosage form at a time, as appropriate. It is also possible to administer the compounds in sustained release formulations.

Additional oral forms in which the compounds described herein may be administered include elixirs, solutions, syrups, and suspensions; each optionally containing flavoring agents and coloring agents.

Alternatively, one or more compounds described herein can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as may be required. An alternative means of administration includes transdermal administration by using a skin or transdermal patch.

The pharmaceutical compositions used in the methods of the present invention (as well as the compounds alone) can also be injected parenterally, for example, intracavernosally, intravenously, intramuscularly, subcutaneously, intradermally, or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing a compound of Formula (I), or a pharmaceutically acceptable salt or a solvate thereof, and at least one other therapeutic agent as an active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also may be optionally coated with substances such as, sugars, or be enterically coated so as to modulate the major site of absorption and disintegration. For parenteral administration, the carrier, excipient and diluent will usually include sterile water, and other ingredients may be added to increase solubility and preservation of the composition. Injectable suspensions or solutions may also be prepared utilizing aqueous carriers along with appropriate additives such as, solubilizers and preservatives.

According to particular embodiments, methods using a therapeutically effective amount of a compound of Formula (I), or a pharmaceutically acceptable salt or a solvate thereof, and at least one other therapeutic agent, may comprise a dose range from about 0.1 mg to about 3000 mg, or any particular amount or range therein, in particular from about 1 mg to about 1000 mg, or any particular amount or range therein, of active ingredient in a regimen of about 1 to about (4×) per day for an average (70 kg) human; although, it is apparent to one skilled in the art that the therapeutically effective amount for a compound of Formula (I), or a pharmaceutically acceptable salt or a solvate thereof, and at least one other therapeutic agent, will vary as will the diseases, syndromes, conditions, and disorders being treated.

An embodiment of the present invention is directed to methods of using pharmaceutical compositions for oral administration, comprising a compound of Formula (I), or a pharmaceutically acceptable salt or a solvate thereof in an amount of from about 1 mg to about 500 mg. Advantageously, a compound of Formula (I), or a pharmaceutically acceptable salt or a solvate thereof may be administered in a single daily dose, or the total daily dosage may be administered in divided doses of two, three and (4×) daily.

Optimal dosages of a compound of Formula (I), or a pharmaceutically acceptable salt or a solvate thereof to be administered may be readily determined and will vary with the particular compound used, the mode of administration, the strength of the preparation, and the advancement of the hematopoietic disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I), or a pharmaceutically acceptable salt or a solvate thereof may be administered in any of the foregoing compositions and dosage regimens or by means of those compositions and dosage regimens established in the art whenever use of a compound of Formula (I), or a pharmaceutically acceptable salt or a solvate thereof is administered to a subject in need thereof.

An embodiment of the present invention is directed to methods of using pharmaceutical compositions for intravenous or subcutaneous administration, comprising a therapeutic agent in an amount of from about 1 mg to about 500 mg. Advantageously, the therapeutic agent may be administered in a single daily dose, or the total daily dosage may be administered in divided doses of two, three and (4×) daily.

Optimal dosages of the therapeutic agent to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

The therapeutic agent may be administered in any of the foregoing compositions and dosage regimens or by means of those compositions and dosage regimens established in the art whenever use of a therapeutic is administered to a subject in need thereof.

As used herein, the term “menin-MLL inhibitor” refers to an inhibitor of the protein-protein interaction between menin and mixed-lineage leukemia 1 (MLL1) (also known as histone-lysine N-methyltransferase 2A (KMT2A) protein in the scientific field (UniProt Accession #Q03164)) which inhibits or reduces menin-MLL 1 activity. Menin-MLL inhibitors described herein are disclosed in PCT/CN2020/137266 (which published as WO 2021/121327 on Jun. 24, 2021), which is incorporated by reference herein in its entirety, and which also discloses corresponding synthetic schemes and analytical characterizations.

As used herein, the term “therapeutic agent” refers to any agent that treats cancer. In the context of this application, in some embodiments, therapeutic agents are limited to the therapeutic agents explicitly listed herein.

As used herein, the term “hypomethylating agent” refers to an agent that inhibits or reduces DNA methylation.

As used herein, the term “cytidine deaminase inhibitor” refers to an agent that inhibits or reduces cytidine deaminase activity.

As used herein, the term “kinase inhibitor” refers to an agent that inhibits or reduce the activity of at least one kinase (e.g., tyrosine and/or serine kinases such as fms-like receptor tyrosine kinase-3 (FLT3), Bruton tyrosine kinase (BTK), an Abelson tyrosine kinase 1 (ABL), an Aurora serine/tyrosine kinase).

As used herein, the term “FLT-3 inhibitor” refers to tyrosine kinase inhibitors (TKI) classified into first and next generation inhibitors based on their potency and specificity for fms-like receptor tyrosine kinase-3 (FLT3) and their associated downstream targets.

As used herein, the term “CD20 inhibitor” refers to any agent that reduces activity of CD20.

As used herein, the term “isocitrate dehydrogenase (IDH) inhibitor” refers to any agent that interferes with the conversion of isocitrate to α-ketoglutarate (α-KG) in the tricarboxylic acid (TCA) cycle.

As used herein, the term “immunomodulatory agent” refers to any agent that stimulates or suppresses the immune system (e.g., via cytokine modulation, co-stimulation of T cells, down-regulation of co-inhibitory molecules, enhancing natural killer cell activity, inhibition of regulatory T cells, and repairing perturbed synapse formation on T cells). In particular embodiments, immunomodulatory agents, such as monoclonal antibodies, cytokines, and vaccines, affect specific parts of the immune system. In other embodiments, immunomodulatory agents, such as Bacillus Calmette-Guérin (BCG) and levamisole, affect the immune system in a general way.

As used herein, the term “programmed cell death protein 1 (PD-1) inhibitor” refers to any agent that inhibits or reduces PD-1 activity.

As used herein, the term “dihydroorotate dehydrogenase (DHODH) inhibitor” refers to any agent that inhibits or reduces dihydroorotate dehydrogenase activity.

As used herein, unless otherwise noted, the term “affect” or “affected” (when referring to a disease, disorder, or medical condition that is affected by the inhibition or alteration of menin-MLL activity) includes a reduction in the frequency and/or severity of one or more symptoms or manifestations of said hematopoietic disorder; and/or includes the prevention of the development of one or more symptoms or manifestations of said hematopoietic disorder or the development of the hematopoietic disorder.

As used herein, the term “hematopoietic disorder” refers to any disorder associated with the production of the cellular components of blood and blood plasma, including but not limited to blood cancers.

According to an embodiment, the invention provides combinations as described herein.

According to an embodiment, the invention provides combinations as described herein for use as a medicament.

According to an embodiment, the invention provides combinations as described herein for the manufacture of a medicament.

According to an embodiment, the invention provides combinations as described herein for the manufacture of a medicament for the treatment or prevention of any one of the disease conditions mentioned herein.

According to an embodiment, the invention provides combinations as described herein for use in the prevention or treatment, in particular treatment, of diseases as described herein.

According to an embodiment, the invention provides combinations as described herein for use in the prevention or treatment, in particular treatment, of cancer.

According to an embodiment, the invention provides combinations as described herein for use in the prevention or treatment, in particular treatment, of a cancer, including but not limited to solid tumors, sarcomas and hematopoietic disorders.

According to an embodiment, the cancer is selected from, but not limited to, breast cancer, colorectal carcinoma, gastric cancer, glioma, head & neck cancer, hepatocellular carcinoma, lung cancer, multiple myeloma, neuroblastoma, ovarian cancer, pancreatic cancer, prostate cancer, renal cell carcinoma and sarcoma.

According to an embodiment, the sarcoma is selected from, but not limited to, sarcoma of the soft tissue, glioma, osteosarcoma, malignant fibrous histiocytoma, lymphosarcoma, and rhabdomyosarcoma.

According to an embodiment, the invention provides combinations as described herein for use in the prevention or treatment, in particular treatment, of a hematopoietic disorder, including but not limited to blood cancers, including but not limited to lymphomas, myelomas and leukemias.

According to an embodiment, the invention provides combinations as described herein for use in the prevention or treatment, in particular treatment, of a hematopoietic disorder.

According to an embodiment, the hematopoietic disorder is selected from, but not limited to, lymphomas, myelomas, myelodysplasia and leukemias.

According to an embodiment, the hematopoietic disorder is a lymphoma selected from Hodgkin's disease lymphomas and Non-Hodgkin's lymphomas.

According to an embodiment, the lymphoma is a Non-Hodgkin's disease that is Burkitt's lymphoma, anaplastic large cell lymphoma, splenic marginal zone lymphoma, hepatosplenic T-cell lymphoma or angioimmunoblastic T-cell lymphoma (AILT).

According to an embodiment, the hematopoietic disorder is a myeloma. According to an embodiment, the hematopoietic disorder is a multiple myeloma, Waldenström macroglobulinemia or plasmacytoma.

According to an embodiment the hematopoietic disorder is a myelodysplasia including, but not limited to, myelodysplastic syndrome (MDS).

According to an embodiment, the hematopoietic disorder is a leukemia.

According to an embodiment, the hematopoietic disorder is a leukemia selected from acute leukemias and chronic leukemias. According to an embodiment, the leukemia is an acute leukemia. According to an embodiment, the leukemia is chronic leukemia.

According to an embodiment, the hematopoietic disorder is a myeloid leukemia, myelogenous leukemia, lymphoblastic leukemia, or lymphocytic leukemia, According to an embodiment, the hematopoietic disorder is a leukemia selected from, but not limited to, acute lymphocytic leukemia (ALL), chronic lymphocytic leukemia (CLL), small lymphocytic leukemia (SLL), acute myeloid leukemia (AML), chronic idiopathic myelofibrosis (MF), chronic myelogenous leukemia (CML), T-cell prolymphocytic leukemia (T-PLL), B-cell prolymphocytic leukemia (B-PLL), chronic neutrophilic leukemia (CNL), Hairy cell leukemia (HCL), T-cell large granular lymphocyte leukemia (T-LGL) and aggressive NK-cell leukemia. According to an embodiment, the AML is acute megakaryoblastic leukemia (AMKL).

According to an embodiment, the leukemia is MDS, CLL, SLL, ALL or AML. According to an embodiment, the leukemia is CLL, SLL or AML. According to an embodiment, the leukemia is CLL or SLL. In some embodiments, the CLL or SLL is a CD20 expressing cancer. According to an embodiment, the leukemia is ALL or AML. According to an embodiment, the leukemia is ALL. According to an embodiment, the leukemia is AML. According to an embodiment, the hematopoietic disorder is Waldenström macroglobulinemia.

According to an embodiment, the hematopoietic disorder is a MLL-rearranged leukemia, MLL-partial tandem duplication (PTD) leukemia, MLL amplified leukemia, MLL-positive leukemia, or leukemia exhibiting elevated HOX/MEIS1 gene expression signatures.

According to an embodiment, the leukemia is a MLL-rearranged leukemia &/or a nucleophosmin 1 (NPM1)-mutated leukemia.

According to an embodiment, the hematopoietic disorder is a MLL-rearranged leukemia.

According to an embodiment, the hematopoietic disorder is a nucleophosmin 1 (NPM1)-mutated leukemia (e.g., NPM1c).

According to an embodiment, the invention provides methods for treatment of a hematopoietic disorder that is myelodysplastic syndrome (MDS), a myeloproliferative neoplasm (MPN), acute lymphocytic leukemia (ALL), acute myeloid leukemia (AML), a small lymphocytic lymphoma (SLL) or chronic lymphocytic leukemia (CLL), comprising administering to a subject in need thereof a therapeutically effective amount of a compound of Formula (I), or a pharmaceutically acceptable salt or a solvate thereof, and at least one other therapeutic agent is a hyponethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

According to an embodiment, the hematopoietic disorder is myelodysplastic syndrome (MDS) or a myeloproliferative neoplasm (MPN).

According to an embodiment, the hematopoietic disorder is acute lymphocytic leukemia (ALL).

According to an embodiment, the hematopoietic disorder is acute myeloid leukemia (AML).

According to an embodiment, the hematopoietic disorder is a small lymphocytic lymphoma (SLL) or chronic lymphocytic leukemia (CLL).

According to an embodiment, the hematopoietic disorder is a SLL or CLL where SLL or CLL is a CD20-expressing cancer.

According to an embodiment, the hematopoietic disorder is myelodysplastic syndrome (MDS).

According to an embodiment, the hematopoietic disorder is a myeloproliferative neoplasm (MPN).

According to an embodiment, the hematopoietic disorder is a NPM1-mutated leukemia with a FLT3 mutation.

According to an embodiment, the hematopoietic disorder is a FLT3-dependent leukemia.

According to an embodiment, the hematopoietic disorder is a MEF2G-dependent leukemia.

According to an embodiment, the hematopoietic disorder harbours one or more MLL1 (KMT2A) gene rearrangements or alterations (e.g., duplications or amplification) and/or NPM1 mutations.

According to an embodiment, the hematopoietic disorder harbours (i) one or more MLL1 (KMT2A) gene rearrangements or alterations (e.g., duplications or amplification) and/or NPM1 mutations plus (ii) a FLT3 mutation.

According to an embodiment, the hematopoietic disorder is an MLL-rearranged leukemia.

According to an embodiment, the hematopoietic disorder is acute myeloid leukemia (AML).

According to an embodiment, the hematopoietic disorder is a small lymphocytic lymphoma (SLL).

According to an embodiment, the hematopoietic disorder is a chronic lymphocytic leukemia (CLL).

According to an embodiment, the hematopoietic disorder is an acute leukemia, chronic leukemia, myeloid leukemia, myelogenous leukemia, lymphoblastic leukemia, lymphocytic leukemia, acute myelogenous leukemia (AML), chronic myelogenous leukemia (CML), acute lymphoblastic leukemia (ALL), chronic lymphocytic leukemia (CLL), T cell prolymphocytic leukemias (T-PLL), large granular lymphocytic leukemia, Hairy cell leukemia (HCL), MLL-rearranged leukemia, MLL-PTD leukemia, MLL amplified leukemia, MLL-positive leukemia, or leukemia exhibiting elevated HOX/MEIS1 gene expression signatures.

According to an embodiment, the hematopoietic disorder is AML, in particular nucleophosmin (NPM1)-mutated AML (i.e., NPM11^{mut} AML), more in particular abstract NPM1-mutated AML.

According to an embodiment, the hematopoietic disorder is a MLL-rearranged leukemia, in particular MLL-rearranged AML or ALL.

According to an embodiment, the hematopoietic disorder includes a MLL gene alteration, in particular the hematopoietic disorder is AML or ALL with MLL gene alteration(s). In certain embodiments, the MLL gene alteration is a duplication. In certain embodiments the MLL gene alteration is an amplification.

According to an embodiment, the hematopoietic disorder includes a NPM1 gene mutation and/or MLL1 (also known as KMT2A) gene mutation.

According to an embodiment, MLL1 gene mutations include, but are not limited to, MLL1 gene rearrangements, duplications or amplification.

According to an embodiment, the hematopoietic disorder is a mixed-lineage leukemia (MLL), MLL-related leukemia, MLL-associated leukemia, MLL-positive leukemia, MLL-induced leukemia, leukemia associated with a MLL, acute leukemia, chronic leukemia, myelodysplastic syndrome (MDS), or myeloproliferative neoplasms (MPN).

All embodiments described herein for methods for treating a disorder, are also applicable for use in treating said disorder.

All embodiments described herein for use in treating a disorder, are also applicable for methods for treating said disorder.

All embodiments described herein for methods for treating a disorder, are also applicable for use in a method for treating said disorder.

All embodiments described herein for use in a method for treating a disorder, are also applicable for methods for treating said disorder.

In an embodiment, the present invention relates to a novel combination comprising a therapeutically effective amount of a menin-MLL inhibitor of Formula (I), or a tautomer or a stereoisomeric form thereof, or a pharmaceutically acceptable salt or a solvate thereof; and a therapeutically effective amount of at least one other therapeutic agent which is a hypornethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

According to an embodiment, compounds of Formula (I) are menin-MLL inhibitors having the structure:

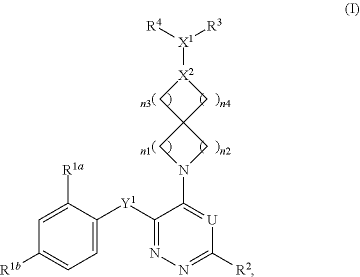

and the tautomers and the stereoisomeric forms thereof, wherein
R^{1a} represents —C(═O)—NR^{xa}R^{xb}; Het; or

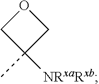

Het represents a 5- or 6-membered monocyclic aromatic ring containing one, two or three nitrogen atoms and optionally a carbonyl moiety;
wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}—, —O— or —NR^{5c}—; R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}, —C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl; wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl; and
R^{9a}, R^{9b}, R^{10a}, R^{10b}, R^{10c}, R¹¹, R^{12a}, and R^{12b} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb}; Het; or

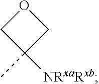

Het represents a 5- or 6-membered monocyclic aromatic ring containing one, two or three nitrogen atoms and optionally a carbonyl moiety;
wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}—, —O— or —NR^{5c}—;
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}, —C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl;
wherein each of the C₁₋₄alkyl or C₁₋₆alkyl moieties in the R³ definitions independently of each other may be substituted with one, two or three substituents each independently selected from the group consisting of cyano, halo or —O—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of cyano, halo, —S(═O)₂—C₁₋₄alkyl, —O—C₁₋₄alkyl, and —C(═O)—NR^{10a}R^{10b}; and
R^{9a}, R^{9b}, R^{10a}, R^{10b}, R¹¹, R^{12a}, and R^{12b} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein

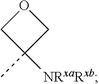

R^{1a} represents —C(═O)—NR^{xa}R^{xb}; Het; or
Het represents a 5- or 6-membered monocyclic aromatic ring containing one, two or three nitrogen atoms and optionally a carbonyl moiety;
wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}—, —O— or —NR^{5c}—;
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}; wherein the C₁₋₆alkyl moiety in the R³ definition may be substituted with one, two or three substituents each independently selected from the group consisting of cyano, halo, OH, and —O—C₁₋₄alkyl;
R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl; and R^{10a}, R^{10b}, R^{10c} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb}; Het; or

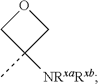

Het represents a 5- or 6-membered monocyclic aromatic ring containing one, two or three nitrogen atoms and optionally a carbonyl moiety;
wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}—, —O— or —NR^{5c}—;
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}; wherein the C₁₋₆alkyl moiety in the R³ definition may be substituted with one, two or three substituents each independently selected from the group consisting of cyano, halo and —O—C₁₋₄alkyl;
R^{8a} and R^{8b} are each independently selected from the group consisting of hydrogen; C₁₋₆alkyl; and C₁₋₆alkyl substituted with one, two or three substituents each independently selected from the group consisting of cyano, halo, —S(═O)₂—C₁₋₄alkyl, —O—C₁₋₄alkyl, and —C(═O)—NR^{10a}R^{10b}; and
R^{10a} and R^{10b} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb} or Het;
Het represents a 6-membered monocyclic aromatic ring containing two nitrogen atoms; wherein said 6-membered monocyclic aromatic ring is substituted with one C₃₋₆cycloalkyl;
R^{xa} and R^{xb} represent C₁₋₄alkyl;
R^{1b} represents F;
Y¹ represents —O—;
R² represents hydrogen;
U represents N or CH;
n1, n2, n3 and n4 are each independently selected from 1 and 2;
X¹ represents CH, and X² represents N;
R⁴ represents isopropyl;
R³ represents —C₁₋₆alkyl-NR^{8a}R^{8b}, —C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl;
wherein each of the C₁₋₄alkyl or C₁₋₆alkyl moieties in the R³ definitions independently of each other may be substituted with one, two or three substituents each independently selected from the group consisting of —OH and —O—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl; and
R^{9a}, R^{9b}, R^{10a}, R^{10b}, R^{10c}, R¹¹, R^{12a}, and R^{12b} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb} or Het;
Het represents a 6-membered monocyclic aromatic ring containing two nitrogen atoms; wherein said 6-membered monocyclic aromatic ring is substituted with one C₃₋₆cycloalkyl;
R^{xa} and R^{xb} represent C₁₋₄alkyl;
R^{1b} represents F;
Y¹ represents —O—;
R² represents hydrogen;
U represents N or CH;
n1, n2, n3 and n4 are each independently selected from 1 and 2;
X¹ represents CH, and X² represents N;
R⁴ represents isopropyl;
R³ represents —C₁₋₆alkyl-NR^{8a}R^{8b}; wherein the C₁₋₆alkyl moiety in the R³ definition may be substituted with one, two or three substituents each independently selected from the group consisting of —OH and —O—C₁₋₄alkyl;
R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl; and
R^{10a}, R^{10b}, and R^{10c} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent C₁₋₄alkyl;
R^{1b} represents F;
Y¹ represents —O—;
R² represents hydrogen;
U represents N or CH;
n1, n2, n3 and n4 are each independently selected from 1 and 2;
X¹ represents CH, and X² represents N;
R⁴ represents isopropyl;
R³ represents —C₁₋₆alkyl-NR^{8a}R^{8b}; wherein the C₁₋₆alkyl moiety in the R³ definition may be substituted with one, two or three substituents each independently selected from the group consisting of —OH and —O—C₁₋₄alkyl;
R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl; and
R^{10a}, R^{10b}, and R^{10c} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb} or Het;
Het represents pyrimidinyl substituted with one C₃₋₆cycloalkyl;
R^{xa} and R^{xb} represent C₁₋₄alkyl;
R^{1b} represents F;
Y¹ represents —O—;
R² represents hydrogen;
U represents N;
n1, n2, n3 and n4 are each independently selected from 1 and 2;
X¹ represents CH, and X² represents N;
R⁴ represents isopropyl;
R³ represents —C₁₋₆alkyl-NR^{8a}R^{8b}; wherein the C₁₋₆alkyl moiety in the R³ definition may be substituted with one —OH;
R^{8a} and R^{8b} are each independently selected from the group consisting of hydrogen; C₁₋₆alkyl; and C₁₋₆alkyl substituted with one or two substituents each independently selected from the group consisting of halo, —O—C₁₋₄alkyl, and —NR^{10c}—C(═O)—C₁₋₄alkyl; and
R^{10a}, R^{10b}, and R^{10c} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb} or Het;
Het represents pyrimidinyl substituted with one C₃₋₆cycloalkyl;
R^{xa} and R^{xb} represent C₁₋₄alkyl;
R^{1b} represents F;
Y¹ represents —O—;
R² represents hydrogen;
U represents N;
n2 is 2;
n1, n3 and n4 are 1;
X¹ represents CH, and X² represents N;
R⁴ represents isopropyl;
R³ represents —C₁₋₆alkyl-NR^{8a}R^{8b}; wherein the C₁₋₆alkyl moiety in the R³ definition may be substituted with one —OH;
R^{8a} and R^{8b} are each independently selected from the group consisting of hydrogen; C₁₋₆alkyl; and C₁₋₆alkyl substituted with one or two substituents each independently selected from the group consisting of halo, —O—C₁₋₄alkyl, and —NR^{10c}—C(═O)—C₁₋₄alkyl; and
R^{10a}, R^{10b}, and R^{10c} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent C₁₋₄alkyl;
R^{1b} represents F;
Y¹ represents —O—;
R² represents hydrogen;
U represents N;
n2 is 2;
n1, n3 and n4 are 1;
X¹ represents CH, and X² represents N;
R⁴ represents isopropyl;
R³ represents —C₁₋₆alkyl-NR^{8a}R^{8b};
R^{8a} and R^{8b} are each independently selected from the group consisting of hydrogen; C₁₋₆alkyl; and C₁₋₆alkyl substituted with one or two substituents each independently selected from the group consisting of halo, —O—C₁₋₄alkyl, and —NR^{10c}—C(═O)—C₁₋₄alkyl; and
R^{10a}, R^{10b}, and R^{10c} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent C₁₋₄alkyl;
R^{1b} represents F;
Y¹ represents —O—;
R² represents hydrogen;
U represents N;
n2 is 2;
n1, n3 and n4 are 1;
X¹ represents CH, and X² represents N;
R⁴ represents isopropyl;
R³ represents —CH₂—CH₂—CH₂—NR^{8a}R^{8b}; R^{8a} and R^{8b} are each independently selected from the group consisting of hydrogen; C₁₋₆alkyl; and C₁₋₆alkyl substituted with one or two substituents each independently selected from the group consisting of halo, —O—C₁₋₄alkyl, and —NR^{10c}—C(═O)—C₁₋₄alkyl; and
R^{10a}, R^{10b}, and R^{10c} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent C₁₋₄alkyl;
R^{1b} represents F;
Y¹ represents —O—;
R² represents hydrogen;
U represents N;
n1, n2, n3 and n4 are each independently selected from 1 and 2;
X¹ represents CH, and X² represents N;
R⁴ represents isopropyl;
R³ represents —C₁₋₆alkyl-NR^{8a}R^{8b};
R^{8a} and R^{8b} are each independently selected from the group consisting of hydrogen; C₁₋₆ alkyl; and C₁₋₆alkyl substituted with one, two or three substituents each independently selected from the group consisting of —OH, cyano, halo, —S(═O)₂-C₁₋₄alkyl, —O—C₁₋₄alkyl, and —C(═O)—NR^{10a}R^{10b}; and
R^{10a} and R^{10b} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent C₁₋₄alkyl;
R^{1b} represents F;
Y¹ represents —O—;
R² represents hydrogen;
U represents N;
n1, n2, n3 and n4 are each independently selected from 1 and 2;
X¹ represents CH, and X² represents N;
R⁴ represents isopropyl;
R³ represents —CH₂—CH₂—CH₂—NR^{8a}R^{8b};
R^{8a} and R^{8b} are each independently selected from the group consisting of hydrogen; C₁₋₆ alkyl; and C₁₋₆alkyl substituted with one, two or three substituents each independently selected from the group consisting of —OH, cyano, halo, —S(═O)₂—C₁₋₄alkyl, —O—C₁₋₄alkyl, and —C(═O)—NR^{10a}R^{10b}; and
R^{10a} and R^{10b} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent hydrogen or C₁₋₄alkyl;
R^{1b} represents F;
Y¹ represents —O—;
R² represents hydrogen;
U represents N;
n1, n2, n3 and n4 are each independently selected from 1 and 2;
X¹ represents CH, and X² represents N;
R⁴ represents isopropyl;
R³ represents —CH₂—CH₂—CH₂—NR^{8a}R^{8b};
R^{8a} and R^{8b} are each independently selected from the group consisting of hydrogen; C₁₋₆ alkyl; and C₁₋₆alkyl substituted with one, two or three substituents each independently selected from the group consisting of —OH, cyano, halo, —S(═O)₂—C₁₋₄alkyl, —O—C₁₋₄alkyl, and —C(═O)—NR^{10a}R^{10b}; and
R^{10a} and R^{10b} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent hydrogen or C₁₋₄alkyl;
R^{1b} represents F;
Y¹ represents —O—;
R² represents hydrogen;
U represents N;
n1, n2, n3 and n4 are each independently selected from 1 and 2;
X¹ represents CH, and X² represents N;
R⁴ represents isopropyl;
R³ represents —CH₂—CH₂—CH₂—NR^{8a}R^{8b}; and
R^{8a} and R^{8b} are each independently selected from the group consisting of hydrogen; C₁₋₆ alkyl; and C₁₋₆alkyl substituted with one, two or three substituents each independently selected from the group consisting of —OH and —O—C₁₋₄alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent C₁₋₄alkyl;
R^{1b} represents F;
Y¹ represents —O—;
R² represents hydrogen;
U represents N;
n1, n2, n3 and n4 are each independently selected from 1 and 2;
X¹ represents CH, and X² represents N;
R⁴ represents isopropyl;
R³ represents —C₁₋₆alkyl-NR^{8a}R^{8b}; and
R^{8a} and R^{8b} are each independently selected from the group consisting of C₁₋₆alkyl; and C₁₋₆alkyl substituted with one —O—C₁₋₄alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent C₁₋₄alkyl;
R^{1b} represents F;
Y¹ represents —O—;
R² represents hydrogen;
U represents N;
n1, n2, n3 and n4 are each independently selected from 1 and 2;
X¹ represents CH, and X² represents N;
R⁴ represents isopropyl;
R³ represents —CH₂—CH₂—CH₂—NR^{8a}R^{8b}; and
R^{8a} and R^{8b} are each independently selected from the group consisting of C₁₋₆alkyl; and C₁₋₆alkyl substituted with one —O—C₁₋₄alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb}; or Het;
Het represents a 6-membered monocyclic aromatic ring containing two nitrogen atoms; wherein said 6-membered monocyclic aromatic ring is optionally substituted with one C₃₋₆cycloalkyl;
R^{xa} and R^{xb} represent C₁₋₄alkyl;
R^{1b} represents F;
Y¹ represents —O—;
R² is hydrogen;
U represents N;
n1, n2, n3 and n4 are each independently selected from 1 and 2;
X¹ represents CH, and X² represents N;
R⁴ represents isopropyl;
R³ represents —C₁₋₆alkyl-NR^{8a}R^{8b}, —C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of cyano, halo, —S(═O)₂—C₁₋₄alkyl, and —O—C₁₋₄alkyl; and
R^{9a}, R^{9b}, R^{12a}, and R^{12b} are each independently selected from the group consisting of hydrogen and C₁₋₆alkyl;
and the pharmaceutically acceptable salts and the solvates thereof.

According to an embodiment, compounds of Formula (I) are as defined herein, and the tautomers and the stereoisomeric forms thereof, wherein
R^{1a} represents —C(═O)—NR^{xa}R^{xb};
R^{xa} and R^{xb} represent C₁₋₄alkyl;
R^{1b} represents F;
Y¹ represents —O—;
R² is hydrogen;
U represents N;
n1, n2, n3 and n4 are each independently selected from 1 and 2;
X¹ represents CH, and X² represents N;
R⁴ represents isopropyl;
R³ represents —C₁₋₆alkyl-NR^{8a}R^{8b}, —C₁₋₆alkyl-C(═O)—NR^{9a}R^{9b}, or —C₁₋₆alkyl-OH;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of cyano, halo, —S(═O)₂—C₁₋₄alkyl, and —O—C₁₋₄alkyl; and
R^{9a}, R^{9b}, R¹², and R^{12b} are each independently selected from the group consisting of hydrogen and C₁₋₆alkyl;
and the pharmaceutically acceptable salts and the solvates thereof.

In an embodiment, the present invention includes compounds of Formula (I) and the pharmaceutically acceptable salts, and the solvates thereof, or any subgroup thereof as mentioned in any of the other embodiments, wherein R^{1b} represents F.

In an embodiment, the present invention includes compounds of Formula (I) and the pharmaceutically acceptable salts, and the solvates thereof, or any subgroup thereof as mentioned in any of the other embodiments, wherein R² represents hydrogen.

In an embodiment, the present invention includes compounds of Formula (I) and the pharmaceutically acceptable salts, and the solvates thereof, or any subgroup thereof as mentioned in any of the other embodiments, wherein n1 is 1, n2 is 2, n3 is 1, and n4 is 1.

In an embodiment, the present invention includes compounds of Formula (I) and the pharmaceutically acceptable salts, and the solvates thereof, or any subgroup thereof as mentioned in any of the other embodiments, wherein Y¹ represents —O—.

In an embodiment, the present invention includes compounds of Formula (I) and the pharmaceutically acceptable salts, and the solvates thereof, or any subgroup thereof as mentioned in any of the other embodiments, wherein Y¹ represents —O—; and U represents N.

In an embodiment, the present invention includes compounds of Formula (I) and the pharmaceutically acceptable salts, and the solvates thereof, or any subgroup thereof as mentioned in any of the other embodiments, wherein Y¹ represents —O—; U represents N; R^{1b} represents F; and R² represents hydrogen.

In an embodiment, the present invention includes compounds of Formula (I) and the pharmaceutically acceptable salts, and the solvates thereof, or any subgroup thereof as mentioned in any of the other embodiments, wherein Het represents

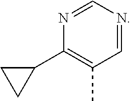

In an embodiment, the present invention includes compounds of Formula (I) and the pharmaceutically acceptable salts, and the solvates thereof, or any subgroup thereof as mentioned in any of the other embodiments, wherein Het represents a monocyclic 5- or 6-membered aromatic ring containing one or two nitrogen atoms;

wherein said monocyclic 5- or 6-membered aromatic ring is substituted with one C₃₋₆cycloalkyl.

In an embodiment, the present invention includes compounds of Formula (I) and the pharmaceutically acceptable salts, and the solvates thereof, or any subgroup thereof as mentioned in any of the other embodiments, wherein Het represents a monocyclic 5- or 6-membered aromatic ring containing one or two nitrogen atoms; wherein said monocyclic 5- or 6-membered aromatic ring is substituted with one C₃₋₆cycloalkyl; and R^{1b} represents F.

In an embodiment, the present invention includes compounds of Formula (I) and the pharmaceutically acceptable salts, and the solvates thereof, or any subgroup thereof as mentioned in any of the other embodiments, wherein Het represents a monocyclic 6-membered aromatic ring containing one or two nitrogen atoms; wherein said monocyclic 6-membered aromatic ring is substituted with one C₃₋₆cycloalkyl.

In an embodiment, the present invention includes compounds of Formula (I) and the pharmaceutically acceptable salts, and the solvates thereof, or any subgroup thereof as mentioned in any of the other embodiments, wherein Het represents a monocyclic 6-membered aromatic ring containing one or two nitrogen atoms; and wherein said monocyclic 6-membered aromatic ring is substituted with one C₃₋₆cycloalkyl; and R^{1b} represents F.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; wherein the C₁₋₆alkyl moiety in the R³ definition may be substituted with one, two or three substituents each independently selected from the group consisting of cyano, halo and —O—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; wherein the C₁₋₆alkyl moiety in the R³ definition may be substituted with one, two or three substituents each independently selected from the group consisting of cyano, halo, —OH, and —O—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; wherein the C₁₋₆alkyl moiety in the R³ definition may be substituted with one, two or three substituents each independently selected from the group consisting of cyano, halo and —O—C₁₋₄alkyl; R^{8a} and R^{8b} are each independently selected from the group consisting of hydrogen; C₁₋₆alkyl; and C₁₋₆ alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; wherein the C₁₋₆alkyl moiety in the R³ definition may be substituted with one, two or three substituents each independently selected from the group consisting of cyano, halo, —OH, and —O—C₁₋₄alkyl; R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; wherein the C₁₋₆alkyl moiety in the R³ definition may be substituted with one, two or three substituents each independently selected from the group consisting of cyano, halo and —O—C₁₋₄alkyl; R^{8a} and R^{8b} are each independently selected from the group consisting of hydrogen; C₁₋₆alkyl; and C₁. 6alkyl substituted with one, two or three substituents each independently selected from the group consisting of cyano, halo, —S(═O)₂—C₁₋₄alkyl, —O—C₁₋₄alkyl, and —C(═O)—NR^{10a}R^{10b}.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; wherein the C₁₋₆alkyl moiety in the R³ definition may be substituted with one, two or three substituents each independently selected from the group consisting of cyano, halo and —O—C₁₋₄alkyl; R^{8a} and R^{8b} are each independently selected from the group consisting of hydrogen; C₁₋₆alkyl; and C₁₋₆ alkyl substituted with one, two or three substituents each independently selected from the group consisting of cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; wherein the C₁₋₆alkyl moiety in the R³ definition may be substituted with one, two or three substituents each independently selected from the group consisting of cyano, halo, —OH, and —O—C₁₋₄alkyl; R^{8a} and R^{8b} are each independently selected from the group consisting of hydrogen; C₁₋₆alkyl; and C₁₋₆alkyl substituted with one, two or three substituents each independently selected from the group consisting of cyano, halo, —S(═O)₂—C₁₋₄alkyl, —O—C₁₋₄alkyl, and —C(═O)—NR^{10a}R^{10b}.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; wherein the C₁₋₆alkyl moiety in the R³ definition may be substituted with one, two or three substituents each independently selected from the group consisting of cyano, halo, —OH, and —O—C₁₋₄alkyl; R^{8a} and R^{8b} are each independently selected from the group consisting of hydrogen; C₁₋₆alkyl; and C₁₋₆alkyl substituted with one, two or three substituents each independently selected from the group consisting of cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₂₋₆alkyl-NR^{8a}R^{8b}; wherein the C₂₋₆alkyl moiety in the R³ definition may be substituted with one, two or three substituents each independently selected from the group consisting of cyano, halo and —O—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₂₋₆alkyl-NR^{8a}R^{8b}; wherein the C₂₋₆alkyl moiety in the R³ definition may be substituted with one, two or three substituents each independently selected from the group consisting of cyano, halo, —OH, and —O—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₂₋₆alkyl-NR^{8a}R^{8b}; wherein the C₂₋₆alkyl moiety in the R³ definition may be substituted with one, two or three substituents each independently selected from the group consisting of cyano, halo and —O—C₁₋₄alkyl; R^{8a} and R^{8b} are each independently selected from the group consisting of hydrogen; C₁₋₆alkyl; and C₁₋₆ alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₂₋₆alkyl-NR^{8a}R^{8b}; wherein the C₂₋₆alkyl moiety in the R³ definition may be substituted with one, two or three substituents each independently selected from the group consisting of cyano, halo, —OH, and —O—C₁₋₄alkyl; R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₂₋₆alkyl-NR^{8a}R^{8b}; wherein the C₂₋₆alkyl moiety in the R³ definition may be substituted with one, two or three substituents each independently selected from the group consisting of cyano, halo, —OH, and —O—C₁₋₄alkyl; R^{8a} and R^{8b} are each independently selected from the group consisting of hydrogen; C₁₋₆alkyl; and C₁₋₆alkyl substituted with one, two or three substituents each independently selected from the group consisting of cyano, halo, —S(═O)₂—C₁₋₄alkyl, —O—C₁₋₄alkyl, and —C(═O)—NR^{10a}R^{10b}.

In an embodiment, the present invention includes compounds of Formula (I) and the pharmaceutically acceptable salts, and the solvates thereof, or any subgroup thereof as mentioned in any of the other embodiments, wherein R³ represents —C₂₋₆alkyl-NR^{8a}R^{8b}; wherein the C₂₋₆alkyl moiety in the R³ definition may be substituted with one, two or three substituents each independently selected from the group consisting of cyano, halo and —O—C₁₋₄alkyl; R^{8a} and R^{8b} are each independently selected from the group consisting of hydrogen; C₁₋₆alkyl; and C₁₋₆ alkyl substituted with one, two or three substituents each independently selected from the group consisting of cyano, halo, —S(═O)₂—C₁₋₄alkyl, —O—C₁₋₄alkyl, and —C(═O)—NR^{10a}R^{10b}.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; R^{8a} and R^{8b} are each independently selected from the group consisting of C₁₋₆alkyl; and C₁₋₆ alkyl substituted with one, two or three substituents each independently selected from the group consisting of —OH, cyano, halo, —S(═O)₂—C₁₋₄alkyl, —O—C₁₋₄alkyl, and —C(═O)—NR^{10a}R^{10b}.

In an embodiment, the present invention includes compounds of Formula (I) and the pharmaceutically acceptable salts, and the solvates thereof, or any subgroup thereof as mentioned in any of the other embodiments, wherein R³ represents —C₂₋₆alkyl-NR^{8a}R^{8b}; wherein the C₂₋₆alkyl moiety in the R³ definition may be substituted with one, two or three substituents each independently selected from the group consisting of cyano, halo, —OH, and —O—C₁₋₄alkyl; R^{8a} and R^{8b} are each independently selected from the group consisting of hydrogen; C₁₋₆alkyl; and C₁₋₆alkyl substituted with one, two or three substituents each independently selected from the group consisting of cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₂₋₆alkyl-NR^{8a}R^{8b}; wherein the C₂₋₆alkyl moiety in the R³ definition may be substituted with one, two or three substituents each independently selected from the group consisting of cyano, halo and —O—C₁₋₄alkyl; R^{8a} and R^{8b} are each independently selected from the group consisting of hydrogen; C₁₋₆alkyl; and C₁₋₆alkyl substituted with one, two or three substituents each independently selected from the group consisting of cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; R^{8a} and R^{8b} are each independently selected from the group consisting of C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; R^{8a} represents C₁₋₆alkyl; and R^{8b} represents C₁₋₆alkyl substituted with one —O—C₁₋₄alkyl.

In an embodiment, the present invention relates to methods of using the compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}, —C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl; wherein each of the C₁₋₄alkyl or C₁₋₆alkyl moieties in the R³ definitions independently of each other may be substituted with one, two or three substituents each independently selected from the group consisting of cyano, halo or —O—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}, —C₁₋₆alkyl-C(═O)—NR^{9a}R^{9b}, or —C₁₋₆alkyl-NR¹¹—C(═O)—O—C₁₋₄alkyl-O—C(═O)—C₁₋₄alkyl; wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R³ represents —CH₂—CH₂—CH₂—NR^{8a}R^{8b}.

In an embodiment, the present invention includes compounds of Formula (I) and the pharmaceutically acceptable salts, and the solvates thereof, or any subgroup thereof as mentioned in any of the other embodiments, wherein R³ represents —CH₂—CH₂—CH₂—NR^{8a}R^{8b}; R^{8a} represents methyl; and R^{8b} represents —CH₂—CH₂—OCH₃.

In an embodiment, the present invention includes compounds of Formula (I) and the pharmaceutically acceptable salts, and the solvates thereof, or any subgroup thereof as mentioned in any of the other embodiments, wherein C₁₋₆alkyl in the R³ definition —C₁₋₆alkyl-NR^{8a}R^{8b} is limited to —CH₂—CH₂—CH₂—.

In an embodiment, the present invention includes compounds of Formula (I) or the pharmaceutically acceptable salts or the solvates thereof, or any subgroup thereof as mentioned in any of the other embodiments, wherein the compounds of Formula (I) are restricted to compounds of Formula (Ia) or Formula (Ib):

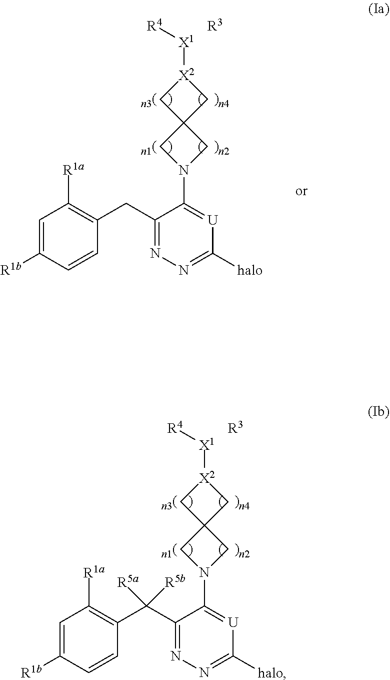

wherein R^{1a}, R^{1b}, R³, R⁴, R^{5a}, R^{5b}, X¹, X², n1, n2, n3, n4 and halo are as defined for the compounds of Formula (I) or any subgroup thereof as mentioned in any of the other embodiments.

In an embodiment, the compounds of Formula (I), or the pharmaceutically acceptable salts or the solvates thereof, or any subgroup thereof as mentioned in any of the other embodiments, are restricted to compounds of Formula (Ia), or the pharmaceutically acceptable salts or the solvates thereof. In an embodiment, the compounds of Formula (I), or the pharmaceutically acceptable salts or the solvates thereof, or any subgroup thereof as mentioned in any of the other embodiments, are restricted to compounds of Formula (Ib), or the pharmaceutically acceptable salts or the solvates thereof.

In an embodiment, the present invention includes compounds of Formula (I), or the pharmaceutically acceptable salts or the solvates thereof, or any subgroup thereof as mentioned in any of the other embodiments, wherein the compounds of Formula (I) are restricted to compounds of Formula (by):

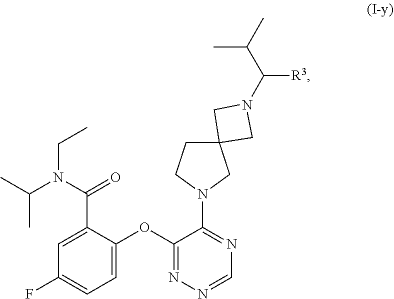

wherein R³ is as defined for the compounds of Formula (I) or any subgroup thereof as mentioned in any of the other embodiments.

In Formula (I-y) n1 is 1, n2 is 2, n3 is 1, and n4 is 1.

In a particular embodiment, the compound of Formula (I) is Compound A:

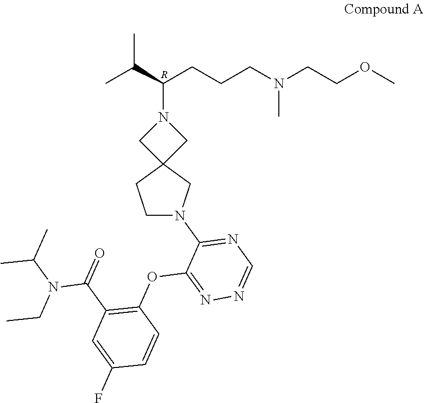

or a pharmaceutically acceptable salt or solvate thereof.

In a particular embodiment, the compound of Formula (I) is Compound A1:

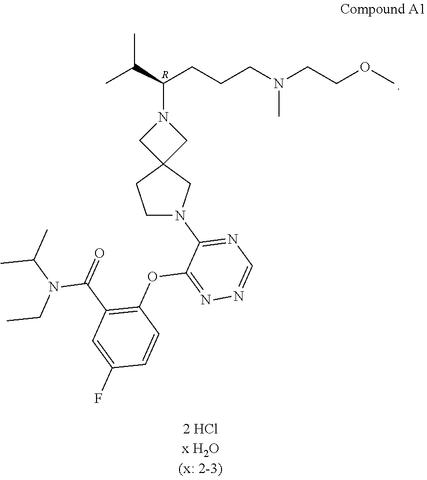

In a particular embodiment, the compound of Formula (I) is Compound A2:

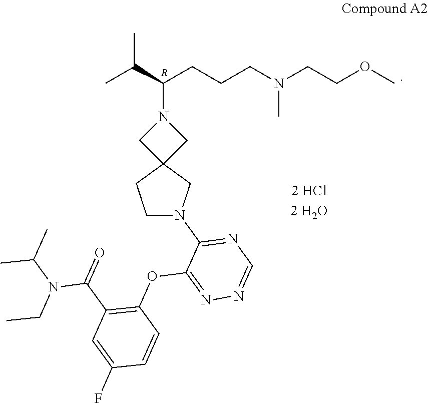

In a particular embodiment, the compound of Formula (I) is Compound A3:

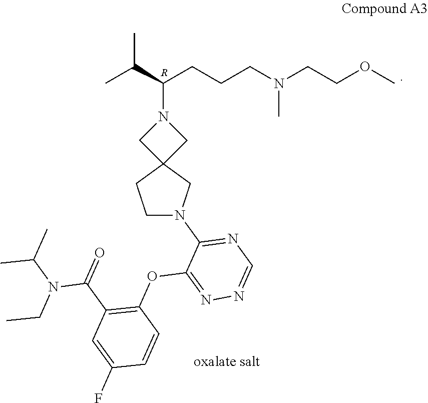

In a particular embodiment, the compound of Formula (I) is Compound A4-a:

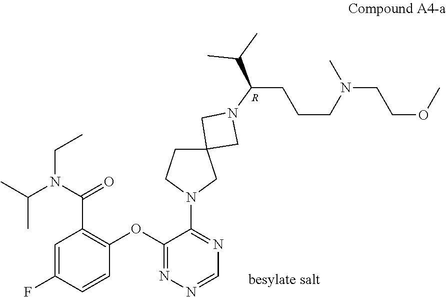

or a solvate thereof.

In a particular embodiment, the menin-MLL inhibitor of Formula (I) is (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt or a hydrate thereof.

In a particular embodiment, the menin-MLL inhibitor of Formula (I) is (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or a solvate thereof.

In a particular embodiment, the menin-MLL inhibitor of Formula (I) is (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt (Compound A4-b) or a hydrate thereof.

In a particular embodiment, the menin-MLL inhibitor of Formula (I) is Compound A4: crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate.

In a particular embodiment, the menin-MLL inhibitor of Formula (I) is a crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt 0.5-2.0 equivalents hydrate.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof, and the free bases, any pharmaceutically acceptable salts, and the solvates thereof.

In some embodiments, provided is a pharmaceutical composition comprising a pharmaceutically acceptable carrier, and as active ingredient a therapeutically effective amount of a combination as described in any of the other embodiments.

In some embodiments, provided is a combination therapy comprising a menin-MLL inhibitor of Formula (I), or a pharmaceutically acceptable salt or a solvate thereof, and at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

According to embodiments, the menin-MLL inhibitor is a compound of Formula (I), or a pharmaceutically acceptable salt or a solvate thereof.

According to particular embodiments, the menin-MLL inhibitor is Compound A or a pharmaceutically acceptable salt or solvate thereof.

According to particular embodiments, the menin-MLL inhibitor is Compound A1.

According to particular embodiments, the menin-MLL inhibitor is Compound A2.

According to particular embodiments, the menin-MLL inhibitor is Compound A3.

According to particular embodiments, the menin-MLL inhibitor is Compound A4-a or a solvate thereof.

According to particular embodiments, the menin-MLL inhibitor is Compound A4-b or a hydrate thereof.

According to particular embodiments, the menin-MLL inhibitor is Compound A4.

In particular embodiments, the menin-MLL inhibitor may have improved metabolic stability properties.

In particular embodiments, the menin-MLL inhibitor may have extended in vivo half-life (T1/2).

In particular embodiments, the menin-MLL inhibitor may have improved oral bioavailability.

In particular embodiments, the menin-MLL inhibitor may reduce tumor growth e.g., tumors harbouring MLL (KMT2A) gene rearrangements/alterations and/or NPM1 mutations.

In particular embodiments, the menin-MLL inhibitor may have improved PD properties in vivo during a prolonged period of time, e.g., inhibition of target gene expression such as MEIS1 and upregulation of differentiation marker over a period of at least 16 hours.

In particular embodiments, the menin-MLL inhibitor may have an improved safety profile (e.g., reduced hERG inhibition; improved cardiovascular safety).

In particular embodiments, the menin-MLL inhibitor may be suitable for Q.D. dosing (once daily).

According to embodiments, at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

According to embodiments, the hypomethylating agent includes, but is not limited to, azacitidine, decitabine, or pharmaceutically acceptable salts or solvates thereof.

According to embodiments, the cytidine deaminase inhibitor includes, but is not limited to, cedazuridine or pharmaceutically acceptable salts or solvates thereof.

According to embodiments, the DNA intercalating agent includes, but is not limited to, an anthracycline (e.g., daunorubicin, doxorubicin, idarubicin).

According to embodiments, the DNA intercalating agent is daunorubicin.

According to embodiments, the DNA intercalating agent is doxorubicin.

According to embodiments, the DNA intercalating agent is idarubicin.

According to embodiments, the pyrimidine analog includes, but is not limited to, cytarabine (ARA-C).

According to embodiments, the purine analog is fludarabine.

According to embodiments, the kinase inhibitor is a FLT-3 inhibitor, a BTK inhibitor, an ABL inhibitor, an Aurora inhibitor or a multi-kinase inhibitor of two or more kinase inhibitors thereof.

According to embodiments, the kinase inhibitor is a multi-kinase inhibitor of FLT-3 inhibitor, ABL inhibitor, and Aurora inhibitor. According to embodiments, such multi-kinase inhibitor includes, but is not limited to KW-2449.

According to embodiments, the kinase inhibitor is a tyrosine kinase inhibitor.

According to embodiments, the tyrosine kinase inhibitor is a FLT-3 inhibitor or a BTK inhibitor.

According to embodiments, the FLT3 inhibitor includes, but is not limited to, sorafenib, sunitinib, midostaurin (PKC412), lestaurtinib (CEP-701), tandutinib (MLN518), quizartinib (AC220), gilteritinib (ASP2215), and KW-2449.

According to embodiments, the FLT3 inhibitor is gilteritinib (ASP2215).

According to embodiments, the FLT3 inhibitor is midostaurin (PKC412).

According to embodiments, the BTK inhibitor includes, but is not limited to, ibrutinib.

According to embodiments, the CD20 inhibitor includes, but is not limited to, an anti-CD20 antibody (e.g., obinutuzumab (GA101)).

According to embodiments, the IDH inhibitor includes, but is not limited to, ivosidenib and enasidenib.

According to embodiments, the isocitrate dehydrogenase-1 inhibitor includes, but is not limited to, ivosidenib.

According to embodiments, the isocitrate dehydrogenase-2 inhibitor includes, but is not limited to, enasidenib.

According to embodiments, the immunomodulatory agent includes, but is not limited to, PD-1 inhibitors (e.g., nivolumab, atezolizumab and pembrolizumab), thalidomide, lenalidomide, pomalidomide, Bacillus Calmette-Guérin (BCG) and levamisole.

According to embodiments, the PD-1 inhibitor includes, but is not limited to, nivolumab, atezolizumab and pembrolizumab.

According to embodiments, the DHODH inhibitor includes, but is not limited to, a compound having the structure of Formula (Z):

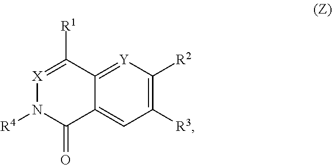

wherein
X is CH or N;
Y is CH or N;
R¹ is selected from the group consisting of C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₂₋₆haloalkenyl; N(CH₃)₂; C₃₋₆cycloalkyl; C₃₋₆cycloalkyl substituted with C₁₋₆alkyl; and phenyl;
R² is

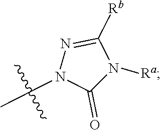

wherein
R^{a} is selected from the group consisting of: C₁₋₆alkyl, C₁₋₆haloalkyl, and C₃₋₆cycloalkyl;
R^{b} is C₁₋₆alkyl or C₁₋₆alkyl substituted with a member selected from the group consisting of: OH, halo, CN, OC₁₋₆alkyl, OC₁₋₆haloalkyl and OC₃₋₆cycloalkyl;
R³ is selected from the group consisting of: H, halo, CH₃ and OCH₃;
R⁴ is selected from the group consisting of:
   C₁₋₆alkyl; C₁₋₆alkyl substituted with one or two OCH₃; C₃₋₆cycloalkyl; C₃₋₆cycloalkyl substituted with CH₃, or OCH₃; CH₂—C₃₋₆cycloalkyl; and

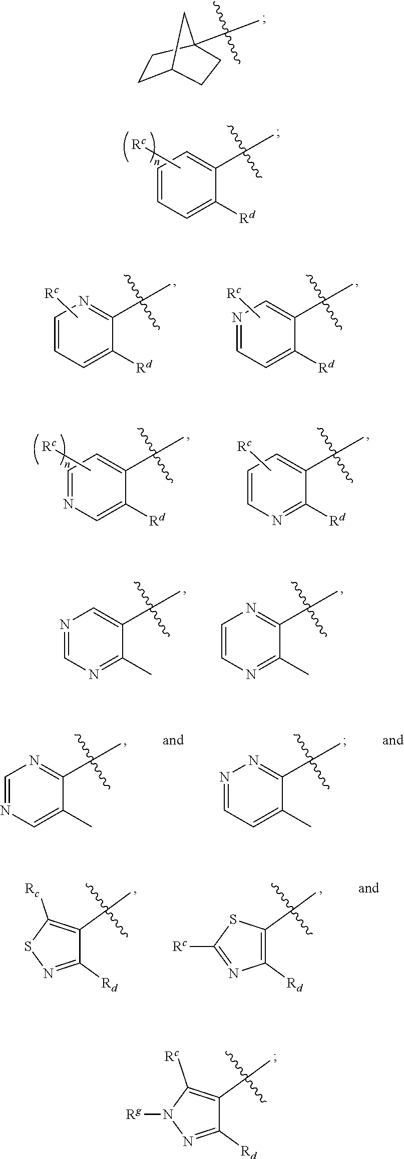

wherein
each R^{c} is independently selected from the group consisting of: H; halo; C₁₋₆alkyl; C₁₋₆alkyl substituted with a member selected from the group consisting of OH, OCH₃, SCH₃, and OCF₃; C₁₋₆haloalkyl; C₁₋₆haloalkyl substituted with a member selected from the group consisting of OH, and OCH₃; NO₂; OH; O—CH₂CH₂OH; and OC₁₋₆alkyl;
R^{d} is selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of OH, and OCH₃; CN; and OC₁₋₆alkyl;
R⁹ is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of OH, and OCH₃; and
n is 1, or 2;
or a pharmaceutically acceptable salt, isotope, N-oxide, solvate, or stereoisomer thereof; or a compound selected from

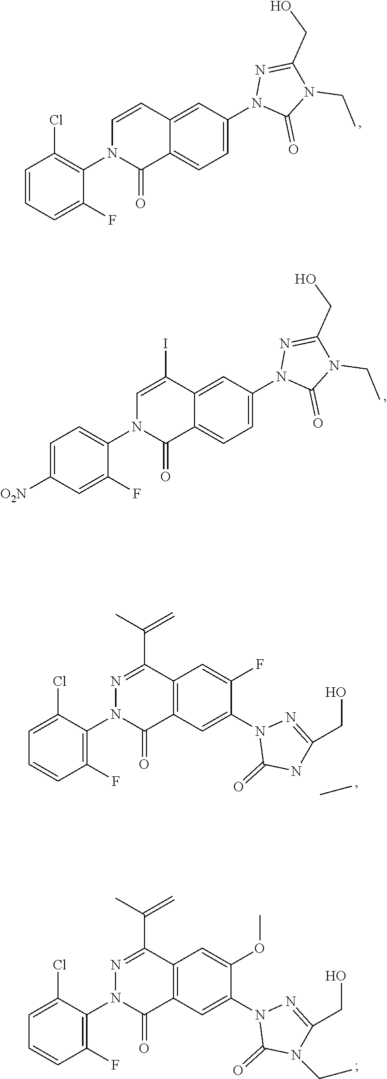

or a pharmaceutically acceptable salt, N-oxide, solvate, or stereoisomer thereof.

According to embodiments, the DHODH inhibitor includes, but is not limited to, a compound having the structure of Formula (Z):

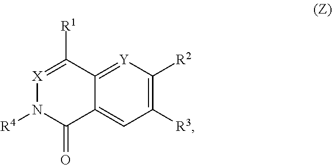

wherein
X is CH or N;
Y is CH or N;
R¹ is selected from the group consisting of C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₂₋₆haloalkenyl; N(CH₃)₂; C₃₋₆cycloalkyl; C₃₋₆cycloalkyl substituted with C₁₋₆alkyl; and phenyl;
R² is

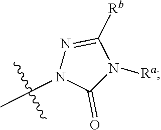

wherein
R^{a} is selected from the group consisting of: C₁₋₆alkyl, C₁₋₆haloalkyl, and C₃₋₆cycloalkyl;
R^{b} is C₁₋₆alkyl or C₁₋₆alkyl substituted with a member selected from the group consisting of OH, halo, CN, OC₁₋₆alkyl, OC₁₋₆haloalkyl and OC₃₋₆cycloalkyl;
R³ is selected from the group consisting of H, halo, CH₃ and OCH₃;
R⁴ is selected from the group consisting of
   C₁₋₆alkyl; C₁₋₆alkyl substituted with one or two OCH₃; C₃₋₆cycloalkyl; C₃₋₆cycloalkyl substituted with CH₃, or OCH₃; CH₂—C₃₋₆cycloalkyl; and;

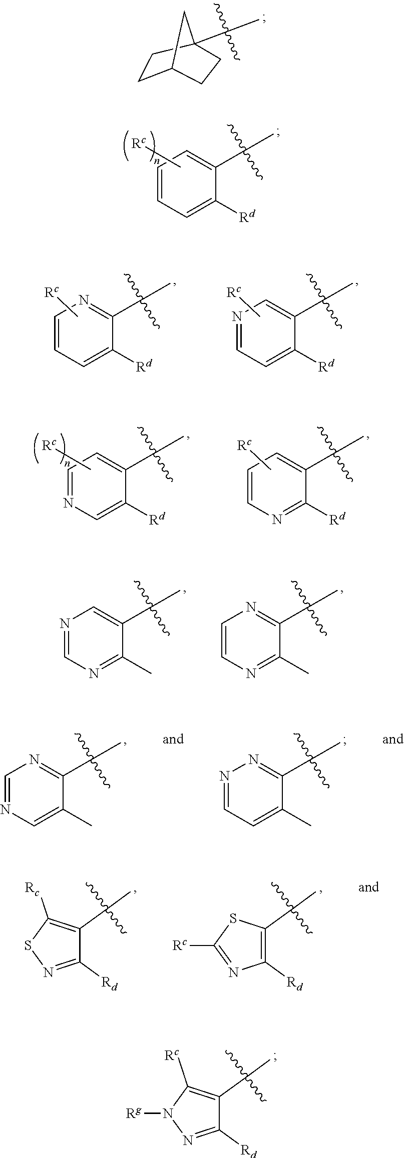

wherein
each R^{c} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; NO₂; OH; O—CH₂CH₂OH; and OC₁₋₆alkyl;
R^{d} is selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; CN; and OC₁₋₆alkyl;
R⁹ is selected from the group consisting of H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
   or a pharmaceutically acceptable salt, isotope, N-oxide, solvate, or stereoisomer thereof.

In the context of Formula (Z), the following definitions apply:

The term “alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl, etc.). The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂₋₆ for straight chain, C₃₋₆ for branched chain).

The term “haloalkyl” refers to a straight- or branched-chain alkyl group having from 1 to 6 carbon atoms in the chain optionally substituting hydrogens with halogens. The term “C₁₋₆ haloalkyl” as used here refers to a straight- or branched-chain alkyl group having from 1 to 6 carbon atoms in the chain, optionally substituting hydrogens with halogens. The term “C₁₋₄ haloalkyl” as used here refers to a straight- or branched-chain alkyl group having from 1 to 4 carbon atoms in the chain, optionally substituting hydrogens with halogens. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “haloalkenyl” includes unsaturated aliphatic groups analogous in length and possible substitution to the alkyls described above, but that contain at least one double bond and having from 1 to 6 carbon atoms in the chain optionally substituting hydrogens with halogens.

The term “aryl” refers to a monocyclic, aromatic carbocycle (ring structure having ring atoms that are all carbon) having 6 atoms per ring. (Carbon atoms in the aryl groups are sp2 hybridized.)

The term “heteroaryl” refers to a monocyclic or fused bicyclic heterocycle (ring structure having ring atoms selected from carbon atoms and up to four heteroatoms selected from nitrogen, oxygen, and sulfur) having from 3 to 9 ring atoms per heterocycle. Illustrative examples of heteroaryl groups include the following entities, in the form of properly bonded moieties:

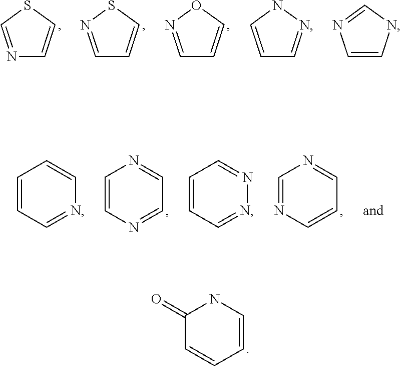

Those skilled in the art will recognize that the species listed or illustrated above are not exhaustive, and that additional species within the scope of these defined terms may also be selected.

The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below.

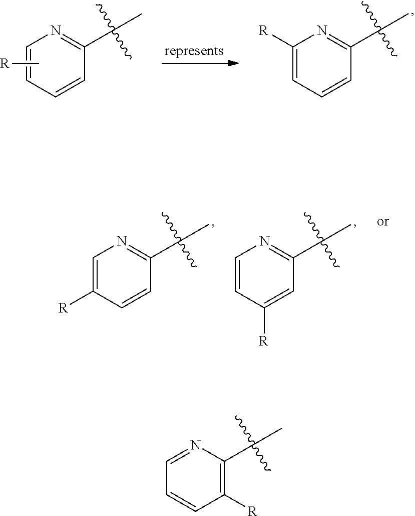

In an embodiment of the invention, the DHODH inhibitor is a compound of Formula (Z) wherein X is CH.

In an embodiment of the invention, the DHODH inhibitor is a compound of Formula (Z) wherein X is N.

In an embodiment of the invention, the DHODH inhibitor is a compound of Formula (Z) wherein Y is CH.

In an embodiment of the invention, the DHODH inhibitor is a compound of Formula (Z) wherein Y is N.

In an embodiment of the invention, the DHODH inhibitor is a compound of Formula (Z) wherein R¹ is C₁₋₄alkyl; C₁₋₄alkyl substituted with OH, or OCH₃; C₂₋₄alkenyl; C₁₋₄haloalkyl; C₁₋₄haloalkyl substituted with OH, or OCH₃; C₂₋₄haloalkenyl; N(CH₃)₂; cyclopropyl; cyclopropyl substituted with C₁₋₄alkyl; or phenyl.

In an embodiment of the invention, the DHODH inhibitor is a compound of Formula (Z) wherein R¹ is CH₃, CH₂CH₃,

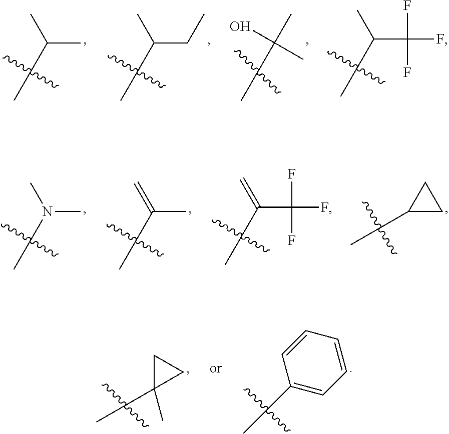

In an embodiment of the invention, the DHODH inhibitor is a compound of Formula (Z) wherein R¹ is

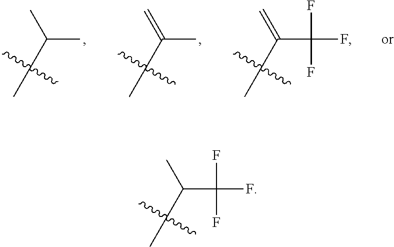

In an embodiment of the invention, the DHODH inhibitor is a compound of Formula (Z) wherein
R² is

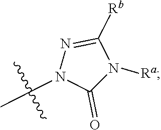

wherein R^{b} is C₁₋₄alkyl substituted with OH, halo, CN, OC₁₋₄alkyl, OC₁₋₄haloalkyl or OC₃₋₆cycloalkyl; and
R^{a} is C₁₋₄alkyl, C₁₋₄haloalkyl, or C₃₋₆cycloalkyl.

In an embodiment of the invention, the DHODH inhibitor is a compound of Formula (Z) wherein R² is

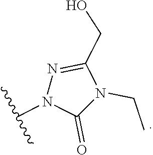 [SMILES: *n1nc(CO)n(CC)c1=O]

In an embodiment of the invention, the DHODH inhibitor is a compound of Formula (Z) wherein R³ is H.

In an embodiment of the invention, the DHODH inhibitor is a compound of Formula (Z) wherein R³ is F.

In an embodiment of the invention, the DHODH inhibitor is a compound of Formula (Z) wherein R³ is CH₃.

In an embodiment of the invention, the DHODH inhibitor is a compound of Formula (Z) wherein R³ is OCH₃.

In an embodiment of the invention, the DHODH inhibitor is a compound of Formula (Z) wherein R⁴ is

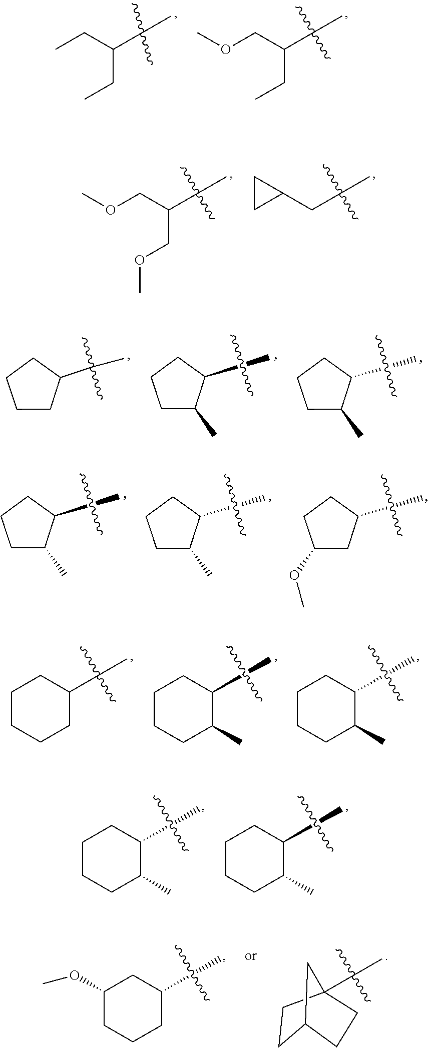

In an embodiment of the invention, the DHODH inhibitor is a compound of Formula (Z) wherein
R⁴ is

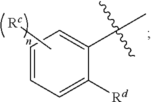

wherein
each R^{c} is independently selected from the group consisting of: H; halo; C₁₋₄alkyl; C₁₋₆alkyl substituted with a member selected from the group consisting of: OH, OCH₃, SCH₃, and OCF₃; C₁₋₄haloalkyl; C₁₋₄haloalkyl substituted with a member selected from the group consisting of: OH, and OCH₃; and NO₂;
R^{d} is selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₄alkyl; CN; and OC₁₋₆alkyl; and
n is 1, or 2.

In an embodiment of the invention, the DHODH inhibitor is a compound of Formula (Z) wherein
R⁴ is

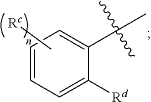

each R^{c} is independently selected from the group consisting of: H, halo, C₁₋₄alkyl, C₁₋₄haloalkyl, NO₂, O—CH₂CH₂OH, and OC₁₋₄alkyl;
R^{d} is selected from the group consisting of: H, halo, C₁₋₄alkyl, CN, and OC₁₋₆alkyl; and
n is 1, or 2.

In an embodiment of the invention, the DHODH inhibitor is a compound of Formula (Z) wherein R⁴ is

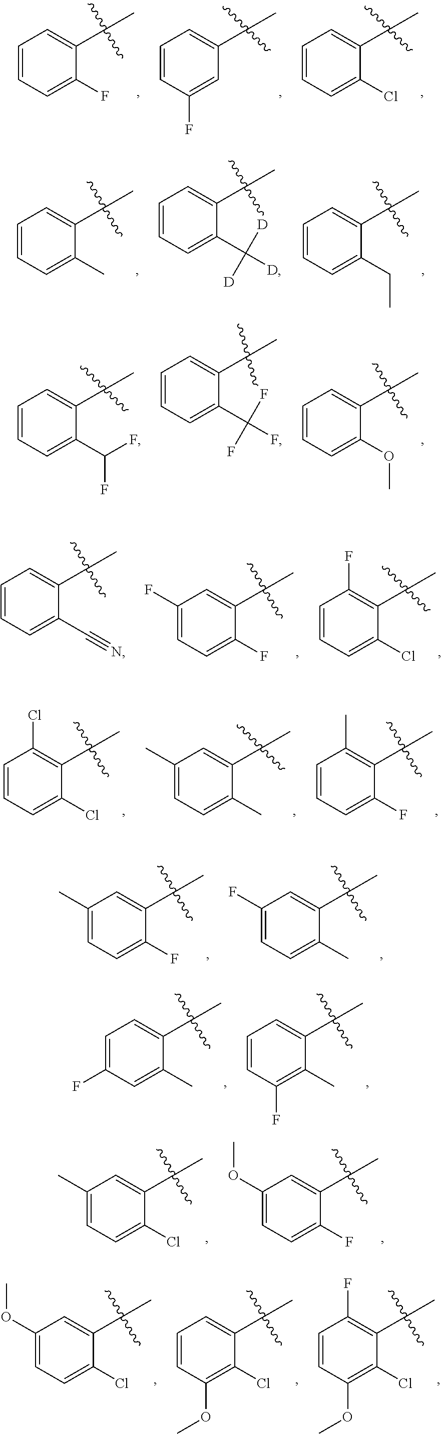

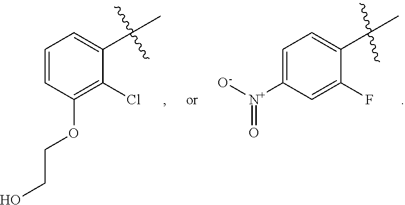 [SMILES: *c1cccc(OCCO)c1Cl ; *c1ccc([N+](=O)[O-])cc1F]

In an embodiment of the invention, the DHODH inhibitor is a compound of Formula (Z) wherein R⁴ is

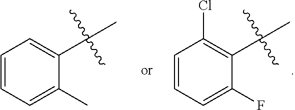

In an embodiment of the invention, the DHODH inhibitor is a compound of Formula (Z) wherein
R⁴ is

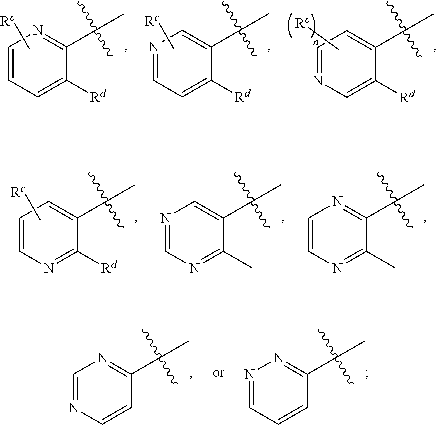

wherein
each R² is independently selected from the group consisting of: H; halo; C₁₋₄alkyl; C₁₋alkyl substituted with a member selected from the group consisting of: OH, OCH₃, SCH₃, and OCF₃; C₁₋₄haloalkyl; C₁₋₄haloalkyl substituted with a member selected from the group consisting of: OH, and OCH₃; and R^{d} is selected from the group consisting of: halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₄alkyl; CN; and OC₁₋₆alkyl.

In an embodiment of the invention, the DHODH inhibitor is a compound of Formula (Z) wherein
R⁴ is

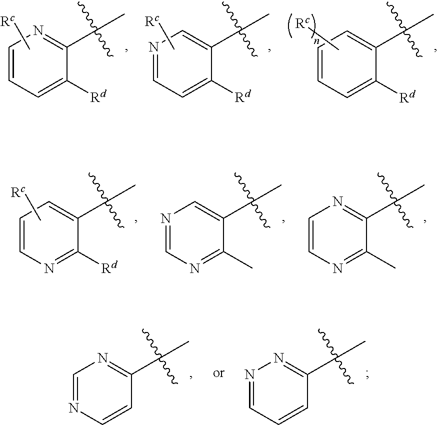

wherein
each R^{c} is independently selected from the group consisting of: H, halo, C₁₋₄alkyl, C₁₋₄haloalkyl, OC₁₋₄alkyl, and OH;
R^{d} is selected from the group consisting of: halo, C₁₋₄alkyl, and OC₁₋₄alkyl; and
n is 1, or 2.

In an embodiment of the invention, the DHODH inhibitor is a compound of Formula (Z) wherein R⁴ is

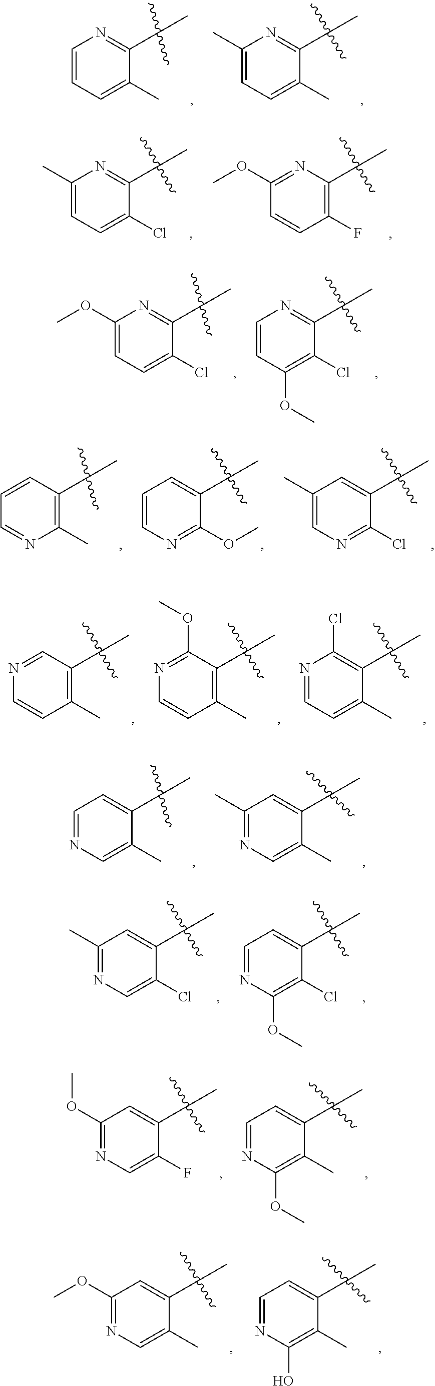

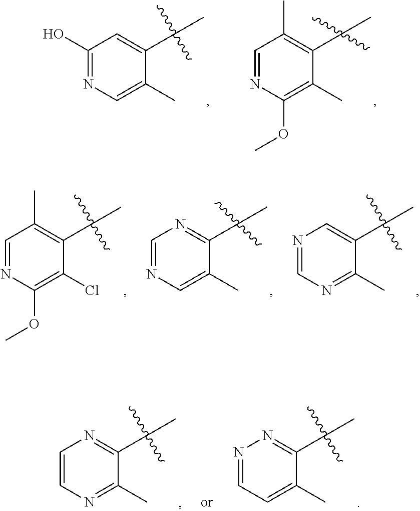

In an embodiment of the invention, the DHODH inhibitor is a compound of Formula (Z) wherein R⁴ is

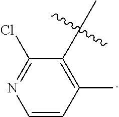 [SMILES: *c1c(C)ccnc1Cl]

In an embodiment of the invention, the DHODH inhibitor is a compound of Formula (Z) wherein
R⁴ is

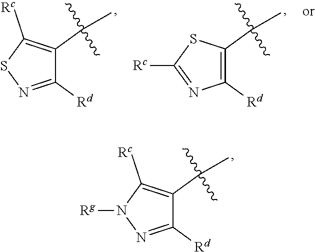

wherein
R^{c} is H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₄alkyl;
R^{d} is halo; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃; and
R^{g} is H; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃.

In an embodiment of the invention, the DHODH inhibitor is a compound of Formula (Z) wherein
R⁴ is

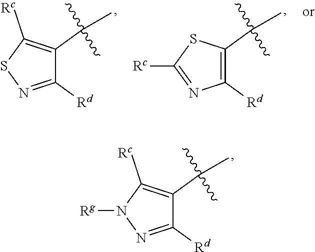

wherein
R^{c} is H or halo;
R^{d} is C₁₋₄alkyl; and
R^{g} is H.

In an embodiment of the invention, the DHODH inhibitor is a compound of Formula (Z) wherein R⁴ is

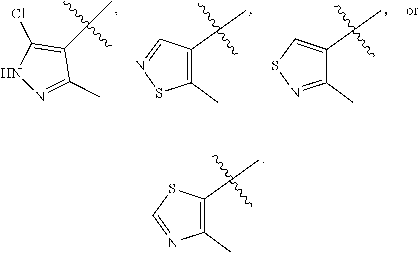

In an embodiment of the invention, the DHODH inhibitor is a compound of Formula (Z) selected from the group consisting of:
6-(4-Ethyl-3-(hydroxymethyl)-5-oxo-4,5-dihydro-1H-1,2,4-triazol-1-yl)-2-(3-fluorophenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-2-(3-fluorophenyl)-4-isopropylisoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-isopropylisoquinolin-1(2H)-one;
6-(4-Ethyl-3-(hydroxymethyl)-5-oxo-4,5-dihydro-1H-1,2,4-triazol-1-yl)-2-(3-fluorophenyl)-4-phenylisoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-(prop-1-en-2-yl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-(3,3,3-trifluoroprop-1-en-2-yl)isoquinolin-1(2H)-one;
2-(2,6-Dichlorophenyl)-6-(4-ethyl-3-(hydroxymethyl)-5-oxo-4,5-dihydro-1H-1,2,4-triazol-1-yl)-4-(1-methylcyclopropyl)isoquinolin-1(2H)-one;
2-(2,6-Dichlorophenyl)-6-(4-ethyl-3-(hydroxymethyl)-5-oxo-4,5-dihydro-1H-1,2,4-triazol-1-yl)-4-(prop-1-en-2-yl)isoquinolin-1(2H)-one;
2-(2-Chloro-6-fluorophenyl)-4-cyclopropyl-6-(4-ethyl-3-(hydroxymethyl)-5-oxo-4,5-dihydro-1H-1,2,4-triazol-1-yl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-4-(prop-1-en-2-yl)-2-(2-(trifluoromethyl)phenyl)isoquinolin-1(2H)-one;
2-(6-(4-Ethyl-3-(hydroxymethyl)-5-oxo-4,5-dihydro-1H-1,2,4-triazol-1-yl)-1-oxo-4-(prop-1-en-2-yl)isoquinolin-2(1H)-yl)benzonitrile;
2-(2-Chlorophenyl)-6-(4-ethyl-3-(hydroxymethyl)-5-oxo-4,5-dihydro-1H-1,2,4-triazol-1-yl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-4-(prop-1-en-2-yl)-2-(o-tolyl)isoquinolin-1(2H)-one;
2-(2-Chlorophenyl)-6-(4-ethyl-3-(hydroxymethyl)-5-oxo-4,5-dihydro-1H-1,2,4-triazol-1-yl)-4-isopropylphthalazin-1(2H)-one;
6-(4-Ethyl-3-(hydroxymethyl)-5-oxo-4,5-dihydro-1H-1,2,4-triazol-1-yl)-2-(3-fluorophenyl)-4-isopropylphthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-isopropylphthalazin-1(2H)-one;
4-Ethyl-6-(4-ethyl-3-(hydroxymethyl)-5-oxo-4,5-dihydro-1H-1,2,4-triazol-1-yl)-2-(3-fluorophenyl)phthalazin-1(2H)-one;
4-Ethyl-6-(4-ethyl-3-(hydroxymethyl)-5-oxo-4,5-dihydro-1H-1,2,4-triazol-1-yl)-2-(o-tolyl)phthalazin-1(2H)-one;
6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one;
2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-(1-methylcyclopropyl)isoquinolin-1(2H)-one;
6-(4-Ethyl-3-(hydroxymethyl)-5-oxo-4,5-dihydro-1H-1,2,4-triazol-1-yl)-2-(3-fluorophenyl)-4-(2-hydroxypropan-2-yl)isoquinolin-1(2H)-one;
4-(Dimethylamino)-6-(4-ethyl-3-(hydroxymethyl)-5-oxo-4,5-dihydro-1H-1,2,4-triazol-1-yl)-2-(o-tolyl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)phthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-methoxy-4-(prop-1-en-2-yl)phthalazin-1(2H)-one;
2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(4-Ethyl-3-(hydroxymethyl)-5-oxo-4,5-dihydro-1H-1,2,4-triazol-1-yl)-3-fluoro-8-(prop-1-en-2-yl)-6-(o-tolyl)-1,6-naphthyridin-5(6H)-one;
2-(4-Ethyl-3-(hydroxymethyl)-5-oxo-4,5-dihydro-1H-1,2,4-triazol-1-yl)-3-fluoro-8-methyl-6-(o-tolyl)pyrido[2,3-d]pyridazin-5(6H)-one;
2-(4-Ethyl-3-(hydroxymethyl)-5-oxo-4,5-dihydro-1H-1,2,4-triazol-1-yl)-3-fluoro-8-isopropyl-6-(o-tolyl)pyrido[2,3-d]pyridazin-5(6H)-one;
6-(2-Chloro-6-fluorophenyl)-2-(4-ethyl-3-(hydroxymethyl)-5-oxo-4,5-dihydro-1H-1,2,4-triazol-1-yl)-3-fluoro-8-(prop-1-en-2-yl)-1,6-naphthyridin-5(6H)-one;
6-(2-Chloro-6-fluorophenyl)-2-(4-ethyl-3-(hydroxymethyl)-5-oxo-4,5-dihydro-1H-1,2,4-triazol-1-yl)-3-fluoro-8-isopropyl-1,6-naphthyridin-5(6H)-one;
(S)-2-(4-Ethyl-3-(hydroxymethyl)-5-oxo-4,5-dihydro-1H-1,2,4-triazol-1-yl)-3-fluoro-6-(o-tolyl)-8-(1,1,1-trifluoropropan-2-yl)-1,6-naphthyridin-5(6H)-one;
(R)-2-(4-Ethyl-3-(hydroxymethyl)-5-oxo-4,5-dihydro-1H-1,2,4-triazol-1-yl)-3-fluoro-6-(o-tolyl)-8-(1,1,1-trifluoropropan-2-yl)-1,6-naphthyridin-5(6H)-one;
6-(4-Ethyl-3-(hydroxymethyl)-5-oxo-4,5-dihydro-1H-1,2,4-triazol-1-yl)-7-fluoro-4-isopropyl-2-(4-methylthiazol-5-yl)isoquinolin-1(2H)-one;
2-(4-Ethyl-3-(hydroxymethyl)-5-oxo-4,5-dihydro-1H-1,2,4-triazol-1-yl)-3-fluoro-8-isopropyl-6-(o-tolyl)-1,6-naphthyridin-5(6H)-one;
6-(4-Ethyl-3-(hydroxymethyl)-5-oxo-4,5-dihydro-1H-1,2,4-triazol-1-yl)-7-fluoro-2-(2-fluoro-5-methylphenyl)-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-5-methylphenyl)-4-(prop-1-en-2-yl)isoquinolin-1(2H)-one;
2-(2-Chloro-5-methylphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-5-methoxyphenyl)-4-(prop-1-en-2-yl)isoquinolin-1(2H)-one;
2-(2-Chlorophenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-4-(prop-1-en-2-yl)-2-(o-tolyl)isoquinolin-1(2H)-one;
2-(2-Chloro-5-methoxyphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
racemic-4-(sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoroisoquinolin-1(2H)-one;
2-(3-Chloro-6-methoxypyridin-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxy-4-methylpyridin-3-yl)isoquinolin-1(2H)-one;
2-(2-Chloro-6-fluoro-3-methoxyphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(o-tolyl)phthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropylphthalazin-1(2H)-one;
Racemic 6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)phthalazin-1(2H)-one;
(S*)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)phthalazin-1(2H)-one;
(R*)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)phthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-(1-methylcyclopropyl)isoquinolin-1(2H)-one;
6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)isoquinolin-1(2H)-one;
2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-5-methoxyphenyl)-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(4-fluoro-2-methylphenyl)-4-isopropylisoquinolin-1(2H)-one;
2-(2-Chloro-3-(2-hydroxyethoxy)phenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluorophenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(5-fluoro-2-methylphenyl)-4-isopropylisoquinolin-1(2H)-one;
2-(2,5-Difluorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-6-methylphenyl)-4-isopropylisoquinolin-1(2H)-one;
2-(2-Chloro-3-methoxyphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)isoquinolin-1(2H)-one;
2-(2,5-Difluorophenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-6-methylphenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(3-fluoro-2-methylphenyl)-4-isopropylisoquinolin-1(2H)-one;
2-(2,5-Dimethylphenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(4-fluoro-2-methylphenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluorophenyl)-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxy-3,5-dimethylpyridin-4-yl)isoquinolin-1(2H)-one;
6-(4-Ethyl-3-(hydroxymethyl)-5-oxo-4,5-dihydro-1H-1,2,4-triazol-1-yl)-7-methyl-4-(prop-1-en-2-yl)-2-(o-tolyl)isoquinolin-1(2H)-one;
2-(2-Chloro-6-fluoro-3-methoxyphenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(o-tolyl)-4-(3,3,3-trifluoroprop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(5-fluoro-2-methylphenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-methoxyphenyl)-4-(prop-1-en-2-yl)isoquinolin-1(2H)-one;
2-(2-Chloro-5-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(5-fluoro-2-methoxypyridin-4-yl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(3-fluoro-2-methylphenyl)-4-(prop-1-en-2-yl)isoquinolin-1(2H)-one;
2-(2,5-Dimethylphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
Racemic-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)isoquinolin-1(2H)-one;
6-(4-Ethyl-3-(hydroxymethyl)-5-oxo-4,5-dihydro-1H-1,2,4-triazol-1-yl)-2-(2-ethylphenyl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxypyridin-3-yl)isoquinolin-1(2H)-one;
6-(4-Ethyl-3-(hydroxymethyl)-5-oxo-4,5-dihydro-1H-1,2,4-triazol-1-yl)-7-fluoro-2-(5-fluoro-2-methoxypyridin-4-yl)-4-isopropylisoquinolin-1(2H)-one;
2-(2-Chloro-5-methylpyridin-3-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-(methyl-d₃)phenyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(4-methylpyrimidin-5-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxyphenyl)isoquinolin-1(2H)-one;
6-(4-Ethyl-3-(hydroxymethyl)-5-oxo-4,5-dihydro-1H-1,2,4-triazol-1-yl)-7-fluoro-2-(3-fluoro-6-methoxypyridin-2-yl)-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(3-methylpyrazin-2-yl)isoquinolin-1(2H)-one;
2-(2-Chloro-5-methylpyridin-3-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(4-Ethyl-3-(hydroxymethyl)-5-oxo-4,5-dihydro-1H-1,2,4-triazol-1-yl)-3-fluoro-6-(2-fluoro-5-methylphenyl)-8-isopropyl-1,6-naphthyridin-5(6H)-one;
6-(4-Ethyl-3-(hydroxymethyl)-5-oxo-4,5-dihydro-1H-1,2,4-triazol-1-yl)-7-fluoro 4-isopropyl-2-(4-methylpyridazin-3-yl)isoquinolin-1(2H)-one;
(S)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)isoquinolin-1(2H)-one;
(R)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-(trifluoromethyl)phenyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(5-methylpyrimidin-4-yl)isoquinolin-1(2H)-one;
2-(2-(Difluoromethyl)phenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(3-Chloro-2-methoxypyridin-4-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-Cyclohexyl-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(3-Chloro-6-methylpyridin-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-Cyclopentyl-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(3-Chloro-4-methoxypyridin-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1R,2S)-2-methylcyclohexyl)isoquinolin-1(2H)-one;
2-(1,3-Dimethoxypropan-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxy-5-methylpyridin-4-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1S,2R)-2-methylcyclohexyl)isoquinolin-1(2H)-one;
2-(Cyclopropylmethyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(1-methoxybutan-2-yl)isoquinolin-1(2H)-one;
2-(2-Chlorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(3-methylpyridin-2-yl)isoquinolin-1(2H)-one;
Racemic 6-(4-Ethyl-3-(hydroxymethyl)-5-oxo-4,5-dihydro-1H-1,2,4-triazol-1-yl)-7-fluoro-4-isopropyl-2-((cis)-3-methoxycyclopentyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1R*,2R*)-2-methylcyclohexyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1S*,2S*)-2-methylcyclohexyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(pentan-3-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1R*,2R*)-2-methylcyclopentyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1S*,2S*)-2-methylcyclopentyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1R*,2S*)-2-methylcyclopentyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1S*,2R*)-2-methylcyclopentyl)isoquinolin-1(2H)-one;
Racemic 6-(4-Ethyl-3-(hydroxymethyl)-5-oxo-4,5-dihydro-1H-1,2,4-triazol-1-yl)-7-fluoro-4-isopropyl-2-((cis)-3-methoxycyclohexyl)isoquinolin-1(2H)-one;
2-(Bicyclo[2.2.1]heptan-1-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxy-3-methylpyridin-4-yl)isoquinolin-1(2H)-one;
2-(4-Ethyl-3-(hydroxymethyl)-5-oxo-4,5-dihydro-1H-1,2,4-triazol-1-yl)-3-fluoro-8-isopropyl-6-(2-methoxyphenyl)-1,6-naphthyridin-5(6H)-one;
6-(4-Ethyl-3-(hydroxymethyl)-5-oxo-4,5-dihydro-1H-1,2,4-triazol-1-yl)-7-fluoro-4-isopropyl-2-(3-methylisothiazol-4-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(5-methylisothiazol-4-yl)isoquinolin-1(2H)-one;
2-(4-Ethyl-3-(hydroxymethyl)-5-oxo-4,5-dihydro-1H-1,2,4-triazol-1-yl)-3-fluoro-8-isopropyl-6-(2-(trifluoromethyl)phenyl)-1,6-naphthyridin-5(6H)-one;
2-(3,6-Dimethylpyridin-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(2,5-Dimethylpyridin-4-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(4-methylpyridin-3-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(3-methylpyridin-4-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methylpyridin-3-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-hydroxy-5-methylpyridin-4-yl)-4-isopropylisoquinolin-1(2H)-one;
6-(2-(Difluoromethyl)phenyl)-2-(4-ethyl-3-(hydroxymethyl)-5-oxo-4,5-dihydro-1H-1,2,4-triazol-1-yl)-3-fluoro-8-isopropyl-1,6-naphthyridin-5(6H)-one;
6-(4-Ethyl-3-(hydroxymethyl)-5-oxo-4,5-dihydro-1H-1,2,4-triazol-1-yl)-7-fluoro-2-(2-hydroxy-3-methylpyridin-4-yl)-4-isopropylisoquinolin-1(2H)-one; and
2-(4-Ethyl-3-(hydroxymethyl)-5-oxo-4,5-dihydro-1H-1,2,4-triazol-1-yl)-3-fluoro-8-isopropyl-6-(o-D₃-tolyl)-1,6-naphthyridin-5(6H)-one;

or a pharmaceutically acceptable salt, isotope, N-oxide, solvate, or stereoisomer thereof; or a compound selected from
2-(2-Chloro-6-fluorophenyl)-6-(4-ethyl-3-(hydroxymethyl)-5-oxo-4,5-dihydro-1H-1,2,4-triazol-1-yl)isoquinolin-1(2H)-one;
6-(4-Ethyl-3-(hydroxymethyl)-5-oxo-4,5-dihydro-1H-1,2,4-triazol-1-yl)-2-(2-fluoro-4-nitrophenyl)-4-iodoisoquinolin-1(2H)-one;
2-(2-Chloro-6-fluorophenyl)-7-(4-ethyl-3-(hydroxymethyl)-5-oxo-4,5-dihydro-1H-1,2,4-triazol-1-yl)-6-fluoro-4-(prop-1-en-2-yl)phthalazin-1(2H)-one;
2-(2-Chloro-6-fluorophenyl)-7-(4-ethyl-3-(hydroxymethyl)-5-oxo-4,5-dihydro-1H-1,2,4-triazol-1-yl)-6-methoxy-4-(prop-1-en-2-yl)phthalazin-1(2H)-one;

or a pharmaceutically acceptable salt, N-oxide, solvate, or stereoisomer thereof.

In an embodiment of the invention, the DHODH inhibitor is a compound of Formula (Z) selected from the group consisting of:
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one;
2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-(1-methylcyclopropyl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)phthalazin-1(2H)-one;
2-(4-Ethyl-3-(hydroxymethyl)-5-oxo-4,5-dihydro-1H-1,2,4-triazol-1-yl)-3-fluoro-8-isopropyl-6-(o-tolyl)-1,6-naphthyridin-5(6H)-one;
6-(4-Ethyl-3-(hydroxymethyl)-5-oxo-4,5-dihydro-1H-1,2,4-triazol-1-yl)-7-fluoro-4-isopropyl-2-(o-tolyl)phthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropylphthalazin-1(2H)-one;
6-(4-Ethyl-3-(hydroxymethyl)-5-oxo-4,5-dihydro-1H-1,2,4-triazol-1-yl)-7-fluoro-4-isopropyl-2-(2-(methyl-d3)phenyl)isoquinolin-1(2H)-one;
(R)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-(trifluoromethyl)phenyl)isoquinolin-1(2H)-one; and
2-(2-(Difluoromethyl)phenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;

or a pharmaceutically acceptable salt, isotope, N-oxide, solvate, or stereoisomer thereof.

In an embodiment of the invention, the DHODH inhibitor is 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one or a pharmaceutically acceptable salt, solvate, stereoisomer, isotopic variant, or N-oxide thereof.

In an embodiment of the invention, the DHODH inhibitor is a compound of Formula (Z) having the Formula (Za):

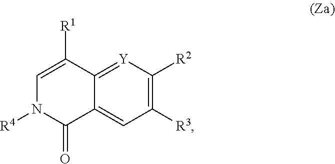

wherein
Y is CH or N;
R¹ is selected from the group consisting of: C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₂₋₆haloalkenyl; N(CH₃)₂; C₃₋₆cycloalkyl; C₃₋₆cycloalkyl substituted with C₁₋₆alkyl; and phenyl;
R² is

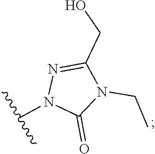 [SMILES: *n1nc(CO)n(CC)c1=O]

R³ is selected from the group consisting of: H, halo, CH₃ and OCH₃;
R⁴ is selected from the group consisting of:
C₁₋₆alkyl; C₁₋₆alkyl substituted with one or two OCH₃; C₃₋₆cycloalkyl; C₃₋₆cycloalkyl substituted with CH₃, or OCH₃; CH₂—C₃₋₆cycloalkyl; and

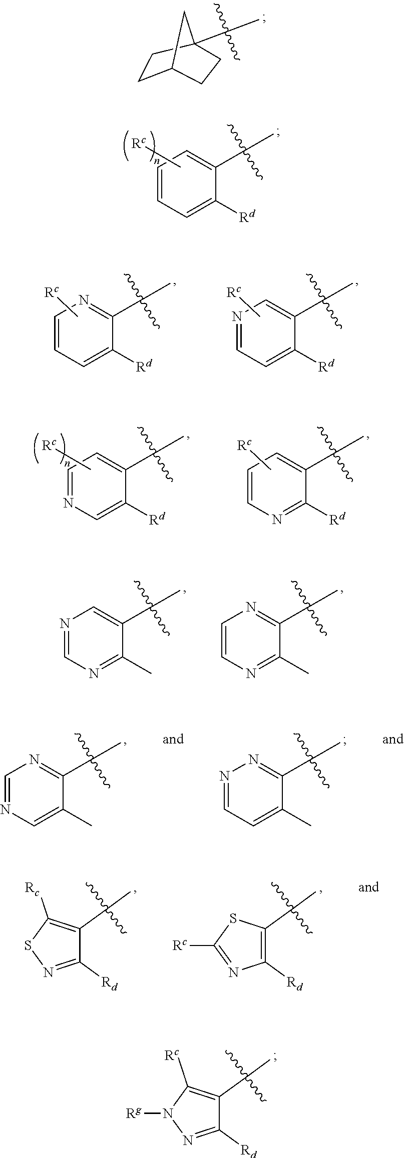

wherein
each R^{c} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; NO₂; OH; O—CH₂CH₂OH; and OC₁₋₆alkyl;
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; CN; and OC₁₋₆alkyl;
R⁹ is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
   or a pharmaceutically acceptable salt, solvate, stereoisomer, isotopic variant, or N-oxide thereof.

In an embodiment of the invention, the DHODH inhibitor is a compound of Formula (Z) having the Formula (Zb):

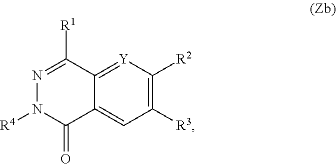

wherein
Y is CH or N:
R¹ is selected from the group consisting of: C₁₋₆alkyl, C₁₋₆haloalkyl and C₂₋₆alkenyl;
R² is

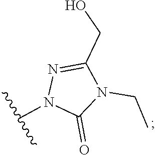 [SMILES: *n1nc(CO)n(CC)c1=O]

R³ is selected from the group consisting of: H, halo and OCH₃;
R⁴ is selected from the group consisting of:

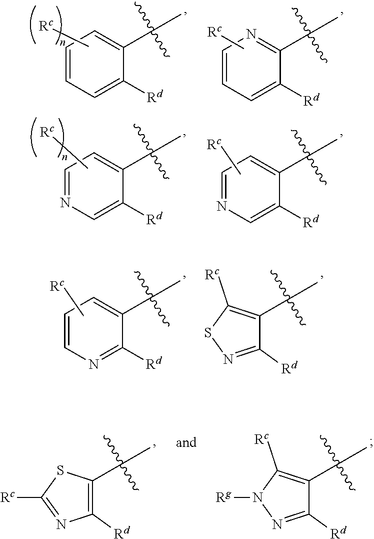

wherein
R^{c} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and NO₂;
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; CN; and OC₁₋₆alkyl;
R^{g} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1;
or a pharmaceutically acceptable salt, solvate, stereoisomer, isotopic variant, or N-oxide thereof.

Exemplary compounds of Formula (Z) useful in methods of the invention will now be described by reference to the illustrative synthetic schemes for their general preparation below and examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (Z). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

All abbreviations used in the general schemes and examples for Formula (Z) are as defined in Table 1A. Variables are as defined in the scope or as specifically defined in the general Schemes.

TABLE 1A
Abbreviations

| Abbreviation | Name |
|---|---|
| Å | angstrom |
| ACN or MeCN | acetonitrile |
| AcOH | glacial acetic acid |
| AcOK | Potassium acetate |
| AgBF₄ | Silver tetrafluoroborate |
| AlMe₃ | Trimethylaluminium |
| Ar | Argon |
| aq. | aqueous |
| AuCl₃ | Gold(III) chloride |
| BCl₃ | Boron trichloride |
| Bn or Bzl | benzyl |
| Boc | tert-butyloxycarbonyl |
| (Boc)₂O | Di-tert-butyl dicarbonate |
| Catacxium A Pd G2 | Chloro[(di(1-adamantyl)-N-butylphosphine)-2-(2- |
| | aminobiphenyl)]palladium(II) |
| CdCl₂ | Cadmium chloride |
| Celite ® | diatomaceous earth |
| conc. | concentrated |
| CO₂ | Carbon dioxide |
| (COCl)₂ | Oxalyl chloride |
| CuI | Copper(I) iodide |
| Cu(OAc)₂ | copper(II) acetate |
| Cy₂NMe | N,N-Dicyclohexylmethylamine |
| Cs₂CO₃ | Cesium carbonate |
| DCC | N,N′-dicyclohexyl-carbodiimide |
| DCE | dichloroethane |
| DCM | dichloromethane |
| DIPEA or DIEA | diisopropyl-ethyl amine |
| DEAD | Diethyl azodicarboxylate |
| DEA | Diethanolamine |
| DHP | 3,4-Dihydropyran |
| DMA | dimethylaniline |
| DMAP | 4-dimethylaminopyridine |
| DME | dimethoxyethane |
| DMF | N,N-dimethylformamide |
| DMP | Dess-Martin periodinane or 1,1,1-Tris(acetyloxy)-1,1-dihydro-1,2- |
| | benziodoxol-3-(1H)-one |
| DMSO | dimethylsulfoxide |
| Dppf or DPPF | 1,1′-Bis(diphenylphosphino)ferrocene |
| EDCI | 1-ethyl-3-(3-dimethylaminopropyl) carbodiimide |
| ES-API | electrospray-atmospheric pressure ionization |
| ESI | electrospray ionization |
| Et₃N•3HF | Triethylamine trihydrofluoride |
| EtOAc or EA | ethyl acetate |
| EtOH | ethanol |
| EtONa | sodium ethoxide |
| EtMgBr | Ethylmagnesium bromide |
| GCMS | gas chromatography-mass spectrometry |
| h or hr(s) | hour or hours |
| H₂ | Hydrogen gas |
| HCl | Hydrogen chloride |
| HPLC | high performance liquid chromatography |
| HPA | hypophosphorous acid |
| H₂SO₄ | Sulfuric acid |
| i-PrMgCl | Isopropylmagnesium chloride |
| IPA | Isopropylamine |
| iPrOH | Isopropyl alcohol or 2-propanol |
| isovaleraldehyde | 3-methylbutanal |
| KHMDS | Potassium bis(trimethylsilyl)amide |
| KI | Potassium iodide |
| K₂OsO₄•2H₂O | Potassium osmate (VI) dihydrate |
| K₂CO₃ | Potassium carbonate |
| K₃PO₄ | Potassium phosphate |
| LCMS or LC/MS | Liquid chromatography-mass spectrometry |
| LiHMDS | Lithium bis(trimethylsilyl)amide |
| MeOH | methanol |
| MeMgBr | methylmagnesium bromide |
| Mg(ClO₄)₂ | Magnesium perchlorate |
| MgSO₄ | Magnesium sulfate |
| MHz | megahertz |
| min | minute or minutes |
| MS | mass spectrometry |
| NaBH₄ | Sodium borohydride |
| NaHMDS | Sodium bis(trimethylsilyl)amide |
| NaOH | Sodium hydroxide |
| NaOEt | Sodium ethoxide |
| NaHCO₃ | Sodium bicarbonate |
| Na₂CO₃ | Sodium carbonate |
| NaIO₄ | Sodium periodate |
| NaNO₂ | Sodium nitrite |
| Na₂SO₄ | Sodium sulfate |
| N₂ | Nitrogen gas |
| NBS | N-Bromosuccinimide |
| NCS | N-chlorosuccinimide |
| NH₂NH₂•H₂O | Hydrazine monohydrate |
| NIS | N-iodosuccinimide |
| NH₄Cl | Ammonium chloride |
| NH₄HCO₃ | Ammonium bicarbonate |
| NH₃•H₂O or NH₄OH | Ammonium hydroxide |
| NMR | nuclear magnetic resonance |
| [Pd(allyl)Cl]₂ | Allylchloropalladium dimer or Bis(allyl)dichlorodipalladium or |
| | Bis(allyl)dichloropalladium or Bis(allylchloropalladium) or |
| | Diallyldichlorodipalladium |
| PdCl₂(PPh₃)₂ or | bis(triphenylphosphine)palladium(II) dichloride |
| Pd(PPh₃)₂Cl₂ | |
| Pd(PPh₃)₄ | tetrakis(triphenylphosphine)palladium |
| Pd(OAc)₂ | palladium (II) acetate |
| P(tBu₃)PdG₂ or | chloro[(tri-tert-butylphosphine)-2-(2-aminobiphenyl)] palladium(II) |
| tBu₃PPdG₂ | |
| PE | petroleum ether |
| PPh₃ | triphenylphosphine |
| ppm | parts per million |
| Pd(dppf)Cl₂•CH₂Cl₂ | 1,1′-bis(diphenylphosphino)ferrocene-palladium(II)dichloride |
| or Pd(dppf)Cl₂•DCM | dichloromethane complex |
| Pd/C | Palladium on carbon |
| PG | Protecting group |
| RP | reverse-phase |
| rt or RT | room temperature |
| Rₜ | retention time |
| RhCl(PPh₃)₃ or | Wilkinson's Catalyst or Chlorotris(triphenylphosphine)rhodium(I) |
| Rh(PPh₃)₃Cl | |
| Sec | second or seconds |
| SFC | supercritical fluid chromatography |
| SiO₂ | silica gel |
| SOCl₂ | Thionyl chloride |
| O₂ | Oxygen gas |
| TBAB | tetrabutylammonium bromide or tetra-n-butylammonium bromide |
| TBDPS | tert-Butyldiphenylchlorosilane |
| TBAF | tetrabutylammonium fluoride |
| TBHP | tert-butyl hydroperoxide |
| TBS | tert-Butyldimethylsilyl |
| TES | triethylsilane |
| TIPS | triisopropylsilane |
| TEA or Et₃N | triethylamine |
| TFA | trifluoroacetic acid |
| THF | tetrahydrofuran |
| TiCl₄ | Titanium tetrachloride |
| TLC | thin layer chromatography |
| TF₂NPh | N-phenylbis(trifluoromethanesufonimide) |
| triflate | trifluoromethanesulfonyl |
| Tf₂O | Triflic anhydride or Trifluoromethanesulfonic anhydride |
| TMSOiPr | Isopropoxytrimethylsilane |
| (PTSA or pTsOH) or | p-Toluenesulfonic acid |
| tosylic acid | |

### PREPARATIVE EXAMPLES

Exemplary compounds of Formula (Z) useful in methods of the invention will now be described by reference to the illustrative synthetic schemes for their general preparation below and the specific examples to follow.

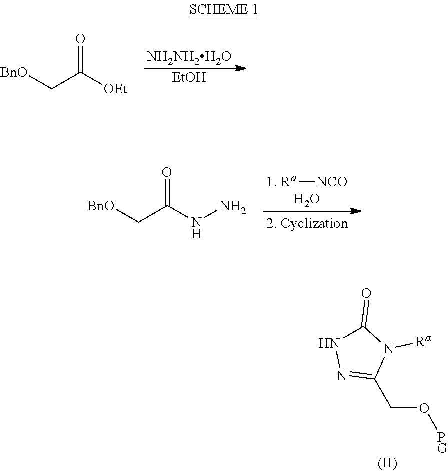

According to SCHEME 1, a 1,2,4-triazol-5(4H)-one compound of formula (II), where PG is Bn, is prepared from ethyl 2-(benzyloxy)acetate in three steps. In a first step 2-(benzyloxy)acetohydrazide is prepared by the reaction of ethyl 2-(benzyloxy)acetate with hydrazine hydrate, in a suitable solvent such as EtOH, and the like; at temperatures ranging from 70-85° C. Reaction of the hydrazide with an isocyanate of formula R^{a}—NCO, where R^{a} is C₁₋₆alkyl, in a suitable solvent such as water, and the like; provides the corresponding semicarbazide. Subsequent cyclization of the semicarbazide with a suitable base such as NaOH, in a suitable solvent such as water, provides a compound of formula (II), where PG is Bn.

A compound of formula (II), where R^{a} is C₁₋₆haloalkyl or C₃₋₆cycloalkyl; may be prepared as previously described employing a suitably substituted compound of formula R^{a}—NCO, where R^{a} is C₁₋₆haloalkyl or C₃₋₆cycloalkyl.

Protecting group exchange of a compound of formula (II), where PG is Bn to a compound of formula (II) where PG is TBDPS, is achieved in two steps employing established methodologies, such as those described in T. W. Greene and P. G. M. Wuts, “Protective Groups in Organic Synthesis,” 3 ed., John Wiley & Sons, 1999. In a first step, deprotection of benzyl group is achieved under hydrogenolytic conditions known to one skilled in the art provides the alcohol. For example, deprotection is achieved employing a palladium catalyst such Pd/C, and the like; under H₂; in a suitable solvent such as EtOH, MeOH, EtOAc, or a mixture thereof, preferably EtOH; with or without the presence HCl; for a period of 4 to 72 hrs. In a second step, protection of the corresponding alcohol as the silyl ether, is achieved with tert-butyldiphenylsilyl chloride, a suitable base such as imidazole, dimethylaminopyridine, pyridine, and the like; in a solvent such as DMF, DCM, and the like; at temperatures ranging from 0° C. to room temperature; affords a compound of formula (II) where PG is TBDPS.

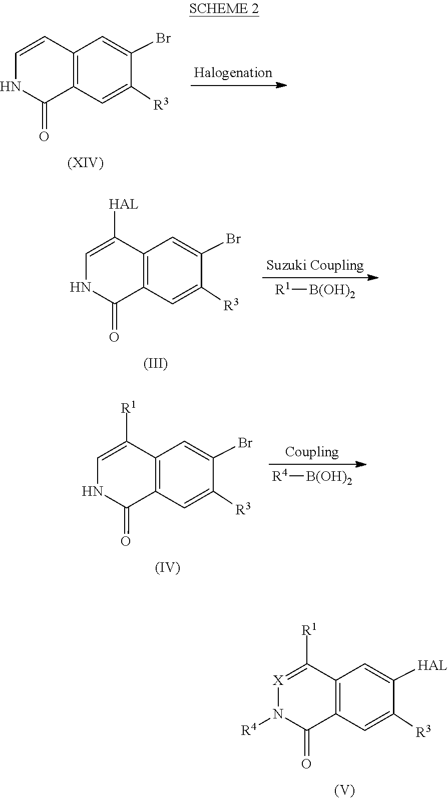

According to SCHEME 2, a compound of formula (XIV), where R³ is H is treated with a halogenating reagent such as N-iodosuccinimide (NIS), and the like; in an aprotic solvent such as acetonitrile, and the like; under heating conditions; to afford the halogenated compound of formula (III), where HAL is iodide. A compound of formula R¹—B(OH)₂; is reacted under Suzuki coupling conditions known to one skilled in the art with a compound of formula (III), to provide a compound of formula (IV). For example, a compound of formula (III), where HAL is iodide, is reacted a commercially available or synthetically accessible boronic acid (or boronic ester) such as R¹—B(OH)₂, where R¹ is an optionally substituted C₂₋₆alkenyl or aryl as defined herein with reference to Formula (Z); a palladium catalyst such as bis(triphenylphosphine)palladium(II) dichloride, tetrakis(triphenylphosphine)palladium, and the like; a suitable base such as potassium phosphate, Cs₂CO₃, and the like; in a suitable solvent such as dioxane, water, ethanol, or a mixture thereof; to provide a compound of formula compound (IV). A compound of formula (IV), where R³ is H, is reacted with a compound of formula R⁴—B(OH)₂; under copper (II) mediated Chan-Lam coupling conditions known to one skilled in the art, to provide a compound of formula (V), where HAL is bromide, X is CH and R³ is H. For example, a compound of formula (IV) is reacted with a compound of formula R⁴—B(OH)₂, where R⁴ is as defined herein with reference to Formula (Z); a catalyst such as copper(II) acetate, and the like; a base such as pyridine, NEt₃, and the like; in a suitable solvent such as DCM, ACN, dioxane, THF, and the like; to afford a compound of formula (V).

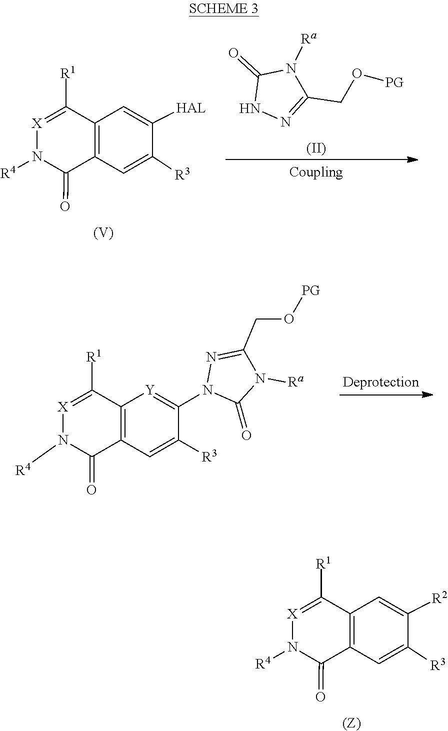

According to SCHEME 3, Ullmann-type aromatic amination reaction of compound of formula (V), where R¹ is optionally substituted C₂₋₆alkenyl, R³ is H, R⁴ is suitably substituted phenyl as described herein with reference to Formula (Z), and HAL is Br; with a commercially available or synthetically accessible nucleophilic compound of formula (II), where R^{a} is C₁₋₆alkyl; such as suitably protected triazolones, where PG is selected from: benzyl, 4-methoxy benzyl, or an alkyl or aryl silane such as TBDPS, TBS, TES, or TIPS; in the presence of catalytic CuI and a diamine such as trans-1,2-diaminocyclohexane, and a base such as K₃PO₄, K₂CO₃, Cs₂CO₃, NaHCO₃, triethylamine, and the like; in a suitable solvent such as 1,4-dioxane, DMSO, DMF, THF, ACN, and the like; provides a compound of formula (VI), where X is CH and Y is CH.

A compound of formula (VI), where PG is Bn and R¹ is C₂₋₆alkenyl, is reacted under Simmons-Smith cyclopropanation reaction conditions known to one skilled in the art to provide a compound of formula (VI) where R¹ is C₃₋₆cycloalkyl substituted with C₁₋₆alkyl. For example, a compound of formula (VI), where R¹ is

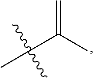 [SMILES: *C(=C)C]

is reacted with diiodomethane; diethylzinc; in a suitable solvent such as toluene, and the like; at temperatures ranging from 0° C. to room temperature; for a period of 3 to 26 h; to provide a compound of formula (VI), where R¹ is cyclopropyl substituted with CH₃.

Subsequent deprotection employing established methodologies, such as those described in T. W. Greene and P. G. M. Wuts, “Protective Groups in Organic Synthesis,” 3 ed., John Wiley & Sons, 1999), provides a compound of Formula (Z), where X and Y are CH. For example, compound of formula (VI), where R³ is H, and PG is TBDPS, is deprotected employing conditions known to one skilled in the art, preferably with TBAF in a suitable solvent such as THF, and the like. In a preferred method, PG is TBDPS, and R^{a} is C₁₋₆alkyl. Alternately, removal of a TBDPS protecting group is achieved employing triethylamine trihydrogen fluoride (Et₃N·3HF).

Removal of the Bn protecting group is achieved in the presence of hydrogen gas, in the presence of a catalyst such as Palladium on carbon (Pd/C). Removal of the protecting group Bn is also achieved employing TFA, at a temperature of about 80° C.

A compound of Formula (Z), where X is CH; Y is CH; R², R³, R⁴ is each defined as described herein with reference to Formula (Z); and R¹ is C₂₋₆alkenyl, is reduced employing hydrogenation conditions known to one skilled in the art, for example, reaction with Pd/C or Wilkinson's Catalyst [RhCl(PPh₃)₃] under H₂; in a suitable solvent such as MeOH, THF, EtOAc, and the like; provides a compound of Formula (Z) where R¹ is C₂₋₆alkyl.

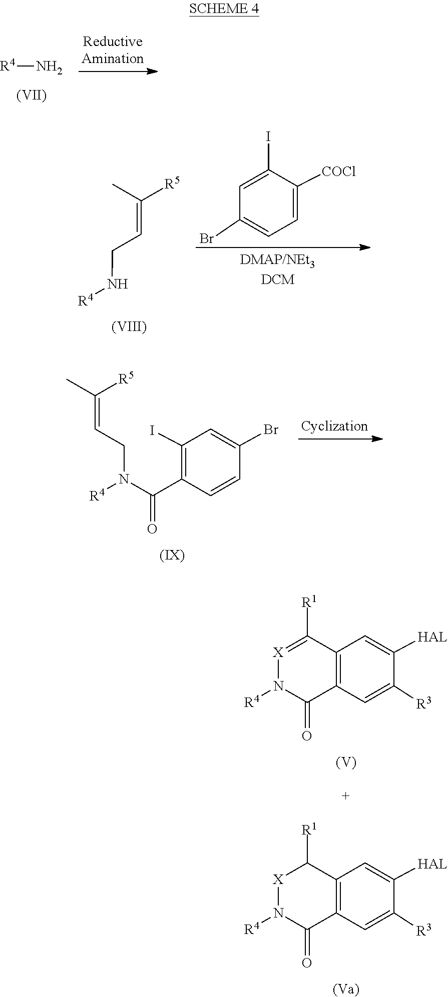

According to SCHEME 4, reductive amination of a compound of formula (VII), with α, β-unsaturated aldehyde such as 3-methyl-2-butenal, 3-methylpent-2-enal, and the like; employing TiCl₄; and a base such as triethylamine; in an aprotic solvent such as dichloromethane (DCM), and the like; provides an enamine intermediate which is subsequently reduced employing a reducing agent such as NaBH₄, and the like; to afford a compound of formula (VIII) where R⁵ is C₁₋₄alkyl, R⁴ is as defined herein with reference to Formula (Z). A compound of formula (VIII) is coupled with commercially available or synthetically accessible 4-bromo-2-iodobenzoyl chloride employing a base such as triethylamine and 4-dimethylaminopyridine (DMAP); in an anhydrous aprotic solvent such as dichloromethane (DCM), and the like; to afford a compound of formula (IX). Treatment of a compound of formula (IX), with palladium (II) acetate, tetrabutylammonium bromide, and potassium acetate under heating Heck reaction conditions, affords the intramolecular cyclized compounds of formula (V), wherein R¹ is optionally substituted C₂₋₆alkyl, R³ is H, X is CH, and HAL is Br; and (Va) wherein R¹ is optionally substituted C₂₋₆alkenyl, R³ is H, X is CH₂, and HAL is Br.

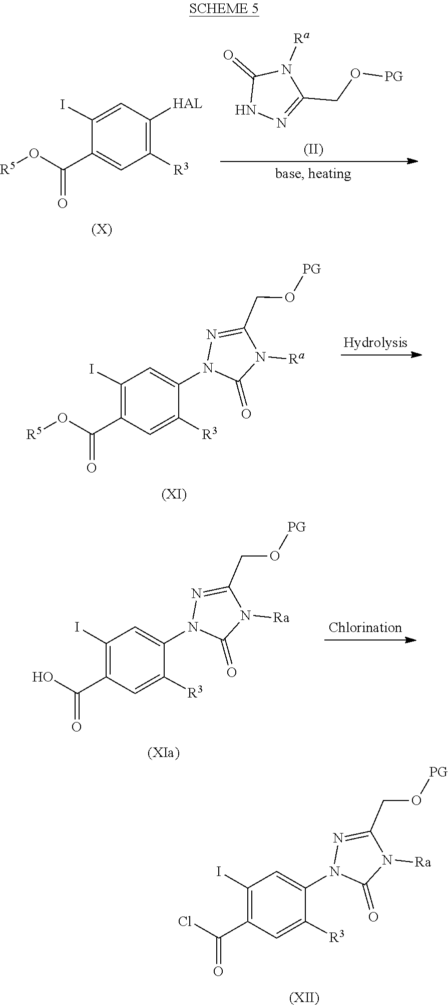

According to SCHEME 5, the reaction of a commercially available or synthetically accessible compound of formula (X), where HAL is F, R³ is F, and R⁵ is H or C₁₋₄alkyl; with a commercially available or synthetically accessible nucleophilic compound of formula (II), where R^{a} is C₁₋₆alkyl, such as suitably protected triazolones, where PG is selected from: benzyl, 4-methoxy benzyl, or an alkyl or aryl silane such as TBDPS, TBS, TES, or TIPS; in the presence of a base such as K₃PO₄, K₂CO₃, Cs₂CO₃, NaHCO₃, triethylamine, and the like; in a suitable solvent such as DMSO, DMF, THF, ACN, and the like; affords a compound of formula (XI). In a preferred method, PG is Bn, and R^{a} is C₁₋₆alkyl. The ester of formula (XI), when R⁵ is C₁₋₄alkyl, is hydrolyzed to its corresponding acid, under acidic or basic conditions. For example, the treatment of tert-butyl ester (R⁵ is tert-Bu) with TFA; or alternately, hydrolysis with a base like NaOH, in an aqueous solvent, affords a compound of formula (XIa), where R⁵ is H. A compound of formula (XIa) is chlorinated, employing conditions known to one skilled in the art, to provide the acyl chloride of formula (XII). For example, a compound of formula (XIa) is heated in SOCl₂; or treated with oxalyl chloride in DCM.

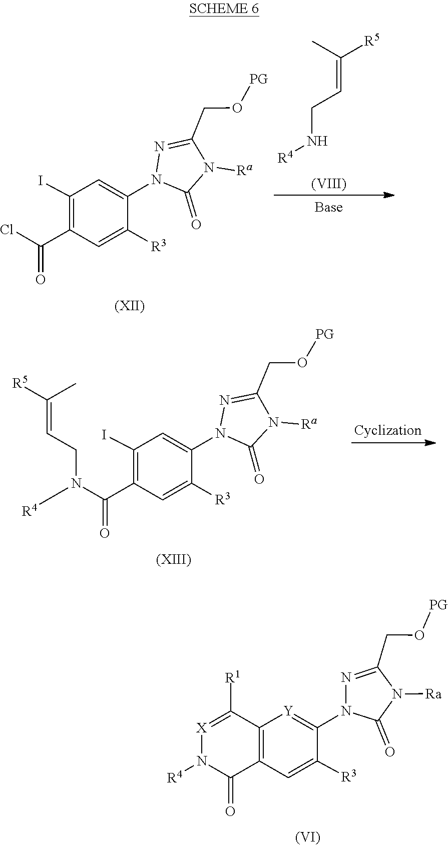

According to SCHEME 6, a compound of formula (XII), where R³ is H or F, PG is Bn, and R^{a} is C₁₋₆alkyl; is reacted with a compound of formula (VIII), where R⁵ is C₁₋₄alkyl, employing a base such as a mixture of triethylamine (TEA) and 4-dimethylaminopyridine (DMAP); in an anhydrous aprotic solvent such as dichloromethane (DCM), and the like; to afford a compound of formula (XIII). A compound of formula (VI), where X is CH and Y is CH, is obtained by treatment of a compound of formula (XIII), where R¹ is optionally substituted C₁₋₆alkyl as described herein with reference to Formula (Z); with palladium (II) acetate, tetrabutylammonium bromide, and potassium acetate under heating Heck reaction conditions, that affords a mixture of intramolecular cyclized compounds, which is then separated to isolate an intermediate compound where R¹ is C₂₋₆alkyl, and R³ is H or F.

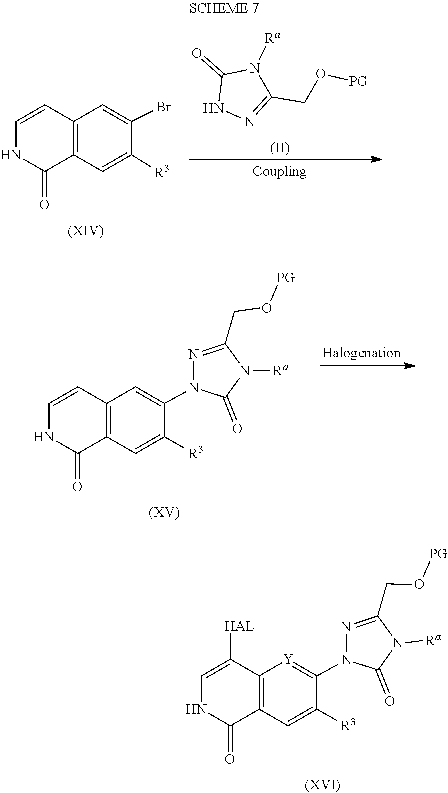

According to SCHEME 7, Ullmann-type aromatic amination reaction of a compound of formula (XIV), where R³ is H or F, with a compound of formula (II); such as suitably protected triazolones, where PG is selected from: benzyl, 4-methoxy benzyl, or an alkyl or aryl silane such as TBDPS, TBS, TES, or TIPS; according to methods previously described; affords a compound of formula (XV). In a preferred method, PG is Bn, and R^{a} is C₁₋₆alkyl. A compound of formula (XV) is treated with a halogenating reagent such as N-iodosuccinimide (NIS), and the like; in an aprotic solvent such as acetonitrile, and the like; under heating conditions; affords a halogenated compound of formula (XVI), where Y is CH and HAL is iodide.

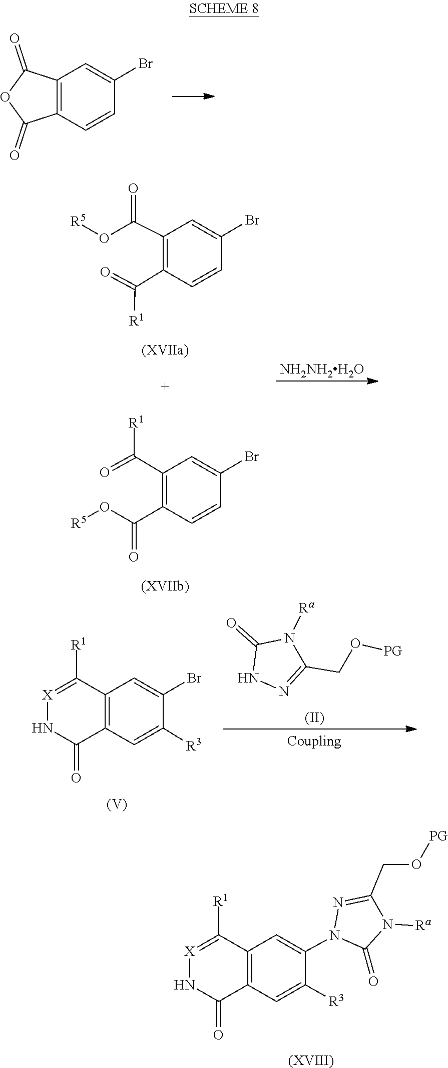

According to SCHEME 8, compounds of formula (XVIIa) and (XVIIb) are prepared from 5-bromoisobenzofuran-1,3-dione in two steps. 5-Bromoisobenzofuran-1,3-dione is reacted with a commercially available or synthetically accessible suitably substituted alkyl Grignard reagent such as i-PrMgCl, EtMgBr, and the like; in the presence of CdCl₂; in aprotic solvent like THF, and the like; followed by subsequent treatment with an alkylating agent of formula R⁵—I, where R⁵ is C₁₋₄alkyl (such as iodomethane or iodoethane); in the presence of base like K₂CO₃, Cs₂CO₃, and the like; in a aprotic solvent such as DMF, DMSO, and the like; affords a mixture of regio-isomeric esters of formula (XVIIa) and (XVIIb), where R¹ is an optionally substituted C₁₋₆alkyl. In a similar fashion, aryl Grignard reagents may be used to provide compounds of formula (XVIIa) and (VXIIb), where R¹ is a suitably substituted phenyl. The regio-isomers of formula (XVIIa) and (XVIIb) are not separated but are used directly and converted into the corresponding phthalazinone (mixture). For example, a mixture of formula (XVIIa) and formula (XVIIb) are treated with excess hydrazine; in a suitable solvent such as ethanol or methanol; at temperatures ranging from room temperature to 90° C.; for a period of 6 to 20 hours. The desired phthalazinone compound of formula (V) can be readily separated from the other regio-isomer by precipitation, crystallization, or purified by flash chromatography. Ullmann-type aromatic amination reaction of a compound of formula (V), with a suitably protected triazolone of formula (II), where R^{a} is C₁₋₆alkyl, and PG is selected from: benzyl, 4-methoxy benzyl, or an alkyl or aryl silane such as TBDPS, TBS, TES, or TIPS; in the presence of catalytic CuI and a diamine such as trans-1,2-diaminocyclohexane, and a base such as K₃PO₄, K₂CO₃, Cs₂CO₃, NaHCO₃, triethylamine, and the like; in a suitable solvent such as 1,4-dioxane, DMSO, DMF, THF, ACN, and the like; affords a compound of formula (XVIII), where X is N.

A compound of formula (XVIII), where R¹ is C₂₋₆alkenyl, is reacted under Simmons-Smith cyclopropanation reaction conditions known to one skilled in the art, to provide a compound of formula (XVIII) where R¹ is C₃₋₆cycloalkyl substituted with C₁₋₆alkyl. For example, a compound of formula (XVIII), where R¹ is C₂₋₆alkenyl, is reacted with diiodomethane; diethylzinc; in a suitable solvent such as toluene, and the like; at temperatures ranging from 0° C. to room temperature; for a period of 24 to 26 h; to provide a compound of formula (XVIII), where R¹ is cyclopropyl substituted with CH₃.

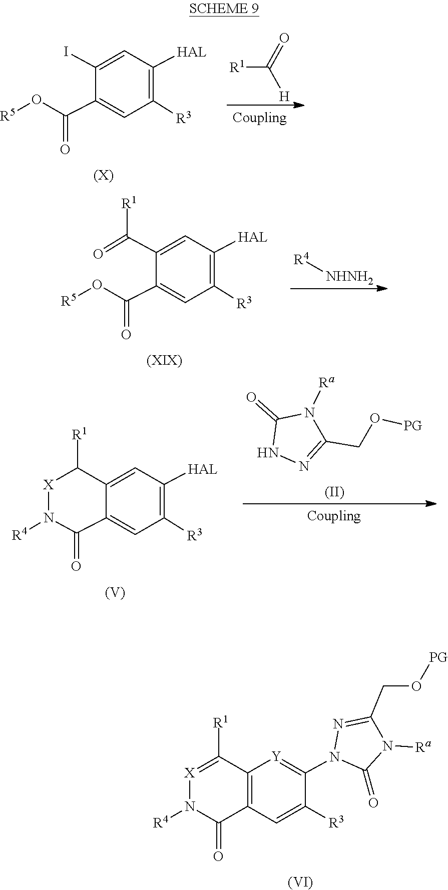

According to SCHEME 9, a compound of formula (X), where HAL is Br, R³ is H, and R⁵ is CH₃, is coupled in a palladium catalyzed carbonylation reaction with a commercially available or synthetically accessible aldehyde of formula R¹—CHO, where R¹ is C₁₋₆alkyl; to afford the corresponding ketone compound of formula (XIX), (similar transformation has been reported by Suchand et al, J. Org. Chem. 2016, 81, 6409-6423). For example, reaction of methyl 4-bromo-2-iodobenzoate with isobutylaldehyde; in the presence of a palladium catalyst such as Pd(OAc)₂; Ag₂O; and an oxidizing agent such as aqueous solution of tert-butyl hydroperoxide (TBHP); at a temperature of about 120° C.; for a period of 10-14 h; provided methyl 4-bromo-2-isobutylbenzoate. A ketone compound of formula (XIX) is reacted with hydrazine R⁴—NHNH₂, where R⁴ is suitably substituted aryl such as 2-chloro-6-fluorophenylhydrazine; to afford a compound of formula (V), where X is N. Ullmann-type aromatic amination reaction of a compound of formula (V) with a suitably protected triazolone (II) as previously described, affords a compound of formula (VI), where Y is CH, and R¹ is selected from C₁₋₆alkyl.

A compound of formula (VI), where Y is CH, and R¹ is phenyl and X is N, may be prepared in a similar fashion, employing methods previously describe by coupling methyl 4-bromo-2-iodobenzoate with a commercially available or synthetically accessible aldehyde of formula R¹—CHO, where R¹ is phenyl.

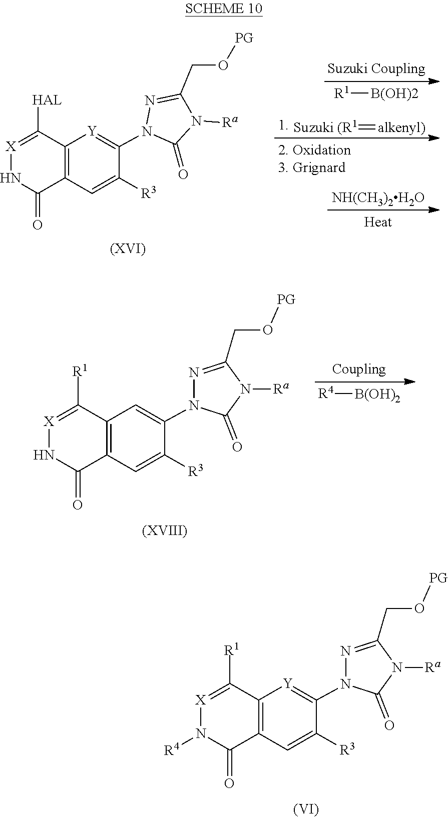

According to SCHEME 10, a compound of formula R¹—B(OH)₂; is reacted under Suzuki coupling conditions known to one skilled in the art, with a compound of formula (XVI), to provide a compound of formula (XVIII), where X is CH. For example, a compound of formula (XVI), where Y is CH and HAL is iodide, is reacted a commercially available or synthetically accessible boronic acid (or boronic ester) such as R¹—B(OH)₂, where R¹ is an optionally substituted C₂₋₆alkenyl, C₃₋₆cycloalkyl or aryl as defined herein with reference to Formula (Z); a palladium catalyst such as bis(triphenylphosphine)palladium(II) dichloride, and the like; a suitable base such a potassium phosphate, Cs₂CO₃, and the like; in a suitable solvent such as dioxane, water, ethanol, or a mixture thereof; to provide a compound of formula compound (XVIII), where X is CH. It has been noticed that during the coupling reaction as described above, loss of the iodide during the reaction conditions afforded a compound of formula compound (XVIII), where X is CH, and R¹ is H. A compound of formula (XVIII), where X is CH or N, is reacted with a compound of formula R⁴—B(OH)₂; under copper (II) mediated Chan-Lam coupling conditions known to one skilled in the art, or as previously described, to provide a compound of formula (VI), where X is CH or N, R¹ is optionally substituted C₂₋₆alkenyl, R³ is H or F, and R⁴ is a suitably substituted phenyl as described herein with reference to Formula (Z).

A compound of formula (XVIII), where R¹ is N(CH₃)₂ is prepared from a compound of formula (XVI), where HAL is Br and PG is Bn. Reaction of a compound of formula (XVI) with an amine bs such as NH(CH₃)₂ in water; at a temperature of about 110° C.; for a period of 96 hours h; affords a compound of formula (XVIII) where R¹ is N(CH₃)₂, and R^{a} is C₁₋₆alkyl. A compound of Formula (Z), where R¹ is N(CH₃)₂ is prepared according to methods described above.

A compound of formula (XVIII), where R¹ is C₁₋₆alkyl substituted with OH, is prepared from a compound of formula (XVIII), where R¹ is C₂₋₆alkenyl, and PG is Bn in two steps. In a

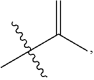 [SMILES: *C(=C)C]

first step, reaction of a compound of formula (XVIII), where R¹ is, under oxidizing conditions such as NaIO₄, and K₂OsO₄·2H₂O or OsO₄; in a suitable solvent such as THF/H₂O; at temperatures ranging from 0° C. to room temperature; for a period of 48 to 72 hours; affords a ketone intermediate compound. In a second step, reaction of the ketone intermediate compound with a Grignard reagent such as methylmagnesium bromide; in a suitable solvent such as diethyl ether; at temperatures ranging from 0° C. to room temperature; for a period of 3 to 30 hours; affords a compound of formula (XVIII), where R¹ is C₁₋₆alkyl substituted with OH.

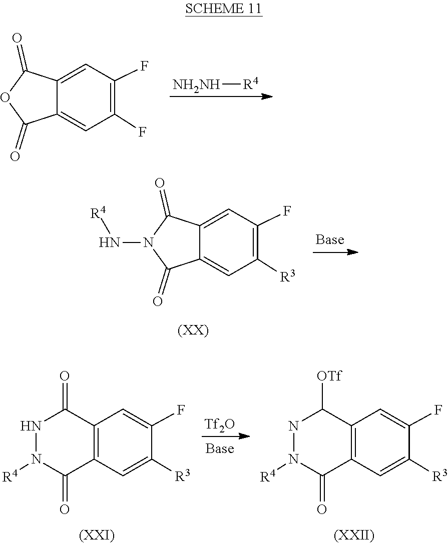

According to SCHEME 11, 4,5-difluorophthalic anhydride is reacted with a hydrazine compound of formula R⁴—NHNH₂, where R⁴ is a suitably substituted phenyl or heteroaryl such as (2-chloro-6-fluorophenyl)hydrazine hydrochloride; in acetic acid; at a temperature of about 125° C.; for a period of about 1.5 h to afford a compound of formula (XX), where R³ is F. Rearrangement of a compound of formula (XX) affords a ring expansion compound of formula (XXI), under basic conditions such as sodium ethoxide or sodium methoxide; in a protic solvent such as ethanol, methanol, and the like; at room temperature; for a period of about 1.5 h. Derivation of a compound of formula (XXI), with a sulfonate-based leaving group such as trifluoromethanesulfonyl (triflate), is achieved by is by reaction with a triflating agent such as trifluoromethanesulfonic anhydride (Tf₂O), a base such as triethylamine (TEA), pyridine, and the like, in a suitable solvent such as DCM and the like, to provide a compound of formula (XXII). Milder triflating agents such as N-phenylbis(trifluoromethanesulfonimide) (TF₂NPh), a base such as TEA, DIEA, and the like, in a suitable solvent such as DCM, and the like; may be used.

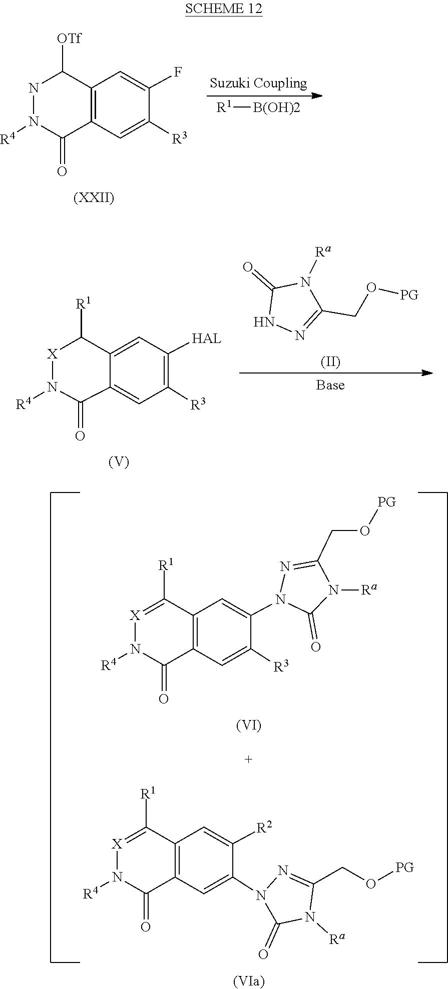

According to SCHEME 12, a compound of formula R¹—B(OH)₂; is reacted under Suzuki coupling conditions previously described, with a compound of formula (XXII), to provide a compound of formula (V), where X is N. For example, a compound of formula (XXII), is reacted a commercially available or synthetically accessible boronic acid (or boronic ester) such as R¹—B(OH)₂, where R¹ is C₂₋₆alkenyl or C₂₋₆haloalkenyl as defined herein with reference to Formula (Z); a palladium catalyst such as 1,1′-bis(diphenylphosphino)ferrocene-palladium(II)dichloride or bis(triphenylphosphine)palladium(II) dichloride, and the like; a suitable base such a potassium phosphate, Cs₂CO₃, K₂CO₃, and the like; in a suitable solvent such as dioxane, water, ethanol, or a mixture thereof; to provide a compound of formula compound (V). A compound of formula (V), where R¹ is C₂₋₆alkyl or C₂₋₆haloalkyl, is readily prepared by selective hydrogenation of a compound of formula (V), where R¹ is C₂₋₆alkenyl or C₂₋₆haloalkenyl. For example, reaction of a compound of formula (V), where R¹ is

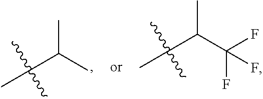

under hydrogenation conditions employing a catalyst such as Pd/C and the like, in a suitable solvent such as EtOAc, and the like; under an atmosphere of hydrogen gas (20-45 psi) at room temperature; for a period of 4 to 24 hours; affords a compound of formula (V), where R¹ is

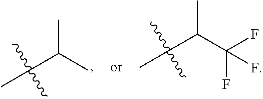

The reaction of a compound of formula (V), with a suitably protected triazolone of formula (II), employing conditions previously described, affords a mixture of compounds of formula (VI) and (VIa) which can be separated before or after deprotection of the protecting group.

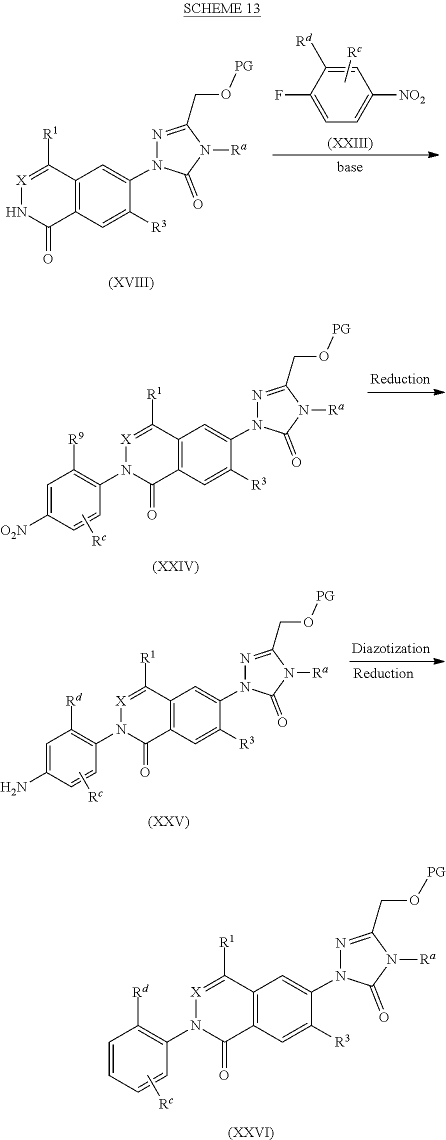

According to SCHEME 13, N-arylation of a compound of formula (XVIII) is achieved by reaction of suitably substituted commercially available or synthetically accessible fluoro compound of formula (XXIII), where R^{c} and R^{d} are as defined herein with reference to Formula (Z). A compound of formula (XVIII), where R¹ is H, C₂₋₆alkenyl, C₂₋₆haloalkenyl, C₃₋₆cycloalkyl, C₃₋₆cycloalkyl substituted with C₁₋₆alkyl, and X is CH or N, is reacted under nucleophilic displacement reaction conditions, with a commercially available or synthetically accessible fluoro compound of formula (XXIII); in the presence of a base like K₂CO₃, Cs₂CO₃, and the like; in aprotic solvent such as DMF, DMSO, and the like; at temperatures ranging from 65 to 100° C.; to afford a compound of formula (XXIV).

Reduction of compound of formula (XXIV) is achieved employing zinc or iron and NH₄Cl; in a mixed solvent of methanol and water; to provide an amino compound of formula (XXV).

Diazotization of a compound in formula (XXV) with NaNO₂; in an acidic aqueous solution or other nitrite reagents; in an organic solvent, such as EtOH, and the like; at a temperature of 0° C.; and subsequent the reduction of diazo group with zinc at temperatures ranging from 0 to 85° C.; or by treatment with H₃PO₂; affords a compound of formula (XXVI), where R^{c} and R^{d} are as defined as described herein with reference to Formula (Z).

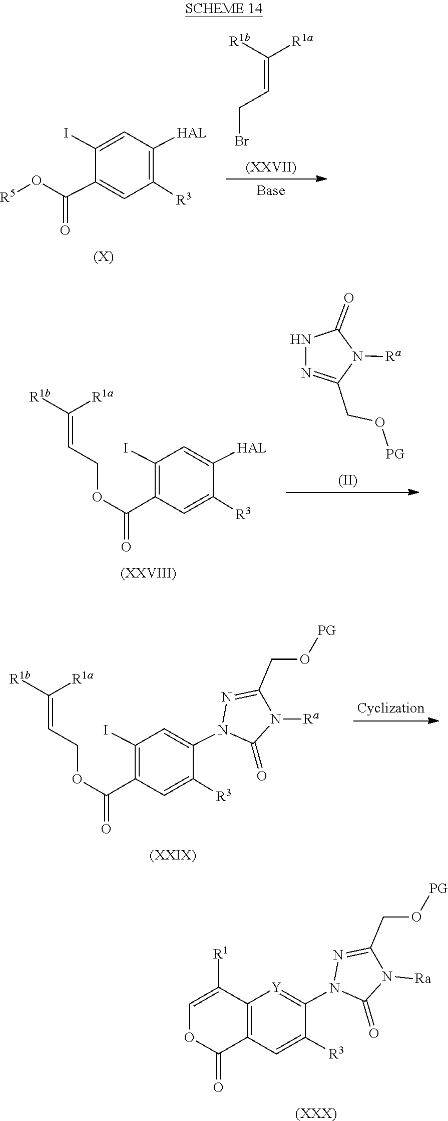

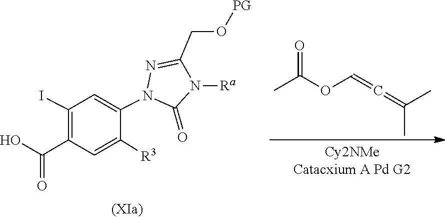

According to SCHEME 14, a compound of formula (X), where HAL is F, R⁵ is H and R³ is F, is reacted with a commercially available or synthetically accessible compound of formula (XXVII), where R^{1a} and R^{1b} are each independently H or C₁₋₄alkyl, such as 1-bromo-3-methyl-2-butene; in the presence of a base such as K₂CO₃, Cs₂CO₃, and the like; in a suitable solvent such as DMSO, DMF, THF, ACN, and the like; to afford an ester compound of formula (XXVIII), where R³ is F, and HAL is F. A compound of formula (XXVIII), where R^{1a} and R^{1b} are each independently selected from C₁₋₄haloalkyl or C₃₋₆cycloalkyl may be made in a similar fashion. The reaction of an ester of formula (XXVIII) with a suitably protected triazolone compound of formula (II); in the presence of a base such as K₃PO₄, K₂CO₃, Cs₂CO₃, NaHCO₃, triethylamine, and the like; in a suitable solvent such as 1,4-dioxane, DMSO, DMF, THF, ACN, and the like; affords a compound of formula (XXIX). In a preferred method, PG is Bn, and R^{a} is C₁₋₆alkyl. A compound of formula (XXIX), where R³ is H or F, undergoes intramolecular cyclization under Heck reaction conditions, such as employing at catalyst such as chloro[(tri-tert-butylphosphine)-2-(2-aminobiphenyl)] palladium(II) (P(tBu₃)PdG₂), N-cyclohexyl-N-methyl-cyclohexanamine, in a suitable solvent such as toluene, and the like; at a temperature of about 15 to 80° C.; for a period of about 18 to 36 hours; to provide an isocoumarin compound of formula (XXX), where Y is CH and R¹ is isopropyl, R³ is H or F, R^{a} and PG are defined as described above.

An isocoumarin compound of formula (XXX), where R¹ is

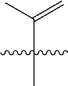 [SMILES: *C(=C)C]

is prepared from a compound of formula (XIa) and methylbuta-1,2-dien-1-yl acetate. Methylbuta-1,2-dien-1-yl acetate is commercially available or prepared in two steps from 2-methyl-3-butyn-2-ol. Acetic anhydride is reacted with 2-methyl-3-butyn-2-ol, in the presence of a catalyst such as Mg(ClO₄)₂; in a suitable solvent such as DCM, and the like; to afford 2-methylbut-3-yn-2-yl acetate. 2-Methylbut-3-yn-2-yl acetate is reacted with a catalytic amount of a Lewis acid such as AgBF₄, AgClO₄, PtCl₂, and the like; to provide 3-methylbuta-1,2-dien-1-yl acetate. 3-Methylbuta-1,2-dien-1-yl acetate is coupled with a compound of formula (XIa), where R⁵ is H, employing intermolecular cyclization under Heck reaction conditions as previously described, such as employing at catalyst such as Catacxium A Pd G2, _{and} Cy₂NMe ₚalladium (II) acetate, phase transfer reagent like tetrabutylammonium bromide, and a base like potassium acetate, in a suitable solvent such as DMF, and the like; at a temperature of 70 to 90° C.; for a period of 10 to 16 hours; to provide the isocoumarin compound of formula (XXX), where Y is CH and R¹ is

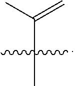 [SMILES: *C(=C)C]

A compound of formula (XXX), where Y is CH and R¹ is

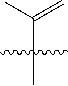 [SMILES: *C(=C)C]

is selectively reduced under hydrogenation conditions employing at catalyst such as Wilkinson's Catalyst [RhCl(PPh₃)₃] and the like, in a suitable solvent such as THF, and the like; at room temperature, provide an isocoumarin compound of formula (XXX), where R¹ is isopropyl.

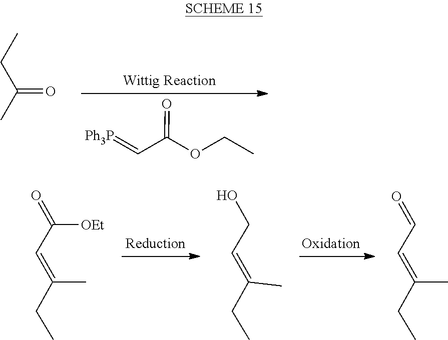

According to SCHEME 15, 2-butanone is converted to ethyl 3-methylpent-2-enoate employing Wittig reaction conditions known to one skilled in the art. For example, 2-butanone is reacted with a triphenyl phosphonium ylide such as (carbethoxymethylene) triphenylphosphorane, with or without an additive such as benzoic acid, LiCl, and sodium dodecyl sulfate (SDS), and the like, in a suitable solvent such as toluene, at temperatures ranging from rt to the reflux temperature of the solvent, for a period of 12-24 h. Ethyl 3-methylpent-2-enoate is reduced to 3-methylpent-2-en-1-ol employing a suitable reducing agent such as DIBAL-H, in a suitable solvent such as toluene, and the like, at temperatures ranging from −78° C. to room temperature. 3-Methylpent-2-en-1-ol is oxidized to 3-methylpent-2-enal employing oxidation conditions known to one skilled in the art, for example, DMP (Dess-Martin periodinane), SO₃-pyridine, Swern conditions [(COCl)₂, DMSO, Et₃N], PCC, and the like, in a solvent such as EtOAc, DMSO, DCM, and the like, at temperatures ranging from about −78° C. to room temperature (about 23° C.). In a preferred method, 3-methylpent-2-en-1-ol is oxidized to 3-methylpent-2-enal with Dess-Martin periodinane, in DCM, at 25° C. for a period of 1-4 h.

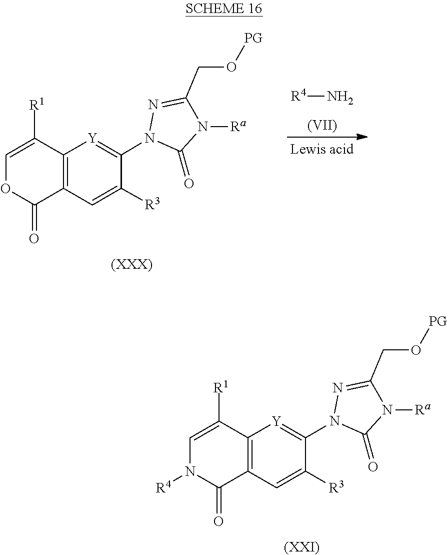

According to SCHEME 16, an isocoumarin of compound of formula (XXX), where Y is CH, is reacted with a commercially available or synthetically accessible amine compound of formula R⁴—NH₂, where R⁴ is as defined herein with reference to Formula (Z); a Lewis acid such as like AlMe₃, AlCl₃, and the like; in a suitable aprotic solvent such as DCM, toluene, and the like; to provide a compound of formula (XXXI), where Y is CH, and R¹, R³, R⁴ and R^{a} are defined as described herein with reference to Formula (Z).

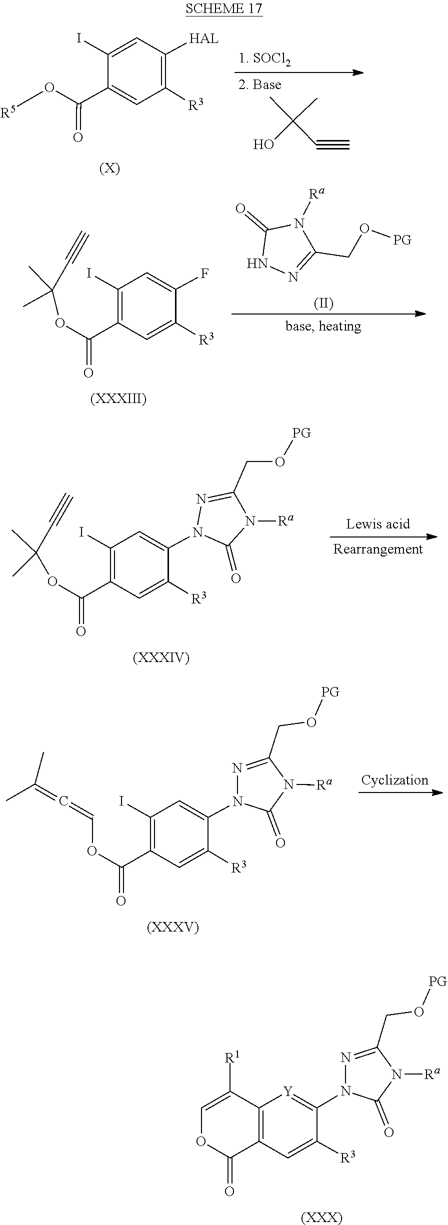

An isocoumarin compound of formula (XXX) may be prepared according to SCHEME 17. 4,5-Difluoro-2-iodobenzoyl chloride is prepared from a compound of formula (X), where HAL is F, R⁵ is H and R³ is F, employing conditions known to one skilled in the art such as oxalyl chloride or thionyl chloride, in the presence of a catalytic amount of DMF, in a suitable solvent such as an aprotic non-polar solvent such as dichloromethane (DCM), tetrahydrofuran (THF), acetonitrile (ACN), toluene, and the like, at a temperatures ranging from 0° C. to room temperature to form 4,5-difluoro-2-iodobenzoyl chloride. 4,5-Difluoro-2-iodobenzoyl chloride may be reacted with commercially available or synthetically accessible 2-methylbut-3-yn-2-ol, in the presence of a base such as triethylamine and DMAP; in a suitable solvent such as DCM, and the like; to afford an ester compound of formula (XXXIII). A compound of formula (XXXIII) may be reacted with a compound of formula (II), employing methods as previously described to afford a compound of formula (XXXIV). Treatment of a compound of formula (XXXIV) with a catalytic amount of a Lewis acid such as AgClO₄, PtCl₂, and the like; may afford the rearranged compound of formula (XXXV). A compound of formula (XXXV), where R³ is H or F, may undergo an intramolecular cyclization under Heck reaction conditions, such as employing a catalyst such as palladium (II) acetate, a phase transfer reagent like tetrabutylammonium bromide, and a base like potassium acetate, in a suitable solvent such as DMF, and the like; at a temperature of 70 to 90° C.; for a period of 1 to 3 hours; to provide an isocoumarin compound of formula (XXX), where Y is CH.

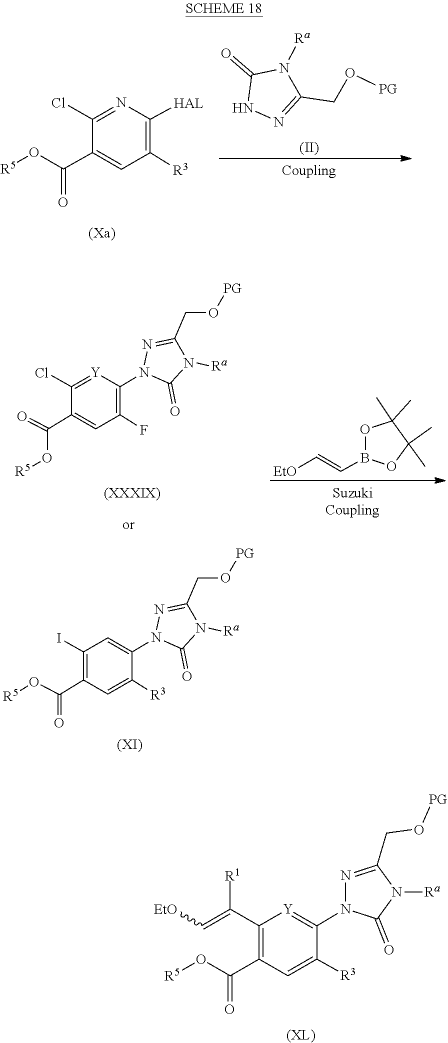

According to SCHEME 18, a compound of formula (Xa), where HAL is C₁, R is CH(CH₃)₂, and R³ is F, is commercially available or synthetically accessible according to methods as described in Chen, et al, US Patent Publication No. US2016-0176869. Reaction of a compound of formula (Xa) with a commercially available or synthetically accessible nucleophilic compound of formula (II), where PG is benzyl, and R^{c} is C₁₋₆alkyl; in the presence of a base such as K₂CO₃, Cs₂CO₃, NaHCO₃, triethylamine, and the like; in a suitable solvent such as dimethylsulfoxide (DMSO), DMF, THF, ACN, and the like; affords a compound of formula (XXXIX), where Y is N. A compound of formula (XXXIX) or formula (XI), where R³ is F, and R⁵ is C₁₋₄alkyl; is reacted a commercially available 1-ethoxyethene-2-boronic acid pinacol ester; a palladium catalyst such as bis(triphenylphosphine)palladium(II) dichloride, 1,1′-bis(diphenylphosphino)ferrocene-palladium(II)dichloride and the like; a suitable base such as Cs₂CO₃, and the like; in a suitable solvent such as dioxane, water, ethanol, or a mixture thereof; employing conventional or microwave heating; to provide a compound of formula (XL), where Y is N or CH.

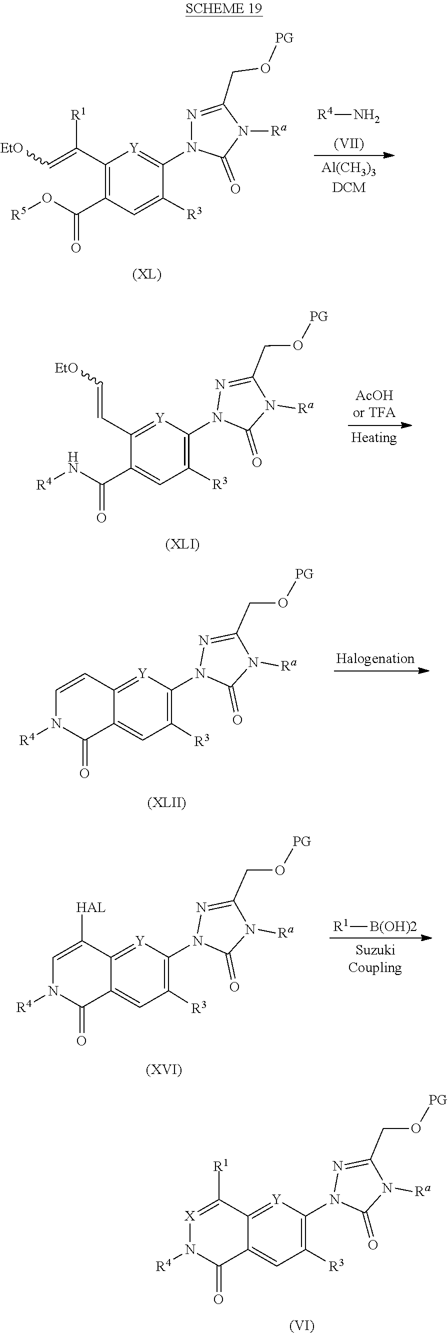

According to SCHEME 19, a compound of formula R⁴—NH₂, where R⁴ is as defined herein with reference to Formula (Z); is reacted with trimethyl aluminum; in a suitable solvent such as dichloromethane, toluene, or a mixture thereof; the resulting solution is combined with a compound of formula (XL), where Y is CH or N; to provide a compound of formula (XLI). A compound of formula (XLI), where Y is CH or N, is treated with acetic acid or trifluoroacetic acid under heating conditions between 50° C. to 90° C., to provide a compound of formula (XLII). A compound of formula (XLII) is halogenated employing N-bromosuccinimide in anhydrous dimethylformamide at room temperature, to provide a compound of formula (XVI), where HAL is Br. A compound of formula R¹—B(OH)₂; is reacted under Suzuki coupling conditions known to one skilled in the art, or as previously described with a compound of formula (XVI), to provide a compound of formula (VI), where R¹ is an optionally substituted C₂₋₆alkenyl, C₂₋₆haloalkenyl, or aryl as defined herein with reference to Formula (Z). A compound of formula (VI), where R¹ is an optionally substituted C₂₋₆alkenyl or C₂₋₆haloalkenyl is reacted under hydrogenation conditions using Wilkinson catalyst ((PPh₃)₃RhCl) to provide a compound of formula (VI), where R¹ is C₂₋₆alkyl or C₂₋₆haloalkyl.

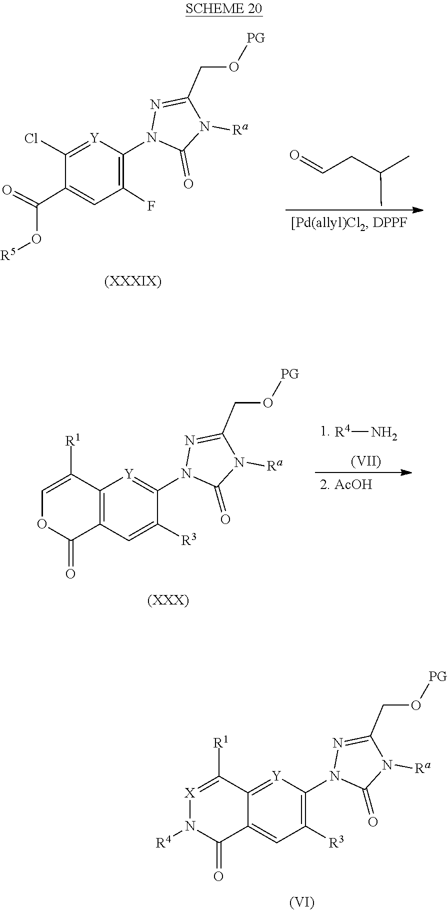

According to SCHEME 20, 3-methylbutanal is reacted with a compound of formula (XXXIX), where Y is N and R⁵ is CH(CH₃)₂, with a palladium catalyst such as allylpalladium(II) chloride dimer, and the like; a ligand such as 1,1′-bis(diphenylphosphino)ferrocene (dppf), and the like; a suitable base such as Cs₂CO₃, and the like; in the presence of water scavenger such as molecular sieve (4A); in a suitable solvent such as dioxane thereof; to provide a compound of formula compound (XXX), where R¹ is isopropyl. A compound of formula R⁴—NH₂, where R⁴ is as defined herein with reference to Formula (Z); is reacted with trimethyl aluminum; in a suitable solvent such as dichloromethane, toluene, or a mixture thereof; the resulting solution is combined with a compound of formula (XXX), followed by subsequent treatment with acetic acid under heating temperature of 80-100° C. for a period of time ranging from 5 to 24 hours; to provide a compound of formula (VI); where X is CH, Y is N, R¹ is isopropyl, R³ is F.

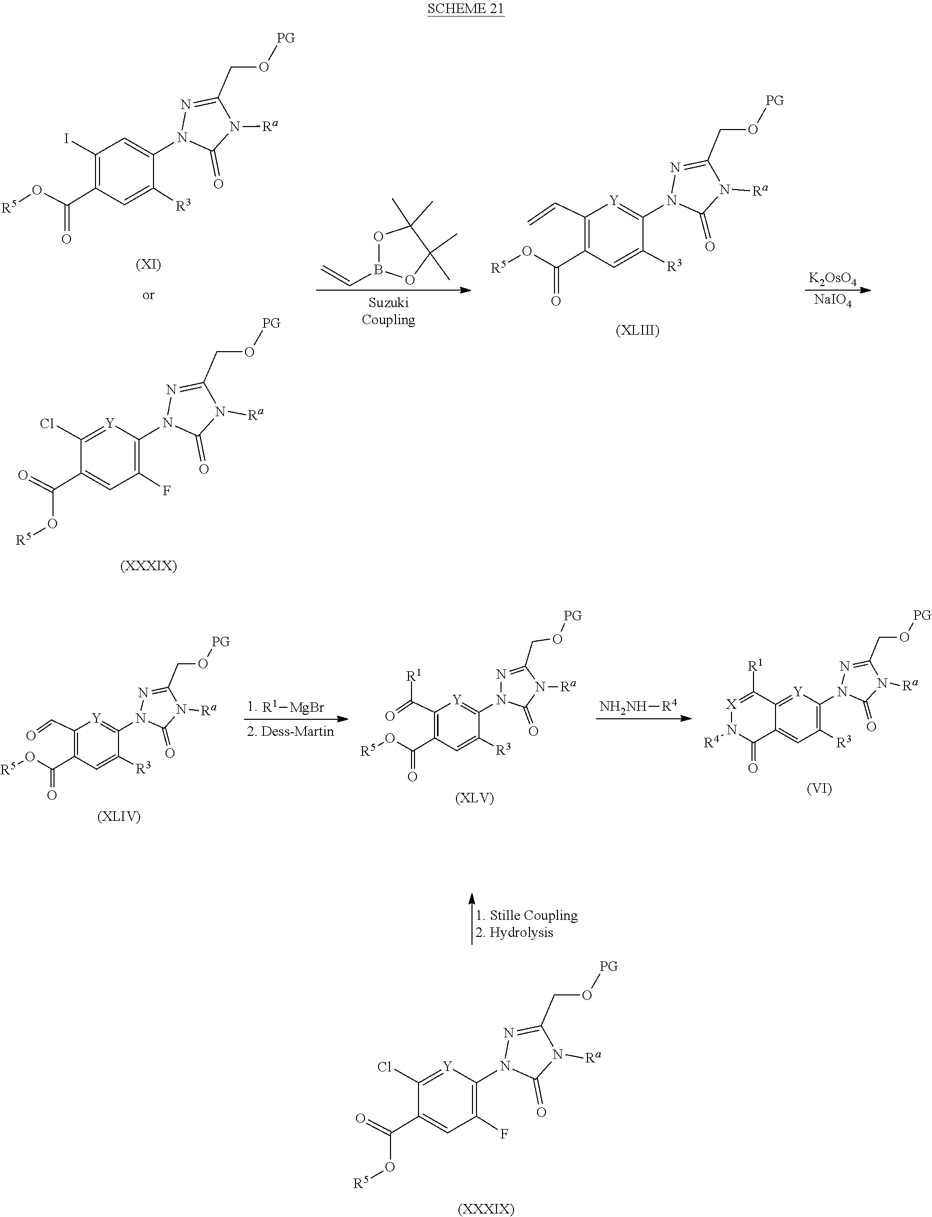

According to SCHEME 21, A compound of formula (XXXIX) or formula (XI), where R³ is F, and R⁵ is C₁₋₄alkyl; is reacted a commercially available vinylboronic acid pinacol ester; a palladium catalyst such as bis(triphenylphosphine)palladium(II) dichloride, or 1,1′-bis(diphenylphosphino)ferrocene-palladium(II)dichloride dichloromethane complex, and the like; a suitable base such as Cs₂CO₃, and the like; in a suitable solvent such as dioxane, water, ethanol, or a mixture thereof; to provide a compound of formula compound (XLIII), where Y is N or CH. The vinyl group in a compound of formula (XLIII) is selectively converted into an aldehyde group of formula (XLIV) employing potassium osmate (VI) dihydrate/sodium periodate, or ozonolysis, and the like. A compound of formula (XLIV) is reacted with a commercially available or synthetically accessible suitably substituted alkyl Grignard reagent such as i-PrMgCl, and the like; in aprotic solvent like THF, and the like; followed by subsequent treatment with an oxidizing reagent such as Dess-Martin reagent, or Swern oxidation conditions, and the like; to afford a ketone compound of formula (XLV).

A compound of formula (XLV) is prepared from a compound of formula (XXXIX) in two steps. A compound of formula (XXXIX), where R³ is F, and R⁵ is C₁₋₄alkyl; is reacted a commercially available tributyl(1-ethoxyvinyl)tin; a palladium catalyst such as bis(triphenylphosphine)palladium(II) dichloride, 1,1′-bis(diphenylphosphino)ferrocene-palladium(II)dichloride and the like; in a suitable solvent such as dioxane, water, ethanol, or a mixture thereof. Subsequent acidic hydrolysis employing conditions such as treatment with aqueous HCl solution at room temperature affords a compound of formula (XLV), where X is N, Y is N or CH, R¹ is methyl.

A commercially or synthetically available hydrazine R⁴—NHNH₂, where R⁴ is as defined herein with reference to Formula (Z), such as 2-chloro-6-fluorophenylhydrazine, o-tolylhydrazine; is condensed with a compound of formula (XLV); in the presence of a base such as potassium carbonate, and the like; under the heating conditions such as 70-120° C.; in a suitable solvent such as toluene, or a mixture thereof; afford a compound of formula (VI), where X is N, Y is CH or N, and R⁴ is as defined herein with reference to Formula (Z).

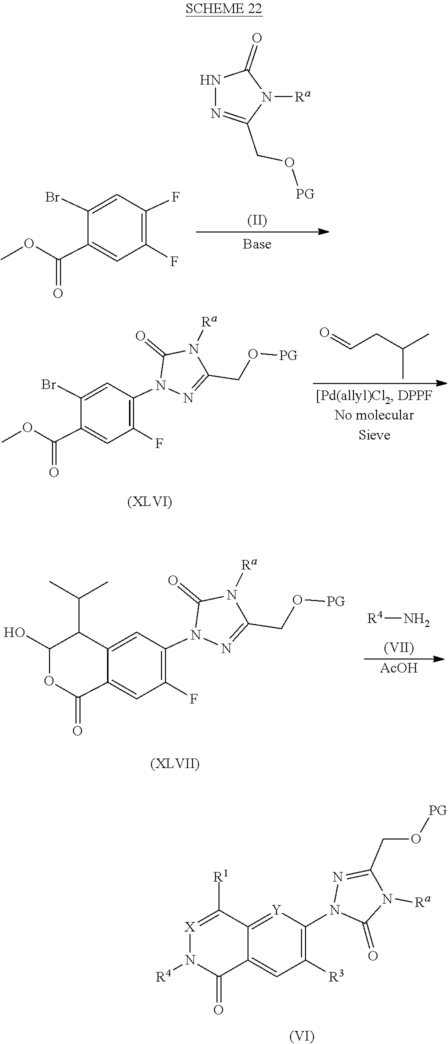

According to SCHEME 22, the reaction of methyl 2-bromo-4,5-difluorobenzoate with a suitably protected triazolone compound of formula (II); in the presence of a base such as K₃PO₄, K₂CO₃, Cs₂CO₃, NaHCO₃, triethylamine, and the like; in a suitable solvent such as 1,4-dioxane, DMSO, DMF, THF, ACN, and the like; affords a compound of formula (XLVI). In a preferred method, PG is Bn, and R^{a} is C₁₋₆alkyl (as previously described in Scheme 14). 3-Methylbutanal is reacted with a compound of formula (XLVI), with a palladium catalyst such as allylpalladium(II) chloride dimer, and the like; a ligand such as 1,1′-bis(diphenylphosphino)ferrocene (dppf), and the like; a suitable base such as Cs₂CO₃, and the like; in the absence of water scavenger such as molecular sieve (4A); in a suitable solvent such as dioxane thereof; to provide a compound of formula compound (XLVII). A compound of formula R⁴—NH₂, where R⁴ is as defined herein with reference to Formula (Z); is reacted with trimethyl aluminum; in a suitable solvent such as dichloromethane, dichloroethane, toluene, or a mixture thereof; the resulting solution is combined with a compound of formula (XLVII), followed by subsequent treatment with acetic acid under heating temperature of 80-100° C. for a period of time ranging from 5 to 24 hours; to provide a compound of formula (VI); where X is CH, Y is CH, R¹ is isopropyl, R³ is F. In certain cases, a compound of formula R⁴—NH₂ such as o-toluidine and the like; is directly condensed with a compound of formula compound (XLVII) in acetic acid under heating temperature of 80-100° C. for a period of time ranging from 10 to 24 hours; to provide a compound of formula (VI); where X, Y, R¹, R³ are defined above.

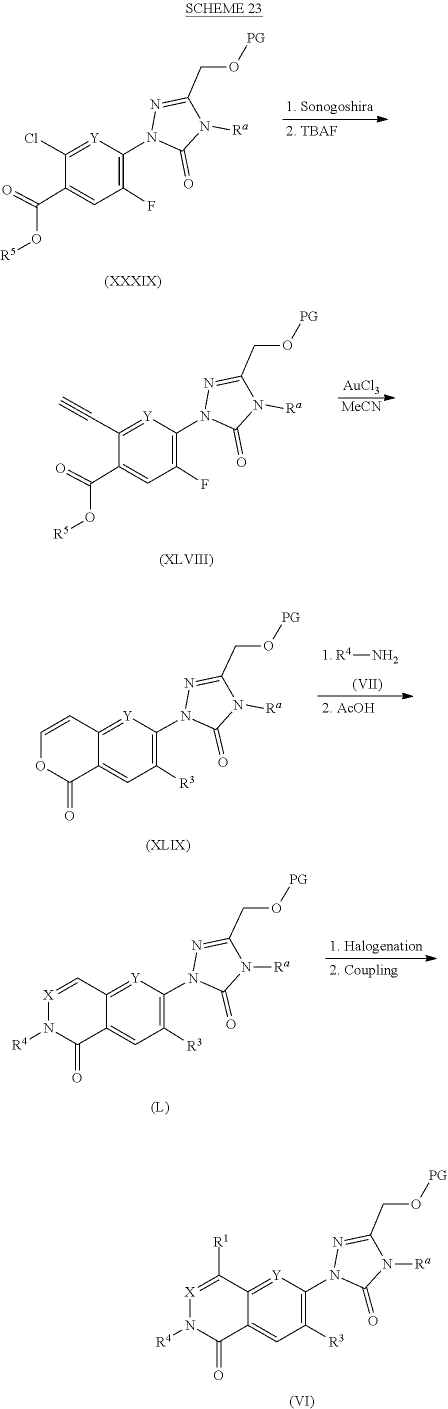

According to SCHEME 23, a compound of formula (XXXIX), where R⁵ is C₁₋₄alkyl, Y is N, and R³ is F, is subjected to a Sonogashira coupling reaction with a silyl protected alkyne, such as trimethylsilylacetylene, a palladium catalyst such as palladium(II)bis(triphenylphosphine) dichloride and the like; a copper catalyst such as copper iodide and the like; with a suitable base, such as triethylamine; in a suitable solvent such as ACN, toluene, and the like. Deprotection reaction employing TBAF in a suitable solvent such as THF, and the like; at room temperature affords a compound of formula (XLVIII). A compound of formula (XLIX) is obtained using a gold catalyst, preferably AuCl₃ in a suitable solvent mixture, such as MeCN. Similar transformation by AuCl₃-catalyzed cyclization has been described by Marchal, E. et al in Tetrahedron 2007, 63, 9979-9990. A compound of formula R⁴—NH₂, where R⁴ is as defined herein with reference to Formula (Z); is reacted with trimethylaluminum; in a suitable solvent such as dichloromethane, dichloroethane, toluene, or a mixture thereof; the resulting solution is combined with a compound of formula (XLIX), followed by subsequent treatment with acetic acid under heating temperature of 80-100° C. for a period of time ranging from 5 to 24 hours; to provide a compound of formula (L). Employing NBS in a suitable solvent, preferably DMF, at room temperature followed by cross coupling using conditions known to one skilled in the art, preferably a palladium catalyst such as palladium(II)bis(triphenylphosphine) dichloride, a base such as Cs₂CO₃, Na₂CO₃, and the like; in a solvent mixture composed of 1,4-dioxane and water; at a temperature of 100° C. provides a compound of formula (VI), where X is CH, Y is N, R³ is F, and R¹, R^{a} and PG are defined as previously described.

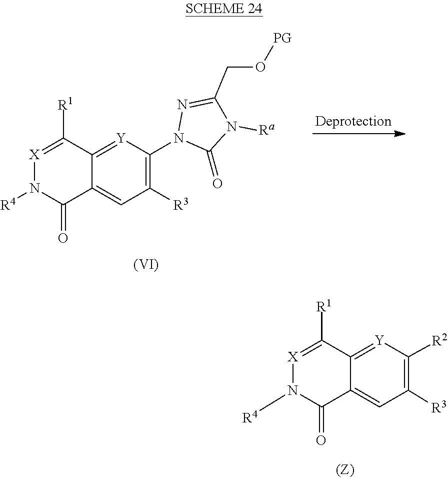

According to SCHEME 24, a compound of formula (VI), where PG is Bn, is deprotected employing conditions known to one skilled in the art, preferably in neat TFA in a sealed tube, at a temperature of about 60 to 90° C.; or employing BCl₃, at a temperature of about −78° C., in a suitable solvent such as in DCM; or treatment with hydrogen gas, in the presence of a catalyst such as Palladium on carbon (Pd/C), affords a compound of Formula (Z).

In a similar fashion, N-arylation and in-situ TBDPS deprotection of a compound of formula (XVIII), where R¹ is I and PG is TBDPS, and X is N; is achieved employing conditions known to one skilled in the art or as previously described, to afford a compound of Formula (Z).

A compound of Formula (Z), where R³ is F is reacted in a nucleophilic aromatic substitution reaction to provide a compound of Formula (Z), where R³ is OCH₃. For example, reaction of a compound of Formula (Z), where R³ is F, with a suitable base such as NaOH, and the like; in a suitable solvent such as MeOH, and the like; to provide a compound of Formula (Z) where Y is CH and R³ is OCH₃.

Compounds of Formula (Z) may be converted to their corresponding salts using methods known to one of ordinary skill in the art. For example, an amine of Formula (Z) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (Z) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate compounds of Formula (Z) and various preferred embodiments.

### Examples

In obtaining the compounds of Formula (Z) described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:
METHOD A. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (with 0.225% FA) over 10 min and then hold at 100% ACN for 2 min, at a flow rate of 25 mL/min; or
METHOD B. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.1% TFA) over 10 min and then hold at 100% ACN for 2 min, at a flow rate of 25 mL/min; or
METHOD C. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.05% HCl) over 10 min and then hold at 100% ACN for 2 min, at a flow rate of 25 mL/min; or
METHOD D. a Gilson GX-281 semi-prep-HPLC with Phenomenex Gemini C18 (10 μm, 150×25 mm), AD (10 μm, 250 mm×30 mm), or Waters XBridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min; or
METHOD E. a Gilson GX-281 semi-prep-HPLC with Phenomenex Gemini C18 (10 μm, 150×25 mm), or Waters XBridge C18 column (5 μm, 150×30 mm), mobile phase of 5-99% ACN in water (10 mM NH₄HCO₃) over 10 min and then hold at 100% ACN for 2 min, at a flow rate of 25 mL/min.

Preparative supercritical fluid high performance liquid chromatography (SFC) was performed either on a Thar 80 Prep-SFC system, or Waters 80Q Prep-SFC system from Waters. The ABPR was set to 100 bar to keep the CO2 in SF conditions, and the flow rate may verify according to the compound characteristics, with a flow rate ranging from 50 g/min to 70 g/min. The column temperature was ambient temperature.

Mass spectra (MS) were obtained on a SHIMADZU LCMS-2020 MSD or Agilent 1200\G6110A MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, dd=doublet of doublets, ddd=doublet of doublet of doublets, td=triplet of doublets, dt=doublet of triplets, spt=septet, quin=quintet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, MA) or ACD/Name Version 10.01 (Advanced Chemistry). Compounds designated as R* or S* are enantiopure compounds where the absolute configuration was not determined.

### Intermediate 1: 3-((Benzyloxy)methyl)-4-ethyl-1H-1,2,4-triazol-5(4H)-one

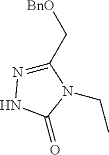 [SMILES: CCn1c(COCc2ccccc2)n[nH]c1=O]

### Step A. 2-(Benzyloxy)acetohydrazide

To a solution of ethyl 2-(benzyloxy)acetate (55 g, 283.17 mmol) in EtOH (500 mL) was added NH₂NH₂·H₂O (28.3 g, 566 mmol, 27.5 mL). The reaction mixture was heated at 78° C. for 6 h. The reaction mixture was concentrated under reduced pressure to afford the title product (52 g, crude) as a colorless oil, which was used directly in the next step without further purification.

### Step B. 3-((Benzyloxy)methyl)-4-ethyl-1H-1,2,4-triazol-5(4H)-one

To a solution of 2-(benzyloxy)acetohydrazide (52 g, 288 mmol) in H₂O (500 mL) was added dropwise isocyanatoethane (25.1 g, 346 mmol, 27.9 mL) at 0° C. After the addition was complete, the mixture was stirred at 25° C. for 12 hr. To the mixture was added H₂O (20 mL), and an aqueous solution (120 mL) of NaOH (57.7 g, 1.44 mol). The mixture was stirred at 95° C. for 12 hr. The reaction mixture was cooled to rt, then quenched with HCl (12 M) at 0° C. and adjusted to “pH” 6. The solid was filtered and dried under reduced pressure to afford the title compound as a white solid (61 g, 91% yield). ¹H NMR (400 MHz, CDCl₃) δ −9.23-9.09 (m, 1H), −7.41-7.31 (m, 5H), −4.58-4.53 (m, 2H), −4.45-4.42 (m, 2H), −3.82-3.75 (m, 2H), −1.33-1.29 (m, 3H) ppm.

### Intermediate 2: 5-(((tert-Butyldiphenylsilyl)oxy)methyl)-4-ethyl-2,4-dihydro-3H-1,2,4-triazol-3-one

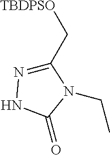

### Step A. 4-Ethyl-5-(hydroxymethyl)-2,4-dihydro-3H-1,2,4-triazol-3-one

To a solution of 5-[(benzyloxy)methyl]-4-methyl-2,4-dihydro-3H-1,2,4-triazol-3-one (8 g, 34.3 mmol, 1.0 eq.) in methanol (200 mL) was added Pd/C (2 g). The resulting mixture was maintained under hydrogen and stirred at rt for 6 h. Then the resulting mixture was filtered and the filtrate was concentrated to afford the crude product 4-ethyl-5-(hydroxymethyl)-2,4-dihydro-3H-1,2,4-triazol-3-one as a white solid (4.3 g, 88% yield). ¹H NMR (400 MHz, DMSO-d₆) δ 11.52 (s, 1H), 5.55 (t, J=5.50 Hz, 1H), 4.32 (d, J=5.50 Hz, 2H), 3.64 (q, J=6.97 Hz, 2H), 1.18 (t, J=6.97 Hz, 3H) ppm.

### Step B. 5-(((tert-Butyldiphenylsilyl)oxy)methyl)-4-ethyl-2,4-dihydro-3H-1,2,4-triazol-3-one

To a solution of 4-ethyl-5-(hydroxymethyl)-2,4-dihydro-3H-1,2,4-triazol-3-one (3 g, 21 mmol, 1.0 eq.) in DCM (30 mL) was added tert-butylchlorodiphenylsilane (6.5 mL, 25 mmol, 1.2 eq.) and pyridine (1.86 mL, 23 mmol, 1.1 eq.). The resulting mixture was stirred at rt overnight. The reaction mixture was quenched with water (100 mL). The resulting mixture was extracted with DCM (3×100 mL). The organic layers were combined, dried over anhydrous sodium sulfate, filtered and concentrated. The residue was purified by silica gel chromatography (SiO₂, 50-80% ethyl acetate/petroleum ether) to afford 5-(((tert-butyldiphenylsilyl)oxy)methyl)-4-ethyl-2,4-dihydro-3H-1,2,4-triazol-3-one as a white solid (4.9 g, 61% yield). LCMS (ES-API): mass calcd. for C₂₁H₂₇N₃O₂Si, 381.2; m/z found, 382.2 [M+H]⁺. ¹H NMR (400 MHz, CDCl₃) δ 9.98 (s, 1H), 7.61-7.72 (m, 4H), 7.32-7.54 (m, 6H), 4.54 (s, 2H), 3.84 (q, J=7.34 Hz, 2H), 1.33 (t, J=7.34 Hz, 3H), 1.07 (s, 9H) ppm.

### Intermediate 3: 5-((Benzyloxy)methyl)-4-ethyl-2-(7-fluoro-1-oxo-4-(prop-1-en-2-yl)-1H-isochromen-6-yl)-2,4-dihydro-3H-1,2,4-triazol-3-one

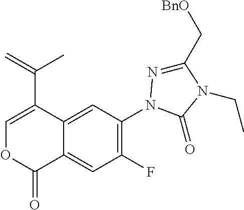 [SMILES: C=C(C)c1coc(=O)c2cc(F)c(-n3nc(COCc4ccccc4)n(CC)c3=O)cc12]

### Step A. tert-Butyl 4,5-difluoro-2-iodobenzoate

4,5-Difluoro-2-iodobenzoic acid (3 g, 11 mmol) was dissolved in THF (30 mL), then di-tert-butyl dicarbonate (4.6 g, 21 mmol) was added followed by DMAP (645 mg, 5.3 mmol). The reaction mixture was stirred under nitrogen at 50° C. overnight, then cooled down to room temperature. The solvent was evaporated under reduced pressure. The residue was diluted with EtOAc then washed with brine. The organic layer was separated, dried with Na₂SO₄, filtered, and concentrated. The residue was purified by silica column chromatography (gradient elution: 0-5% EtOAc in petroleum ether) to give the title compound as a yellow oil (2.9 g, yield: 79%).

¹H NMR (400 MHz, CDCl₃) δ 7.77 (dd, J=10.2, 7.9 Hz, 1H), 7.63 (dd, J=10.2, 7.9 Hz, 1H), 1.62 (s, 9H) ppm; ¹⁹F NMR (376 MHz, CDCl₃) δ −131.55-−131.13 (m, 1 F), −136.97-−136.65 (m, 1 F) ppm.

### Step B. tert-Butyl 4-(3-((benzyloxy)methyl)-4-ethyl-5-oxo-4,5-dihydro-1H-1,2,4-triazol-1-yl)-5-fluoro-2-iodobenzoate

A mixture of tert-butyl 4,5-difluoro-2-iodobenzoate (3.2 g, 9.4 mmol), 3-((benzyloxy)methyl)-4-ethyl-1H-1,2,4-triazol-5(4H)-one (Intermediate 1, 2.6 g, 11.2 mmol) and Cs₂CO₃ (6.1 g, 18.7 mmol) in anhydrous DMF (30 mL) was stirred under nitrogen at 75° C. for 1 h, then cooled to room temperature. The mixture was filtered through a pad of Celite®, and the pad was washed with EtOAc. The filtrate was combined, washed with brine, and concentrated. The residue was purified by silica column chromatography (gradient elution: 0-40% EtOAc in petroleum ether) to give the title compound as a colorless amorphous solid (5 g, yield: 96%). ESI-MS: mass calcd. for C₂₃H₂₅FIN₃O₄, 553.1; m/z found, 554.1 [M+H]⁺. ¹H NMR (400 MHz, CDCl₃) δ 8.16 (d, J=7.1 Hz, 1H), 7.62 (d, J=10.8 Hz, 1H), 7.29-7.45 (m, 5H), 4.61 (s, 2H), 4.50 (s, 2H), 3.84 (q, J=7.2 Hz, 2H), 1.63 (s, 9H), 1.35 (t, J=7.2 Hz, 3H) ppm; ¹⁹F NMR (376 MHz, CDCl₃) δ −119.09 (dd, J=10.6, 7.0 Hz, 1 F) ppm.

### Step C. 4-(3-((Benzyloxy)methyl)-4-ethyl-5-oxo-4,5-dihydro-1H-1,2,4-triazol-1-yl)-5-fluoro-2-iodobenzoic acid

To a solution of tert-butyl 4-(3-((benzyloxy)methyl)-4-ethyl-5-oxo-4,5-dihydro-1H-1,2,4-triazol-1-yl)-5-fluoro-2-iodobenzoate (5 g, 9 mmol) in DCM (50 mL) was slowly added TFA (10 mL). The reaction mixture was stirred at room temperature overnight. The reaction mixture was concentrated under vacuum. The obtained residue was triturated with petroleum ether at room temperature for 30 min. The mixture was filtered and the solid was rinsed with petroleum ether. The precipitate was collected and dried in vacuo to give the title compound as a white solid (4.1 g, yield: 91%). ESI-MS: mass calcd. for C₁₉H₁₇FIN₃O₄, 497.0; m/z found, 498.0 [M+H]⁺. ¹H NMR (400 MHz, DMSO-d₆) δ 8.16 (d, J=7.3 Hz, 1H), 7.78 (d, J=11.0 Hz, 1H), 7.28-7.43 (m, 5H), 4.60 (s, 2H), 4.57 (s, 2H), 3.74 (q, J=7.2 Hz, 2H), 1.23 (t, J=7.2 Hz, 3H) ppm; ¹⁹F NMR (376 MHz, DMSO-d₆) δ −119.91 (s, 1 F) ppm.

### Step D. 5-((Benzyloxy)methyl)-4-ethyl-2-(7-fluoro-1-oxo-4-(prop-1-en-2-yl)-1H-isochromen-6-yl)-2,4-dihydro-3H-1,2,4-triazol-3-one

To a mixture of 3-methylbuta-1,2-dien-1-yl acetate (Intermediate 12, 280 mg, 2.2 mmol), 4-(3-((benzyloxy)methyl)-4-ethyl-5-oxo-4,5-dihydro-1H-1,2,4-triazol-1-yl)-5-fluoro-2-iodobenzoic acid (1.1 g, 2.2 mmol) and Cy₂NMe (867 mg, 4.4 mmol) in DMF (7 mL) was added Catacxium A Pd G2 (74.2 mg, 0.11 mmol) under nitrogen. The reaction mixture was stirred under nitrogen at 90° C. for overnight. The mixture was then cooled to room temperature, diluted with EtOAc and washed with brine. The organic layer was separated and the aqueous layer was combined and extracted with EtOAc. The combined organic layer was dried over Na₂SO₄, filtered and concentrated. The residue was purified by silica column chromatography (gradient elution: 0-80% EtOAc in petroleum ether) to give the title compound as yellow solid (240 mg, yield: 25%). ESI-MS: mass calcd. for C₂₄H₂₂FN₃O₄, 435.2; m/z 436.2 [M+H]⁺. ¹H NMR (400 MHz, CDCl₃) δ 8.15 (d, J=10.5 Hz, 1H), 7.86 (d, J=6.8 Hz, 1H), 7.30-7.45 (m, 5H), 7.19 (s, 1H), 5.37-5.39 (m, 1H), 5.18 (s, 1H), 4.62 (s, 2H), 4.53 (s, 2H), 3.87 (q, J=7.1 Hz, 2H), 2.11 (s, 3H), 1.37 (t, J=7.2 Hz, 3H) ppm.

### Intermediate 4: 5-((Benzyloxy)methyl)-4-ethyl-2-(7-fluoro-4-isopropyl-1-oxo-1H-isochromen-6-yl)-2,4-dihydro-3H-1,2,4-triazol-3-one

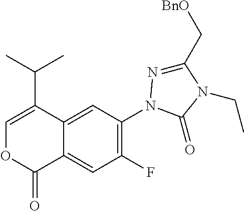 [SMILES: CCn1c(COCc2ccccc2)nn(-c2cc3c(C(C)C)coc(=O)c3cc2F)c1=O]

### Method I:

### Step A. 3-Methylbut-2-en-1-yl 4,5-difluoro-2-iodobenzoate

To the mixture of 4,5-difluoro-2-iodobenzoic acid (1.4 g, 4.9 mmol) and Cs₂CO₃ (4.8 g, 14.8 mmol) in anhydrous DMF (20 mL) was added 1-bromo-3-methyl-2-butene (1.5 g, 9.9 mmol). The reaction mixture was stirred at room temperature for 18 h. The mixture was diluted with water, and the mixture was extracted with DCM and EtOAc. The combined organic extract was dried over Na₂SO₄, filtered and concentrated. The residue was purified by flash chromatography (SiO₂, gradient elution: 10-20% EtOAc in heptane) to give the desired product as a colorless oil (1.6 g, yield: 92%). ¹H NMR (400 MHz, CDCl₃) δ 7.80 (dd, J=7.58, 9.54 Hz, 1H), 7.73 (dd, J=7.83, 10.76 Hz, 1H), 5.42-5.52 (m, 1H), 4.82 (d, J=7.34 Hz, 2H), 1.80 (s, 3H), 1.78 (s, 3H) ppm.

### Step B. 3-Methylbut-2-en-1-yl 4-(3-((benzyloxy)methyl)-4-ethyl-5-oxo-4,5-dihydro-1H-1,2,4-triazol-1-yl)-5-fluoro-2-iodobenzoate

To a mixture of 3-methylbut-2-en-1-yl 4,5-difluoro-2-iodobenzoate (1.6 g, 4.5 mmol), 3-((benzyloxy)methyl)-4-ethyl-1H-1,2,4-triazol-5(4H)-one (Intermediate 1, 2.1 g, 9.1 mmol) in anhydrous DMF (25 mL) was added Cs₂CO₃ (2.9 g, 9.1 mmol). The reaction mixture was heated under nitrogen at 85° C. for 1 h, then cooled to room temperature. The mixture was diluted with water, and the mixture was extracted with DCM and EtOAc. The combined organic extract was dried over Na₂SO₄, filtered and concentrated. The residue was purified by flash chromatography (SiO₂, gradient elution: 20-50% EtOAc in heptane) to give the title compound as a white solid (2.4 g, yield: 93%). LCMS (ES-API): mass calcd. for C₂₄H₂₅FIN₃O₄, 565.1; m/z found, 566.2 [M+H]⁺. ¹H NMR (400 MHz, CDCl₃) δ 8.21 (d, J=6.85 Hz, 1H), 7.73 (d, J=11.25 Hz, 1H), 7.29-7.44 (m, 5H), 5.41-5.53 (m, 1H), 4.84 (d, J=7.34 Hz, 2H), 4.60 (s, 2H), 4.50 (s, 2H), 3.84 (q, J=7.22 Hz, 2H), 1.80 (s, 3H), 1.78 (d, J=0.98 Hz, 3H), 1.34 (t, J=7.22 Hz, 3H) ppm.

### Step C. 5-((Benzyloxy)methyl)-4-ethyl-2-(7-fluoro-4-isopropyl-1-oxo-1H-isochromen-6-yl)-2,4-dihydro-3H-1,2,4-triazol-3-one

To a mixture of 3-methylbut-2-en-1-yl 4-(3-((benzyloxy)methyl)-4-ethyl-5-oxo-4,5-dihydro-1H-1,2,4-triazol-1-yl)-5-fluoro-2-iodobenzoate (4 g, 6.86 mmol, 1 eq) in toluene (200 mL) was added (tBu₃P)PdG₂ (351 mg, 0.69 mmol, 0.1 eq), N-cyclohexyl-N-methyl-cyclohexanamine (1.60 mL, 7.54 mmol, 1.1 eq) respectively. The reaction mixture was degassed with nitrogen for three times, and then heated under nitrogen atmosphere at 80° C. for 18 h. LCMS analysis showed ˜18% of starting material remained. The mixture was cooled to 15° C., and additional N-cyclohexyl-N-methyl-cyclohexanamine (0.72 mL, 3.43 mmol, 0.5 eq) and tBu₃PPdG₂ (176 mg, 0.34 mmol, 0.05 eq) were added. The reaction mixture was degassed with nitrogen, and then heated under nitrogen atmosphere at 80° C. for 16 h. The mixture was concentrated under reduced pressure, then diluted with H₂O (200 mL), and extracted with EtOAc (150 mL×3). The combined organic layers were washed with brine (100 mL×2), dried over anhydrous Na₂SO₄, filtered, and concentrated under reduced pressure. The residue was purified by flash chromatography (SiO₂, Petroleum ether/Ethyl acetate=5/1 to 3/1) to give the title compound as a yellow oil (1.1 g, yield: 35%). ESI-MS: mass calcd. for C₂₄H₂₄FN₃O₄, 437.2; m/z found, 438.5 [M+H]⁺. ¹H NMR (400 MHz, CDCl₃) δ 8.16 (d, J=6.6 Hz, 1H), 7.96 (d, J=6.6 Hz, 1H), 7.42-7.34 (m, 5H), 7.13 (s, 1H), 4.63 (s, 2H), 4.54 (s, 2H), 3.88 (dd, J=7.2, 14.4 Hz, 2H), 3.13-3.06 (m, 1H), 1.38 (t, J=7.2 Hz, 3H), 1.32 (d, J=6.8 Hz, 6H) ppm.

### Method II:

To a mixture of 5-((benzyloxy)methyl)-4-ethyl-2-(7-fluoro-1-oxo-4-(prop-1-en-2-yl)-1H-isochromen-6-yl)-2,4-dihydro-3H-1,2,4-triazol-3-one (Intermediate 3, 5.9 g, 13.5 mmol) in THF (100 mL) at room temperature was added Wilkinson's Catalyst [RhCl(PPh₃)₃] (3.8 g, 4.1 mmol). The mixture was degassed and purged with hydrogen gas. The reaction mixture was stirred under an atmosphere of hydrogen (15 Psi) at room temperature for 12 h. The mixture was concentrated. The residue was purified by silica column chromatography (elution: 0-25% EtOAc in petroleum ether) to give the title compound as a yellow solid (1.5 g, yield: 77%). ESI-MS: mass calcd. for C₂₄H₂₄FN₃O₄, 437.2; m/z found 438.2 [M+H]⁺. ¹H NMR (400 MHz, DMSO-d₆) δ 8.10 (d, J=10.5 Hz, 1H), 8.01 (d, J=7.0 Hz, 1H), 7.46 (s, 1H), 7.26-7.42 (m, 5H), 4.61 (s, 2H), 4.59 (s, 2H), 3.77 (q, J=7.3 Hz, 2H), 3.08 (dt, J=13.4, 6.8 Hz, 1H), 1.22-1.28 (m, 9H) ppm; ¹⁹F NMR (376 MHz, DMSO-d₆) δ −118.29 (br s, 1 F) ppm.

### Intermediate 5: 4-(3-((Benzyloxy)methyl)-4-ethyl-5-oxo-4,5-dihydro-1H-1,2,4-triazol-1-yl)-5-fluoro-2-iodobenzoyl chloride

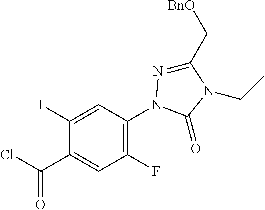 [SMILES: CCn1c(COCc2ccccc2)nn(-c2cc(I)c(C(=O)Cl)cc2F)c1=O]

### Step A. tert-Butyl 4,5-difluoro-2-iodobenzoate

4,5-Difluoro-2-iodobenzoic acid (3 g, 11 mmol) was dissolved in THF (30 mL), then di-tert-butyl dicarbonate (4.6 g, 21 mmol) was added followed by DMAP (645 mg, 5.3 mmol). The reaction mixture was stirred under nitrogen at 50° C. overnight, then cooled down to room temperature. The solvent was evaporated under reduced pressure. The residue was diluted with EtOAc then washed with brine. The organic layer was separated, dried with Na₂SO₄, filtered, and concentrated. The residue was purified by silica column chromatography (gradient elution: 0-5% EtOAc in petroleum ether) to give the title compound as a yellow oil (2.9 g, yield: 79%).

¹H NMR (400 MHz, CDCl₃) δ 7.77 (dd, J=10.2, 7.9 Hz, 1H), 7.63 (dd, J=10.2, 7.9 Hz, 1H), 1.62 (s, 9H) ppm; ¹⁹F NMR (376 MHz, CDCl₃) δ −131.55-−131.13 (m, 1 F), −136.97-−136.65 (m, 1 F) ppm.

### Step B. tert-Butyl 4-(3-((benzyloxy)methyl)-4-ethyl-5-oxo-4,5-dihydro-1H-1,2,4-triazol-1-yl)-5-fluoro-2-iodobenzoate

A mixture of tert-butyl 4,5-difluoro-2-iodobenzoate (3.2 g, 9.4 mmol), 3-((benzyloxy)methyl)-4-ethyl-1H-1,2,4-triazol-5(4H)-one (Intermediate 1, 2.6 g, 11.2 mmol) and Cs₂CO₃ (6.1 g, 18.7 mmol) in anhydrous DMF (30 mL) was stirred under nitrogen at 75° C. for 1 h, then cooled to room temperature. The mixture was filtered through a pad of Celite®, and the pad was washed with EtOAc. The filtrate was combined, washed with brine, and concentrated. The residue was purified by silica column chromatography (gradient elution: 0-40% EtOAc in petroleum ether) to give the title compound as a colorless amorphous solid (5 g, yield: 96%). ESI-MS: mass calcd. for C₂₃H₂₅FIN₃O₄, 553.1; m/z found, 554.1 [M+H]⁺. ¹H NMR (400 MHz, CDCl₃) δ 8.16 (d, J=7.1 Hz, 1H), 7.62 (d, J=10.8 Hz, 1H), 7.29-7.45 (m, 5H), 4.61 (s, 2H), 4.50 (s, 2H), 3.84 (q, J=7.2 Hz, 2H), 1.63 (s, 9H), 1.35 (t, J=7.2 Hz, 3H) ppm; ¹⁹F NMR (376 MHz, CDCl₃) δ −119.09 (dd, J=10.6, 7.0 Hz, 1 F) ppm.

### Step C. 4-(3-((Benzyloxy)methyl)-4-ethyl-5-oxo-4,5-dihydro-1H-1,2,4-triazol-1-yl)-5-fluoro-2-iodobenzoic acid

To a solution of tert-butyl 4-(3-((benzyloxy)methyl)-4-ethyl-5-oxo-4,5-dihydro-1H-1,2,4-triazol-1-yl)-5-fluoro-2-iodobenzoate (5 g, 9 mmol) in DCM (50 mL) was slowly added TFA (10 mL). The reaction mixture was stirred at room temperature overnight. The reaction mixture was concentrated under vacuum. The obtained residue was triturated with petroleum ether at room temperature for 30 min. The mixture was filtered and the solid was rinsed with petroleum ether. The precipitate was collected and dried in vacuo to give the title compound as a white solid (4.1 g, yield: 91%). ESI-MS: mass calcd. for C₁₉H₁₇FIN₃O₄, 497.0; m/z found, 498.0 [M+H]⁺. ¹H NMR (400 MHz, DMSO-d₆) δ 8.16 (d, J=7.3 Hz, 1H), 7.78 (d, J=11.0 Hz, 1H), 7.28-7.43 (m, 5H), 4.60 (s, 2H), 4.57 (s, 2H), 3.74 (q, J=7.2 Hz, 2H), 1.23 (t, J=7.2 Hz, 3H) ppm; ¹⁹F NMR (376 MHz, DMSO-d₆) δ −119.91 (s, 1 F) ppm.

### Step D. 4-(3-((Benzyloxy)methyl)-4-ethyl-5-oxo-4,5-dihydro-1H-1,2,4-triazol-1-yl)-5-fluoro-2-iodobenzoyl chloride

A solution of 4-(3-((benzyloxy)methyl)-4-ethyl-5-oxo-4,5-dihydro-1H-1,2,4-triazol-1-yl)-5-fluoro-2-iodobenzoic acid (3.5 g, 7 mmol) in SOCl₂ (14 mL) was heated at reflux for 15 min. The reaction mixture was cooled to room temperature and concentrated. To the residue was added anhydrous toluene, then the mixture was evaporated to give the crude product as a yellow gum (3.6 g), which was directly used for the next step without further purification.

### Intermediate 6: 2-Chloro-6-fluoro-N-(3-methylpent-2-en-1-yl)aniline

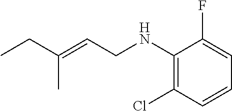

### Step A. Ethyl 3-methylpent-2-enoate

To a solution of 2-butanone (52 g, 717.6 mmol) and (carbethoxymethylene) triphenylphosphorane (50 g, 143.5 mmol) in toluene (65 mL) was added benzoic acid (3.5 g, 28.7 mmol). The reaction mixture was heated at reflux for 16 h. The mixture was diluted with petroleum ether and filtered through a short pad of silica gel. The silica gel was washed with hexane. The filtrate was concentrated under reduced pressure at 0-2° C. The residue was purified by silica column chromatography (elution: 0-10% EtOAc in petroleum ether) to give the title compound as a colorless liquid (23.3 g crude). ¹H NMR (400 MHz, CDCl₃) δ 5.58-5.69 (m, 1H), 4.13 (qd, J=7.1, 4.9 Hz, 2H), 2.62 (q, J=7.5 Hz, 1H), 2.09-2.20 (m, 3H), 1.86 (d, J=1.2 Hz, 1H), 1.24-1.28 (m, 3H), 1.01-1.09 (m, 3H) ppm.

### Step B. 3-Methylpent-2-en-1-ol

To a toluene solution (1 M) of DIBAL-H (118 mL, 118 mmol) at −78° C. was added a toluene solution (40 mL) of ethyl 3-methylpent-2-enoate (20 g crude) dropwise under nitrogen. The reaction mixture was stirred at −78° C. for 2 h. The mixture was warmed to room temperature and slowly poured into saturated aqueous potassium sodium tartrate solution at 0° C. The mixture was stirred for 2 h and filtered through a short pad of Celite®. The pad was washed with DCM/EtOAc (v/v, 3/1), and the filtrate was extracted with DCM. The organic extract was separated, dried over Na₂SO₄, filtered and concentrated. The residue was purified by silica column chromatography (elution: 0-100% DCM in petroleum ether, then 0-30% EtOAc in DCM) to give the title compound as a colorless liquid (7 g, yield of two steps: 57%).

¹H NMR (400 MHz, CDCl₃) δ 5.35-5.46 (m, 1H), 4.11-4.21 (m, 2H), 2.02-2.13 (m, 2H), 1.67-1.76 (m, 3H), 0.98-1.06 (m, 3H) ppm.

### Step C. 3-Methylpent-2-enal

To a solution of 3-methylpent-2-en-1-ol (2 g, 20.0 mmol) in DCM (20 mL) was added Dess-martin periodinane (10 g, 24.0 mmol). The reaction mixture was stirred at room temperature for 1 h. The mixture was filtered through a short pad of Celite®. The pad was washed with DCM. The combined filtrate was washed with saturated aqueous NaHCO₃ solution. The organic layer was separated, dried over Na₂SO₄, filtered and concentrated under reduced pressure at 0-2° C. The crude was purified by silica column chromatography (elution: DCM) to give the title compound as a colorless liquid (1.5 g, yield: 77%). ¹H NMR (400 MHz, CDCl₃) δ 9.91-10.04 (m, 1H), 5.78-5.90 (m, 1H), 2.58 (q, J=7.6 Hz, 1H), 2.23 (d, J=7.3 Hz, 1H), 2.16 (s, 2H), 1.96 (d, J=1.1 Hz, 1H), 1.16 (t, J=7.6 Hz, 1H), 1.09 (t, J=7.4 Hz, 2H) ppm.

### Step D. N-(2-Chloro-6-fluorophenyl)-3-methylpent-2-en-1-imine

To a mixture of 2-chloro-6-fluoroaniline (1.2 g, 8.2 mmol) and 3-methylpent-2-enal (0.97 g, 9.9 mmol) in DCM (18 mL) under nitrogen at 0° C. was added triethylamine (4.6 mL, 33 mmol), followed by the addition of a DCM solution (1 M) of TiCl₄ (5 mL, 5 mmol) dropwise. The resulting mixture was stirred at 0° C. for 1 h, then warmed to room temperature and stirred for 4 h. The mixture was poured into saturated aqueous NH₄Cl solution. The mixture became cloudy and filtered through a pad of Celite®. The pad was washed with EtOAc. The combined filtrate was diluted with DCM and water. The organic layer was separated, and aqueous layer was extracted with DCM. The combined organic layers were washed with brine, dried over Na₂SO₄, filtered and concentrated. The residue product was purified by silica gel column chromatography (gradient elution: 0-5% DCM in petroleum ether) to give the title compound as a pale yellow oil (1.3 g, yield: 70%).

### Step E. 2-Chloro-6-fluoro-N-(3-methylpent-2-en-1-yl)aniline

To a solution of N-(2-chloro-6-fluorophenyl)-3-methylpent-2-en-1-imine (1.3 g, 5.76 mmol) in MeOH (20 mL) was added NaBH₄ (218 mg, 5.8 mmol), and after 1 h, another batch of NaBH₄ (218 mg, 5.8 mmol) was added. A total of NaBH₄ (1.1 g, 29 mmol) was added. The reaction mixture was stirred at room temperature overnight. The mixture was concentrated, and then diluted with water and extracted with EtOAc. The organic layer was separated, washed with brine, dried over Na₂SO₄, filtered and concentrated. The residue was purified by combi flash column chromatography over silica gel (eluent: 0-5% DCM in petroleum ether) to give the title compound as a yellow oil (430 mg, yield: 33%). ¹H NMR (400 MHz, CDCl₃) δ 6.95-7.02 (m, 1H), 6.84 (ddd, J=12.2, 8.3, 1.3 Hz, 1H), 6.52-6.63 (m, 1H), 5.17-5.28 (m, 1H), 3.85 (d, J=5.6 Hz, 2H), 3.73 (s, 1H), 1.91-2.07 (m, 2H), 1.58-1.68 (m, 3H), 0.89-0.96 (m, 3H).

### Intermediate 7: 5-Chloro-3-methyl-1-(tetrahydro-2H-pyran-2-yl)-1H-pyrazol-4-amine

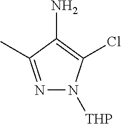

### Step A. 5-Chloro-3-methyl-4-nitro-1-(tetrahydro-2H-pyran-2-yl)-1H-pyrazole

To a solution of 3-methyl-4-nitropyrazole (2 g, 15.7 mmol) in EtOAc (20 mL) was added DHP (2 g, 23.6 mmol) and TsOH·H₂O (150 mg, 0.79 mmol) at room temperature. The mixture was stirred at room temperature for overnight. Et₃N (0.4 mL) was added and the mixture was washed with brine. Then the organic layer was separated, dried over anhydrous Na₂SO₄, filtered and concentrated. The residue was dissolved in THF (45 mL) and the temperature was lowered to −78° C. A THF (1 M) solution of LiHMDS (10.6 mL, 13.8 mmol) was added to the mixture under nitrogen. After 45 minutes at −78° C., the solution of hexachloroethane (8.9 g, 37.8 mmol) in THF (20 mL) was added dropwise. The reaction mixture was warmed to room temperature and stirred for overnight. The mixture was poured into saturated aqueous NH₄Cl solution and extracted with EtOAc. The organic phase was separated, washed with brine, dried over anhydrous Na₂SO₄, filtered and concentrated. The residue was purified by silica column chromatography (gradient elution: 0-40% EtOAc in petroleum ether) to give the title compound as white solid (1.8 g, yield: 58%). ¹H NMR (400 MHz, CDCl₃) δ 5.52 (dd, J=10.0, 2.7 Hz, 1H), 4.07-4.15 (m, 1H), 3.70 (td, J=11.3, 2.8 Hz, 1H), 2.57 (s, 3H), 2.37-2.47 (m, 1H), 2.11-2.19 (m, 1H), 1.86-1.90 (m, 1H), 1.72-1.75 (m, 1H), 1.64 (d, J=2.0 Hz, 1H), 1.53 (d, J=6.6 Hz, 1H) ppm.

### Step B. 5-Chloro-3-methyl-1-(tetrahydro-2H-pyran-2-yl)-1H-pyrazol-4-amine

To a mixture of 5-chloro-3-methyl-4-nitro-1-(tetrahydro-2H-pyran-2-yl)-1H-pyrazole (100 mg, 0.4 mmol) in MeOH/THF/H₂O (v/v/v, 1/1/1, 3 mL) was added iron powder (114 mg, 2.0 mmol) and NH₄Cl (109 mg, 2.0 mmol). The mixture was stirred at 70° C. for 1.5 h. The mixture was cooled to room temperature and filtered through a pad of Celite®. The pad was washed with EtOAc. The combined filtrate was washed with saturated aqueous NaHCO₃ solution. The organic layer was separated, and the aqueous layer was extracted with EtOAc. The combined organic extract was washed with brine, dried over anhydrous Na₂SO₄, filtered and concentrated. The residue was purified by silica column chromatography (gradient elution: 0-50% EtOAc in petroleum ether) to give the title compound as a yellow oil (70 mg, yield: 79%). ESI-MS: mass calcd. for C₉H₁₄C₁N₃O, 215.1; m/z found, 216.1 [M+H]⁺.

### Intermediate 8: 3-(2-((tert-Butyldiphenylsilyl)oxy)ethoxy)-2-chloroaniline

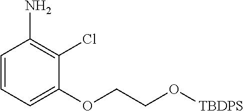

### Step A. tert-Butyl(2-(2-chloro-3-nitrophenoxy)ethoxy)diphenylsilane

To a mixture of 2-chloro-3-nitrophenol (200 mg, 1.2 mmol), 2-((tert-butyldiphenylsilyl)oxy)ethan-1-ol (554 mg, 1.8 mmol) and PPh₃ (453 mg, 1.7 mmol) in THF (10 mL) was added DEAD (281 mg, 161 mmol) at 0° C. under nitrogen. The mixture was warmed to room temperature and stirred at room temperature for 12 h. Saturated aqueous NH₄Cl solution was added, and the mixture was extracted with EtOAc. The organic was separated, washed with brine, dried over anhydrous Na₂SO₄, filtered and concentrated. The residue was purified by silica column chromatography (gradient elution: 0-10% EtOAc in petroleum ether) to give the title compound as a yellow oil (240 mg, yield: 46%). ¹H NMR (400 MHz, CDCl₃) δ 7.72 (dd, J=7.8, 1.5 Hz, 4H), 7.35-7.49 (m, 7H), 7.30 (t, J=8.2 Hz, 1H), 7.12 (dd, J=8.3, 1.2 Hz, 1H), 4.20-4.25 (m, 2H), 4.06 (t, J=4.9 Hz, 2H), 1.06 (s, 9H) ppm.

### Step B. 3-(2-((tert-Butyldiphenylsilyl)oxy)ethoxy)-2-chloroaniline

To a mixture of tert-butyl(2-(2-chloro-3-nitrophenoxy)ethoxy)diphenylsilane (220 mg, 0.5 mmol), NH₄Cl (258 mg, 4.8 mmol) in THF (3 mL), MeOH (3 mL) and H2O (3 mL) was added iron powder (269 mg, 4.8 mmol). The reaction mixture was stirred at 70° C. for 2 h. The mixture was cooled to room temperature, diluted with EtOAc, and filtered through a pad of Celite®. The Celite® was washed with EtOAc. The combined filtrate was washed with brine, dried over Na₂SO₄, filtered and concentrated. The residue was purified by silica column chromatography (gradient elution: 0-11% EtOAc in petroleum ether) to give the title compound as a yellow solid (192 mg, yield: 92%). ESI-MS: mass calcd. for C₂₄H₂₈ClNO₂Si, 425.2; m/z found, 426.1[M+H]⁺. ¹H NMR (400 MHz, CDCl₃) δ 7.75 (dd, J=7.9, 1.6 Hz, 4H), 7.35-7.48 (m, 6H), 6.97 (t, J=8.1 Hz, 1H), 6.42 (dd, J=8.2, 1.1 Hz, 1H), 6.33 (dd, J=8.2, 1.1 Hz, 1H), 4.13-4.17 (m, 2H), 4.07-4.13 (m, 2H), 4.01-4.06 (m, 2H), 1.06 (s, 9H) ppm.

### Intermediate 9: 5-((Benzyloxy)methyl)-4-ethyl-2-(7-methyl-1-oxo-4-(prop-1-en-2-yl)-1H-isochromen-6-yl)-2,4-dihydro-3H-1,2,4-triazol-3-one

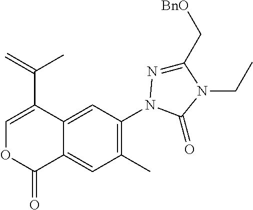 [SMILES: C=C(C)c1coc(=O)c2cc(C)c(-n3nc(COCc4ccccc4)n(CC)c3=O)cc12]

### Step A. tert-Butyl 2-bromo-4-fluoro-5-methylbenzoate

To a solution of 2-bromo-4-fluoro-5-methylbenzoic acid (1 g, 4.3 mmol) in THF (10 mL) was added (Boc)₂O (1.9 g, 8.6 mmol), followed by the addition of DMAP (262 mg, 2.1 mmol). The reaction mixture turned orange and was stirred under nitrogen at 50° C. for overnight. The mixture was cooled to room temperature, diluted with EtOAc, and then washed with brine. The organic layer was separated, dried over Na₂SO₄, filtered and concentrated. The residue was purified by silica column chromatography (elution: 0-3% EtOAc in petroleum ether) to give the title compound as colorless oil (900 mg, yield: 72%). ¹H NMR (400 MHz, CDCl₃) δ 7.57 (d, J=8.1 Hz, 1H), 7.24 (d, J=4.6 Hz, 1H), 2.22 (d, J=1.5 Hz, 3H), 1.58 (s, 9H) ppm.

### Step B. tert-Butyl 4-(3-((benzyloxy)methyl)-4-ethyl-5-oxo-4,5-dihydro-1H-1,2,4-triazol-1-yl)-2-bromo-5-methylbenzoate

A mixture of tert-butyl 2-bromo-4-fluoro-5-methylbenzoate. (750 mg, 2.6 mmol), 5-((benzyloxy)methyl)-4-ethyl-2,4-dihydro-3H-1,2,4-triazol-3-one (800 mg, 3.4 mmol) and Cs₂CO₃ (1.7 g, 5.2 mmol) in DMF (8 mL) was stirred at 90° C. for 16 h. The reaction was quenched by the addition of aqueous saturated NH₄Cl solution. The mixture was extracted with EtOAc. The organic layer was separated, washed with brine, dried over Na₂SO₄, filtered and concentrated. The residue was purified by combi-flash chromatography (SiO₂, eluent: 0-22% EtOAc in petroleum ether) to give the title compound as colorless gum (1 g, yield: 71%). ESI-MS: mass calcd. for C₂₄H₂₈BrN₃O₄, 501.1; m/z found, 502.1 [M+H]⁺. ¹H NMR (400 MHz, CDCl₃) δ 7.64 (d, J=6.1 Hz, 2H), 7.33-7.43 (m, 5H), 4.61 (s, 2H), 4.50 (s, 2H), 3.85 (q, J=7.3 Hz, 2H), 2.31 (s, 3H), 1.62 (s, 9H), 1.36 (t, J=7.2 Hz, 3H) ppm.

### Step C. 4-(3-((Benzyloxy)methyl)-4-ethyl-5-oxo-4,5-dihydro-1H-1,2,4-triazol-1-yl)-2-bromo-5-methylbenzoic acid

To a mixture of tert-butyl 4-(3-((benzyloxy)methyl)-4-ethyl-5-oxo-4,5-dihydro-1H-1,2,4-triazol-1-yl)-2-bromo-5-methylbenzoate (500 mg, 0.90 mmol) in DCM (5 mL) was added TFA (1 mL). The mixture was stirred at room temperature for 12 h. The mixture was concentrated. The residue was dissolved with DCM, and petroleum ether was added slowly. The mixture was stirred at room temperature for 30 min. The mixture was filtered, and the precipitate was rinsed with petroleum ether. The solid was collected and dried in vacuo to give the title compound as a white solid (360 mg, yield: 86%). ESI-MS: mass calcd. for C₂₀H₂₀BrN₃O₄, 445.1; m/z found, 446.0 [M+H]⁺. ¹H NMR (400 MHz, DMSO-d₆) δ 7.77 (s, 1H), 7.70 (s, 1H), 7.29-7.41 (m, 5H), 4.59 (s, 2H), 4.56 (s, 2H), 3.74 (q, J=7.0 Hz, 2H), 2.24 (s, 3H), 1.23 (t, J=7.2 Hz, 3H) ppm.

### Step D. 5-((Benzyloxy)methyl)-4-ethyl-2-(7-methyl-1-oxo-4-(prop-1-en-2-yl)-1H-isochromen-6-yl)-2,4-dihydro-3H-1,2,4-triazol-3-one

To a mixture of 4-(3-((benzyloxy)methyl)-4-ethyl-5-oxo-4,5-dihydro-1H-1,2,4-triazol-1-yl)-2-bromo-5-methylbenzoic acid (560 mg, 1.26 mmol), 3-methylbuta-1,2-dien-1-yl acetate (Intermediate 12, 1.58 g, 12.5 mmol), AcOK (369 mg, 3.76 mmol) and TBAB (809 mg, 2.51 mmol) in DMF (3.9 mL) under nitrogen was added Pd(OAc)₂ (141 mg, 0.63 mmol). The reaction mixture was stirred under nitrogen at 90° C. for overnight. The mixture was cooled to room temperature, diluted with EtOAc, and washed with brine. The organic layer was separated, and the aqueous layer was extracted with EtOAc. The combined organic layers were combined, dried over Na₂SO₄, filtered, and concentrated. The residue was purified by silica column chromatography (gradient elution: 0-70% EtOAc in petroleum ether) to give the title compound as a yellow solid (410 mg, yield: 73%). ESI-MS: mass calcd. for C₂₅H₂₅N₃O₄, 431.2; m/z found, 432.1 [M+H]⁺. ¹H NMR (400 MHz, CDCl₃) δ 8.28 (s, 1H), 7.59 (s, 1H), 7.31-7.46 (m, 5H), 7.17 (s, 1H), 5.30-5.37 (m, 1H), 5.15 (s, 1H), 4.63 (s, 2H), 4.52 (s, 2H), 3.87 (q, J=7.1 Hz, 2H), 2.46 (s, 3H), 2.10 (s, 3H), 1.38 (t, J=7.2 Hz, 3H) ppm.

### Intermediate 10: Isopropyl 6-(3-((benzyloxy)methyl)-4-ethyl-5-oxo-4,5-dihydro-1H-1,2,4-triazol-1-yl)-2-chloro-5-fluoronicotinate

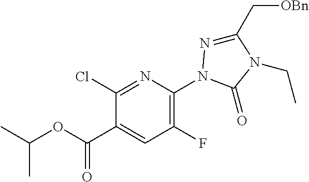 [SMILES: CCn1c(COCc2ccccc2)nn(-c2nc(Cl)c(C(=O)OC(C)C)cc2F)c1=O]

### Step A. 2,6-Dichloro-5-fluoronicotinoyl chloride

To a solution of 2,6-dichloro-5-fluoronicotinic acid (20 g, 95 mmol) in THF (200 mL) was added (COCl)₂ (12.7 g, 10.0 mmol) and DMF (69.6 mg, 0.952 mmol) at 0° C. dropwise. The mixture was stirred at 0° C. for 30 min, then warmed to 25° C., and stirred for 1 h. The reaction mixture was concentrated under reduced pressure to afford desired product (21.7 g, crude) as a colorless oil, which was used without further purification.

### Step B. Isopropyl 2,6-dichloro-5-fluoronicotinate

To a mixture of propan-2-ol (8.56 g, 142 mmol, 10.9 mL) and pyridine (9.02 g, 114 mmol) in THF (200 mL) was added a solution of 2,6-dichloro-5-fluoronicotinoyl chloride (21.7 g, 96.0 mmol) in THF (50 mL) at 0° C. The mixture was stirred at 25° C. for 1 h. The mixture was poured into water (300 mL). The aqueous phase was extracted with ethyl acetate (300 mL). The combined organic phase was dried with anhydrous Na₂SO₄, filtered and concentrated. The residue was purified by column chromatography (SiO₂, Petroleum ether/Ethyl acetate=1/1 to 10:1) to afford the title compound (21 g, 86.82% yield). MS (ESI): mass calcd. for C₉HsCl₂FNO₂, 250.1; m/z found, 252.0 [M+H]⁺. ¹H NMR (400 MHz, CDCl₃) δ 7.97-7.95 (d, J=7.2 Hz, 1H), 5.32-5.25 (m, 1H), 1.58-1.39 (m, 6H) ppm.

### Step C. Isopropyl 6-(3-((benzyloxy)methyl)-4-ethyl-5-oxo-4,5-dihydro-1H-1,2,4-triazol-1-yl)-2-chloro-5-fluoronicotinate

To a mixture of isopropyl 2,6-dichloro-5-fluoronicotinate (4 g, 15.87 mmol) in DMSO (40 mL) was added 3-((benzyloxy)methyl)-4-ethyl-1H-1,2,4-triazol-5(4H)-one (3.89 g, 16.66 mmol) and K₂CO₃ (3.29 g, 23.80 mmol). The mixture was stirred at 80° C. for 3 hr. LCMS showed the starting material was consumed and desired mass was detected. The mixture was diluted with H₂O (30 mL) and extracted with EtOAc (50 mL×3). The combined organic layers were washed with brine (100 mL), dried over Na₂SO₄, filtered and concentrated under reduced pressure. The residue was purified by column chromatography (SiO₂, Petroleum ether/Ethyl acetate=1/0 to 1:1) to afford the title compound (5.7 g, 79.86% yield). MS (ESI): mass calcd. for C₂₁H₂₂C₁FN₄O₄, 448.1; m/z found, 449.2 [M+H]⁺. ¹H NMR (400 MHz, CDCl₃) δ 8.10 (d, J=8.8 Hz, 1H), 7.43-7.31 (m, 5H), 5.30 (td, J=6.3, 12.5 Hz, 1H), 4.61 (s, 2H), 4.54 (s, 2H), 3.85 (q, J=7.2 Hz, 2H), 1.41 (d, J=6.2 Hz, 6H), 1.37-1.31 (m, 3H) ppm.

### Intermediate 11: 5-((Benzyloxy)methyl)-4-ethyl-2-(7-fluoro-3-hydroxy-4-isopropyl-1-oxoisochroman-6-yl)-2,4-dihydro-3H-1,2,4-triazol-3-one

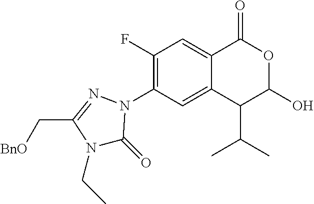 [SMILES: CCn1c(COCc2ccccc2)nn(-c2cc3c(cc2F)C(=O)OC(O)C3C(C)C)c1=O]

### Step A. Methyl 4-(3-((benzyloxy)methyl)-4-ethyl-5-oxo-4,5-dihydro-1H-1,2,4-triazol-1-yl)-2-bromo-5-fluorobenzoate

To a flask charged with methyl 2-bromo-4,5-difluorobenzoate (100.0 g, 398 mmol), 5-((benzyloxy)methyl)-4-ethyl-2,4-dihydro-3H-1,2,4-triazol-3-one (Intermediate 1, 113.5 g, 508 mmol) and K₂CO₃ (100.0 g, 724 mmol) was added anhydrous DMF (1000 mL). The reaction mixture was heated under nitrogen at 50° C. for 16 h, and then additional 5-((benzyloxy)methyl)-4-ethyl-2,4-dihydro-3H-1,2,4-triazol-3-one (11 g, 51 mmol) was added. The reaction mixture continued to be stirred at 50° C. The mixture was cooled to room temperature and stirred for 10 min. Water (1000 mL) was added dropwise, and the mixture was stirred at room temperature for 2 h. The precipitate was collected by filtration and dried to give the crude product (190 g). The product was stirred in DMF (500 mL) for 30 min, then water (500 mL) was added. The mixture was stirred for 2 h. The precipitate was collected by filtration and dried to give the title compound (180 g, yield: 90%). ¹H NMR (300 MHz, CDCl₃) δ 7.95 (d, J=6.7 Hz, 1H), 7.73 (d, J=10.7 Hz, 1H), 7.44-7.27 (m, 5H), 4.60 (s, 2H), 4.50 (s, 2H), 3.95 (s, 3H), 3.84 (q, J=7.2 Hz, 2H), 1.34 (t, J=7.2 Hz, 3H) ppm.

### Step B. 5-((Benzyloxy)methyl)-4-ethyl-2-(7-fluoro-3-hydroxy-4-isopropyl-1-oxoisochroman-6-yl)-2,4-dihydro-3H-1,2,4-triazol-3-one

To a mixture of methyl 4-(3-((benzyloxy)methyl)-4-ethyl-5-oxo-4,5-dihydro-1H-1,2,4-triazol-1-yl)-2-bromo-5-fluorobenzoate (30 g, 65.9 mmol), Xantphos (4.02 g, 6.6 mmol), [Pd(allyl)Cl]₂ (1.38 g, 3.77 mmol), Cs₂CO₃ (42.6 g, 130 mmol) in dimethylacetamide (300 mL) was added 3-methylbutanal (41.4 mL, 386 mmol) slowly. The reaction mixture was heated under nitrogen at 80° C. for 22 h. The mixture was filtered and quenched with aqueous NH₄Cl solution until “pH” turned 7-8. The mixture was extracted with ethyl acetate (2000 mL×2). The combined organic extract was concentrated. The residue was purified by column chromatography (SiO₂, gradient elution: 1-33% ethyl acetate in petroleum ether) to give the title compound as an oil (61.7 g, yield: 56%). ¹H NMR (300 MHz, CDCl₃) δ 7.93 (d, J=10.4 Hz, 1H), 7.55 (d, J=6.7 Hz, 1H), 7.44-7.28 (m, 5H), 5.92 (s, 1H), 4.61 (s, 2H), 4.51 (s, 2H), 4.37 (br s, 1H), 3.85 (q, J=7.2 Hz, 2H), 2.80 (d, J=7.1 Hz, 1H), 1.92 (spt, J=6.8 Hz, 1H), 1.35 (t, J=7.2 Hz, 3H), 1.05 (d, J=6.8 Hz, 3H), 0.93 (d, J=6.8 Hz, 3H) ppm.

### Intermediate 12: 3-Methylbuta-1,2-dien-1-yl acetate

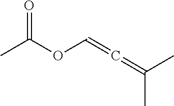

### Step A. 2-Methylbut-3-yn-2-yl acetate

To a mixture of Mg(ClO₄)₂ (796 mg, 3.6 mmol) in acetic anhydride (38 g, 371 mmol) at 0° C. was added 2-methyl-3-butyn-2-ol (30 g, 357 mmol) dropwise. The reaction mixture was stirred at 0° C. for 10 min, then warmed to room temperature and stirred for overnight. The reaction mixture was diluted with DCM, then washed with aqueous saturated NaHCO₃ solution and aqueous saturated Na₂CO₃ solution. The organic layer was separated, dried over Na₂SO₄, filtered and concentrated at 0° C. The residue was purified by silica column chromatography (elution: DCM) to give the title compound as pale yellow oil (35.8 g, yield: 80%). ¹H NMR (400 MHz, CDCl₃) δ 2.54 (s, 1H), 2.03 (s, 3H), 1.67 (s, 6H) ppm.

### Step B. 3-Methylbuta-1,2-dien-1-yl acetate

To a solution of 2-methylbut-3-yn-2-yl acetate (2.5 g, 20 mmol) in DCM (20 mL) was added AgBF₄ (117 mg, 0.6 mmol) under nitrogen. The resulting colorless solution was stirred under nitrogen at 35° C. for 2 h until the mixture turned into a black solution. The mixture was washed with aqueous ammonia (10%). The organic layer was separated, and the aqueous layer was extracted with DCM. The combined organic layer was dried over Na₂SO₄, filtered and concentrated. The residue was purified by silica column chromatography (gradient elution: 0-3% EtOAc in petroleum ether) to give the title compound as yellow oil (650 mg, yield: 26%).

¹H NMR (400 MHz, CDCl₃) δ 7.20 (dt, J=4.1, 2.0 Hz, 1H), 2.11 (s, 3H), 1.81 (d, J=2.0 Hz, 6H) ppm.

### Example 22 of PCT/IB2020/053601 (Which Published as WO 2020/212897 on Oct. 22, 2020) discloses 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one (Compound 22)

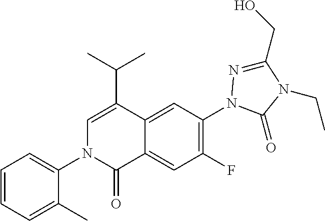 [SMILES: CCn1c(CO)nn(-c2cc3c(C(C)C)cn(-c4ccccc4C)c(=O)c3cc2F)c1=O]

### Step A. 6-(3-((Benzyloxy)methyl)-4-ethyl-5-oxo-4,5-dihydro-1H-1,2,4-triazol-1-yl)-7-fluoro-4-isopropyl-2-(o-tolyl)isoquinolin-1(2H)-one

To a mixture of 5-((benzyloxy)methyl)-4-ethyl-2-(7-fluoro-3-hydroxy-4-isopropyl-1-oxoisochroman-6-yl)-2,4-dihydro-3H-1,2,4-triazol-3-one (Intermediate 11, 56 g, 123 mmol) in AcOH (160 mL) was added o-toluidine (14.8 g, 138 mmol). The reaction mixture was heated at 80° C. for 16 h. The mixture was concentrated, and then the “pH” was adjusted to 7-8 with aqueous NaHCO₃ solution. The mixture was extracted with ethyl acetate (160 mL×2). The combined organic extract was concentrated. The residue was purified by flash chromatography (SiO₂, 0-20% Ethyl acetate in DCM) to give the title compound as an oil (32.5 g, yield: 50%). MS (ESI): mass calcd. for C₃₁H₃₁FN₄O₃, 526.2; m/z found, 527.4 [M+H]⁺. ¹H NMR (300 MHz, CDCl₃) δ 8.33 (d, J=10.9 Hz, 1H), 8.07 (d, J=6.8 Hz, 1H), 7.44-7.27 (m, 9H), 6.84 (s, 1H), 4.64 (s, 2H), 4.55 (s, 2H), 3.89 (q, J=7.2 Hz, 2H), 3.23 (spt, J=6.8 Hz, 1H), 2.16 (s, 3H), 1.39 (t, J=7.2 Hz, 3H), 1.31 (dd, J=6.8, 2.1 Hz, 6H) ppm.

### Step B. 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one

To a stirred solution of 6-(3-((benzyloxy)methyl)-4-ethyl-5-oxo-4,5-dihydro-1H-1,2,4-triazol-1-yl)-7-fluoro-4-isopropyl-2-(o-tolyl)isoquinolin-1(2H)-one (26.5 g, 50.3 mmol) in DCM (230 mL) at −78° C. was added a DCM solution (1 M) of BCl₃ (290 mL, 290 mL) under nitrogen. The reaction mixture was stirred at 15° C. for 0.5 h. The reaction was quenched by MeOH (100 mL) at −78° C. to −20° C. The mixture was partitioned between water and DCM. The organic layer was separated, and the aqueous layer was extracted with DCM (110 mL×2). The combined organic extract was washed with brine (30 mL×2), dried with anhydrous Na₂SO₄, filtered, and concentrated to give a crude product (27.5 g). The product was triturated with methyl ethyl ketone (82 mL) and heptane (290 mL) to give a pure product (17.5 g), which was re-crystallized in ethanol and water to give the title compound as a white solid (16 g, yield: 73%). MS (ESI): mass calcd. for C₂₄H₂₅FN₄O₃, 436.2; m/z found, 437.2 [M+H]⁺. ¹H NMR (400 MHz, CDCl₃) δ 8.33 (d, J=11.2, 1H), 8.08 (d, J=6.8, 1H), 7.39-7.33 (m, 3H), 7.28 (s, 1H), 6.85 (s, 1H), 4.69 (br s, 2H), 3.94 (q, J=7.11 Hz, 2H), 3.27 (td, J=13.66, 6.82 Hz, 1H), 2.32 (br s, 1H), 2.17 (s, 3H), 1.45 (t, J=7.11 Hz, 3H), 1.32 (dd, J=6.82, 1.83 Hz, 6H) ppm.

It is noted that compounds of Formula (Z) described herein are described in PCT/IB2020/053601 (which published as WO 2020/212897 on Oct. 22, 2020), which is incorporated by reference herein, in its entirety for all purposes.

In some embodiments, provided is a combination therapy comprising a menin-MLL inhibitor of Formula (I), or a pharmaceutically acceptable salt or a solvate thereof, and at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a hypomethylating agent.

According to embodiments, the hypomethylating agent is azacitidine, decitabine, or pharmaceutically acceptable salts or solvates thereof.

According to particular embodiments, the hypomethylating agent is azacitidine, or a pharmaceutically acceptable salt or solvate thereof.

In some embodiments, provided is a combination therapy comprising a menin-MLL inhibitor of Formula (I), or a pharmaceutically acceptable salt or a solvate thereof, and at least one other therapeutic agent is azacitidine, or a pharmaceutically acceptable salt or solvate thereof.

In some embodiments, provided is a combination therapy comprising Compound A or a pharmaceutically acceptable salt or solvate thereof, and at least one other therapeutic agent is azacitidine, or a pharmaceutically acceptable salt or solvate thereof.

In some embodiments, provided is a combination therapy comprising Compound A1, and at least one other therapeutic agent is azacitidine, or a pharmaceutically acceptable salt or solvate thereof.

In some embodiments, provided is a combination therapy comprising Compound A2, and at least one other therapeutic agent is azacitidine, or a pharmaceutically acceptable salt or solvate thereof.

In some embodiments, provided is a combination therapy comprising Compound A3, and at least one other therapeutic agent is azacitidine, or a pharmaceutically acceptable salt or solvate thereof.

In some embodiments, provided is a combination therapy comprising Compound A4-a or a solvate thereof, and at least one other therapeutic agent is azacitidine, or a pharmaceutically acceptable salt or solvate thereof.

In some embodiments, provided is a combination therapy comprising Compound A4-b or a hydrate thereof, and at least one other therapeutic agent is azacitidine, or a pharmaceutically acceptable salt or solvate thereof.

In some embodiments, provided is a combination therapy comprising Compound A4, and at least one other therapeutic agent is azacitidine, or a pharmaceutically acceptable salt or solvate thereof.

In some embodiments, provided is a combination therapy comprising a menin-MLL inhibitor of Formula (I), or a pharmaceutically acceptable salt or a solvate thereof, and at least one other therapeutic agent is azacitidine, or a pharmaceutically acceptable salt or solvate thereof.

In some embodiments, provided is a combination therapy comprising Compound A or a pharmaceutically acceptable salt or solvate thereof, and azacitidine, or a pharmaceutically acceptable salt or solvate thereof.

In some embodiments, provided is a combination therapy comprising Compound A1, and azacitidine, or a pharmaceutically acceptable salt or solvate thereof.

In some embodiments, provided is a combination therapy comprising Compound A2, and azacitidine, or a pharmaceutically acceptable salt or solvate thereof.

In some embodiments, provided is a combination therapy comprising Compound A3, and azacitidine, or a pharmaceutically acceptable salt or solvate thereof.

In some embodiments, provided is a combination therapy comprising Compound A4-a or a solvate thereof, and azacitidine, or a pharmaceutically acceptable salt or solvate thereof.

In some embodiments, provided is a combination therapy comprising Compound A4-b or a hydrate thereof, and azacitidine, or a pharmaceutically acceptable salt or solvate thereof.

In some embodiments, provided is a combination therapy comprising Compound A4, and azacitidine, or a pharmaceutically acceptable salt or solvate thereof.

In some embodiments, provided is a combination therapy comprising a menin-MLL inhibitor of Formula (I), or a pharmaceutically acceptable salt or a solvate thereof, and at least one other therapeutic agent is decitabine, or a pharmaceutically acceptable salt or solvate thereof.

In some embodiments, provided is a combination therapy comprising Compound A or a pharmaceutically acceptable salt or solvate thereof, and at least one other therapeutic agent is decitabine, or a pharmaceutically acceptable salt or solvate thereof.

In some embodiments, provided is a combination therapy comprising Compound A1, and at least one other therapeutic agent is decitabine, or a pharmaceutically acceptable salt or solvate thereof.

In some embodiments, provided is a combination therapy comprising Compound A2, and at least one other therapeutic agent is decitabine, or a pharmaceutically acceptable salt or solvate thereof.

In some embodiments, provided is a combination therapy comprising Compound A3, and at least one other therapeutic agent is decitabine, or a pharmaceutically acceptable salt or solvate thereof.

In some embodiments, provided is a combination therapy comprising Compound A4-a or a solvate thereof, and at least one other therapeutic agent is decitabine, or a pharmaceutically acceptable salt or solvate thereof.

In some embodiments, provided is a combination therapy comprising Compound A4-b or a hydrate thereof, and at least one other therapeutic agent is decitabine, or a pharmaceutically acceptable salt or solvate thereof.

In some embodiments, provided is a combination therapy comprising Compound A4, and at least one other therapeutic agent is decitabine, or a pharmaceutically acceptable salt or solvate thereof.

In some embodiments, provided is a combination therapy comprising a menin-MLL inhibitor of Formula (I), or a pharmaceutically acceptable salt or a solvate thereof, and at least one other therapeutic agent is a DNA intercalating agent.

According to embodiments, the DNA intercalating agent is an anthracycline.

In some embodiments, provided is a combination therapy comprising a menin-MLL inhibitor of Formula (I), or a pharmaceutically acceptable salt or a solvate thereof, and at least one other therapeutic agent is an anthracycline.

In some embodiments, provided is a combination therapy comprising Compound A or a pharmaceutically acceptable salt or solvate thereof, and at least one other therapeutic agent is an anthracycline.

In some embodiments, provided is a combination therapy comprising Compound A1, and at least one other therapeutic agent is an anthracycline.

In some embodiments, provided is a combination therapy comprising Compound A2, and at least one other therapeutic agent is an anthracycline.

In some embodiments, provided is a combination therapy comprising Compound A3, and at least one other therapeutic agent is an anthracycline.

In some embodiments, provided is a combination therapy comprising Compound A4-a or a solvate thereof, and at least one other therapeutic agent is an anthracycline.

In some embodiments, provided is a combination therapy comprising Compound A4-b or a hydrate thereof, and at least one other therapeutic agent is an anthracycline.

In some embodiments, provided is a combination therapy comprising Compound A4, and at least one other therapeutic agent is an anthracycline.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a pyrimidine analog.

According to embodiments, the pyrimidine analog is cytarabine.

In some embodiments, provided is a combination therapy comprising a menin-MLL inhibitor of Formula (I), or a pharmaceutically acceptable salt or a solvate thereof, and at least one other therapeutic agent is cytarabine.

In some embodiments, provided is a combination therapy comprising Compound A or a pharmaceutically acceptable salt or solvate thereof, and at least one other therapeutic agent is cytarabine.

In some embodiments, provided is a combination therapy comprising Compound A1, and at least one other therapeutic agent is cytarabine.

In some embodiments, provided is a combination therapy comprising Compound A2, and at least one other therapeutic agent is cytarabine.

In some embodiments, provided is a combination therapy comprising Compound A3, and at least one other therapeutic agent is cytarabine.

In some embodiments, provided is a combination therapy comprising Compound A4-a or a solvate thereof, and at least one other therapeutic agent is cytarabine.

In some embodiments, provided is a combination therapy comprising Compound A4-b or a hydrate thereof, and at least one other therapeutic agent is cytarabine.

In some embodiments, provided is a combination therapy comprising Compound A4, and at least one other therapeutic agent is cytarabine.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a purine analog.

According to embodiments, the purine analog is fludarabine.

In some embodiments, provided is a combination therapy comprising a menin-MLL inhibitor of Formula (I), or a pharmaceutically acceptable salt or a solvate thereof, and at least one other therapeutic agent is fludarabine.

In some embodiments, provided is a combination therapy comprising Compound A or a pharmaceutically acceptable salt or solvate thereof, and at least one other therapeutic agent is fludarabine.

In some embodiments, provided is a combination therapy comprising Compound A1, and at least one other therapeutic agent is fludarabine.

In some embodiments, provided is a combination therapy comprising Compound A2, and at least one other therapeutic agent is fludarabine.

In some embodiments, provided is a combination therapy comprising Compound A3, and at least one other therapeutic agent is fludarabine.

In some embodiments, provided is a combination therapy comprising Compound A4-a or a solvate thereof, and at least one other therapeutic agent is fludarabine.

In some embodiments, provided is a combination therapy comprising Compound A4-b or a hydrate thereof, and at least one other therapeutic agent is fludarabine.

In some embodiments, provided is a combination therapy comprising Compound A4, and at least one other therapeutic agent is fludarabine.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is an IDH inhibitor.

In some embodiments, the IDH inhibitor is an isocitrate dehydrogenase-1 inhibitor (e.g., ivosidenib).

According to embodiments, the IDI inhibitor is ivosidenib.

In some embodiments, provided is a combination therapy comprising a menin-MLL inhibitor of Formula (I), or a pharmaceutically acceptable salt or a solvate thereof, and at least one other therapeutic agent is ivosidenib.

In some embodiments, provided is a combination therapy comprising Compound A or a pharmaceutically acceptable salt or solvate thereof, and at least one other therapeutic agent is ivosidenib.

In some embodiments, provided is a combination therapy comprising Compound A1, and at least one other therapeutic agent is ivosidenib.

In some embodiments, provided is a combination therapy comprising Compound A2, and at least one other therapeutic agent is ivosidenib.

In some embodiments, provided is a combination therapy comprising Compound A3, and at least one other therapeutic agent is ivosidenib.

In some embodiments, provided is a combination therapy comprising Compound A4-a or a solvate thereof, and at least one other therapeutic agent is ivosidenib.

In some embodiments, provided is a combination therapy comprising Compound A4-b or a hydrate thereof, and at least one other therapeutic agent is ivosidenib.

In some embodiments, provided is a combination therapy comprising Compound A4, and at least one other therapeutic agent is ivosidenib.

In some embodiments, the IDH is an isocitrate dehydrogenase-2 inhibitor (e.g., enasidenib).

According to embodiments, the IDH inhibitor is enasidenib.

In some embodiments, provided is a combination therapy comprising a menin-MLL inhibitor of Formula (I), or a pharmaceutically acceptable salt or a solvate thereof, and at least one other therapeutic agent is enasidenib.

In some embodiments, provided is a combination therapy comprising Compound A or a pharmaceutically acceptable salt or solvate thereof, and at least one other therapeutic agent is enasidenib.

In some embodiments, provided is a combination therapy comprising Compound A1, and at least one other therapeutic agent is enasidenib.

In some embodiments, provided is a combination therapy comprising Compound A2, and at least one other therapeutic agent is enasidenib.

In some embodiments, provided is a combination therapy comprising Compound A3, and at least one other therapeutic agent is enasidenib.

In some embodiments, provided is a combination therapy comprising Compound A4-a or a solvate thereof, and at least one other therapeutic agent is enasidenib.

In some embodiments, provided is a combination therapy comprising Compound A4-b or a hydrate thereof, and at least one other therapeutic agent is enasidenib.

In some embodiments, provided is a combination therapy comprising Compound A4, and at least one other therapeutic agent is enasidenib.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is an immunomodulatory agent.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is an immunomodulatory agent which is a PD-1 inhibitor.

According to embodiments, the immunomodulatory agent is nivolumab, atezolizumab, pembrolizumab, thalidomide, lenalidomide, pomalidomide, Bacillus Calmette-Guérin (BCG) or levamisole.

In some embodiments, provided is a combination therapy comprising a menin-MLL inhibitor of Formula (I), or a pharmaceutically acceptable salt or a solvate thereof, and at least one other therapeutic agent is nivolumab.

In some embodiments, provided is a combination therapy comprising Compound A or a pharmaceutically acceptable salt or solvate thereof, and at least one other therapeutic agent is nivolumab.

In some embodiments, provided is a combination therapy comprising Compound A1, and at least one other therapeutic agent is nivolumab.

In some embodiments, provided is a combination therapy comprising Compound A2, and at least one other therapeutic agent is nivolumab.

In some embodiments, provided is a combination therapy comprising Compound A3, and at least one other therapeutic agent is nivolumab.

In some embodiments, provided is a combination therapy comprising Compound A4-a or a solvate thereof, and at least one other therapeutic agent is nivolumab.

In some embodiments, provided is a combination therapy comprising Compound A4-b or a hydrate thereof, and at least one other therapeutic agent is nivolumab.

In some embodiments, provided is a combination therapy comprising Compound A4, and at least one other therapeutic agent is nivolumab.

In some embodiments, provided is a combination therapy comprising a menin-MLL inhibitor of Formula (I), or a pharmaceutically acceptable salt or a solvate thereof, and at least one other therapeutic agent is atezolizumab.

In some embodiments, provided is a combination therapy comprising Compound A or a pharmaceutically acceptable salt or solvate thereof, and at least one other therapeutic agent is atezolizumab.

In some embodiments, provided is a combination therapy comprising Compound A1, and at least one other therapeutic agent is atezolizumab.

In some embodiments, provided is a combination therapy comprising Compound A2, and at least one other therapeutic agent is atezolizumab.

In some embodiments, provided is a combination therapy comprising Compound A3, and at least one other therapeutic agent is atezolizumab.

In some embodiments, provided is a combination therapy comprising Compound A4-a or a solvate thereof, and at least one other therapeutic agent is atezolizumab.

In some embodiments, provided is a combination therapy comprising Compound A4-b or a hydrate thereof, and at least one other therapeutic agent is atezolizumab.

In some embodiments, provided is a combination therapy comprising Compound A4, and at least one other therapeutic agent is atezolizumab.

In some embodiments, provided is a combination therapy comprising a menin-MLL inhibitor of Formula (I), or a pharmaceutically acceptable salt or a solvate thereof, and at least one other therapeutic agent is pembrolizumab.

In some embodiments, provided is a combination therapy comprising Compound A or a pharmaceutically acceptable salt or solvate thereof, and at least one other therapeutic agent is pembrolizumab.

In some embodiments, provided is a combination therapy comprising Compound A1, and at least one other therapeutic agent is pembrolizumab.

In some embodiments, provided is a combination therapy comprising Compound A2, and at least one other therapeutic agent is pembrolizumab.

In some embodiments, provided is a combination therapy comprising Compound A3, and at least one other therapeutic agent is pembrolizumab.

In some embodiments, provided is a combination therapy comprising Compound A4-a or a solvate thereof, and at least one other therapeutic agent is pembrolizumab.

In some embodiments, provided is a combination therapy comprising Compound A4-b or a hydrate thereof, and at least one other therapeutic agent is pembrolizumab.

In some embodiments, provided is a combination therapy comprising Compound A4, and at least one other therapeutic agent is pembrolizumab.

In some embodiments, provided is a combination therapy comprising a menin-MLL inhibitor of Formula (I), or a pharmaceutically acceptable salt or a solvate thereof, and at least one other therapeutic agent is thalidomide.

In some embodiments, provided is a combination therapy comprising Compound A or a pharmaceutically acceptable salt or solvate thereof, and at least one other therapeutic agent is thalidomide.

In some embodiments, provided is a combination therapy comprising Compound A1, and at least one other therapeutic agent is thalidomide.

In some embodiments, provided is a combination therapy comprising Compound A2, and at least one other therapeutic agent is thalidomide.

In some embodiments, provided is a combination therapy comprising Compound A3, and at least one other therapeutic agent is thalidomide.

In some embodiments, provided is a combination therapy comprising Compound A4-a or a solvate thereof, and at least one other therapeutic agent is thalidomide.

In some embodiments, provided is a combination therapy comprising Compound A4-b or a hydrate thereof, and at least one other therapeutic agent is thalidomide.

In some embodiments, provided is a combination therapy comprising Compound A4, and at least one other therapeutic agent is thalidomide.

In some embodiments, provided is a combination therapy comprising a menin-MLL inhibitor of Formula (I), or a pharmaceutically acceptable salt or a solvate thereof, and at least one other therapeutic agent is lenalidomide.

In some embodiments, provided is a combination therapy comprising Compound A or a pharmaceutically acceptable salt or solvate thereof, and at least one other therapeutic agent is lenalidomide.

In some embodiments, provided is a combination therapy comprising Compound A1, and at least one other therapeutic agent is lenalidomide.

In some embodiments, provided is a combination therapy comprising Compound A2, and at least one other therapeutic agent is lenalidomide.

In some embodiments, provided is a combination therapy comprising Compound A3, and at least one other therapeutic agent is lenalidomide.

In some embodiments, provided is a combination therapy comprising Compound A4-a or a solvate thereof, and at least one other therapeutic agent is lenalidomide.

In some embodiments, provided is a combination therapy comprising Compound A4-b or a hydrate thereof, and at least one other therapeutic agent is lenalidomide.

In some embodiments, provided is a combination therapy comprising Compound A4, and at least one other therapeutic agent is lenalidomide.

In some embodiments, provided is a combination therapy comprising a menin-MLL inhibitor of Formula (I), or a pharmaceutically acceptable salt or a solvate thereof, and at least one other therapeutic agent is pomalidomide.

In some embodiments, provided is a combination therapy comprising Compound A or a pharmaceutically acceptable salt or solvate thereof, and at least one other therapeutic agent is pomalidomide.

In some embodiments, provided is a combination therapy comprising Compound A1, and at least one other therapeutic agent is pomalidomide.

In some embodiments, provided is a combination therapy comprising Compound A2, and at least one other therapeutic agent is pomalidomide.

In some embodiments, provided is a combination therapy comprising Compound A3, and at least one other therapeutic agent is pomalidomide.

In some embodiments, provided is a combination therapy comprising Compound A4-a or a solvate thereof, and at least one other therapeutic agent is pomalidomide.

In some embodiments, provided is a combination therapy comprising Compound A4-b or a hydrate thereof, and at least one other therapeutic agent is pomalidomide.

In some embodiments, provided is a combination therapy comprising Compound A4, and at least one other therapeutic agent is pomalidomide.

In some embodiments, provided is a combination therapy comprising a menin-MLL inhibitor of Formula (I), or a pharmaceutically acceptable salt or a solvate thereof, and at least one other therapeutic agent is BCG.

In some embodiments, provided is a combination therapy comprising Compound A or a pharmaceutically acceptable salt or solvate thereof, and at least one other therapeutic agent is BCG.

In some embodiments, provided is a combination therapy comprising Compound A1, and at least one other therapeutic agent is BCG.

In some embodiments, provided is a combination therapy comprising Compound A2, and at least one other therapeutic agent is BCG.

In some embodiments, provided is a combination therapy comprising Compound A3, and at least one other therapeutic agent is BCG.

In some embodiments, provided is a combination therapy comprising Compound A4-a or a solvate thereof, and at least one other therapeutic agent is BCG.

In some embodiments, provided is a combination therapy comprising Compound A4-b or a hydrate thereof, and at least one other therapeutic agent is BCG.

In some embodiments, provided is a combination therapy comprising Compound A4, and at least one other therapeutic agent is BCG.

In some embodiments, provided is a combination therapy comprising a menin-MLL inhibitor of Formula (I), or a pharmaceutically acceptable salt or a solvate thereof, and at least one other therapeutic agent is levamisole.

In some embodiments, provided is a combination therapy comprising Compound A or a pharmaceutically acceptable salt or solvate thereof, and at least one other therapeutic agent is levamisole.

In some embodiments, provided is a combination therapy comprising Compound A1, and at least one other therapeutic agent is levamisole.

In some embodiments, provided is a combination therapy comprising Compound A2, and at least one other therapeutic agent is levamisole.

In some embodiments, provided is a combination therapy comprising Compound A3, and at least one other therapeutic agent is levamisole.

In some embodiments, provided is a combination therapy comprising Compound A4-a or a solvate thereof, and at least one other therapeutic agent is levamisole.

In some embodiments, provided is a combination therapy comprising Compound A4-b or a hydrate thereof, and at least one other therapeutic agent is levamisole.

In some embodiments, provided is a combination therapy comprising Compound A4, and at least one other therapeutic agent is levamisole.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a DHODH inhibitor.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a DHODH inhibitor compound as described herein.

According to embodiments, the DHODH inhibitor is a compound of Formula (Z), or a pharmaceutically acceptable salt, isotope, N-oxide, solvate, or stereoisomer thereof.

In some embodiments, provided is a combination therapy comprising a menin-MLL inhibitor of Formula (I), or a pharmaceutically acceptable salt or a solvate thereof, and at least one other therapeutic agent is a DHODH inhibitor of Formula (Z), or a pharmaceutically acceptable salt, isotope, N-oxide, solvate, or stereoisomer thereof.

In some embodiments, provided is a combination therapy comprising Compound A or a pharmaceutically acceptable salt or solvate thereof, and at least one other therapeutic agent is a DHODH inhibitor of Formula (Z), or a pharmaceutically acceptable salt, isotope, N-oxide, solvate, or stereoisomer thereof.

In some embodiments, provided is a combination therapy comprising Compound A1, and at least one other therapeutic agent is a DHODH inhibitor of Formula (Z), or a pharmaceutically acceptable salt, isotope, N-oxide, solvate, or stereoisomer thereof.

In some embodiments, provided is a combination therapy comprising Compound A2, and at least one other therapeutic agent is a DHODH inhibitor of Formula (Z), or a pharmaceutically acceptable salt, isotope, N-oxide, solvate, or stereoisomer thereof.

In some embodiments, provided is a combination therapy comprising Compound A3, and at least one other therapeutic agent is a DHODH inhibitor of Formula (Z), or a pharmaceutically acceptable salt, isotope, N-oxide, solvate, or stereoisomer thereof

In some embodiments, provided is a combination therapy comprising Compound A4-a or a solvate thereof, and at least one other therapeutic agent is a DHODH inhibitor of Formula (Z), or a pharmaceutically acceptable salt, isotope, N-oxide, solvate, or stereoisomer thereof.

In some embodiments, provided is a combination therapy comprising Compound A4-b or a hydrate thereof, and at least one other therapeutic agent is a DHODH inhibitor of Formula (Z), or a pharmaceutically acceptable salt, isotope, N-oxide, solvate, or stereoisomer thereof.

In some embodiments, provided is a combination therapy comprising Compound A4, and at least one other therapeutic agent is a DHODH inhibitor of Formula (Z), or a pharmaceutically acceptable salt, isotope, N-oxide, solvate, or stereoisomer thereof.

In some embodiments, provided is a combination therapy comprising a menin-MLL inhibitor of Formula (I), or a pharmaceutically acceptable salt or a solvate thereof, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination therapy comprising Compound A or a pharmaceutically acceptable salt or solvate thereof, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination therapy comprising Compound A1, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination therapy comprising Compound A2, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination therapy comprising Compound A3, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination therapy comprising Compound A4-a or a solvate thereof, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination therapy comprising Compound A4-b or a hydrate thereof, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination therapy comprising Compound A4, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a kinase inhibitor.

According to embodiments, the kinase inhibitor is a serine and/or tyrosine kinase inhibitor.

According to embodiments, the kinase inhibitor is an inhibitor of FLT3 and/or BTK.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a FLT3 inhibitor.

According to embodiments, the FLT3 inhibitor is sorafenib, sunitinib, midostaurin (PKC412), lestaurtinib (CEP-701), tandutinib (MLN518), quizartinib (AC220), gilteritinib (ASP2215), or KW-2449.

In some embodiments, provided is a combination therapy comprising a menin-MLL inhibitor of Formula (I), or a pharmaceutically acceptable salt or a solvate thereof, and at least one other therapeutic agent is sorafenib.

In some embodiments, provided is a combination therapy comprising Compound A or a pharmaceutically acceptable salt or solvate thereof, and at least one other therapeutic agent is sorafenib.

In some embodiments, provided is a combination therapy comprising Compound A1, and at least one other therapeutic agent is sorafenib.

In some embodiments, provided is a combination therapy comprising Compound A2, and at least one other therapeutic agent is sorafenib.

In some embodiments, provided is a combination therapy comprising Compound A3, and at least one other therapeutic agent is sorafenib.

In some embodiments, provided is a combination therapy comprising Compound A4-a or a solvate thereof, and at least one other therapeutic agent is sorafenib.

In some embodiments, provided is a combination therapy comprising Compound A4-b or a hydrate thereof, and at least one other therapeutic agent is sorafenib.

In some embodiments, provided is a combination therapy comprising Compound A4, and at least one other therapeutic agent is sorafenib.

In some embodiments, provided is a combination therapy comprising a menin-MLL inhibitor of Formula (I), or a pharmaceutically acceptable salt or a solvate thereof, and at least one other therapeutic agent is sunitinib.

In some embodiments, provided is a combination therapy comprising Compound A or a pharmaceutically acceptable salt or solvate thereof, and at least one other therapeutic agent is sunitinib.

In some embodiments, provided is a combination therapy comprising Compound A1, and at least one other therapeutic agent is sunitinib.

In some embodiments, provided is a combination therapy comprising Compound A2, and at least one other therapeutic agent is sunitinib.

In some embodiments, provided is a combination therapy comprising Compound A3, and at least one other therapeutic agent is sunitinib.

In some embodiments, provided is a combination therapy comprising Compound A4-a or a solvate thereof, and at least one other therapeutic agent is sunitinib.

In some embodiments, provided is a combination therapy comprising Compound A4-b or a hydrate thereof, and at least one other therapeutic agent is sunitinib.

In some embodiments, provided is a combination therapy comprising Compound A4, and at least one other therapeutic agent is sunitinib.

In some embodiments, provided is a combination therapy comprising a menin-MLL inhibitor of Formula (I), or a pharmaceutically acceptable salt or a solvate thereof, and at least one other therapeutic agent is midostaurin.

In some embodiments, provided is a combination therapy comprising Compound A or a pharmaceutically acceptable salt or solvate thereof, and at least one other therapeutic agent is midostaurin.

In some embodiments, provided is a combination therapy comprising Compound A1, and at least one other therapeutic agent is midostaurin.

In some embodiments, provided is a combination therapy comprising Compound A2, and at least one other therapeutic agent is midostaurin.

In some embodiments, provided is a combination therapy comprising Compound A3, and at least one other therapeutic agent is midostaurin.

In some embodiments, provided is a combination therapy comprising Compound A4-a or a solvate thereof, and at least one other therapeutic agent is midostaurin.

In some embodiments, provided is a combination therapy comprising Compound A4-b or a hydrate thereof, and at least one other therapeutic agent is midostaurin.

In some embodiments, provided is a combination therapy comprising Compound A4, and at least one other therapeutic agent is midostaurin.

In some embodiments, provided is a combination therapy comprising a menin-MLL inhibitor of Formula (I), or a pharmaceutically acceptable salt or a solvate thereof, and at least one other therapeutic agent is lestaurtinib.

In some embodiments, provided is a combination therapy comprising Compound A or a pharmaceutically acceptable salt or solvate thereof, and at least one other therapeutic agent is lestaurtinib.

In some embodiments, provided is a combination therapy comprising Compound A1, and at least one other therapeutic agent is lestaurtinib.

In some embodiments, provided is a combination therapy comprising Compound A2, and at least one other therapeutic agent is lestaurtinib.

In some embodiments, provided is a combination therapy comprising Compound A3, and at least one other therapeutic agent is lestaurtinib.

In some embodiments, provided is a combination therapy comprising Compound A4-a or a solvate thereof, and at least one other therapeutic agent is lestaurtinib.

In some embodiments, provided is a combination therapy comprising Compound A4-b or a hydrate thereof, and at least one other therapeutic agent is lestaurtinib.

In some embodiments, provided is a combination therapy comprising Compound A4, and at least one other therapeutic agent is lestaurtinib.

In some embodiments, provided is a combination therapy comprising a menin-MLL inhibitor of Formula (I), or a pharmaceutically acceptable salt or a solvate thereof, and at least one other therapeutic agent is tandutinib.

In some embodiments, provided is a combination therapy comprising Compound A or a pharmaceutically acceptable salt or solvate thereof, and at least one other therapeutic agent is tandutinib.

In some embodiments, provided is a combination therapy comprising Compound A1, and at least one other therapeutic agent is tandutinib.

In some embodiments, provided is a combination therapy comprising Compound A2, and at least one other therapeutic agent is tandutinib.

In some embodiments, provided is a combination therapy comprising Compound A3, and at least one other therapeutic agent is tandutinib.

In some embodiments, provided is a combination therapy comprising Compound A4-a or a solvate thereof, and at least one other therapeutic agent is tandutinib.

In some embodiments, provided is a combination therapy comprising Compound A4-b or a hydrate thereof, and at least one other therapeutic agent is tandutinib.

In some embodiments, provided is a combination therapy comprising Compound A4, and at least one other therapeutic agent is tandutinib.

In some embodiments, provided is a combination therapy comprising a menin-MLL inhibitor of Formula (I), or a pharmaceutically acceptable salt or a solvate thereof, and at least one other therapeutic agent is AC220.

In some embodiments, provided is a combination therapy comprising Compound A or a pharmaceutically acceptable salt or solvate thereof, and at least one other therapeutic agent is AC220.

In some embodiments, provided is a combination therapy comprising Compound A1, and at least one other therapeutic agent is AC220.

In some embodiments, provided is a combination therapy comprising Compound A2, and at least one other therapeutic agent is AC220.

In some embodiments, provided is a combination therapy comprising Compound A3, and at least one other therapeutic agent is AC220.

In some embodiments, provided is a combination therapy comprising Compound A4-a or a solvate thereof, and at least one other therapeutic agent is AC220.

In some embodiments, provided is a combination therapy comprising Compound A4-b or a hydrate thereof, and at least one other therapeutic agent is AC220.

In some embodiments, provided is a combination therapy comprising Compound A4, and at least one other therapeutic agent is AC220.

In some embodiments, provided is a combination therapy comprising a menin-MLL inhibitor of Formula (I), or a pharmaceutically acceptable salt or a solvate thereof, and at least one other therapeutic agent is ASP2215.

In some embodiments, provided is a combination therapy comprising Compound A or a pharmaceutically acceptable salt or solvate thereof, and at least one other therapeutic agent is ASP2215.

In some embodiments, provided is a combination therapy comprising Compound A1, and at least one other therapeutic agent is ASP2215.

In some embodiments, provided is a combination therapy comprising Compound A2, and at least one other therapeutic agent is ASP2215.

In some embodiments, provided is a combination therapy comprising Compound A3, and at least one other therapeutic agent is ASP2215.

In some embodiments, provided is a combination therapy comprising Compound A4-a or a solvate thereof, and at least one other therapeutic agent is ASP2215.

In some embodiments, provided is a combination therapy comprising Compound A4-b or a hydrate thereof, and at least one other therapeutic agent is ASP2215.

In some embodiments, provided is a combination therapy comprising Compound A4, and at least one other therapeutic agent is ASP2215.

In some embodiments, provided is a combination therapy comprising a menin-MLL inhibitor of Formula (I), or a pharmaceutically acceptable salt or a solvate thereof, and at least one other therapeutic agent is KW-2449. In some embodiments, provided is a combination therapy comprising Compound A or a pharmaceutically acceptable salt or solvate thereof, and at least one other therapeutic agent is KW-2449.

In some embodiments, provided is a combination therapy comprising Compound A1, and at least one other therapeutic agent is KW-2449.

In some embodiments, provided is a combination therapy comprising Compound A2, and at least one other therapeutic agent is KW-2449.

In some embodiments, provided is a combination therapy comprising Compound A3, and at least one other therapeutic agent is KW-2449.

In some embodiments, provided is a combination therapy comprising Compound A4-a or a solvate thereof, and at least one other therapeutic agent is KW-2449.

In some embodiments, provided is a combination therapy comprising Compound A4-b or a hydrate thereof, and at least one other therapeutic agent is KW-2449.

In some embodiments, provided is a combination therapy comprising Compound A4, and at least one other therapeutic agent is KW-2449.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a BTK inhibitor.

In some embodiments, provided is a combination therapy comprising Compound A or a pharmaceutically acceptable salt or solvate thereof, and at least one other therapeutic agent is a BTK inhibitor.

In some embodiments, provided is a combination therapy comprising Compound A1, and at least one other therapeutic agent is a BTK inhibitor.

In some embodiments, provided is a combination therapy comprising Compound A2, and at least one other therapeutic agent is a BTK inhibitor.

In some embodiments, provided is a combination therapy comprising Compound A3, and at least one other therapeutic agent is a BTK inhibitor.

In some embodiments, provided is a combination therapy comprising Compound A4-a or a solvate thereof, and at least one other therapeutic agent is a BTK inhibitor.

In some embodiments, provided is a combination therapy comprising Compound A4-b or a hydrate thereof, and at least one other therapeutic agent is a BTK inhibitor.

In some embodiments, provided is a combination therapy comprising Compound A4, and at least one other therapeutic agent is a BTK inhibitor.

According to embodiments, the BTK inhibitor is ibrutinib.

In some embodiments, provided is a combination therapy comprising a menin-MLL inhibitor of Formula (I), or a pharmaceutically acceptable salt or a solvate thereof, and at least one other therapeutic agent is ibrutinib.

In some embodiments, provided is a combination therapy comprising Compound A or a pharmaceutically acceptable salt or solvate thereof, and at least one other therapeutic agent is ibrutinib.

In some embodiments, provided is a combination therapy comprising Compound A1, and at least one other therapeutic agent is ibrutinib.

In some embodiments, provided is a combination therapy comprising Compound A2, and at least one other therapeutic agent is ibrutinib.

In some embodiments, provided is a combination therapy comprising Compound A3, and at least one other therapeutic agent is ibrutinib.

In some embodiments, provided is a combination therapy comprising Compound A4-a or a solvate thereof, and at least one other therapeutic agent is ibrutinib.

In some embodiments, provided is a combination therapy comprising Compound A4-b or a hydrate thereof, and at least one other therapeutic agent is ibrutinib.

In some embodiments, provided is a combination therapy comprising Compound A4, and at least one other therapeutic agent is ibrutinib.

In some embodiments, provided is a combination therapy comprising a menin-MLL inhibitor of Formula (I), or a pharmaceutically acceptable salt or a solvate thereof, and at least one other therapeutic agent, wherein at least one other therapeutic agent is a CD20 inhibitor.

According to embodiments, the CD20 inhibitor is an anti-CD20 antibody, in particular obinutuzumab (GA101).

In some embodiments, provided is a combination therapy comprising Compound A or a pharmaceutically acceptable salt or solvate thereof, and at least one other therapeutic agent is a CD20 inhibitor.

In some embodiments, provided is a combination therapy comprising Compound A1, and at least one other therapeutic agent is a CD20 inhibitor.

In some embodiments, provided is a combination therapy comprising Compound A2, and at least one other therapeutic agent is a CD20 inhibitor.

In some embodiments, provided is a combination therapy comprising Compound A3, and at least one other therapeutic agent is a CD20 inhibitor.

In some embodiments, provided is a combination therapy comprising Compound A4-a or a solvate thereof, and at least one other therapeutic agent is a CD20 inhibitor.

In some embodiments, provided is a combination therapy comprising Compound A4-b or a hydrate thereof, and at least one other therapeutic agent is a CD20 inhibitor.

In some embodiments, provided is a combination therapy comprising Compound A4, and at least one other therapeutic agent is a CD20 inhibitor.

In some embodiments, provided is a combination therapy comprising a menin-MLL inhibitor of Formula (I), or a pharmaceutically acceptable salt or a solvate thereof, and at least one other therapeutic agent is GA101.

In some embodiments, provided is a combination therapy comprising Compound A or a pharmaceutically acceptable salt or solvate thereof, and at least one other therapeutic agent is GA101.

In some embodiments, provided is a combination therapy comprising Compound A1, and at least one other therapeutic agent is GA101.

In some embodiments, provided is a combination therapy comprising Compound A2, and at least one other therapeutic agent is GA101.

In some embodiments, provided is a combination therapy comprising Compound A3, and at least one other therapeutic agent is GA101.

In some embodiments, provided is a combination therapy comprising Compound A4-a or a solvate thereof, and at least one other therapeutic agent is GA101.

In some embodiments, provided is a combination therapy comprising Compound A4-b or a hydrate thereof, and at least one other therapeutic agent is GA101.

In some embodiments, provided is a combination therapy comprising Compound A4, and at least one other therapeutic agent is GA101.

All possible combinations of the above indicated embodiments are considered to be embraced within the scope of the invention

In some embodiments, provided are methods for treating a subject who has been diagnosed with a hematopoietic disorder. The present invention relates, for example, to novel methods that comprise administering to the subject a therapeutically effective amount of a menin-MLL inhibitor as described herein; and a therapeutically effective amount of at least one other therapeutic agent.

An additional embodiment of the invention relates to methods as described herein wherein the compound of Formula (I), or a pharmaceutically acceptable salt or a solvate thereof is administered orally to a subject.

An additional embodiment of the invention relates to methods as described herein wherein the compound of Formula (I), or a pharmaceutically acceptable salt or a solvate thereof is administered in a dose of from about 1 mg/kg to about 50 mg/kg to the subject.

An additional embodiment of the invention relates to methods as described herein wherein the compound of Formula (I), or a pharmaceutically acceptable salt or a solvate thereof is administered in a dose of from about 2.5 mg/kg to about 25 mg/kg to the subject.

An additional embodiment of the invention relates to methods as described herein wherein the compound of Formula (I), or a pharmaceutically acceptable salt or a solvate thereof is administered in a dose of from about 7.5 mg/kg to about 12.5 mg/kg to the subject.

An additional embodiment of the invention relates to methods as described herein wherein the compound of Formula (I), or a pharmaceutically acceptable salt or a solvate thereof is administered in a dose of from about 8 mg/kg to about 10 mg/kg to the subject.

An additional embodiment of the invention relates to methods as described herein wherein the compound of Formula (I), or a pharmaceutically acceptable salt or a solvate thereof is administered in a dose of from about 0.1 mg to about 5 mg to the subject.

An additional embodiment of the invention relates to methods as described herein wherein the at least one other therapeutic agent is administered orally to a subject.

An additional embodiment of the invention relates to methods as described herein wherein the at least one other therapeutic agent is administered orally in a dose of from about 1 mg to about 500 mg to the subject.

An additional embodiment of the invention relates to methods as described herein wherein at least one other therapeutic agent is administered to the subject intravenously or subcutaneously.

An additional embodiment of the invention relates to methods as described herein wherein at least one other therapeutic agent is administered intravenously or subcutaneously in a dose of from about 10 mg/m² to about 250 mg/m² to the subject.

An additional embodiment of the invention relates to methods as described herein wherein at least one other therapeutic agent is administered intravenously or subcutaneously in a dose of from about 50 mg/m² to about 150 mg/m² to the subject.

An additional embodiment of the invention relates to methods as described herein wherein at least one other therapeutic agent is administered intravenously or subcutaneously in a dose of from about 60 mg/m² to about 100 mg/m² to the subject.

An additional embodiment of the invention relates to methods as described herein wherein at least one other therapeutic agent is administered intravenously or subcutaneously in a dose of about 75 mg/m² to the subject.

An additional embodiment of the invention relates to methods for treating a subject who has been diagnosed with cancer (e.g., wherein the cancer is a hematopoietic disorder, such as myelodysplastic syndrome (MDS), acute myeloid leukemia (AML), acute lymphoblastic leukemia (ALL), a small lymphocytic lymphoma (SLL) or chronic lymphocytic leukemia (CLL)), wherein the method comprises administering to the subject:
a therapeutically effective amount of a menin-MLL inhibitor of Formula (I), or a tautomer or a stereoisomeric form thereof, or a pharmaceutically acceptable salt or a solvate thereof; and
a therapeutically effective amount of a DHODH inhibitor of Formula (Z), or a pharmaceutically acceptable salt, isotope, N-oxide, solvate, or stereoisomer thereof;
for example, wherein the menin-MLL inhibitor and the DHODH inhibitor are administered in accordance with their dosing schedules for a time period, e.g., (i) simultaneously or sequentially in either order on the same day(s) within a time period (e.g., a 21-day period, or a 28-day period, or a 3-month period, or a 6-month period, or a one-year period, etc.), and/or (ii) on different days within a time period.

An additional embodiment of the invention relates to methods for treating a subject who has been diagnosed with cancer (e.g., wherein the cancer is a hematopoietic disorder, such as myelodysplastic syndrome (MDS), acute myeloid leukemia (AML), acute lymphoblastic leukemia (ALL), a small lymphocytic lymphoma (SLL) or chronic lymphocytic leukemia (CLL)), wherein the method comprises administering to the subject:
a therapeutically effective amount of a menin-MLL inhibitor that is Compound A or a pharmaceutically acceptable salt or solvate thereof (e.g., Compound A1, or Compound A2, or Compound A3, or Compound A4-a or a solvate thereof, or Compound A4-b or a hydrate thereof, or Compound A4); and
a therapeutically effective amount of a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one);
for example, wherein the menin-MLL inhibitor and the DHODH inhibitor are administered in accordance with their dosing schedules for a time period, e.g., (i) simultaneously or sequentially in either order on the same day(s) within a time period (e.g., a 21-day period, or a 28-day period, or a 3-month period, or a 6-month period, or a one-year period, etc.), and/or (ii) on different days within a time period.

An additional embodiment of the invention relates to methods as described herein wherein compound of Formula (I), or a pharmaceutically acceptable salt or a solvate thereof is administered to the subject daily.

An additional embodiment of the invention relates to methods as described herein wherein compound of Formula (I), or a pharmaceutically acceptable salt or a solvate thereof is administered to the subject daily for at least 7 days.

An additional embodiment of the invention relates to methods as described herein wherein the at least one other therapeutic agent is administered to the subject daily.

An additional embodiment of the invention relates to methods as described herein wherein the at least one other therapeutic agent is administered to the subject daily for at least 7 days.

An additional embodiment of the invention relates to methods as described herein wherein at least one other therapeutic agent is administered to the subject daily.

An additional embodiment of the invention relates to methods as described herein wherein at least one other therapeutic agent is administered to the subject daily for at least 21 days.

Optimal dosages of any of the therapeutic compounds described herein to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect.

The above dosages are thus exemplary of the average case. There can be, of course, individual instances wherein higher or lower dosage ranges are merited, and such are within the scope of this invention.

The therapeutic compounds described herein may be administered in any of the foregoing compositions and dosage regimens or by means of those compositions and dosage regimens established in the art whenever use of the therapeutic compounds described herein is administered to a subject in need thereof.

The therapeutic compounds described herein may be administered to the subject simultaneously or sequentially. When administered sequentially, the menin-MLL inhibitor of Formula (I) may be administered first. When administration is simultaneous, the combination may be administered either in the same or a different pharmaceutical composition. For instance, the menin-MLL inhibitor of Formula (I) may be administered prior to, simultaneous with, or after the administration of at least one other therapeutic agent. Likewise, at least one other therapeutic agent may be administered prior to, simultaneous with, or after the administration of another therapeutic agent. Adjunctive therapy, i.e., where one or two agent(s) are used as the primary treatment and the other agent is used to assist that primary treatment, is also an embodiment of the present invention.

An embodiment of the invention relates to a therapeutically effective amount of a menin-MLL inhibitor including the compounds of Formula (I) and the pharmaceutically acceptable salts, and the solvates thereof, or any subgroup thereof as mentioned in any of the embodiments, for use in combination with a therapeutically effective amount of at least one other therapeutic agent, wherein the at least one other therapeutic agent is a hypomethylating agent, cytidine deaminase inhibitor, a DNA intercalating agent, a pyrimidine analog, a purine analog, a kinase inhibitor, a CD20 inhibitor, an isocitrate dehydrogenase inhibitor, an immunomodulatory agent or a dihydroorotate dehydrogenase inhibitor. In certain embodiments, the aforementioned therapeutically effective amounts are administered in separate dosage forms for use in treating a subject who has been diagnosed with a hematopoietic disorder.

An embodiment of the invention relates to a pharmaceutical product including a menin-MLL inhibitor of Formula (I) and the pharmaceutically acceptable salts, and the solvates thereof, or any subgroup thereof as mentioned in any of the embodiments, and at least one other therapeutic agent is a hypomethylating agent, cytidine deaminase inhibitor, a DNA intercalating agent, a pyrimidine analog, a purine analog, a kinase inhibitor, a CD20 inhibitor, an isocitrate dehydrogenase inhibitor, an immunomodulatory agent or a dihydroorotate dehydrogenase inhibitor as a combined preparation for simultaneous, separate or sequential use in treating a subject who has been diagnosed with a hematopoietic disorder.

The following Examples are provided to illustrate some of the concepts described within this disclosure. While the Example is considered to provide an embodiment, it should not be considered to limit the more general embodiments described herein.

### Examples

### General Synthetic Schemes

### Azacitidine

Azacitidine is available commercially.

### Compounds of Formula I

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or prepared by standard synthetic processes commonly used by those skilled in the art of organic chemistry. The following schemes are only meant to represent examples of the invention and are in no way meant to be a limit of the invention.

Alternatively, compounds of Formula (I) may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups (PG) can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

All abbreviations used in the general schemes for Formula (I) are as defined in Table 1B below in the part Examples. Variables are as defined in the scope or as specifically defined in the general Schemes.

### Part A) Schemes 1a, 1b, 1c, 2a, 2b and 3

Rn=C₁₋₆alkyl-NR^{8a}PG or C₁₋₆alkyl-OPG or C₁₋₆alkyl-C(═O)OR^{9a}, PG=protecting group

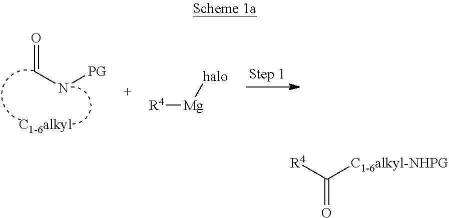

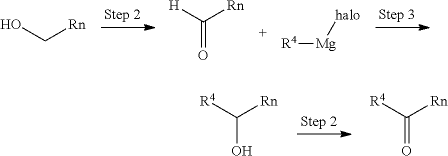

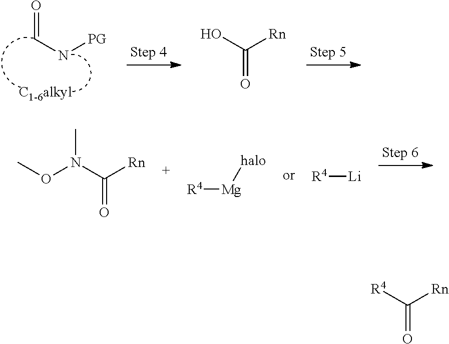

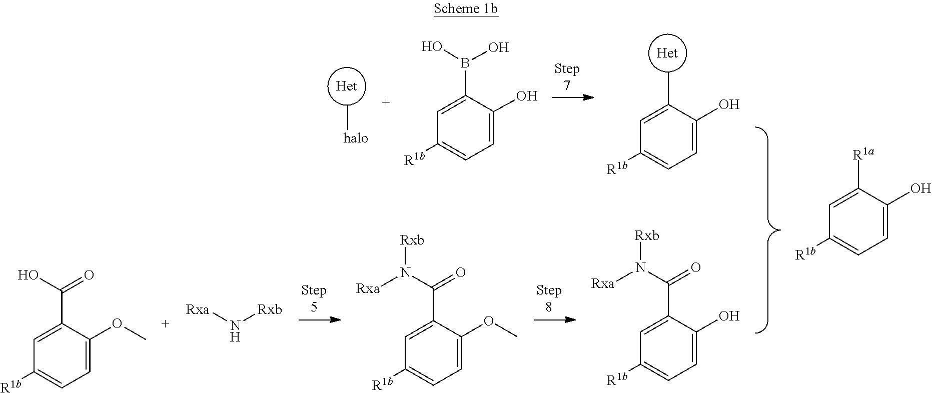

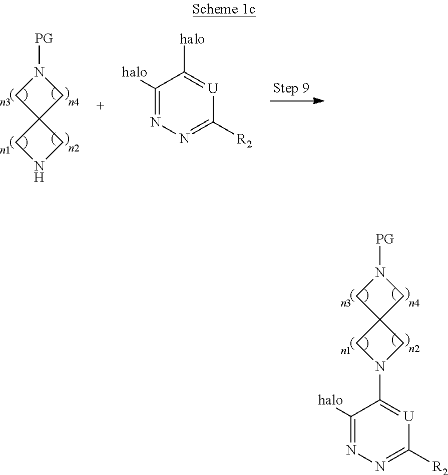

In Scheme 1a, 1b and 1c the following reaction conditions apply:
Step 1: at a suitable temperature such as for example −70° C., in the presence of a suitable base such as for example TMEDA and a suitable organometallic reagent such as for example isopropylmagnesium bromide, in a suitable solvent such as for example THF;
Step 2: at a suitable temperature such as for example from 0° C. to RT, in the presence of a suitable oxidative reagent such as for example DMP, in a suitable solvent such as for example DCM;
Step 3: at a suitable temperature such as for example from −20° C. to RT, in the presence of a suitable organometallic reagent such as for example isopropylmagnesium bromide, in a suitable solvent such as for example THF;
Step 4: at a suitable temperature such as for example 80° C., in the presence of a suitable base such as for example NaOH, in suitable solvents such as for example THF and H₂O;
Step 5: at a suitable temperature such as for example RT, in the presence of suitable amide condensation reagents such as for example EDCI and HOBt, in the presence of a suitable base such as for example NMM, in a suitable solvent such as for example DCM;
Step 6: at a suitable temperature such as for example −70° C., in the presence of a suitable organometallic reagent such as for example isopropyllithium, in a suitable solvent such as for example THF;
Step 7: at a suitable temperature such as for example 90° C., in the presence of a suitable organometallic catalyst such as for example Pd(dppf)Cl₂, in the presence of a suitable base such as for example Na₂CO₃, in suitable solvents such as for example 1,4-dioxane and H₂O;
Step 8: at a suitable temperature such as for example from 0° C. to RT, in the presence of a suitable Lewis acid such as for example BBr₃, in a suitable solvent such as for example DCM;
Step 9: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;

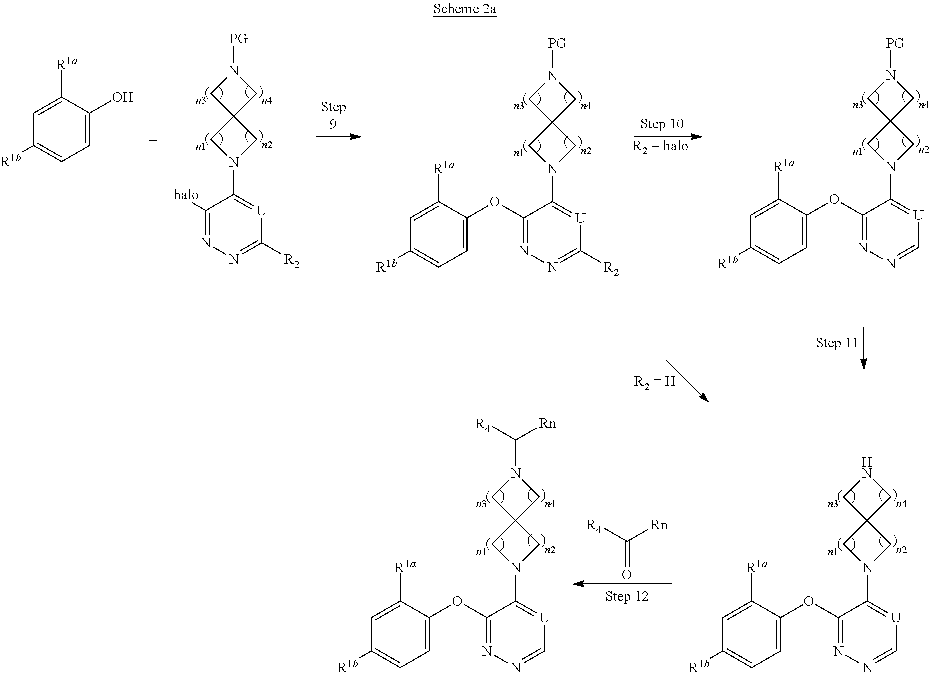

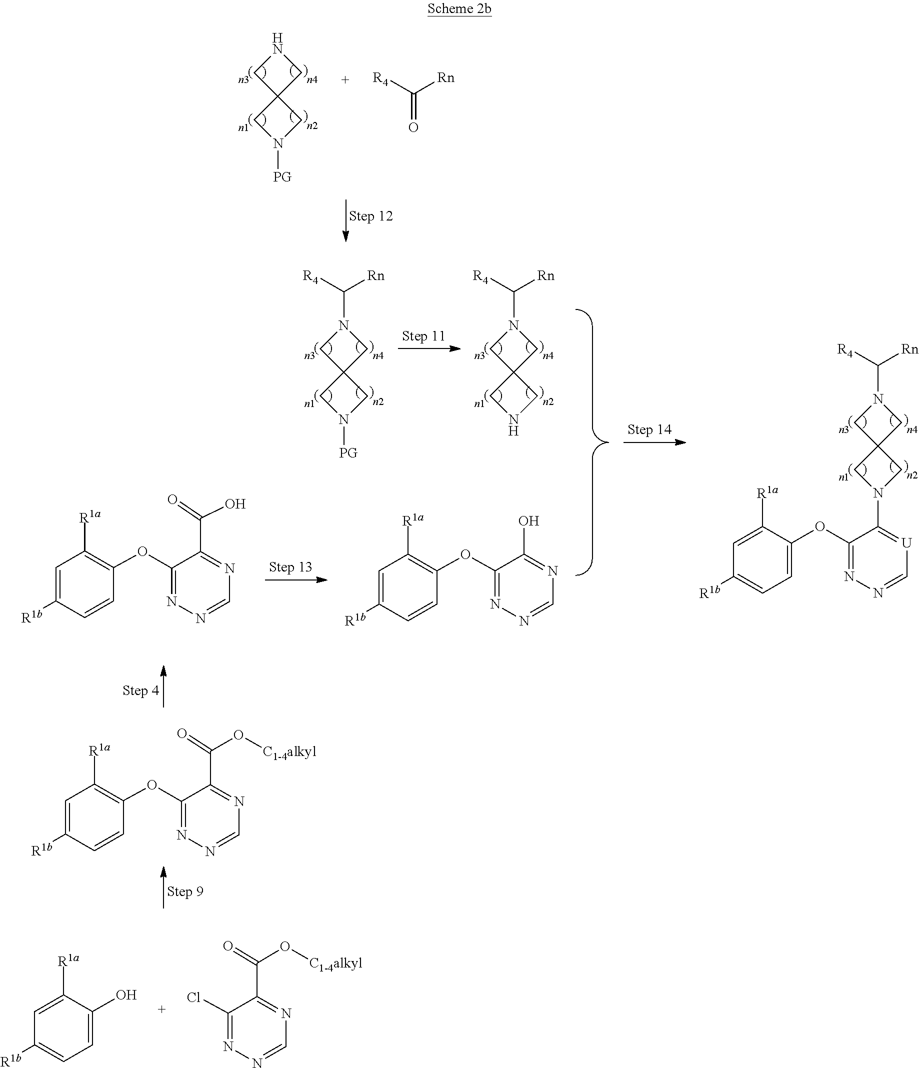

In Scheme 2a and 2b, the following reaction conditions apply:
Step 9: See Step 9 in Scheme 1;
Step 10: at a suitable temperature such as for example RT, in the presence of a suitable catalyst such as for example Pd/C, in the presence of a suitable reductive reagent such as for example H₂, optionally in the presence of a suitable base such as for example TEA, in a suitable solvent such as for example THF; Alternatively, at a suitable temperature such as RT, in the presence of a suitable catalyst such as for example Pd(dppf)Cl₂·DCM complex, a suitable reducing agent such NaBH₄, a suitable base such as for example TMEDA, in a suitable solvent such as for example THF.
Step 11: for N deprotection, at a suitable temperature such as for example RT, in the presence of a suitable acid as for example TFA, in a suitable solvent such as for example DCM; for O deprotection, at a suitable temperature such as for example RT, in the presence of a suitable acid as for example 4-methylbenzenesulfonic acid, in a suitable solvent such as for example MeOH;
Step 12: at a suitable temperature such as for example 80° C., optionally in the presence of a suitable Lewis acid such as for example ZnCl₂, in the presence of a suitable reductive reagent such as for example NaBH₃CN, in a suitable solvent such as for example MeOH;
Step 13: at a suitable temperature such as for example RT, in the presence of a suitable organometallic catalyst such as for example Ag(Phen)₂OTf, in the presence of a suitable brominating reagent such as for example 1,3-dibromo-1,3,5-triazinane-2,4,6-trione, in a suitable solvent such as for example DCE;
Step 14: at a suitable temperature such as for example RT, in the presence of a suitable chlorinating reagent such as for example oxalyl chloride, in the presence of DMF, in a suitable solvent such as for example DCM.

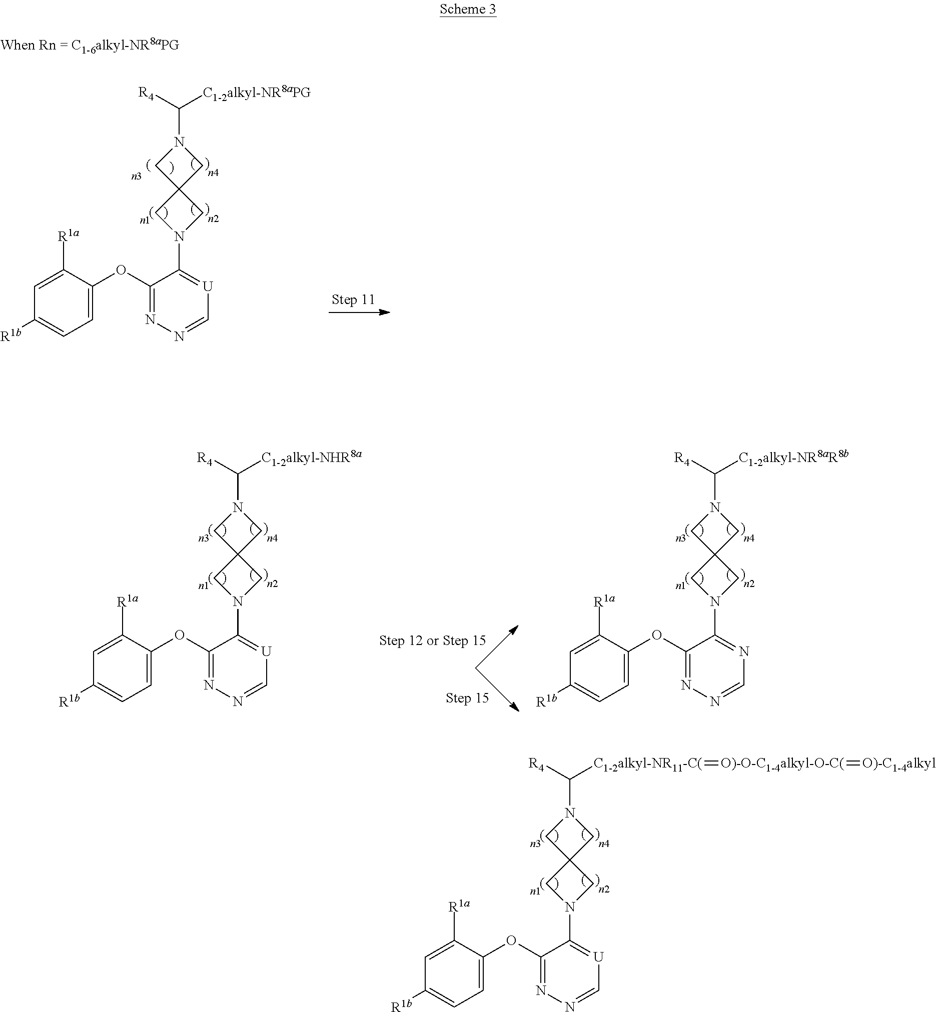

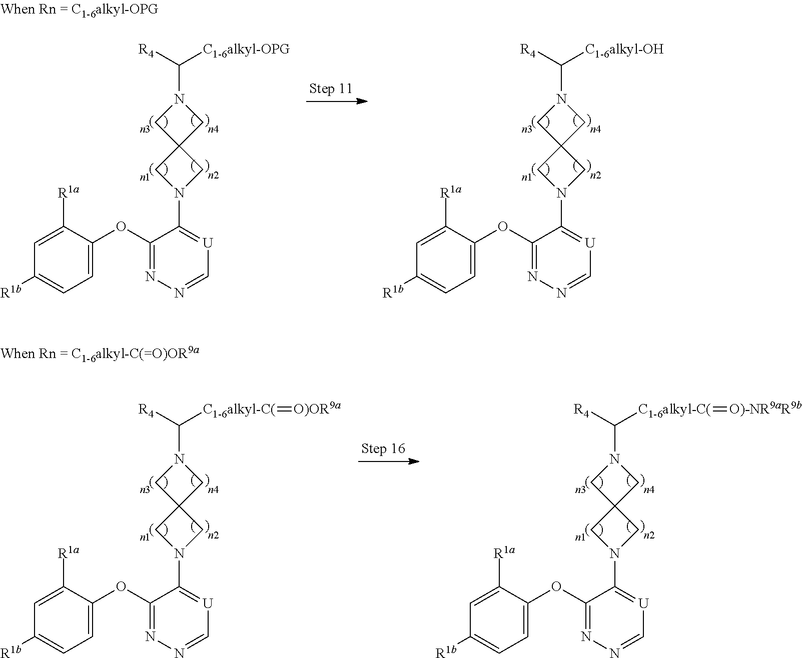

In Scheme 3, the following reaction conditions apply:
Step 11-12: See Step 11-12 in Scheme 2;
Step 15: at a suitable temperature such as for example 80° C., in the presence of a suitable base such as for example Cs₂CO₃, in suitable solvent such as for example DMF;
Step 16: at a suitable temperature such as for example 40° C., in the presence of a suitable base such as for example ammonia, in suitable solvent such as for example 1,4-dioxane.

### Part B) Schemes 4, 5, 6, 7, 8, 9, 10, 11 and 12

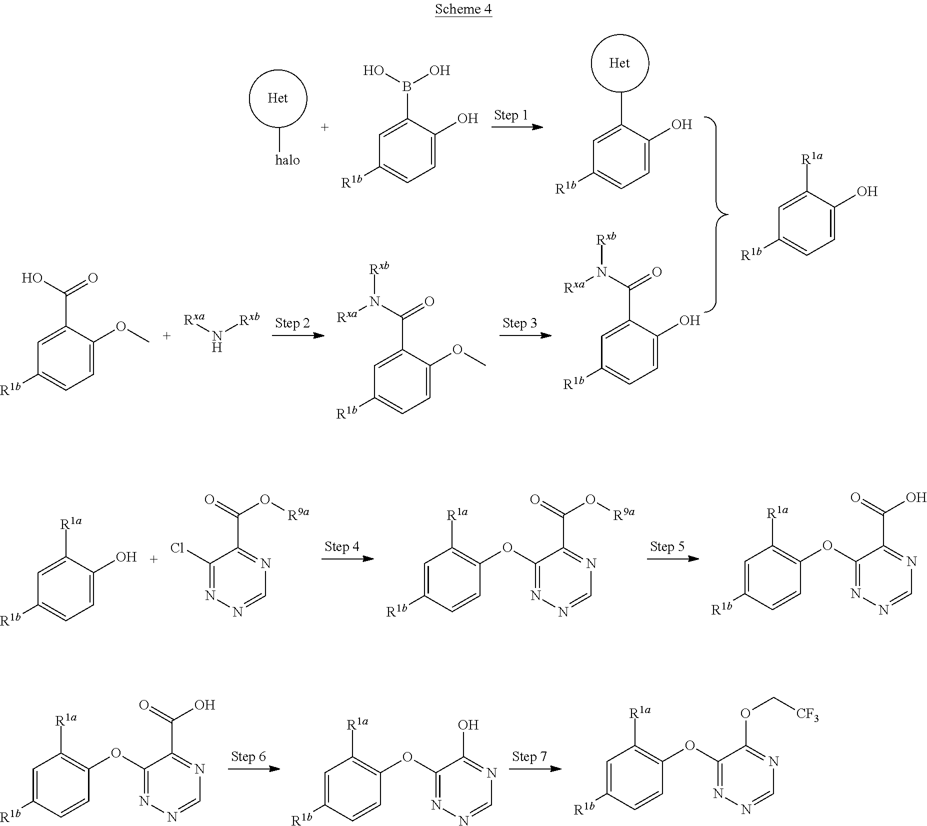

In Scheme 4, the following reaction conditions apply:
Step 1: at a suitable temperature such as for example 90° C., in the presence of a suitable organometallic catalyst such as for example Pd(dppf)Cl₂, in the presence of a suitable base such as for example Na₂CO₃, in suitable solvents such as for example 1,4-dioxane and H₂O;
Step 2: at a suitable temperature such as for example RT, in the presence of suitable amide condensation reagent such as for example HATU, in the presence of a suitable base such as for example DIEA, in a suitable solvent such as for example DCM;
Step 3: at a suitable temperature such as for example from −78° C. to RT, in the presence of a suitable Lewis acid such as for example BBr₃, in a suitable solvent such as for example DCM;
Step 4: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;
Step 5: at a suitable temperature such as for example RT, in the presence of a suitable base such as for example LiOH·H₂O, in suitable solvents such as for example THF and H₂O;
Step 6: at a suitable temperature such as for example RT, in the presence of a suitable organometallic catalyst such as for example Ag(Phen)₂OTf, in the presence of a suitable brominating reagent such as for example 1,3-dibromo-1,3,5-triazinane-2,4,6-trione, in a suitable solvent such as for example DCE;
Step 7: at a suitable temperature such as for example RT, in the presence of a suitable brominating reagent such as 1,3-dibromo-1,3,5-triazinane-2,4,6-trione, in the presence of 2,2,2-trifluoroethan-1-ol as solvent.

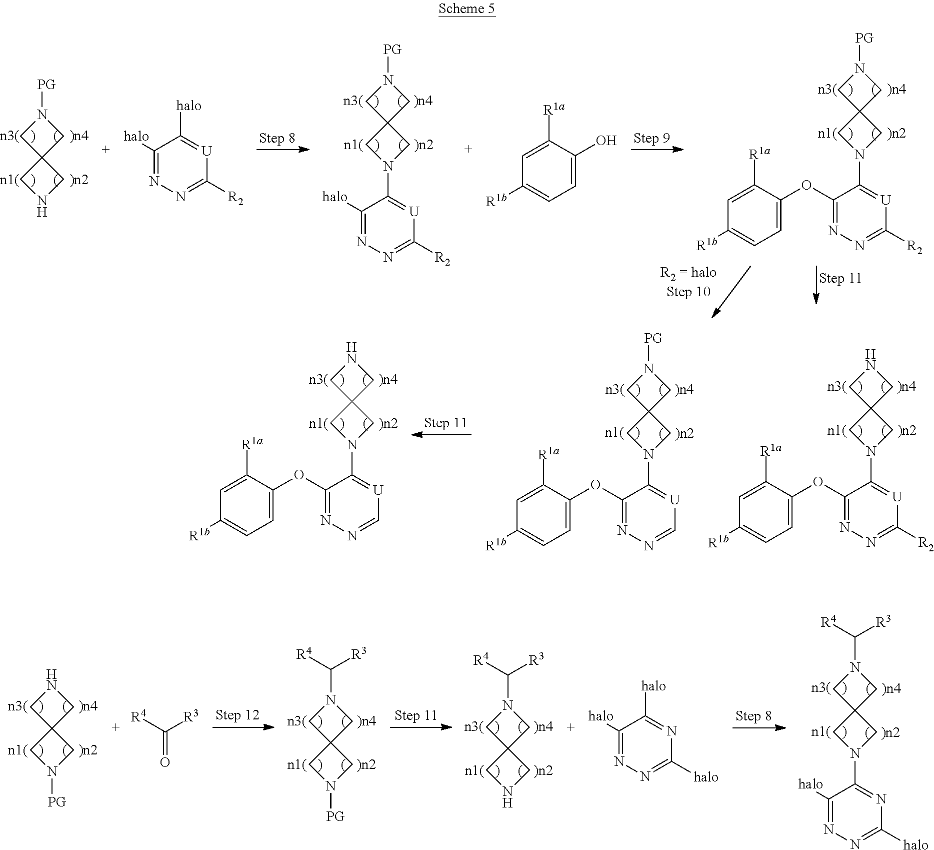

In Scheme 5, the following reaction conditions apply:
Step 8: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;
Step 9: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;
Step 10: at a suitable temperature such as for example RT, in the presence of a suitable organometallic catalyst as for example Pd/C and a suitable base as for example TEA, in a suitable solvent such as for example MeOH under H₂ atmosphere;
Step 11: When PG is Boc, at a suitable temperature such as for example RT, in the presence of a suitable acid as for example TFA, in a suitable solvent such as for example DCM.

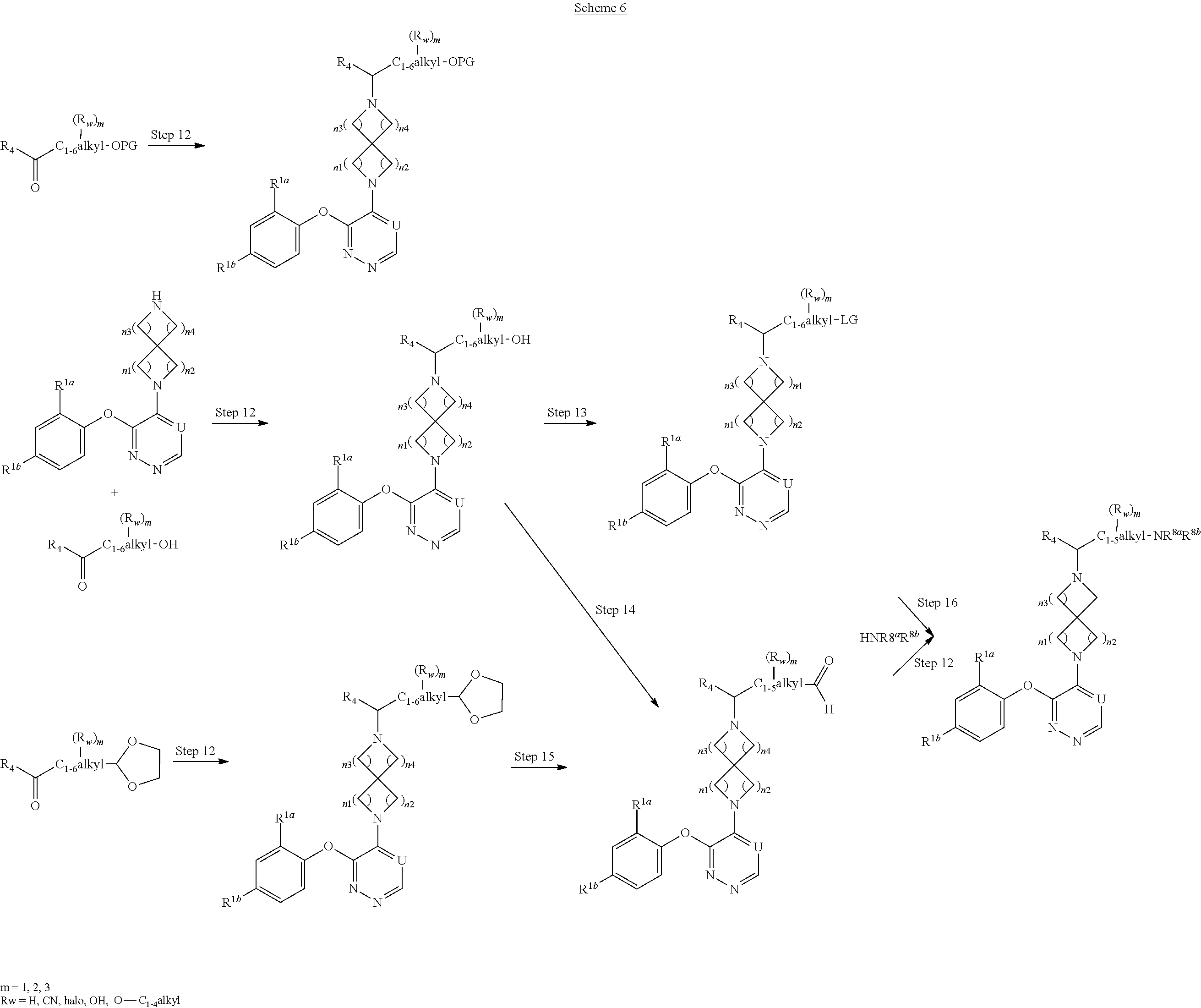

In Scheme 6, the following reaction conditions apply:
Step 12: reductive amination condition, at a suitable temperature such as for example from RT to 80° C., in the presence or absence of a suitable Lewis acid such as for example ZnCl₂ or an acid for example AcOH, in the presence of a suitable reducing agent such as for example NaBH₃CN, in a suitable solvent such as for example MeOH;
Step 13: at a suitable temperature such as for example 0° C., in the presence of a suitable electrophile as for example MsCl, in the presence of a suitable base such as for example TEA, in a suitable solvent such as for example DCM;
Step 14: at a suitable temperature such as for example from 0° C. to RT, in the presence of a suitable oxidizing agent as for example DMP, in a suitable solvent such as for example DCM;
Step 15: at a suitable temperature such as for example 50° C., in the presence of a suitable acid as for example HCl, in a suitable solvent such as for example ACN;
Step 16: at a suitable temperature such as for example RT, in the presence or absence of a suitable base as for example TEA, in a suitable solvent such as for example THF.

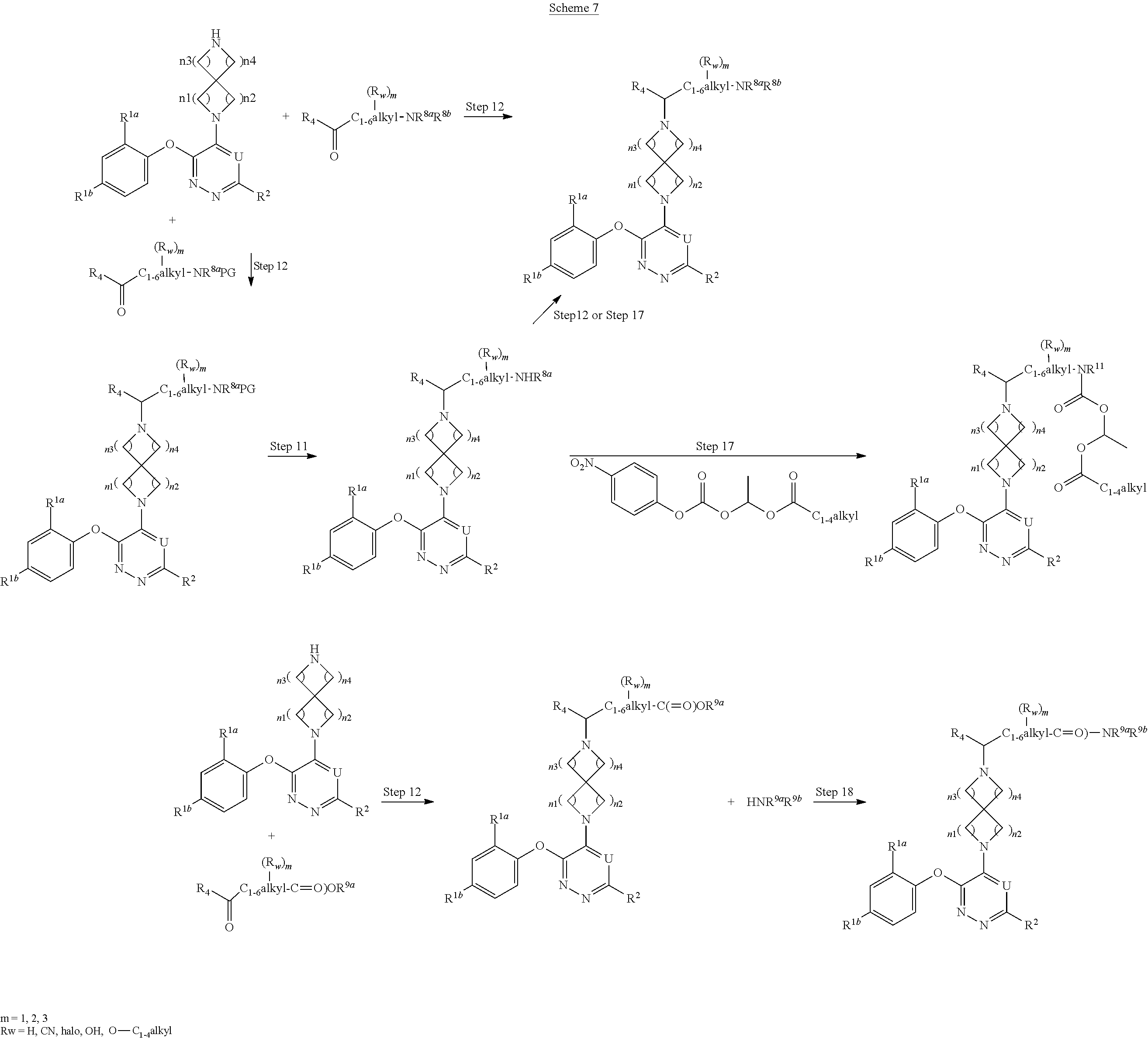

In Scheme 7, the following reaction conditions apply:
Step 11: When PG is Boc, at a suitable temperature such as for example RT, in the presence of a suitable acid as for example TFA, in a suitable solvent such as for example DCM;
Step 12: reductive amination condition, at a suitable temperature such as for example from RT to 80° C., in the presence or absence of a suitable Lewis acid such as for example ZnCl₂ or an acid for example AcOH, in the presence of a suitable reducing agent such as for example NaBH₃CN, in a suitable solvent such as for example MeOH;
Step 17: at a suitable temperature such as for example from RT to 80° C., in the presence of a suitable base such as for example DIEA or Cs₂CO₃, in suitable solvent such as for example DCM or DMF;
Step 18: at a suitable temperature such as for example 40° C., in the presence of a suitable base such as for example ammonia, in suitable solvent such as for 1,4-dioxane.

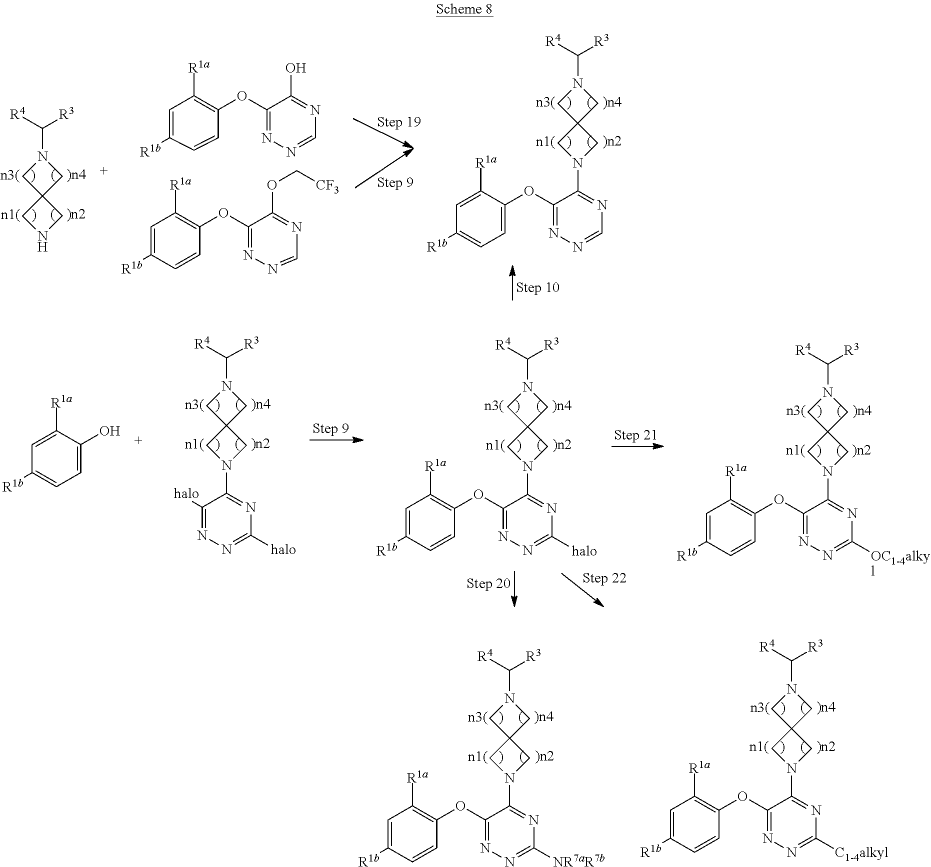

In Scheme 8, the following reaction conditions apply:
Step 9: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;
Step 10: at a suitable temperature such as for example RT, in the presence of a suitable organometallic catalyst as for example Pd/C, optionally in the presence of a suitable base as for example TEA, in a suitable solvent such as for example MeOH under H₂ atmosphere;
Step 19: at a suitable temperature such as for example RT, in the presence of a suitable chlorinating reagent such as for example oxalyl chloride, in the presence of DMF, in a suitable solvent such as for example DCM;
Step 20: at a suitable temperature such as for example 90° C., in the presence of a suitable nucleophilic amine, in a suitable solvent such as for example EtOH;
Step 21: at a suitable temperature such as for example RT, in the presence of a suitable acid such as for example HCl in dioxane, in a suitable solvent such as for example MeOH;
Step 22: at a suitable temperature such as for example 110° C., in the presence of a suitable boron reagent such as for example trimethylboroxine, in the presence of a suitable organometallic catalyst such as for example tetrakis(triphenylphosphine)palladium(0), in the presence of a suitable base such as for example K₂CO₃, in a suitable solvent such as for example 1,4-dioxane;

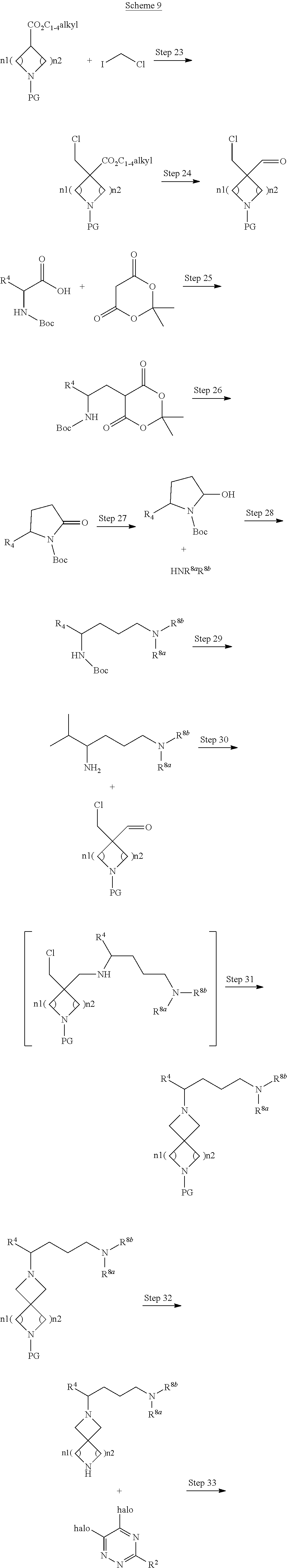

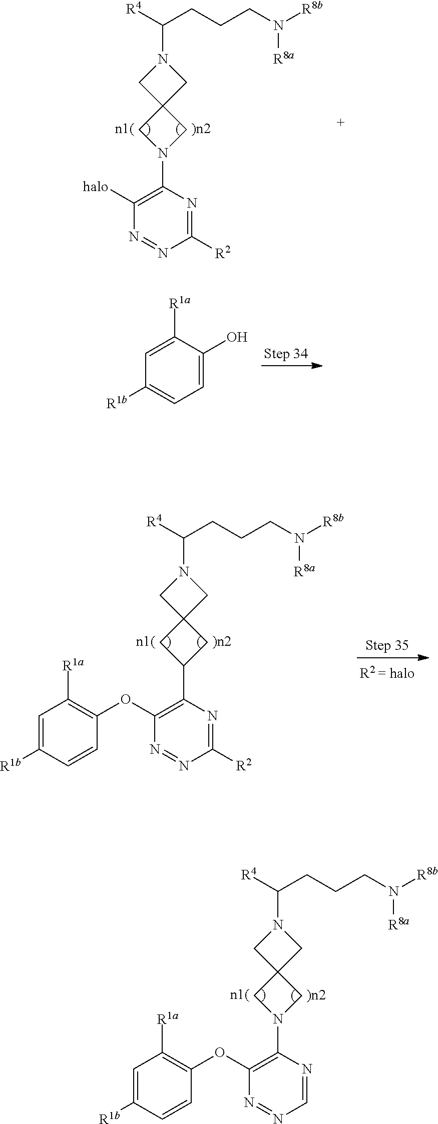

In Scheme 9, the following reaction conditions apply:
Step 23: at a suitable temperature such as for example from −78° C. to −25° C., in the presence of suitable bases such as for example DIEA and n-BuLi, in a suitable solvent such as for example THF;
Step 24: at a suitable temperature such as for example between −65° C. and −55° C., in the presence of suitable reducing agent such as for example DIBAL-H, in a suitable solvent such as for example toluene, preferably conducted in a suitable flow chemistry system;
Step 25: first at a suitable temperature such as for example from −10° C. to 10° C., in the presence of a suitable base such as for example DMAP, in the presence of a suitable condensation agent such as for example DCC, in a suitable solvent such as for example DCM; then at a suitable temperature such as for example from −10° C. to 0° C., in the presence of a suitable acid such as for example AcOH, in the presence of a suitable reducing agent such as for example NaBH₄, in a suitable solvent such as for example DCM;
Step 26: in a suitable solvent such as for example toluene and heated to reflux;
Step 27: at a suitable temperature such as for example from −5° C. to 5° C., in the presence of suitable reducing agent such as for example LiBH₄, in a suitable solvent such as for example 2-methyltetrahydrofuran;
Step 28: at a suitable temperature such as for example from 15° C. to 25° C., in the presence of a suitable reducing agent such as for example NaBH(OAc)₃, in a suitable solvent such as for example DCM;
Step 29: at a suitable temperature such as for example from 15° C. to 25° C., in the presence of a suitable acid such as for HCl, in a suitable solvent such as for example IPA;
Step 30: at a suitable temperature such as for example from 5° C. to 30° C., in the presence of a suitable base such as for example TEA, in the presence of suitable reducing agent such as for example NaBH(OAc)₃, in a suitable solvent such as for example toluene;
Step 31: at a suitable temperature such as for example from 50° C. to 55° C., in the presence of a suitable base such as for example K₂HPO₄, in a suitable solvent such as for example H₂O;
Step 32: When PG is Bn at a suitable temperature such as for example from −5° C. to 45° C., under a hydrogen atmosphere within a suitable pressure range such as for example from 0.27 to 0.40 MPa, in the presence of a suitable catalyst such as for example palladium hydroxide on carbon, in the presence of a suitable acid as for example MSA in a suitable solvent such as EtOH;
Step 33: at a suitable temperature such as for example from −50° C. to −40° C., in the presence of suitable base such as for example TEA, in a suitable solvent such as 2-methyltetrahydrofuran;
Step 34: at a suitable temperature such as for example from 20° C. to 30° C., in the presence of suitable base such as for example TMG, in a suitable solvent such as 2-methyltetrahydrofuran;
Step 35: at a suitable temperature such as for example from 20° C. to 30° C., under a hydrogen atmosphere within a suitable pressure range such as for example from 0.20 to 0.30 Mpa, in the presence of a suitable catalyst such as for example palladium on carbon, in a suitable solvent such as MeOH;
alternatively, at a suitable temperature such as room temperature, in the presence of a suitable catalyst such as for example 1,1′-Bis(diphenylphosphino)ferrocene-palladium(II) dichloride dichloromethane complex, a suitable reducing agent such sodium borohydride, a suitable base such as for example N,N,N′,N′-tetramethylethylenediamine, in a suitable solvent such as for example tetrahydrofuran.

### Scheme 10

In general, compounds of Formula (I) wherein Y¹ is limited to —CH₂—, and R² is limited to W¹, hereby named compounds of Formula (Ia), can be prepared according to the following reaction Scheme 10. In Scheme 10, W¹ represents chloro, bromo or iodo; all other variables are defined according to the scope of the present invention.

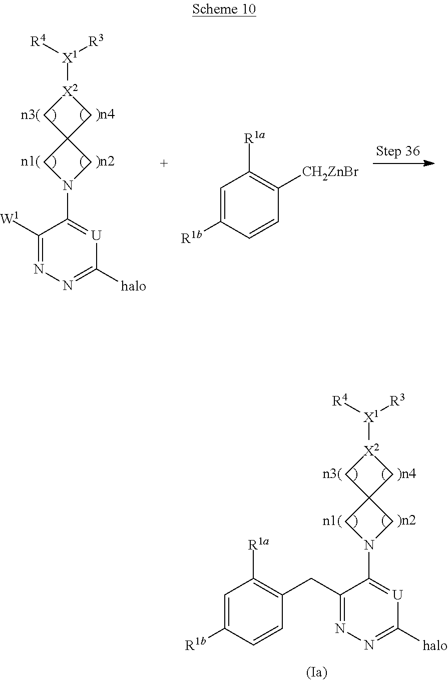

In Scheme 10, the following reaction conditions apply:
Step 36: at a suitable temperature ranged from 60° C. to 100° C., in presence of a suitable catalyst such as palladium acetate (Pd(OAc)₂) or tris(dibenzylideneacetone)dipalladium(0) (Pd₂(dba)₃) or tetrakis(triphenylphosphine)palladium(0), in a suitable solvent such as for example tetrahydrofuran or dioxane.
The skilled person will realize that starting from compound (Ia), analogous chemistry as reported in step 10 in scheme 5 and in steps 20, 21 and 22 in scheme 8 could be performed.

### Scheme 11

In general, compounds of Formula (I) wherein Y¹ is limited to —CR^{5a}R^{5b}— and R² is limited to W¹, hereby named compounds of Formula (Ib), can be prepared according to the following reaction Scheme 11. In Scheme 11 at least one of R^{5a} and R^{5b} is other than hydrogen. All other variables are defined according to the scope of the present invention.

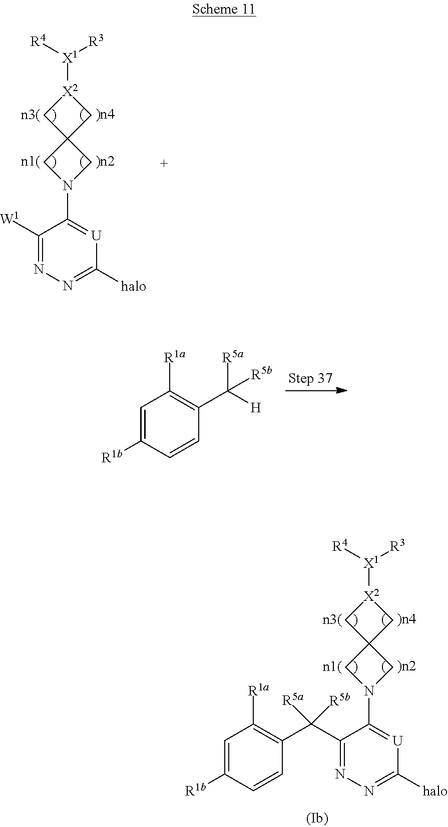

In Scheme 11, the following reaction condition apply:

Step 37: at a suitable temperature ranged from 80° C. to 200° C., in presence of a suitable catalyst such as palladium acetate (Pd(OAc)₂), in the presence of a suitable ligand such as for example triphenylphosphine or tricyclohexylphosphine, in a suitable solvent such as for example dioxane, preferably in sealed conditions, optionally under microwave irradiation.

The skilled person will realize that starting from compound (Ib), analogous chemistry as reported in step 10 in scheme 5 and in steps 20, 21 and 22 in scheme 8 could be performed.

### Scheme 12

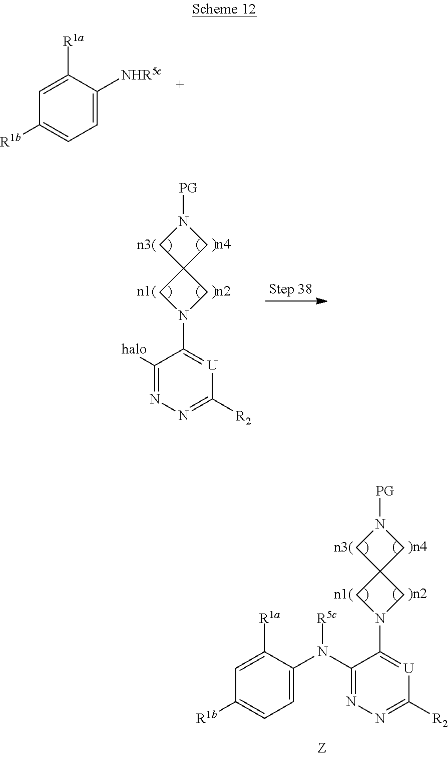

In Scheme 12, the following reaction condition apply:

Step 38: at a suitable temperature such as for example from RT to 80° C., in the presence of a suitable base such as for example DIEA, Cs₂CO₃ or DBU, in suitable solvent such as for example DCM, THF or DMF;

Alternatively, at a suitable temperature such as for example RT to 100° C., in the presence of a suitable catalyst such as for example Pd₂dba₃, in the presence of a suitable ligand such as for example Xantphos, in the presence of a suitable base such as Cs₂CO₃ or Na₂CO₃, in a suitable solvent such dioxane or a mixture of dioxane and water.

The skilled person will realize that starting from intermediate Z, analogous chemistry as reported in case Y¹ represents O can be performed.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of Formula (I), protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenyl-methyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, New Jersey, 2007.

Several methods for preparing the compounds of Formula (I) are illustrated in the following examples. Unless otherwise noted, all starting materials were obtained from commercial suppliers and used without further purification, or alternatively can be synthesized by a skilled person by using well-known methods.

TABLE 1B
Abbreviations

| Abbreviation | Meaning |
|---|---|
| Ag(Phen)₂OTf | silver triflate-bis(1,10-phenanthroline) complex |
| 2-MeTHF | 2-methyltetrahydrofuran |
| ACN | acetonitrile |
| AcCl | acetyl chloride |
| AcOH | acetic acid |
| Ac₂O | acetic anhydride |
| aq. | aqueous |
| Ar | argon |
| BBr₃ | tribromoborane |
| bn | benzyl |
| Boc | tert-butyloxycarbonyl |
| Boc₂O | di-tert-butyl dicarbonate |
| n-BuLi | n-butyllithium |
| Cbz | benzyloxycarbonyl |
| CD₃OD | Methanol-d₄ |
| CHCl₃ | chloroform |
| Cs₂CO₃ | cesium carbonate |
| conc. | concentrated |
| DBU | 1,8-diazabicyclo[5.4.0]undec-7-ene |
| DCC | dicyclohexylcarbodiimide |
| DCE | dichloroethane |
| DCM | dichloromethane |
| DDQ | 4,5-dichloro-3,6-dioxocyclohexa-1,4-diene-1,2-dicarbonitrile |
| DEA | diethylamine |
| DIBAL-H | diisobutylaluminum hydride |
| DIEA or DIPEA | N,N-diisopropylethylamine |
| DMAP | N,N-dimethylpyridin-4-amine |
| DMF | N,N-dimethylformamide |
| DMP | Dess-Martin periodinane |
| DMSO | dimethyl sulfoxide |
| dppf | 1,1′-ferrocenediyl-bis(diphenylphosphine) |
| EDCI | N-(3-Dimethylaminopropyl)-N′-ethylcarbodiimide hydrochloride |
| EA or EtOAc | ethyl acetate |
| EtOH | ethanol |
| eq. | equivalent(s) |
| FA | formic acid |
| FCC | flash column chromatography |
| h | hour(s) |
| H₂ | hydrogen |
| HATU | 1-[bis(dimethylamino)methylene]-1H-1,2,3-triazolo[4,5-b] |
| | pyridinium 3-oxid hexafluorophosphate |
| H₂O | water |
| HCl | hydrochloric acid |
| HOBt | 1-Hydroxybenzotriazole |
| HPLC | high performance liquid chromatography |
| ICH₂Cl | chloroiodomethane |
| IPA | isopropyl alcohol |
| IPAc | isopropyl acetate |
| K₂CO₃ | potassium carbonate |
| KI | potassium iodide |
| K₂HPO₄ | dipotassium phosphate |
| K₃PO₄ | tripotassium phosphate |
| LiAlD₄ | lithium aluminum deuteride |
| LAH | lithium aluminum hydride |
| LiBH₄ | lithium borohydride |
| LDA | lithium diisopropylamide |
| LiCl | lithium chloride |
| LG | leaving group |
| Me | methyl |
| MeOH | methanol |
| 2-MeTHF | 2-methyltetrahydrofuran |
| min | minute(s) |
| mL | milliliters |
| mmol | millimoles |
| mg | milligram |
| MgSO₄ | magnesium sulfate |
| MSA | methanesulfonic acid |
| MsCl | methanesulfonyl chloride |
| MS | molecular sieve |
| MTBE | methyl tert-butyl ether |
| N₂ | nitrogen |
| NA | not available |
| NaBH₃CN | sodium cyanoborohydride |
| NaBH(OAc)₃ | sodium triacetoxyborohydride |
| NaBD₃CN | sodium cyanoborodeuteride |
| Na₂CO₃ | sodium carbonate |
| NaH | sodium hydride |
| NaHCO₃ | sodium bicarbonate |
| NaI | sodium iodide |
| NaOAc | sodium acetate |
| NaOH | sodium hydroxide |
| Na₂SO₃ | sodium sulfite |
| Na₂SO₄ | sodium sulfate |
| NH₄Cl | ammonium chloride |
| NMM | 1-4-Methylmorpholine |
| Pd₂dba₃ | tris(dibenzylideneacetone)dipalladium(0) |
| Pd(dppf)Cl₂•DCM | [1,1′-bis(diphenylphosphino)ferrocene] dichloropalladium(II), |
| | complex with dichloromethane |
| Pd(PPh₃)₄ | tetrakis(triphenylphosphine)palladium(0) |
| PE | petroleum ether |
| PG | protecting group |
| Phen | phenanthroline |
| psi | pound per square inch |
| p-TsOH | p-toluenesulfonic acid |
| p-TsOH•H₂O | p-toluenesulfonic acid monohydrate |
| Rₜ | retention time |
| Rochelle's salt | potassium sodium tartrate tetrahydrate |
| RT | room temperature |
| sat. | saturated |
| SFC | supercritical fluid chromatography |
| TBAF | tetrabutylammonium fluoride |
| TBDMS | tert-butyldimethylsilyl |
| TBDPS | tert-butyldiphenylsilyl |
| t-BuOK | potassium tert-butoxide |
| TEA | triethylamine |
| Tf | trifluoromethanesulfonyl |
| TFA | trifluoroacetic acid |
| THF | tetrahydrofuran |
| Ti(OiPr)₄ | titanium(IV) isopropoxide |
| TLC | thin layer chromatography |
| TMEDA | N,N,N′,N′-tetramethylethylenediamine |
| TMG | 1,1,3,3-tetramethylguanidine |
| TMSI | iodotrimethylsilane |
| Ts | p-toluenesulfonyl |
| TsCl | p-toluenesulfonyl chloride |
| v/v | volume per volume |
| vol. | volume(s) |
| wt | weight |
| Xantphos | 4,5-bis(diphenylphosphino)-9,9-dimethylxanthene |

As understood by a person skilled in the art, compounds synthesized using the protocols as indicated may exist as a solvate e.g., hydrate, and/or contain residual solvent or minor impurities. Compounds or intermediates isolated as a salt form, may be integer stoichiometric i.e., mono- or di-salts, or of intermediate stoichiometry. When an intermediate or compound in the experimental part below is indicated as ‘HCl salt’ without indication of the number of equivalents of HCl, this means that the number of equivalents of HCl was not determined. The same principle will also apply to all other salt forms referred to in the experimental part, such as e.g., ‘oxalate salt’, ‘formate salt’ or

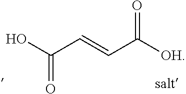

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

When a stereocenter is indicated with ‘RS’ this means that a racemic mixture was obtained at the indicated center, unless otherwise indicated.

### Example 1—Synthesis of (R)-N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A)—Preparation Method A

### Preparation of Intermediate 1—tert-butyl (5-methyl-4-oxohexyl)carbamate

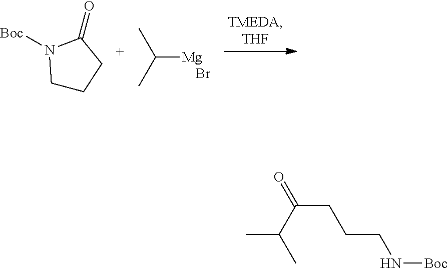

To a solution of tert-butyl 2-oxopyrrolidine-1-carboxylate (5.0 g, 27 mmol) and TMEDA (5.0 mL, 33 mmol) in THF (60 mL) cooled at −70° C. was slowly added isopropylmagnesium bromide solution (19 mL, 55 mmol, 2.9 M in 2-methyltetrahydrofuran), the resulting mixture was slowly warmed to RT and stirred for 12 h. The mixture was poured into sat. aq. NH₄Cl (50 mL) solution and extracted with EtOAc (50 mL×3). The combined organic layers were dried over anhydrous Na₂SO₄, filtered, and concentrated under reduced pressure to give the crude product, which was further purified by FCC (PE/EtOAc=1:0 to 100:1) to afford the title intermediate (3.7 g, 60% yield) as a yellow oil.

### Preparation of Intermediate 13—tert-butyl 6-(3,6-dichloro-1,2,4-triazin-5-yl)-2,6-diazaspiro [3.4]octane-2-carboxylate

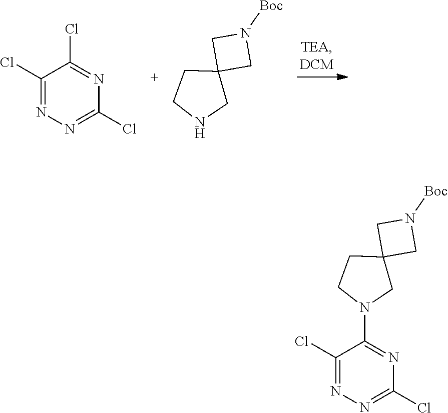 [SMILES: Clc1nnc(Cl)c(Cl)n1 ; CC(C)(C)OC(=O)N1CC2(CCNC2)C1 ; CC(C)(C)OC(=O)N1CC2(CCN(c3nc(Cl)nnc3Cl)C2)C1]

To the solution of 3,5,6-trichloro-1,2,4-triazine (10.0 g, 54.2 mmol) and TEA (15.2 mL, 109 mmol) in DCM (100 mL) cooled at 0° C. was added tert-butyl 2,6-diazaspiro[3.4]octane-2-carboxylate (9.21 g, 43.4 mmol), the mixture was warmed to RT and stirred for 1 h. The mixture was diluted with water (20 mL) and extracted with DCM (30 mL×3). The combined organic layers were washed with brine, dried over Na₂SO₄, filtered and concentrated under reduced pressure to give the crude product which was purified by FCC on silica gel (PE/EtOAc=1:0 to 3:1) to afford the title intermediate (12.0 g, 58% yield) as a yellow solid.

### Preparation of Intermediate 27—N-ethyl-5-fluoro-N-isopropyl-2-methoxybenzamide

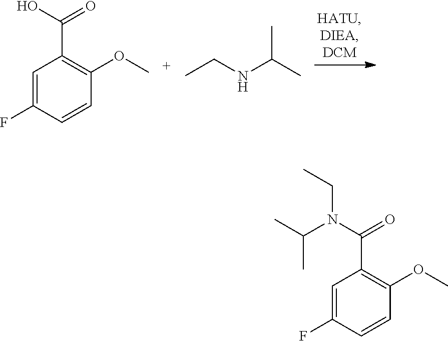

To the mixture of 5-fluoro-2-methoxybenzoic acid (8.00 g, 47.0 mmol) and N-ethylpropan-2-amine (8.19 g, 94.0 mmol) in dry DCM (150 mL) cooled at 0° C., were slowly added HATU (21.5 g, 56.5 mmol) and DIEA (9.10 g, 70.4 mmol) in portions. The resulting mixture was slowly warmed to RT and stirred for 8 h. The organic layer was washed with water (20 mL×3) and dried over anhydrous Na₂SO₄. After filtration, the solvent was removed under reduced pressure and the crude product was purified by FCC (EtOAc/PE=0% to 20%) to afford the title intermediate (12.0 g, 96% yield) as a white solid.

### Preparation of Intermediate 28—N-ethyl-5-fluoro-2-hydroxy-N-isopropylbenzamide

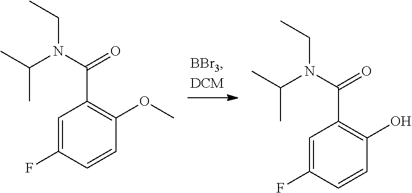 [SMILES: CCN(C(=O)c1cc(F)ccc1OC)C(C)C ; CCN(C(=O)c1cc(F)ccc1O)C(C)C]

To the solution of N-ethyl-5-fluoro-N-isopropyl-2-methoxybenzamide (intermediate 27) (12.0 g, 50.1 mmol) in dry DCM (100 mL) cooled at −78° C. was slowly added BBr₃ (14.4 mL, 152 mmol), the resulting mixture was slowly warmed to RT and stirred for 8 h. The mixture was cooled to −78° C. again and MeOH (5 mL) was added dropwise to quench the reaction. The resulting mixture was slowly warmed to RT and the pH value was adjusted to about 8 by adding sat. aq. NaHCO₃ solution. The aqueous layer was extracted by DCM (50 mL×3) and the combined organic layers were dried over anhydrous Na₂SO₄, filtered and concentrated under reduced pressure to give the crude product which was purified by FCC (EtOAc/PE=0% to 20%) to afford the title intermediate (9.0 g, 78% yield) as a white solid.

### Alternative Preparation of Intermediate 28

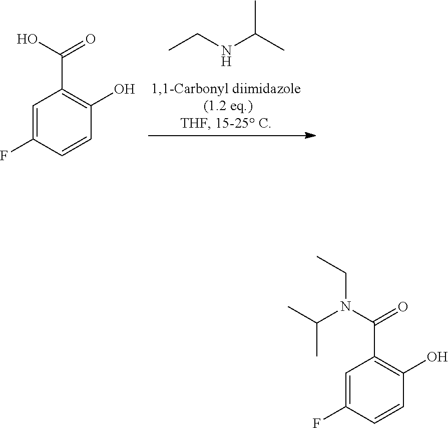

A mixture of 5-fluoro-2-hydroxy-benzoic acid (14.0 kg, 89.68 mol, 1.0 equiv.) in THF (168 L, 12 volumes) was adjusted to between 15-25° C., and 1,1-carbonyldiimidazole, (17.45 kg, 107.62 mol, 1.2 equiv.) was added over a period of 1 hour. After addition, the mixture was stirred for 18 hours at 15-25° C. After this time N-ethylpropan-2-amine (14.85 kg, 170.39 mol, 1.9 equiv.) was added to the mixture at 15-25° C. over a period of 2 hours. The resulting mixture was further aged for between 18-24 hours at 15-25° C. The pH was the adjusted to between pH4-5 with aq. 10% H₂SO₄ (140 kg, 10 volumes) and the layers were separated. The organic phase was concentrated to between 42-56 L maintaining a temperature below 40° C., and then n-heptane (43 kg, 4.5 volumes) was added to the mixture at 15-25° C. over a period of 3 hours. The mixture was then cooled to 0-10° C. and stirred for an additional 6 hours. The resulting slurry was filtered and the cake was washed with a tert-butyl methyl ether (MTBE):n-heptane mixture (25 kg of a 2:3 volume/volume mixture of MTBE:n-heptane, 2.5 volumes). The cake wash was repeated a further two times and the resulting solid was dried in-vacuo at 50° C. to afford intermediate 28 (16.5 kg, purity: 99.1%, yield: 80.4%).

### Preparation of Intermediate 14—tert-butyl 6-(3-chloro-6-(2-(ethyl(isopropyl)carbamoyl)-4-fluorophenoxy)-1,2,4-triazin-5-yl)-2,6-diazaspiro[3.4]octane-2-carboxylate

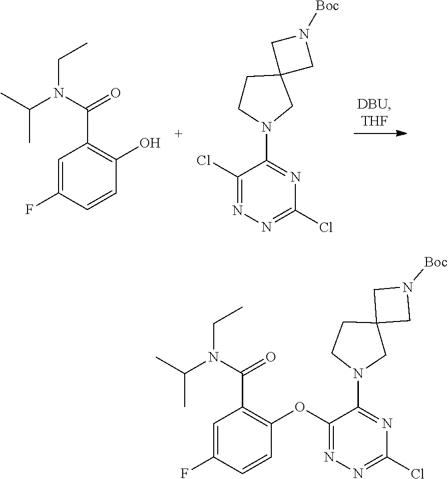 [SMILES: CCN(C(=O)c1cc(F)ccc1O)C(C)C ; CC(C)(C)OC(=O)N1CC2(CCN(c3nc(Cl)nnc3Cl)C2)C1 ; CCN(C(=O)c1cc(F)ccc1Oc1nnc(Cl)nc1N1CCC2(CN(C(=O)OC(C)(C)C)C2)C1)C(C)C]

The mixture of tert-butyl 6-(3,6-dichloro-1,2,4-triazin-5-yl)-2,6-diazaspiro[3.4]octane-2-carboxylate (intermediate 13) (12.0 g, 33.3 mmol), N-ethyl-5-fluoro-2-hydroxy-N-isopropylbenzamide (intermediate 28) (7.5 g, 33.3 mmol) and DBU (6.1 g, 40.1 mmol) in THF (120 mL) was stirred at 25° C. for 8 h. The mixture was diluted with water (30 mL) and extracted with DCM (30 mL×3). The combined organic layers were washed with brine, dried over Na₂SO₄, filtered and concentrated under reduced pressure to give the crude product which was purified by FCC (PE/EtOAc=1:0 to 3:1) to afford the title intermediate (14.0 g, 73% yield) as green solid.

### Preparation of intermediate 2—tert-butyl 6-(6-(2-(ethyl(isopropyl)carbamoyl)-4-fluoro-phenoxy)-1,2,4-triazin-5-yl)-2,6-diazaspiro[3.4]octane-2-carboxylate

### Synthesis Method a for Intermediate 2

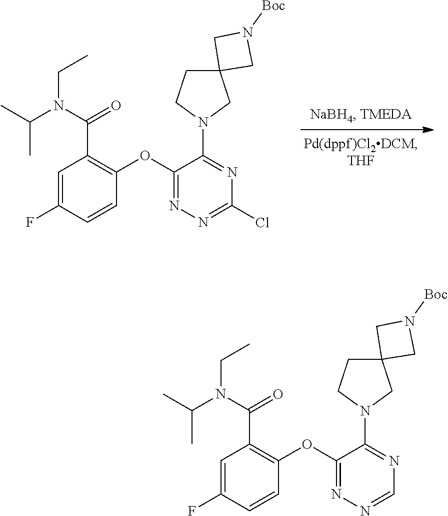 [SMILES: CCN(C(=O)c1cc(F)ccc1Oc1nnc(Cl)nc1N1CCC2(CN(C(=O)OC(C)(C)C)C2)C1)C(C)C ; CCN(C(=O)c1cc(F)ccc1Oc1nncnc1N1CCC2(CN(C(=O)OC(C)(C)C)C2)C1)C(C)C]

To the mixture of tert-butyl 6-(3-chloro-6-(2-(ethyl(isopropyl)carbamoyl)-4-fluorophenoxy)-1,2,4-triazin-5-yl)-2,6-diazaspiro[3.4]octane-2-carboxylate (intermediate 14) (20 g, 36.4 mmol), NaBH₄ (2.48 g, 65.7 mmol) and TMEDA (8.54 g, 73.5 mmol) in THF (500 mL) was added Pd(dppf)Cl₂DCM (1.70 g, 2.08 mmol) under N₂ atmosphere. After addition, the reaction mixture was stirred at 25° C. for 14 h. The reaction mixture was filtered and the filtrate was concentrated, the residue was purified by FCC on silica gel (EtOAc) to afford the title intermediate (15 g, 93% purity, 74% yield) as brown solid.

### Synthesis Method B for Intermediate 2

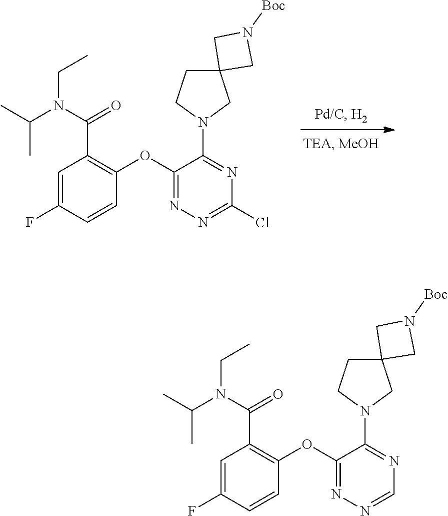 [SMILES: CCN(C(=O)c1cc(F)ccc1Oc1nnc(Cl)nc1N1CCC2(CN(C(=O)OC(C)(C)C)C2)C1)C(C)C ; CCN(C(=O)c1cc(F)ccc1Oc1nncnc1N1CCC2(CN(C(=O)OC(C)(C)C)C2)C1)C(C)C]

To the solution of tert-butyl 6-(3-chloro-6-(2-(ethyl(isopropyl)carbamoyl)-4-fluorophenoxy)-1,2,4-triazin-5-yl)-2,6-diazaspiro[3.4]octane-2-carboxylate (intermediate 14) (22.0 g, 40.1 mmol), TEA (15 mL) in MeOH (100 mL) was added Pd/C (wet, 5.0 g, 10%) The resulting mixture was stirred under H₂ atmosphere (30 psi) at 25° C. for 8 h. The reaction mixture was filtered through a celite pad and the filtrate was concentrated in vacuo to afford the title intermediate (25.0 g, crude), which was used directly in next step without further purification.

### Preparation of Intermediate 3—2-((5-(2,6-diazaspiro[3.4]octan-6-yl)-1,2,4-triazin-6-yl)oxy)-N-ethyl-5-fluoro-N-isopropylbenzamide

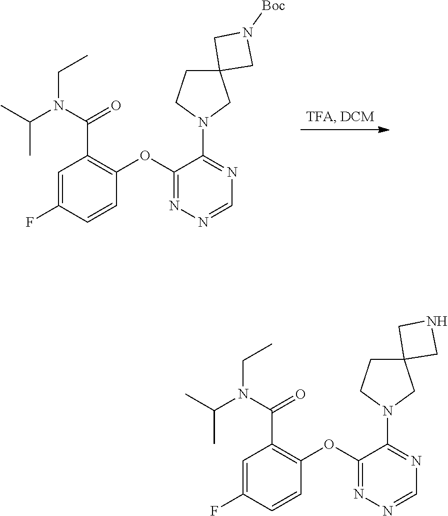 [SMILES: CCN(C(=O)c1cc(F)ccc1Oc1nncnc1N1CCC2(CN(C(=O)OC(C)(C)C)C2)C1)C(C)C ; CCN(C(=O)c1cc(F)ccc1Oc1nncnc1N1CCC2(CNC2)C1)C(C)C]

To the solution of tert-butyl 6-(6-(2-(ethyl(isopropyl)carbamoyl)-4-fluorophenoxy)-1,2,4-triazin-5-yl)-2,6-diazaspiro[3.4]octane-2-carboxylate (intermediate 2) (300 mg, 0.583 mmol) in DCM (5 mL) was added TFA (0.5 mL, 6.4 mmol), the resulting mixture was stirred at RT for 3 h. Then 10% NaOH (5 mL) solution was slowly added into the mixture to adjust the pH value to about 12, the resulting mixture was extracted with DCM (10 mL×3). The combined organic layers were dried over anhydrous Na₂SO₄, filtered, and concentrated in vacuo to afford the title intermediate (220 mg, 90% yield) as a white solid.

### Preparation of Compound 61—tert-butyl (4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate

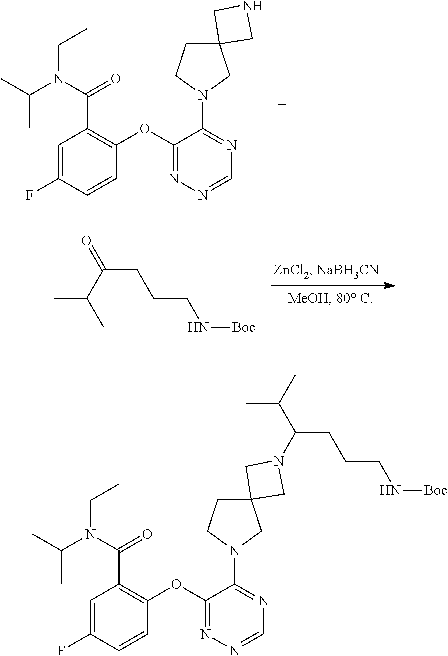

The mixture 2-((5-(2,6-diazaspiro[3.4]octan-6-yl)-1,2,4-triazin-6-yl)oxy)-N-ethyl-5-fluoro-N-isopropylbenzamide (intermediate 3) (1.0 g, 2.4 mmol), tert-butyl (5-methyl-4-oxohexyl)carbamate (intermediate 1) (830 mg, 3.62 mmol) and ZnCl₂ (660 mg, 4.84 mmol) in MeOH (15 mL) was stirred at 80° C. for 0.5 h. Then NaBH₃CN (310 mg, 4.93 mmol) was added and the resulting mixture was stirred at 80° C. for 6 h. After cooled to RT, the mixture was concentrated under reduced pressure to give the crude product, which was further purified by preparative HPLC using a Waters Xbridge Prep OBD (column: C18 150×40 mm 10 um; eluent: ACN/H₂O (0.05% ammonia) from 45% to 75% v/v) to afford the title compound (700 mg, 46% yield) as colorless oil.

### Preparation of Compounds 62 and 63—tert-butyl (R)-(4-(6-(6-(2-(ethyl(isopropyl) carbamoyl)-4-fluorophenoxy)-1,2,4-triazin-5-yl)-2,6-diazaspiro[3.4]octan-2-yl)-5-methylhexyl)carbamate and tert-butyl (S)-(4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate

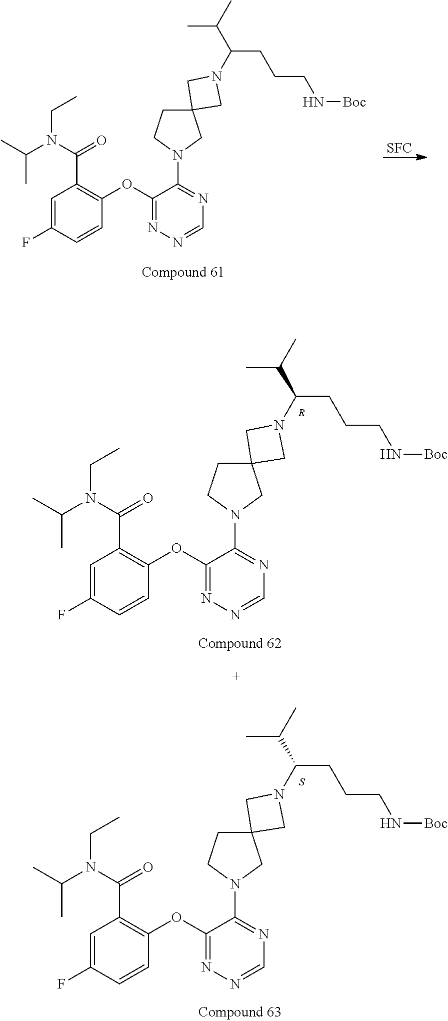

tert-butyl (4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate (compound 61) (200 mg, 0.319 mmol) was purified by SFC over DAICEL CHIRALPAK IG (column: 250×30 mm 10 um; isocratic elution: EtOH (containing 0.1% of 25% ammonia): supercritical CO₂, 40%: 60% (v/v)) to afford the title compounds (compound 62) (85 mg, 42% yield) and (compound 63) (80 mg, 40% yield) both as light yellow oil.

### Compound 64—(R)-2-((5-(2-(6-amino-2-methylhexan-3-yl)-2,6-diazaspiro[3.4]octan-6-yl)-1,2,4-triazin-6-yl)oxy)-N-ethyl-5-fluoro-N-isopropylbenzamide

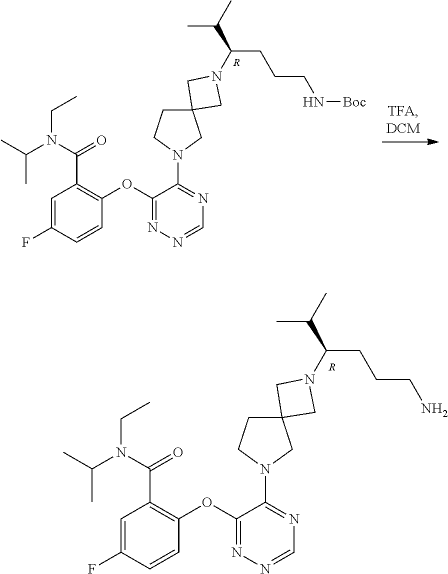

To the solution of tert-butyl (R)-(4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate (compound 62) (550 mg, 0.876 mmol) in DCM (4 mL) was slowly added TFA (4 mL), and the resulting mixture was stirred at 25° C. for 1 h. The reaction mixture was concentrated under reduced pressure to give a residue. The residue was diluted in DCM (40 mL) and the pH value was adjusted to around 12 by aq. NaOH (2 M, 16 mL) solution. The aqueous layer was extracted with DCM (10 mL×2). The combined organic layers were dried over anhydrous Na₂SO₄, filtered and concentrated in vacuo to afford the title compound (460 mg, crude) as yellow solid, which was used directly in next step without further purification.

### Compound 11—(R)-N-ethyl-5-fluoro-N-isopropyl-2-((5-(2-(6-((2-methoxyethyl)amino)-2-methylhexan-3-yl)-2,6-diazaspiro[3.4]octan-6-yl)-1,2,4-triazin-6-yl)oxy)benzamide

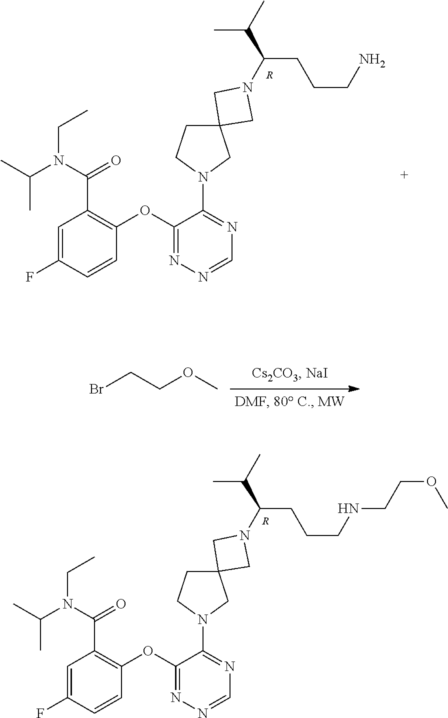

The mixture of (R)-2-((5-(2-(6-amino-2-methylhexan-3-yl)-2,6-diazaspiro[3.4]octan-6-yl)-1,2,4-triazin-6-yl)oxy)-N-ethyl-5-fluoro-N-isopropylbenzamide (compound 64) (120 mg, crude), 1-bromo-2-methoxyethane (32 mg, 0.23 mmol), Cs₂CO₃ (222 mg, 0.681 mmol), NaI (102 mg, 0.680 mmol) in DMF (1 mL) was stirred at 80° C. via microwave irradiation for 1 h. After cooling to RT, the mixture was diluted with H₂O (10 mL) and extracted with EtOAc (3×10 mL). The combined organic layers were washed with H₂O (10 mL), dried over Na₂SO₄, filtered and concentrated under reduced pressure to afford the crude product which was further purified by HPLC over a Phenomenex Gemini-NX (column: 150×30 mm 5 μm; eluent: ACN/H₂O (10 mM NH₄HCO₃) from 51% to 71% (v/v)) and further purified by SFC over DAICEL CHIRALCEL OD-H (column: 250×30 mm 5 um; eluent: supercritical CO₂ in EtOH (0.1% v/v ammonia) 25/25, v/v) to afford the title compound (5.13 mg, 96% purity) as yellow solid.

LC-MS (ESI) (Method 1): Rₜ=2.997 min, m/z found 586.3 [M+H]⁺.

### Compound A—(R)-N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide

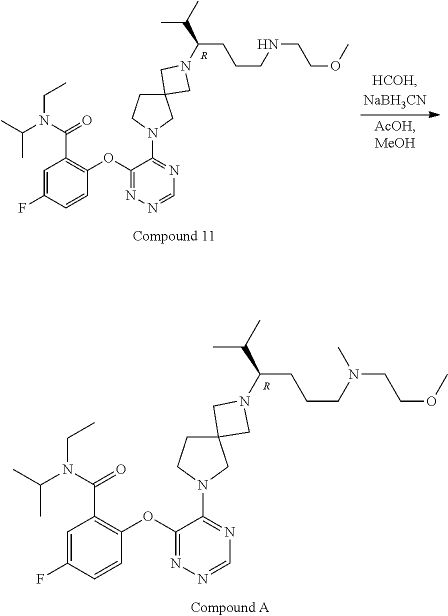

The mixture of (R)-N-ethyl-5-fluoro-N-isopropyl-2-((5-(2-(6-((2-methoxyethyl)amino)-2-methylhexan-3-yl)-2,6-diazaspiro [3.4]octan-6-yl)-1,2,4-triazin-6-yl)oxy)benzamide (compound 11) (40.0 mg, 0.068 mmol), formaldehyde (55.4 mg, 0.683 mol, 37% in water) and AcOH (8.2 mg, 0.137 mmol) in anhydrous MeOH (2 mL) was stirred at 45° C. for 1 h. Then, NaBH₃CN (8.6 mg, 0.137 mmol) was added to the mixture and the resulting mixture was stirred at 45° C. for another 1 h. After cooling to RT, the reaction mixture was treated with sat. aq. NaHCO₃ (40 mL) to adjust the pH value to about 8 and further extracted with DCM (20 mL×3). The combined organic layers were dried over anhydrous Na₂SO₄, filtered and concentrated under reduced pressure to give the crude which was purified by preparative HPLC over Boston Prime (column: C18 150×30 mm Sum, Mobile Phase A: H₂O (0.04% ammonia+10 mM NH₄HCO₃), Mobile Phase B: ACN, Flow rate: 25 m/min, gradient condition B/A from 50% to 80% (50% B to 80% B)) to afford the title compound (9.62 mg, 99.10% purity, 23.3% yield) as yellow oil.

### Example 2—Synthesis of (R)-N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A)—Preparation Method B

### Preparation of Intermediate 7—4-((tert-butoxycarbonyl)(methyl)amino)butanoic acid

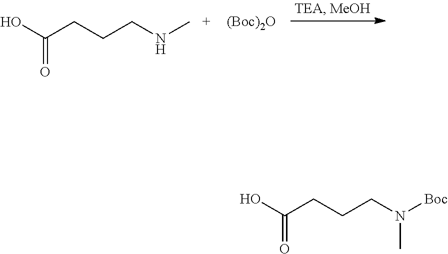

To a solution of 4-(methylamino)butanoic acid hydrochloride (3.0 g, 19.5 mmol) and TEA (7.78 mL, 58.6 mmol) in MeOH (30 mL) was added Boc₂O (4.69 g, 21.5 mmol) dropwise. The mixture was stirred at RT for 2 h. The mixture was concentrated under reduced pressure and the residue was diluted with EtOAc (100 mL), washed with cooled 0.1 N HCl (70 mL×2), H₂O (50 mL×2) and brine (50 mL), dried over Na₂SO₄, filtered and concentrated to afford the title intermediate (1.80 g, crude) as colorless oil.

### Preparation of Intermediate 8—tert-butyl (4-(methoxy(methyl)amino)-4-oxobutyl)(methyl) carbamate

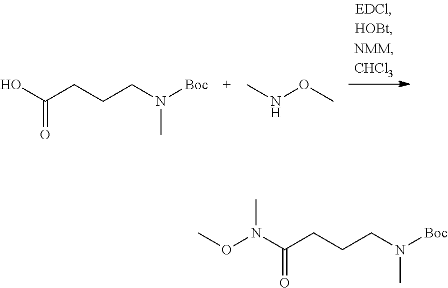

To a solution of 4-((tert-butoxycarbonyl)(methyl)amino)butanoic acid (intermediate 7) (1.80 g, crude) in CHCl₃ (30 mL) was added N,O-dimethylhydroxylamine hydrochloride (960 mg, 9.84 mmol), HOBt (1.24 g, 9.18 mmol) and NMM (2.80 mL, 25.1 mmol). And, then EDCI (2.23 g, 11.6 mmol) was added and the reaction mixture was stirred at RT for 4 h. The reaction mixture was diluted with DCM (100 mL), washed with 1N HCl (30 mL×3), sat. aq. NaHCO₃ (30 mL×3) and brine (30 mL), dried over Na₂SO₄, filtered and concentrated under in vacuo to afford the title intermediate (1.70 g, crude) as colorless oil.

### Preparation of Intermediate 9—tert-butyl methyl(5-methyl-4-oxohexyl)carbamate

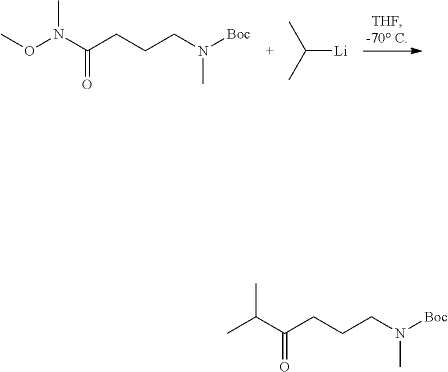

To a solution of tert-butyl (4-(methoxy(methyl)amino)-4-oxobutyl)(methyl)carbamate (intermediate 8) (200 mg, crude) in THF (5 mL) cooled at −70° C. under N₂ atmosphere was added dropwise isopropyllithium (3.2 mL, 2.24 mmol, 0.7M in pentane). The resulting mixture was stirred at −70° C. for 2 h. The mixture was quenched with sat. aq. NH₄Cl (15 mL), extracted with EtOAc (30 mL×2). The combined organic layers were washed with brine (30 mL), dried over Na₂SO₄, filtered and concentrated under reduced pressure to give a crude product. The crude product was further purified by FCC (PE/EtOAc=10:1) to afford the title intermediate (60 mg) as colorless oil.

### Preparation of Compound 60—tert-butyl (4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl) (methyl) carbamate

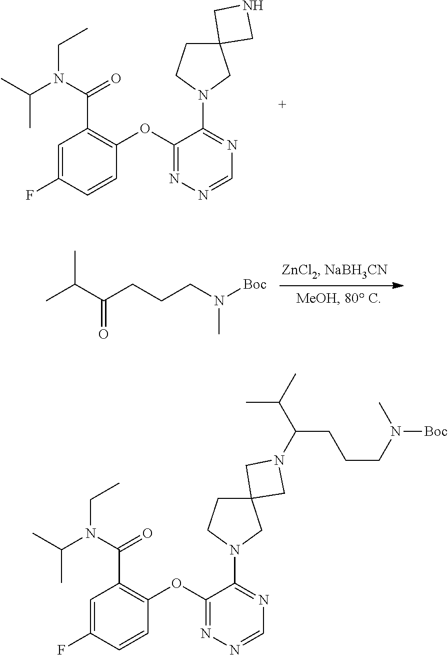 [SMILES: CCN(C(=O)c1cc(F)ccc1Oc1nncnc1N1CCC2(CNC2)C1)C(C)C ; CC(C)C(=O)CCCN(C)C(=O)OC(C)(C)C ; CCN(C(=O)c1cc(F)ccc1Oc1nncnc1N1CCC2(C1)CN(C(CCCN(C)C(=O)OC(C)(C)C)C(C)C)C2)C(C)C]

To a solution of 2-((5-(2,6-diazaspiro[3.4]octan-6-yl)-1,2,4-triazin-6-yl)oxy)-N-ethyl-5-fluoro-N-isopropylbenzamide (intermediate 3) (600 mg, 1.45 mmol) and tert-butyl methyl(5-methyl-4-oxohexyl)carbamate (intermediate 9) (330 mg, 1.37 mmol) in MeOH (50 mL) was added ZnCl₂ (789 mg, 5.79 mmol). The resulting mixture was stirred at 80° C. for 2 h. Then NaBH₃CN (729 mg, 11.6 mmol) was added and the reaction mixture was stirred at 80° C. overnight. After cooling to RT, the mixture was concentrated under reduced pressure to give a crude residue, which was diluted with DCM (50 mL), quenched with sat. aq. NH₄Cl (50 mL) and extracted with DCM (50 mL×3). The combined organic layers were washed with brine (50 mL), dried over Na₂SO₄, filtered and the filtrate was concentrated under reduced pressure to give a crude product which was further purified by FCC (DCM/MeOH=10:1) to afford the title compound (400 mg, 42% yield) as white solid.

### Compound 67—N-ethyl-5-fluoro-N-isopropyl-2-((5-(2-(2-methyl-6-(methylamino)hexan-3-yl)-2,6-diazaspiro[3.4]octan-6-yl)-1,2,4-triazin-6-yl)oxy)benzamide hydrochloride

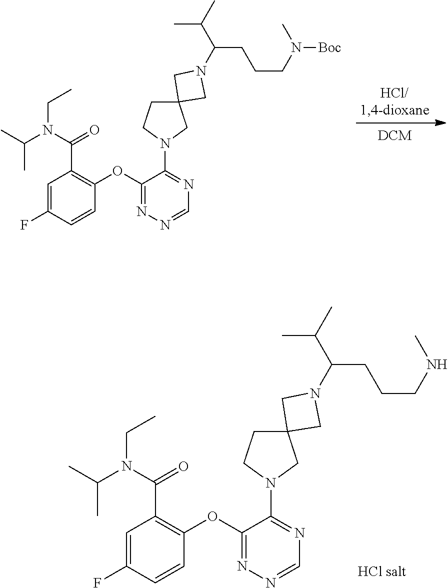 [SMILES: CCN(C(=O)c1cc(F)ccc1Oc1nncnc1N1CCC2(C1)CN(C(CCCN(C)C(=O)OC(C)(C)C)C(C)C)C2)C(C)C ; CCN(C(=O)c1cc(F)ccc1Oc1nncnc1N1CCC2(C1)CN(C(CCCNC)C(C)C)C2)C(C)C]

To a solution of tert-butyl (4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)(methyl)carbamate (compound 60) (1 g, 1.56 mmol) in DCM (10 mL) was added 4M HCl in dioxane (5 mL, 20 mmol), the resulting mixture was stirred at RT for 1 h. The reaction mixture was concentrated in vacuo to afford the title compound (960 mg, crude, HCl salt) which was used directly in next step without further purification.

### Compound A—(R)-N-ethyl-5-fluoro-N-isopropyl-2-((5-(2-(6-((2-methoxyethyl) (methyl) amino)-2-methylhexan-3-yl)-2,6-diazaspiro[3.4]octan-6-yl)-1,2,4-triazin-6-yl)oxy) benzamide

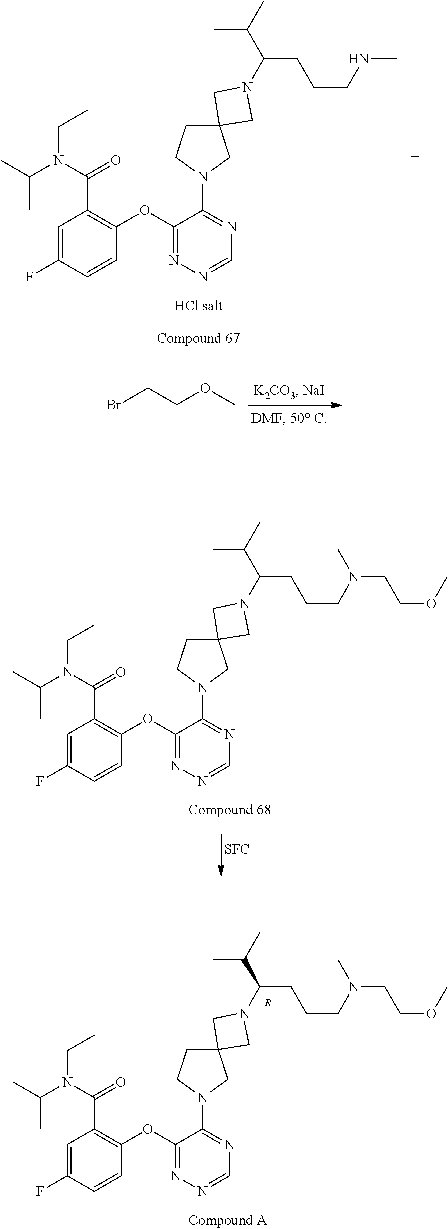

To the mixture of N-ethyl-5-fluoro-N-isopropyl-2-((5-(2-(2-methyl-6-(methylamino) hexan-3-yl)-2,6-diazaspiro[3.4]octan-6-yl)-1,2,4-triazin-6-yl)oxy)benzamide hydrochloride (compound 67) (480 mg, crude), K₂CO₃ (700 mg, 5.07 mmol) and NaI (400 mg, 2.67 mmol) in DMF (5 mL) was added 1-bromo-2-methoxyethane (230 mg, 1.65 mmol). The resulting mixture was stirred at 50° C. overnight. After cooled to RT, the reaction mixture was quenched with H₂O (30 mL) and extracted with DCM (30 mL×3). The combined organic layers were washed with brine (30 mL×3), dried over Na₂SO₄, filtered and concentrated to give a crude residue. The residue was purified by FCC (DCM/MeOH=10:1) to afford N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide (compound 68) (250 mg, 48% yield) as yellow oil.

The N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide (compound 68) (960 mg, combined from several batches obtained by Method B) was first separated by SFC using DAICEL CHIRALPAK IG (column: 250×30 mm 10 um; Mobile phase: A: Supercritical CO₂, B: EtOH (0.1% ammonia), A:B=40:60 at 60 mL/min) and further purified by preparative HPLC using Boston Prime (column: 150×30 mm Sum, Mobile Phase A: H₂O (10 mM NH₄HCO₃), Mobile Phase B: ACN, Flow rate: 25 mL/min, gradient condition B/A from 55% to 85%) to afford the title compound (270 mg) as colorless oil.

¹H NMR (400 MHz, Methanol-d₄): δ=8.40 (s, 1H), 7.47-7.32 (m, 1H), 7.30-7.10 (m, 2H), 4.24-4.01 (m, 2H), 3.89-3.60 (m, 3H), 3.48 (br s, 3H), 2.63-2.51 (m, 2H), 2.43-2.32 (m, 2H), 2.29-2.07 (m, 6H), 1.86-1.72 (m, 1H), 1.62-1.44 (m, 2H), 1.39-1.02 (m, 10H), 0.99-0.66 (m, 9H). Some protons were hidden by the solvent peak and are not reported.

LCMS (ESI) (Method 2): Rₜ=1.965 min, m/z found 600.3 [M+H]⁺.

SFC (Method 11): Rₜ=4.904 min.

### Example 3—Synthesis of (R)-N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A)—Preparation Method C

### Preparation of Intermediate 227—tert-butyl (R)-(1-(2,2-dimethyl-4,6-dioxo-1,3-dioxan-5-yl)-3-methylbutan-2-yl)carbamate

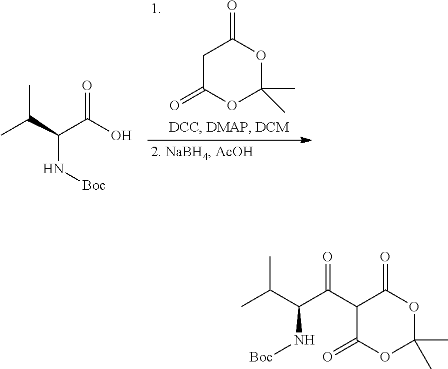

Boc-L-valine (44.9 kg), 2,2-dimethyl-1,3-dioxane-4,6-dione (32.9 kg) and DMAP (35.5 kg) in DCM (607 kg) pre-cooled at −10 to 0° C. were added to a solution of DCC (55.5 kg) in DCM (613 kg) over 3 h and aged for 16 h at −10 to 0° C. 10% citric acid aqueous solution (449 kg) was added whilst maintaining a temperature below 10° C. The resulting slurry was aged for 2 h at 0 to 10° C. then filtered. The filter cake was washed with DCM (91 kg). The filtrate was separated and the organic layer was washed with 10% citric acid aqueous solution (two times 450 kg) and 10% NaCl aqueous solution (449 kg). To organic phase (1200 kg), was added acetic acid (75.0 kg) whilst maintaining a temperature between −10 to 0° C. Sodium Borohydride (18.0 kg) was added in portions over 5 h whilst maintaining a temperature in the range −10 to 0° C. and then resulting mixture was aged at −10 to 0° C. for an additional 16 h. The mixture was warmed to 15 to 25° C., and aged for 2 h. The mixture was then washed with 14% NaCl aqueous solution (450 kg) followed by a second wash with 14% NaCl aqueous solution (432 kg) and a final water wash (444 kg). The organic phase was concentrated under reduced pressure to 2-4 vol. Iso-propanol (143 kg) was added to the residue and concentrated to 4-5 vol. under reduced pressure. After cooling to −10 to 0° C. and aging for 8 h, the resulting slurry was filtered, washed with IPA (38 kg) and dried to afford the title intermediate (46.7 kg, 69% yield) as a white solid.

### Preparation of Intermediate 228—tert-butyl (R)-2-isopropyl-5-oxopyrrolidine-1-carboxylate

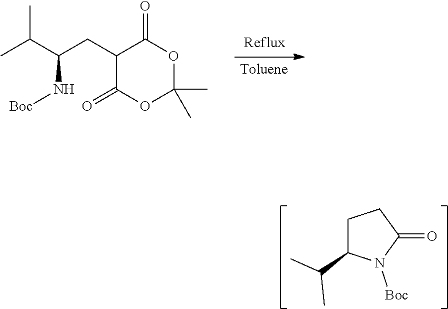

tert-butyl (R)-(1-(2,2-dimethyl-4,6-dioxo-1,3-dioxan-5-yl)-3-methylbutan-2-yl)carbamate (intermediate 227) (46.7 kg) in toluene (333 kg) was heated to reflux and aged for 4 h. The mixture was cooled to ambient temperature, filtered and washed with toluene (20 kg). The combined filtrates were concentrated to dryness at reduced pressure to afford the desired compound (31.05 kg, 96% yield) as an oil which was used directly without further purification.

### Preparation of Intermediate 229—tert-butyl (5R)-2-hydroxy-5-isopropylpyrrolidine-1-carboxylate

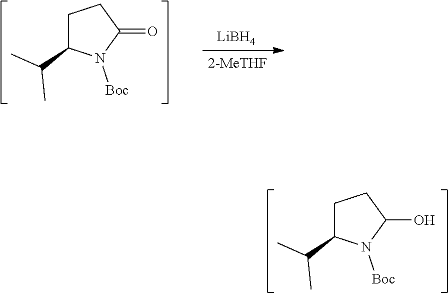

tert-butyl (R)-2-isopropyl-5-oxopyrrolidine-1-carboxylate (intermediate 228) (30.9 kg) in 2-MeTHF (26.7 kg) was cooled to −5 to 5° C. A solution of LiBH₄ in 2-MeTHF (1M, 45.2 kg, 54.4 mol) was added over 3 h and the mixture was aged for 4 h. A cold aqueous solution of 5% NaHCO₃ (163 kg) was added at −5 to 5° C. over 3 h and aged for an additional 2 h. The mixture was warmed to ambient temperature and aged for a further 2 h. The aqueous layer was separated and the organic layer was washed with 10% NaCl aqueous solution (170 kg) and water (155 kg). During the water wash, an emulsion formed and solid NaCl (3.1 kg) was added to affect the separation. After removal of the aqueous layer, the organic layer was concentrated under reduced pressure to dryness to afford the desired compound (28.5 kg, 91% yield) as an oil, which was used directly without further purification.

### Preparation of Intermediate 230—tert-butyl (R)-(6-((2-methoxyethyl)(methyl)amino)-2-methylhexan-3-yl)carbamate

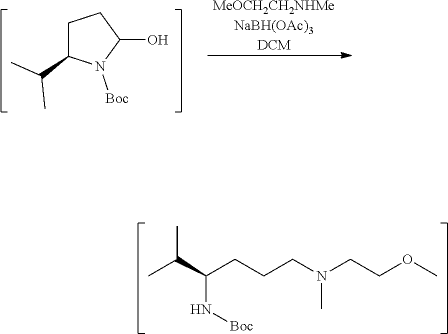

tert-butyl (5R)-2-hydroxy-5-isopropylpyrrolidine-1-carboxylate (intermediate 229) (28.55 kg) in DCM (344 kg), at 15 to 25° C. was treated with 2-methoxy-N-methylethan-1-amine (12.3 kg, 138.0 mol) and the resulting mixture was aged for 1 h. Sodium triacetoxyborohydride (40.12 kg) was added in portions over 5 h whilst maintaining a temperature between 15 to 25° C. and the resulting mixture was aged for 48 h. The reaction mixture was quenched by the addition of 8% NaOH aqueous solution (184 kg) over 2 h whilst maintaining a temperature between 15 to 25° C. and the mixture was aged for a further 2 h. The water layer was separated, and the organic layer was washed with water (169 kg). The organic layer was then concentrated under reduced pressure to dryness to afford the title intermediate (33.26 kg, 88% yield) as an oil which was used directly without further purification.

### Preparation of Intermediate 231—(R)-N¹-(2-methoxyethyl)-N¹,5-dimethylhexane-1,4-diamine, dihydrochloride

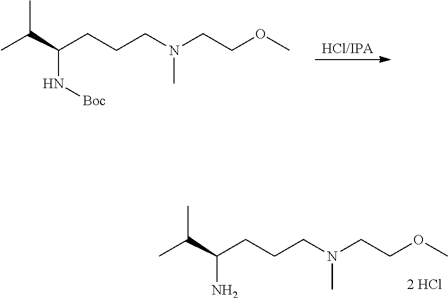

To 4 molar solution of HCl in iso-propanol (84.80 kg) at ambient temperature was added a solution of tert-butyl (R)-(6-((2-methoxyethyl)(methyl)amino)-2-methylhexan-3-yl)carbamate (intermediate 230) (32.38 kg) in iso-propanol (25.6 kg) over 3 h and the mixture was aged at ambient temperature for an additional 19 h. Methyl tert-butyl ether (95.25 kg) was then added over 1 h and the mixture was aged for 2.5 h. The resulting slurry was filtered and washed with MTBE (53 kg). The filter cake was dried to afford the title compound (23.92 kg, 81% yield) as a white solid.

### Preparation of Intermediate 232—ethyl 1-benzyl-3-(chloromethyl)pyrrolidine-3-carboxylate

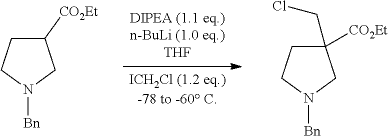 [SMILES: CCOC(=O)C1CCN(Cc2ccccc2)C1 ; CCOC(=O)C1(CCl)CCN(Cc2ccccc2)C1]

To a solution of DIPEA (952 g, 1.1 eq.) in THF (6 L) which was cooled to −35 to −25° C. was added n-BuLi (2.33 kg, 2.5 M in hexane, 1.0 eq.) whilst maintaining a temperature below −25° C. The resulting mixture was aged at −35 to −25° C. for an additional 30 min then cooled to between −78 to −60° C. A solution of ethyl 1-benzylpyrrolidine-3-carboxylate (2 kg, 1.0 eq.) in THF (2 L) at −78 to −60° C. was added and stirred for an addition 30 min. Chloroiodomethane (1.81 kg, 1.2 eq.) was then charged at −78 to −60° C. The reaction mixture was aged at −60 to −40° C. for 2 h. To the reaction mixture was added to citric acid aqueous solution (660 g in 6 L H₂O) at a temperature between 0 to 10° C. and the resulting mixture was aged at 20 to 30° C. for an additional 20 min. After separating the layers, the aqueous layer was extracted with EtOAc (6 L) and the combined organic layers washed with brine (6 L) then warmed to 50 to 60° C. Oxalic acid (2.22 kg) was charged at 50 to 60° C. The resulting mixture was stirred at 50 to 60° C. for 3 h then cooled to 20 to 30° C. and aged overnight. The resulting solid was filtered and the cake was washed with ethyl acetate (2 L). The wet cake was added to toluene (4 L), H₂O (8 L) and K₃PO₄ (1.5 eq.) and the resulting mixture was aged at 20 to 30° C. for 20 min. After separating the layers, the aqueous layer was extracted with toluene (2 L). The organic layers were combined and washed twice with water (2 L). The organic phase was concentrated under reduced pressure to afford 4.2 kg of the desired compound as a toluene solution (46 wt % by assay, giving an assay yield of 80%).

### Preparation of Intermediate 233—1-benzyl-3-(chloromethyl)pyrrolidine-3-carbaldehyde

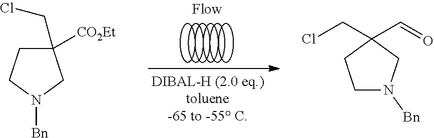 [SMILES: CCOC(=O)C1(CCl)CCN(Cc2ccccc2)C1 ; O=CC1(CCl)CCN(Cc2ccccc2)C1]

Reaction conducted in a flow chemistry system: A solution of ethyl 1-benzyl-3-(chloromethyl)pyrrolidine-3-carboxylate (intermediate 232) (4.4 kg) in toluene (26 L) was pumped at 26.7 mL/min and cooled to −60° C. After cooling, it was then mixed with a cooled solution of DIBAL-H (28.1 mol) in toluene at −60° C. (28 L) with a pumping rate of 32.1 m/min. The mixture was passed through a Perfluoroalkoxy (PFA) coil tube reactor at −60° C. (total flow rate of 58.8 mL/min with a residence time of 5 seconds). The resulting mixture was mixed with cooled MeOH (−60° C.) which was pumped at the rate of 15.2 mL/min. This mixed solution was pumped to another PFA coil tube reactor at −60° C. (total flow rate of 74 mL/min with a residence time of 5 seconds). The resulting mixture was collected into a receiver which contained 20 wt % aq. solution Rochelle's salt (20 V). The layers were separated, and the organic phase was twice washed with water (2×44 L). The organic phase was combined with another 3.0 kg batch prepared in an analogous manner and concentrated under reduced pressure to afford 20.8 kg of a toluene solution of the desired compound (25.5 wt % assay by HPLC, giving an assay yield of 85%) which was used directly without further purification.

¹H NMR (300 MHz, Chloroform-d): δ 9.62 (s, 1H), 7.39-7.20 (m, 5H), 3.83-3.57 (m, 4H), 2.96 (d, J=10.2 Hz, 1H), 2.80-2.55 (m, 3H), 2.17 (ddd, J=13.9, 7.9, 6.1 Hz, 1H), 1.83 (ddd, J=13.4, 7.8, 5.5 Hz, 1H).

### Preparation of Intermediate 234—(R)-4-(6-benzyl-2,6-diazaspiro[3.4]octan-2-yl)-N-(2-methoxy ethyl)-N,5-dimethylhexan-1-amine

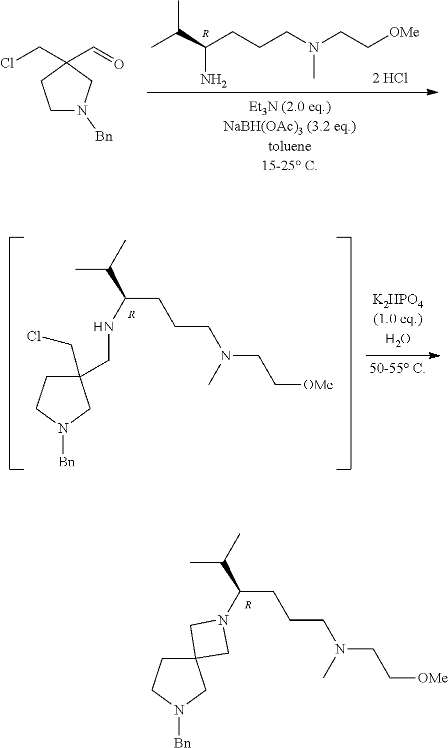

To a solution of 1-benzyl-3-(chloromethyl)pyrrolidine-3-carbaldehyde (intermediate 233) in toluene (3.0 kg, 10 wt %) diluted with toluene (30 L) and (R)-N¹-(2-methoxyethyl)-N¹,5-dimethylhexane-1,4-diamine, dihydrochloride (intermediate 231) (3.47 kg) was added triethylamine (2.55 kg, 25.2 mol) at 20 to 30° C. The resulting mixture was aged for 2 h at 20 to 30° C. Then sodium triacetoxyborohydride (9.0 kg) was charged at 20 to 30° C. and the mixture was aged for 12 h. The reaction mixture was cooled to 5 to 15° C. and 25 wt % NaOH aqueous solution (25 L, ˜16.75 eq.) was added maintaining a temperature below 35° C. The resulting mixture was aged at 20 to 30° C. for 25 mins and the layers were separated. The organic layer was washed with 15 wt % aq. NaCl (10 L) and the layers were again separated and water (18 L) was charged to the organic phase. The pH of the aqueous phase was adjusted to 6-7 with 4M aq. HCl whilst maintaining an internal temperature below 35° C. The organic phase was then discarded and the aqueous phase was separated and basified to pH 8-9 with K₂HPO₄.

The resulting mixture was warmed to 50 to 55° C. and aged for 3 h. The reaction mixture was then cooled to ambient temperature and combined with other two batches (2.4 kg+3.0 kg). The combined streams were washed with methyl tert-butyl ether three times (3×40 L). To the resulting aqueous layer was added additional methyl tert-butyl ether (83 L) and the aqueous phase was basified to pH 9˜10 using 8 wt % aq. NaOH whilst maintaining a temperature between 15 to 35° C. The aqueous layer was separated, and the organic layer was washed with three times water (3×30 L). The organic layer was then concentrated under reduced pressure to approximately 3 volumes and then flushed with methanol three times (3×30 L) and concentrated to dryness to afford the desired intermediate (12.4 kg, 90% isolated yield) as light-yellow oil, which was used directly without further purification.

### Preparation of Intermediate 234a (citric acid salt of intermediate 234)

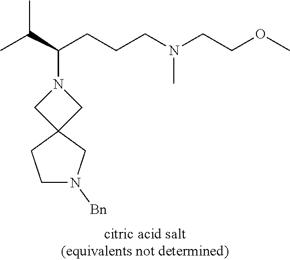 [SMILES: COCCN(C)CCC[C@H](C(C)C)N1CC2(CCN(Cc3ccccc3)C2)C1]

EtOH (80 ml) and intermediate 234 (20 g) were added in a round bottom flask. Next, a 0.5 M solution of citric acid in EtOH (100 ml; 1 equivalent) was added to the mixture in the round bottom flask at room temperature. Subsequently, the mixture was evaporated till dryness (Rotavap, 40° C.). Acetonitrile (200 ml) was added to the residue and the mixture was evaporated till dryness (Rotavap, 40° C.). Acetonitrile (100 ml) was added to the residue and stirred overnight on a magnetic heating plate at room temperature. Finally, intermediate 234a was filtered off and dried at room temperature.

### Preparation of Crystalline Form of Citric Acid Salt of Intermediate 234 (Intermediate 234b)

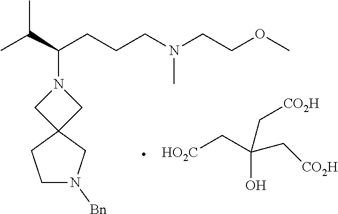 [SMILES: COCCN(C)CCC[C@H](C(C)C)N1CC2(CCN(Cc3ccccc3)C2)C1 ; O=C(O)CC(O)(CC(=O)O)CC(=O)O]

crystalline
ratio intermediate/citric acid 3/2

Intermediate 234a (3.72 g) was added to acetonitrile (20 ml) at room temperature and the mixture was stirred. The mixture was heated to 60° C. until the reaction mixture became homogeneous (about 10 minutes). Next, the mixture was cooled to 50° C. at a rate of 0.5° C./min. Next, seeds were added (19 mg of intermediate 234a; 0.5 w/w %) and the mixture was aged while stirring during 3 hours and 30 minutes. Next, the mixture was cooled non-linear to 20° C. over 8 hours with an exponent of 2,3. The obtained mixture was stirred overnight and the product was filtered off and dried (overnight at room temperature in hood). After isolation, intermediate 234b was obtained (2.75 g; yield 73.9%) as the crystalline form of the citric acid salt of intermediate 234. The obtained ratio of the intermediate/citric acid is 3/2 (NMR).

The non-linear cooling referred to above was done according to the formula below:
A new linear ramp is started every 30 seconds during the defined duration of the cooling. The ramp is calculated according to the following equation:

$${T}_{s e t} = {T}_{s t artvalue} - [ ( {T}_{s t artνalue} - {T}_{endvalue} ) * {( \frac{{t}_{action} + 30 s}{Dura t i o n} )}^{n} ]$$

Tₛₑₜ: Set value for each new ramp
T_{start value}: Measured mixture temperature at the start of the cooling trajectory
T_{end value}: Defined end value of cooling trajectory
t_{action}: Actual time from the start of the cooling
Duration: Defined cooling duration
n: Exponent

¹H NMR (400 MHz, MeOH-d₄) δ ppm 0.91 (3H, d, J=6.88 Hz) 0.98 (3H, d, J=6.88 Hz) 1.46-1.57 (2H, m) 1.67-1.87 (2H, m) 1.94-2.03 (1H, m) 2.20-2.29 (2H, m) 2.62-2.69 (2H, m) 2.72-2.77 (4H, m) 2.77-2.82 (2H, m) 2.90 (2H, t, J=7.32 Hz) 2.95-3.02 (2H, m) 3.07-3.16 (2H, m) 3.16-3.22 (2H, m) 3.37 (3H, s) 3.68-3.72 (2H, m) 3.83-3.89 (2H, m) 3.90-3.92 (2H, m) 3.94-4.06 (2H, m) 7.32-7.43 (5H, m).

### Preparation of Intermediate 224—(R)-N-(2-methoxyethyl)-N,5-dimethyl-4-(2,6-diazaspiro [3.4]octan-2-yl)hexan-1-amine

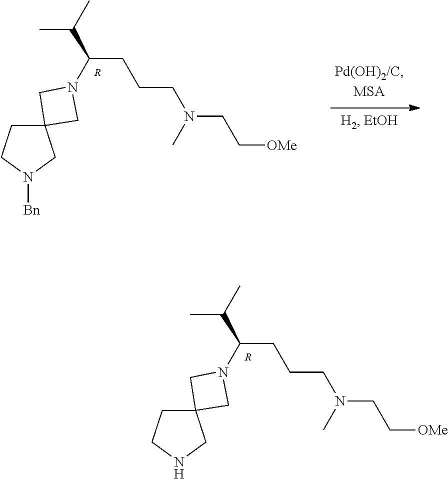

To palladium hydroxide on carbon (1.2 kg) in EtOH (1.47 kg) cooled to −5 to 5° C. were added methanesulfonic acid (MSA) (11 kg), (R)-4-(6-benzyl-2,6-diazaspiro[3.4]octan-2-yl-N-(2-methoxyethyl)-N,5-dimethylhexan-1-amine (intermediate 234) (10 kg) and EtOH (250 L). The mixture was warmed to 35-45° C. and stirred under a hydrogen atmosphere (0.27 to 0.40 MPa) for 16-20 h. The mixture was filtered over diatomite (20 kg) and the pad was washed with EtOH (24 L). The filtrate was concentrated under reduced pressure (<40° C.) to 2-3 vol. and then flushed twice with 2-MeTHF (73 kg and 47 kg) to give a 2˜3 vol. solution. After dilution with 2-MeTHF (65 kg), 10% aq. sodium sulfate (30 kg) was added and the mixture was cooled to 0 to 10° C., followed by the addition of 16% aq. NaOH (50 kg) to adjust the pH to 13˜14. The temperature was adjusted to 15 to 25° C. and stirred for 30 to 60 min. The aqueous layer was separated and extracted twice with 2-MeTHF (47 kg×2). The combined organic layers were concentrated under reduced pressure (<40° C.) to 3˜4 vol. and 2-MeTHF (950 g) was added. After concentration under reduced pressure (<40° C.) to 3˜4 vol., the resulting solution was diluted with 2-MeTHF (30 kg), dried by passing through 4A molecular sieves (25 kg) and washed with 2-MeTHF (30 kg). The final solution was concentrated to afford the desired compound (6.7 kg) as an oil with 90.1% assay purity in a 79% corrected yield.

### Preparation of Intermediate 225—(R)-4-(6-(3,6-dichloro-1,2,4-triazin-5-yl)-2,6-diazaspiro [3.4]octan-2-yl)-N-(2-methoxyethyl)-N,5-dimethylhexan-1-amine

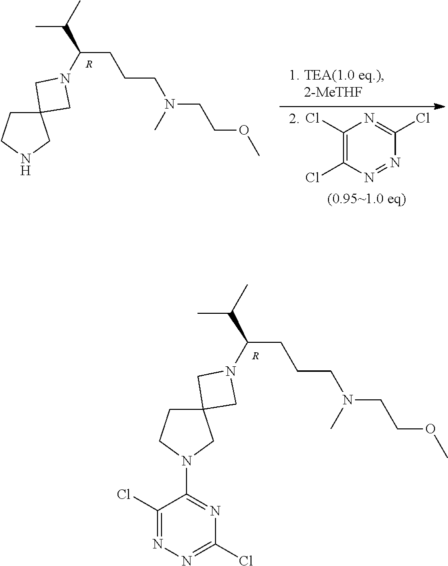

To (R)-N-(2-methoxyethyl)-N,5-dimethyl-4-(2,6-diazaspiro[3.4]octan-2-yl)hexan-1-amine (intermediate 224) (100 g) was added 2-MeTHF (430 g) and TEA (68 g) and the mixture was cooled to −50 to −40° C. 3,5,6-trichloro-1,2,4-triazine (62 g) in 2-MeTHF (172 g) was added and the mixture was stirred for 1 to 3 h. The resulting mixture was warmed to −20 to −10° C. and a 7% NaHCO₃ aqueous solution was added, the mixture was warmed to 20 to 30° C. and stirred for 30 to 60 min. The aqueous layer was removed and the organic layer was washed with 10% Na₂SO₄ (500 g). The organic layer was dried by passing through 4A molecular sieves (220 g) and washed with 2-MeTHF (180 g). The title intermediate was afforded in 90% assay yield as a solution 14.8 wt % in 2-MeTHF.

### Compound 393—(R)-2-((3-chloro-5-(2-(6-((2-methoxyethyl)(methyl)amino)-2-methylhexan-3-yl)-2,6-diazaspiro[3.4]octan-6-yl)-1,2,4-triazin-6-yl)oxy)-N-ethyl-5-fluoro-N-isopropyl-benzamide

### Synthesis Method a for Compound 393

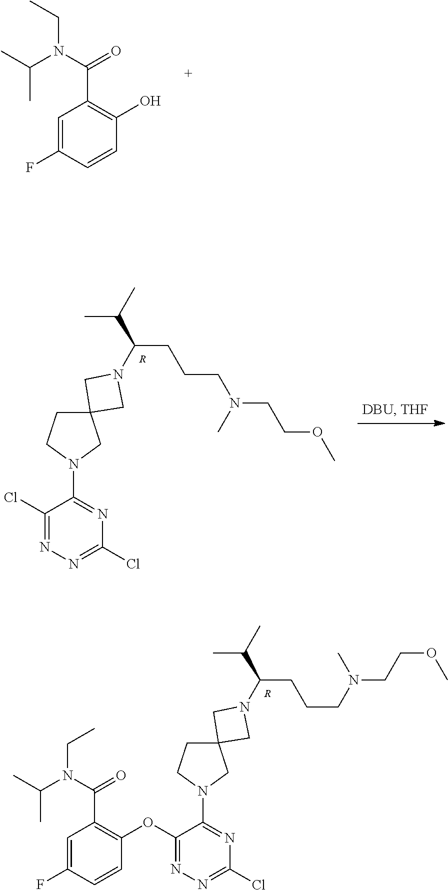

The mixture of N-ethyl-5-fluoro-2-hydroxy-N-isopropylbenzamide (intermediate 28) (1.10 g, 4.88 mmol), (R)-4-(6-(3,6-dichloro-1,2,4-triazin-5-yl)-2,6-diazaspiro[3.4]octan-2-yl)-N-(2-methoxyethyl)-N,5-dimethylhexan-1-amine (intermediate 225) (1.70 g, 3.82 mmol) and DBU (750 mg, 4.93 mmol) in anhydrous THF (15 mL) was stirred at 40° C. for 8 h. After cooled to RT, the mixture was concentrated under reduced pressure, the resulting residue was diluted with DCM (60 mL) and washed with H₂O (20 mL×3). The organic layer was dried over anhydrous Na₂SO₄, filtered and concentrated under reduced pressure to give the crude product which was purified FCC (MeOH/DCM=0% to 10%) to afford a yellow oil (1.40 g), which was further separated by SFC over DAICEL CHIRALPAK AD (column: 250×50 mm, 10 um; Mobile phase: A: Supercritical CO₂, B: EtOH (0.1% ammonia), A:B=50:50 at 70 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm) to afford the title compound (1.0 g).

### Synthesis Method a for Compound 393

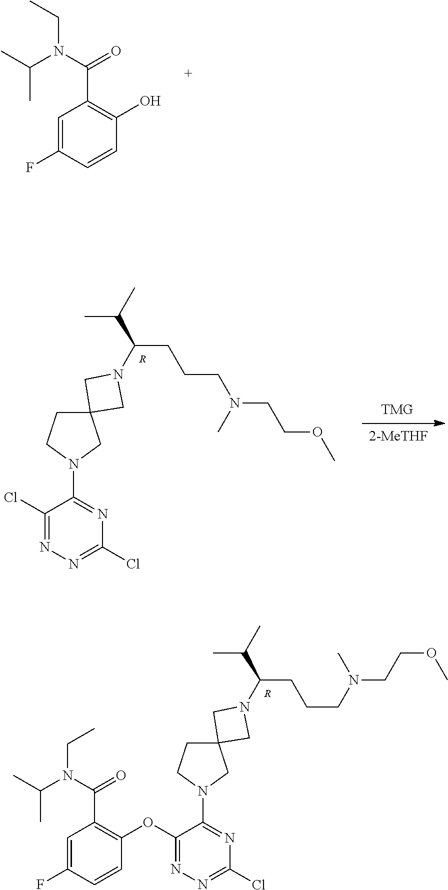

To a 2-MeTHF solution of (R)-4-(6-(3,6-dichloro-1,2,4-triazin-5-yl)-2,6-diazaspiro[3.4]octan-2-yl)-N-(2-methoxyethyl)-N,5-dimethylhexan-1-amine (intermediate 225) (676 g of a 14.8 wt % solution in 2-MeTHF, 100 g corrected of intermediate 225) and N-ethyl-5-fluoro-2-hydroxy-N-isopropylbenzamide (intermediate 28) (50.6 g) in 2-MeTHF (40 g) at 20 to 30° C. was added tetramethylguanidine (31 g) and the mixture was stirred for 40 to 48 h. A 7% NaHCO₃ aqueous solution (500 g) was added and the mixture was stirred for 30 to 60 min. The aqueous layer was removed and the organic layer was washed with twice with 4% NaOH aqueous solution (2×500 g) and once with 10% Na₂SO₄ aqueous solution (500 g). The organic layer was concentrated under reduced pressure (<40° C.) to 2.2˜3.0 vol. and flushed three times with MeOH (1×790 g and 2×395 g) until both 2-MeTHF and water content were both ≤1.0% to afford the desired compound in 86% assay yield as a 60.1 wt % solution in methanol.

### Compound A—(R)-N-ethyl-5-fluoro-N-isopropyl-2-((5-(2-(6-((2-methoxyethyl) (methyl) amino)-2-methylhexan-3-yl)-2,6-diazaspiro[3.4]octan-6-yl)-1,2,4-triazin-6-yl)oxy) benzamide

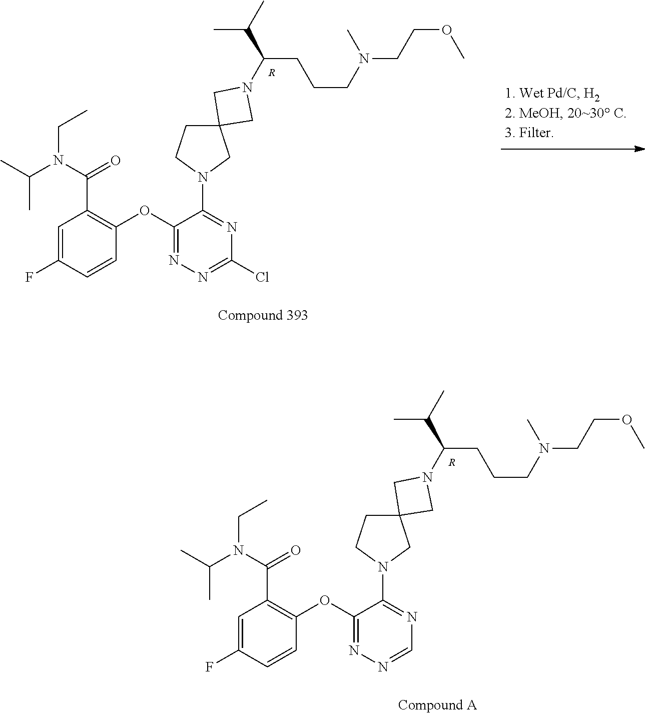

A methanol solution of (R)-2-((3-chloro-5-(2-(6-((2-methoxyethyl)(methyl)amino)-2-methylhexan-3-yl)-2,6-diazaspiro[3.4]octan-6-yl)-1,2,4-triazin-6-yl)oxy-N-ethyl-5-fluoro-N-isopropylbenzamide (compound 393) (163.93 g of a 60.1 wt % solution in MeOH, 100 g corrected of compound 393), palladium on carbon (10 g) and MeOH (316 g) was stirred at 20 to 30° C. under a hydrogen atmosphere (0.20 to 0.30 Mpa) for 18 h. The mixture was filtered over diatomite (75 g) and the cake was washed with MeOH (158 g). The filtrate was concentrated under reduced pressure (≤40° C.) to ˜3 vol., then flushed with isopropyl acetate (IPAc, 870 g) concentrating to ˜3 vol. The mixture was then diluted with IPAc (696 g) and a 20% Na₂CO₃ aqueous solution was added (500 g). The mixture was stirred for 30 to 60 min. The aqueous layer was removed. The organic layer was washed with water (500 g) then concentrated under reduced pressure <45° C. to ˜3 vol. The title intermediate was afforded in approximately 90% assay yield as a 48.1 wt % solution in IPAc.

### Example 4—Synthesis of (R)-N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide oxalate (Compound A3)

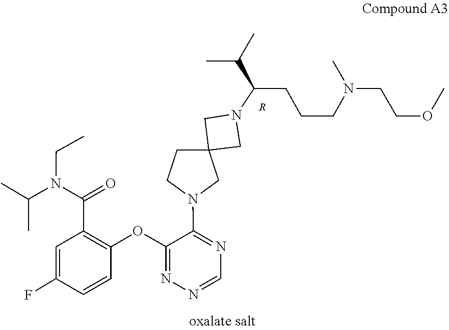

To a solution of (R)-N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A) (270 mg, 0.450 mmol) in 20 mL of ACN (20 mL) was added oxalic acid (81.0 mg, 0.900 mmol). After addition, the reaction mixture was stirred at RT for 1 h. Then the reaction mixture was concentrated, the residue was re-dissolved in ACN and deionized water, and lyophilized to afford the title compound (350 mg) as white solid.

¹H NMR (400 MHz, Methanol-d₄): δ=8.48 (s, 1H), 7.52-7.11 (m, 3H), 4.54-3.64 (m, 12H), 3.40-3.34 (m, 5H), 3.23-3.13 (m, 2H), 2.90 (s, 3H), 2.54-2.27 (m, 2H), 2.19-2.03 (m, 1H), 1.97-1.77 (m, 2H), 1.75-1.50 (m, 2H), 1.35-0.65 (m, 17H).

¹H NMR (400 MHz, DMSO-d₆): δ=8.51 (s, 1H), 7.51-7.29 (m, 3H), 4.29-3.34 (m, 12H), 3.23-2.84 (m, 7H), 2.70 (s, 3H), 2.35-2.09 (m, 2H), 2.05-1.85 (m, 1H), 1.81-1.58 (m, 2H), 1.56-1.33 (m, 2H), 1.18-0.60 (m, 17H).

LCMS (ESI) (Method 2): Rₜ=1.969 min, m/z found 600.4 [M+H]⁺.

### Example 5—Synthesis of Compound A1

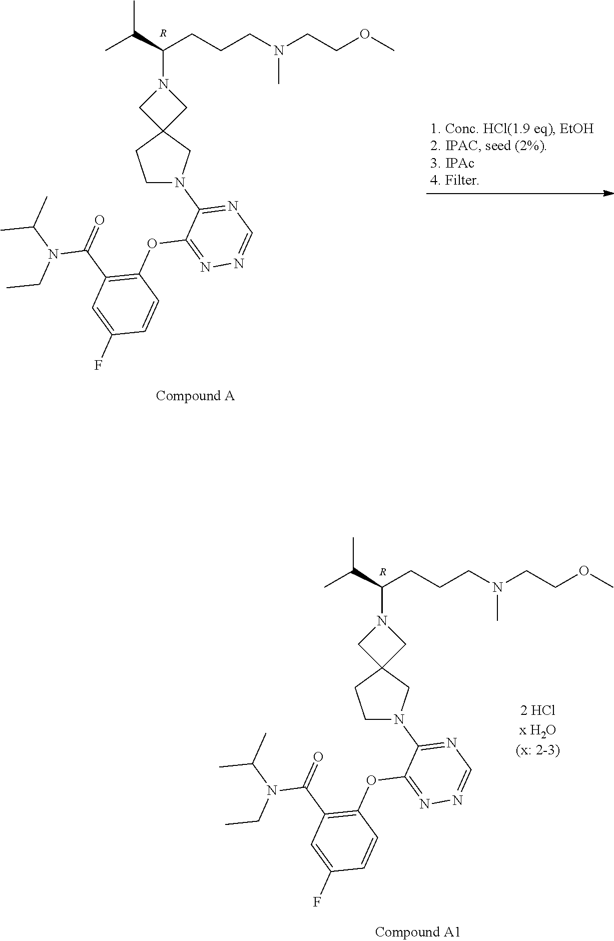

To a solution of Compound A (207.90 g of a 48 wt % solution in IPAc, 100 g of active Compound A) in IPAc (360 g) was added EtOH (63 g) at 20 to 25° C. The solution was then treated with conc. HCl (32.9 g) in EtOH (49.5 g) over −15 min. The mixture was seeded with crystalline Compound A1 seed (2 g, 2% seed load) then aged for 18 h. IPAc (870 g) was added slowly over 4 h at between 20 to 25° C. and the slurry was stirred for an additional 18 h. After cooling to −5° C., the product was filtered, washed with IPAc (522 g) and dried under vac at 20-30° C. to afford the weakly crystalline Compound A1 as a white solid (91.0% yield, 115.4 g). (Note: A small amount of seed material used in the reaction was obtained via an analogous reaction protocol on small-scale.)

Recrystallisation: A solution of weakly crystalline Compound A1 (100 g), EtOH (166 g), purified water (21.5 g) and IPAc (178 g) was stirred at 20 to 30° C. for 0.5-2 h to get a clear solution. Extra IPAc (522 g) was added dropwise over 1-2 h, and then the mixture was seeded with crystalline Compound A1 seed (2 g, 2% seed load). Then the mixture was aged for 18-20 h, IPAc (348 g) was added slowly over 12 h at between 20 to 30° C., and the slurry was stirred for an additional 55-60 h. The product was filtered, washed with IPAc (158 g) and dried in vacuo at 20-30° C. to afford Compound A1 as a white solid (85% yield, 85.0 g, net).

¹HNMR (DMSO-d₆, 400 MHz): δ=11.60 (1H, brs), 10.8 (1H, brs), 8.52 (1H, s), 7.36 (3H, m), 3.97-4.20 (7H, m), 3.64-3.71 (4H, m), 3.47 (7H, m), 3.25 (2H, m), 3.05 (3H, m), 2.73 (3H, s), 2.10-2.45 (1H, m), 1.99 (1H, m), 1.78 (2H, m), 1.55 (2H, m), 0.83-1.12 (12H, m), 0.70 (2H, m).

LCMS (Method 7): Rₜ=0.669 min, m/z found 600.5 [M+H]⁺.

### Example 6—Synthesis of crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate (Compound A4) (equivalent water not determined)

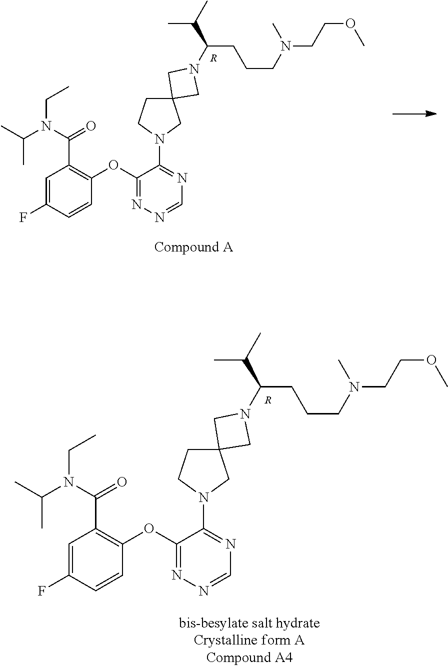

43.06 g benzenesulfonic acid (2 equivalents with respect to the free base Compound A) was added to 840 ml of an acetone/water 95/5 v/v mixture and dissolved. 192.8 g of a solution of Compound A (containing 80 g API) in IPAc was added. The material was dissolved, resulting in a clear solution. A further 80 ml of IPAc is added and the temperature was adjusted to 25° C. 2% of seeds were added and the mixture is stirred for an hour at 25° C. Then 28.8 V (2312 ml) of IPAc was added over a period of 8 hours. Afterwards the suspension was stirred for 18 hours at 25° C. The suspension was filtered and washed with 320 ml of a mixture of acetone/water/IPAc 23.75/1.75/75 v/v/v. 122.91 g of crystalline form A bis-besylate hydrate (equivalent water not determined) was obtained.

A skilled person will understand that a small amount of initial seed material used in the reaction above can be obtained via an analogous reaction protocol on small-scale without addition of seeds and wait for spontaneous nucleation. Initial seeds of the besylate salt were also obtained during salt screening experiments. In these experiments 100 mg of the free base was weighed into 2 mL vials, and then 200 μL of ethyl acetate or acetone was added to dissolve the free base. 1 eq counter-ions (benzenesulfonic acid) were added to the samples, and the samples were stirred at 25° C. for 3 days. The suspension obtained was centrifuged and yielded initial seeds.

An appropriate amount of crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate was dissolved in deuterated DMSO and the 1D ¹H NMR spectrum was recorded.

A Bruker AVANCE NEO-600 MHz NMR spectrometer equipped with a Bruker 5 mm PA BBO 60053 BB-H-D-05 Z-GRD high resolution probe and running TOPSPIN 4.0 software, was used to collect a 1-dimensional proton experiment at 300K on the sample in deuterated DMSO.

¹H NMR (600 MHz, DMSO-d₆) δ ppm 0.69 (br s, 2H) 0.82-0.98 (m, 9H) 1.07 (br s, 4H) 1.31-1.46 (m, 1H) 1.51 (br d, J=2.91 Hz, 1H) 1.69 (br d, J=3.45 Hz, 2H) 1.98 (br s, 1H) 2.06-2.45 (m, 2H) 2.77 (br s, 3H) 2.87-3.19 (m, 3H) 3.24 (br s, 1H) 3.31 (s, 6H) 3.64 (br s, 4H) 3.71-4.59 (m, 7H) 7.24-7.54 (m, 9H) 7.61 (br d, J=7.27 Hz, 4H) 8.45-8.60 (m, 1H) 9.24 (br s, 1H) 9.44-9.82 (m, 1H).

### Example 7—Alternative synthesis of crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate (Compound A4) (equivalent water not determined)

A mixture of isopropanol/water 95/5 (24 ml) was charged in a flask and heated to 40° C. Benzenesulfonic acid (4.31 g; 98%) was added. Subsequently, 19.3 g of a solution of Compound A (containing 8 g of Compound A) in IPAc was added. Another 16 ml of IPAc was added. 2% of seeds were added and the mixture was stirred for 1 hour at 40° C. Then IPAc was added (115.2 ml) dropwise over a period of 8 hours. Next, the mixture was cooled to 0° C. for 15 hours. The suspension was filtered and the wet cake was washed with (IPA/H₂O 95/5)/IPAc 1/6 (32 ml). The wet cake was dried at 25° C. for 16 hours to obtain 11.44 g of crystalline form A bis-besylate hydrate (equivalent water not determined).

### Crystalline Form A

Crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate may be characterised by an X-ray powder diffraction pattern.

X-ray powder diffraction (XRPD) analysis was carried out on a PANalytical Empyrean diffractometer. The instrument is equipped with a Cu-Ku X-ray tube using iCore and dCore tunable optics for the incident and the diffracted beam, respectively. The compound was loaded into the cavity of a 16 mm sample holder using the back loading technique.

Samples were run on XRPD using the method below:
Tube: Cu: K-Alpha (λ=1.541874 Å)
Generator: Voltage: 45 kV; Current: 40 mA
Geometry: Bragg-Brentano
Scan mode: Continuous Scan
Scan Range: 3 to 35 deg.
Step size: 0.0131 deg.
Counting time: 30 s
Spinner revolution time: 1 sec
   Incident Beam Path (iCore)
Program. divergence slit: automatic
Irradiated length: 10 mm
Soller slit: 0.03 rad
Mask 1: 14 mm
Mask 2: 6 mm
Width: 7.7 mm
   Diffracted Beam Path (dCore)
Anti scatter slit: automatic
Irradiated length: 10 mm
Soller slit: 0.04 rad
Detector: PIXcel3D-Medipix3 1×1

One skilled in the art will recognize that diffraction patterns and peak positions are typically substantially independent of the diffractometer used and whether a specific calibration method is utilized. Typically, the peak positions may differ by about ±0.2° two theta, or less. The intensities (and relative intensities) of each specific diffraction peak may also vary as a function of various factors, including, but not limited to particle size, orientation, sample purity, etc.

The X-ray powder diffraction pattern comprises peaks at 5.4, 7.2, 11.1, 11.9 and 21.7 degrees two theta ±0.2 degrees two theta. The X-ray powder diffraction pattern may further comprise at least one peak selected from 13.7, 14.5, 14.7, 15.0, 16.5, 17.8, 19.0, 19.4, 20.1 degrees two theta ±0.2 degrees two theta.

Form A may further be characterized by an X-ray powder diffraction pattern having four, five, six, seven, eight, nine or more peaks selected from those peaks identified in Table 2.

Form A may further be characterized by an X-ray powder diffraction pattern comprising those peaks identified in Table 2, wherein the relative intensity of the peaks is greater than about 2%, preferably greater than about 5%, more preferably greater than about 10%, more preferably greater than about 15%. However, a skilled person will realize that the relative intensity of the peaks may vary between different samples and different measurements on the same sample.

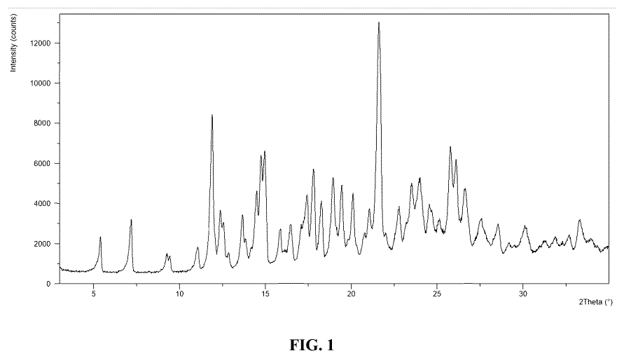

Form A may further be characterized by an X-ray powder diffraction pattern substantially as depicted in FIG. 1.

Table 2 provides peak listings and relative intensity for the XPRD of Crystalline form A of (R)-N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate BSA salt (FIG. 1).

TABLE 2
Peak Listings and Relative Intensity for XRPD of Form A

| Pos. | Rel. Int. |
|---|---|
| [°2Th.] | [%] |
| 5.3965 | 16.30 |
| 7.1906 | 23.69 |
| 9.2513 | 8.14 |
| 9.4433 | 7.39 |
| 11.0719 | 11.34 |
| 11.9144 | 73.29 |
| 12.3921 | 29.17 |
| 12.5717 | 22.93 |
| 12.8791 | 8.93 |
| 13.6790 | 26.58 |
| 13.8694 | 15.67 |
| 14.4793 | 38.19 |
| 14.7398 | 55.26 |
| 14.9599 | 56.99 |
| 15.8715 | 20.66 |
| 16.4606 | 22.37 |
| 17.0459 | 20.43 |
| 17.4421 | 34.59 |
| 17.8203 | 46.78 |
| 18.2871 | 30.73 |
| 18.9573 | 43.91 |
| 19.4485 | 41.00 |
| 20.1190 | 35.53 |
| 20.7356 | 18.05 |
| 21.0535 | 30.09 |
| 21.6801 | 100.00 |
| 22.0236 | 18.06 |
| 22.7925 | 29.92 |
| 23.5044 | 41.92 |
| 23.9959 | 43.96 |
| 24.5555 | 31.47 |
| 25.1401 | 25.01 |
| 25.7588 | 59.24 |
| 26.0910 | 53.05 |
| 26.6137 | 39.47 |
| 27.5409 | 24.89 |
| 28.5493 | 22.44 |
| 29.1699 | 13.97 |
| 30.1441 | 21.01 |
| 31.2560 | 14.66 |
| 31.8783 | 16.47 |
| 32.7054 | 17.11 |
| 33.2797 | 24.40 |
| 33.9762 | 15.63 |

Compound A4 described herein is disclosed in PCT/CN2021/100466 (filed Jun. 17, 2021), which is incorporated by reference herein in its entirety, for all purposes.

### Analytical Methods

The analytical information in the compounds above or in the Tables below, was generated by using the analytical methods described below.

### NMR-Methods

Some NMR experiments were carried out using a Bruker Avance III 400 spectrometer at ambient temperature (298.6 K), using internal deuterium lock and equipped with BBO 400 MHz S1 5 mm probe head with z gradients and operating at 400 MHz for the proton and 100 MHz for carbon. Chemical shifts (6) are reported in parts per million (ppm). J values are expressed in Hz.

Some NMR experiments were carried out using a Varian 400-MR spectrometer at ambient temperature (298.6 K), using internal deuterium lock and equipped with Varian 400 4NUC PFG probe head with z gradients and operating at 400 MHz for the proton and 100 MHz for carbon. Chemical shifts (6) are reported in parts per million (ppm). J values are expressed in Hz.

Some NMR experiments were carried out using a Varian 400-VNMRS spectrometer at ambient temperature (298.6 K), using internal deuterium lock and equipped with Varian 400 ASW PFG probe head with z gradients and operating at 400 MHz for the proton and 100 MHz for carbon. Chemical shifts (6) are reported in parts per million (ppm). J values are expressed in Hz.

Some NMR experiments were carried out using a Bruker AVANCE III HD 300 spectrometer at ambient temperature (298.6 K), using internal deuterium lock and equipped with PA BBO 300S1 BBF-H-D-05 Z 5 mm probe head with z gradients and operating at 300 MHz for the proton and 75 MHz for carbon. Chemical shifts (d) are reported in parts per million (ppm). J values are expressed in Hz.

### LCMS (Liquid Chromalography/Mass Spectrometry)

### General Procedure

The High Performance Liquid Chromatography (HPLC) measurement was performed using a LC pump, a diode-array (DAD) or a UV detector and a column as specified in the respective methods. HPLC details are provided below in Table 3. If necessary, additional detectors were included (see Tables 3 and 4 below).

Flow from the column was brought to the Mass Spectrometer (MS) which was configured with an atmospheric pressure ion source. It is within the knowledge of the skilled person to set the tune parameters (e.g., scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e., [M+NH₄]⁺, [M+HCOO]⁻, etc. . . . ). For molecules with multiple isotopic patterns (Br, Cl, etc.), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

TABLE 3
LCMS Method Codes

| Method | | | | | | | Flow | Run |
|---|---|---|---|---|---|---|---|---|
| code | Instrument | Column | Mobile phase | Gradient | | | Column T | time |
| 1 | Agilent | Waters XBridge | Mobile phase A: | 100% A was held for 1 min, A | | | 0.8 | 10 |
| | | C18 (2.0 × 50 | H₂O with 0.04% | gradient from 100% A to 40% | | | 50 | |
| | | mm, 5 uM) | TFA; Mobile | A is applied in 4 min, and | | | | |
| | | | phase B: ACN | 40% A down to 15% A in 2.5 | | | | |
| | | | with 0.02% TFA | min. And then return to 100% | | | | |
| | | | | A in 2 min and held for 0.5 | | | | |
| | | | | min. The post time is 0.5 min. | | | | |
| 2 | Agilent | Waters XBridge | Mobile phase A: | First, 90% A was held for 0.8 | | | 0.8 | 10 |
| | | C18 (2.0 × 50 | H₂O with 0.04% | min. Then a gradient was | | | 50 | |
| | | mm, 5 um) | TFA; Mobile | applied to 20% A and 80% B | | | | |
| | | | phase B: ACN | in 3.7 min and held for 3 min. | | | | |
| | | | with 0.02% TFA | And then return to 90% A in 2 | | | | |
| | | | | min and held for 0.5 min. The | | | | |
| | | | | post time is 0.5 min. | | | | |
| 7 | Agilent LC | XBridge C18, | Mobile phase A | Time (min) | A % | B % | 1.5 | 20 |
| | 1260 with | 4.6 × 150 | 0.05% TFA in | Initial | 95 | 5 | 45 | |
| | MS6120 | mm, 3.5 μm | H₂O | 11.0 | 65 | 35 | | |
| | | | Mobile phase B | 13.0 | 5 | 95 | | |
| | | | 0.05% TFA in | 15.0 | 5 | 95 | | |
| | | | ACN | 16.0 | 95 | 5 | | |
| | | | | 20.0 | 95 | 5 | | |

Flow expressed in mL/min;
column temperature (T) in ° C.;
run time in minutes

### Analytical SFC

### General Procedure for SFC Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. Analytical SFC details are provided below in Table 4. If configured with a Mass Spectrometer (MS) the flow from the column was brought to the (MS). It is within the knowledge of the skilled person to set the tune parameters (e.g., scanning range, dwell time . . . ) in order to obtain ions allowing the identification of the compound's nominal monoisotopic molecular weight (MW). Data acquisition was performed with appropriate software.

TABLE 4
Analytical SFC Details

| | | | | | Run |
|---|---|---|---|---|---|
| Method | | | | Flow | Time |
| Code | Column | Mobile Phase | Gradient | Col T | BPR |
| 11 | Waters UPCC | A: Supercritical | from 5% to 40% of | 2.8 | 8 |
| | with PDA | CO₂ B: EtOH | B in 4 min and | | |
| | (Chiralpak IG-3 | (0.05% DEA) | hold 40% for 2.5 | 35 | 1500 psi. |
| | 100 × 4.6 mm | | min, then 5% of B | | |
| | I.D., 3 um) | | for 1.5 min | | |

Flow expressed in mL/min; column temperature (T) in ° C.; run time in minutes, Backpressure (BPR) in bars or pound-force per square inch (psi). “ACN” means acetonitrile; “MeOH” means methanol; “EtOH” means ethanol; “DEA” means diethylamine. All other abbreviations used in Table 4 above are as defined before.

### Pharmacological Part

### 1) Menin/MLL Homogenous Time-Resolved Fluorescence (HTRF) Assay

To an untreated, white 384-well microtiter plate was added 40 nL 200× test compound in DMSO and 4 μL 2× terbium chelate-labeled menin (vide infra for preparation) in assay buffer (40 mM Tris·HCl, pH 7.5, 50 mM NaCl, 1 mM DTT (dithiothreitol) and 0.05% Pluronic F-127). After incubation of test compound and terbium chelate-labeled menin for 30 min at ambient temperature, 4 μL 2×FITC-MBM1 peptide (FITC-β-alanine-SARWRFPARPGT-NH₂) (“FITC” means fluorescein isothiocyanate) in assay buffer was added, the microtiter plate centrifuged at 1000 rpm for 1 min and the assay mixtures incubated for 15 min at ambient temperature. The relative amount of menin.FITC-MBM1 complex present in an assay mixture is determined by measuring the homogenous time-resolved fluorescence (HTRF) of the terbium/FITC donor/acceptor fluorophore pair using an EnVision microplate reader (ex. 337 nm/terbium em. 490 nm/FITC em. 520 nm) at ambient temperature. The degree of fluorescence resonance energy transfer (the HTRF value) is expressed as the ratio of the fluorescence emission intensities of the FITC and terbium fluorophores (F^{em} 520 nm/F^{em} 490 nm). The final concentrations of reagents in the binding assay are 200 pM terbium chelate-labeled menin, 75 nM FITC-MBM1 peptide and 0.5% DMSO in assay buffer. Dose-response titrations of test compounds are conducted using an 11 point, four-fold serial dilution scheme, starting typically at 10 μM.

Compound potencies were determined by first calculating % inhibition at each compound concentration according to equation 1:

$$\% inhibition = ( ( HC - LC ) - ( {HTRF}^{compound} - LC ) ) / ( HC - LC ) ) * 100 \qquad ( Eqn 1 )$$

where LC and HC are the HTRF values of the assay in the presence or absence of a saturating concentration of a compound that competes with FITC-MBM1 for binding to menin, and HTRF^{compound} is the measured HTRF value in the presence of the test compound. HC and LC HTRF values represent an average of at least 10 replicates per plate. For each test compound, % inhibition values were plotted vs. the logarithm of the test compound concentration, and the IC₅₀ value derived from fitting these data to equation 2:

$$\% inhibition = Bottom + ( Top - Bottom ) / ( 1 + 10 \hat{} ( ( log {IC}_{50} - log [ cmpd ] ) * h ) ) \qquad ( Eqn 2 )$$

where Bottom and Top are the lower and upper asymptotes of the dose-response curve, respectively, IC₅₀ is the concentration of compound that yields 50% inhibition of signal and h is the Hill coefficient.

Preparation of Terbium cryptate labeling of Menin: Menin (a.a 1-610-6×his tag, 2.3 mg/mL in 20 mM Hepes (2-[4-(2-Hydroxyethyl)-1-piperazinyl]ethane sulfonic acid), 80 mM NaCl, 5 mM DTT (Dithiothreitol), pH 7.5) was labeled with terbium cryptate as follows. 200 μg of Menin was buffer exchanged into 1× Hepes buffer. 6.67 μM Menin was incubated with 8-fold molar excess NHS (N-hydroxysuccinimide)-terbium cryptate for 40 minutes at room temperature. Half of the labeled protein was purified away from free label by running the reaction over a NAPS column with elution buffer (0.1M Hepes, pH 7+0.1% BSA (bovine serum albumin)). The other half was eluted with 0.1M phosphate buffered saline (PBS), pH7. 400 μl of eluent was collected for each, aliquoted and frozen at −80° C. The final concentration of terbium-labeled Menin protein was 115 μg/mL in Hepes buffer and 85 μg/mL in PBS buffer, respectively.

MENIN Protein Sequence (SEQ ID NO: 1):
MGLKAAQKTLFPLRSIDDVVRLFAAELGREEPDLVLLSLVLGFVEHFLAVNRV
IPTNVPELTFQPSPAPDPPGGLTYFPVADLSIIAALYARFTAQIRGAVDLSLYPREGGVS
SRELVKKVSDVIWNSLSRSYFKDRAHIQSLFSFITGTKLDSSGVAFAVVGACQALGLR
DVHLALSEDHAWVVFGPNGEQTAEVTWHGKGNEDRRGQTVNAGVAERSWLYLKG
SYMRCDRKMEVAFMVCAINPSIDLHTDSLELLQLQQKLLWLLYDLGHLERYPMALG
NLADLEELEPTPGRPDPLTLYHKGIASAKTYYRDEHIYPYMYLAGYHCRNRNVREAL
QAWADTATVIQDYNYCREDEEIYKEFFEVANDVIPNLLKEAASLLEAGEERPGEQSQ
GTQSQGSALQDPECFAHLLRFYDGICKWEEGSPTPVLHVGWATFLVQSLGRFEGQVR
QKVRIVSREAEAAEAEEPWGEEAREGRRRGPRRESKPEEPPPPKKPALDKGLGTGQG
AVSGPPRKPPGTVAGTARGPEGGSTAQVPAPAASPPPEGPVLTFQSEKMKGMKELLV
ATKINSSAIKLQLTAQSQVQMKKQKVSTPSDYTLSFLKRQRKGLHHHHHH

### 2a) Proliferation Assay

The anti-proliferative effect of menin/MLL protein/protein interaction inhibitor test compounds was assessed inhuman leukemia cell lines. The cell line MOLM-14 harbors a MLL translocation and expresses the MLL fusion protein MLL-AF9, respectively, as well as the wildtype protein from the second allele. OCI-AML3 cells that carry the NPM1c gene mutation were also tested. MLL rearranged cell lines (e.g., MOLM-14) and NPM1c mutated cell lines exhibit stem cell-like elevated HOXA/MEIS1 gene expression signatures. KO-52 was used as a control cell line containing two MLL (KMT2A) wildtype alleles in order to exclude compounds that display general cytotoxic effects.

MOLM-14 cells were cultured in RPMI-1640 (Sigma Aldrich) supplemented with 10% heat-inactivated fetal bovine serum (HyClone), 2 mM L-glutamine (Sigma Aldrich) and 50 μg/ml gentamycin (Gibco). KO-52 and OCI-AML3 cell lines were propagated in alpha-MEM (Sigma Aldrich) supplemented with 20% heat-inactivated fetal bovine serum (HyClone), 2 mM L-glutamine (Sigma Aldrich) and 50 μg/ml gentamycin (Gibco). Cells were kept at 0.3-2.5 million cells per ml during culturing and passage numbers did not exceed 20.

In order to assess the anti-proliferative effects, 200 MOLM-14 cells, 200 OCI-AML3 cells or 300 KO-52 cells were seeded in 200 μl media per well in 96-well round bottom, ultra-low attachment plates (Costar, catalogue number 7007). Cell seeding numbers were chosen based on growth curves to ensure linear growth throughout the experiment. Test compounds were added at different concentrations and the DMSO content was normalized to 0.3%. Cells were incubated for 8 days at 37° C. and 5% CO₂. Spheroid like growth was measured in real-time by live-cell imaging (IncuCyteZOOM, Essenbio, 4× objective) acquiring images at day 8. Confluence (%) as a measure of spheroid size was determined using an integrated analysis tool.

In order to determine the effect of the test compounds over time, the confluence in each well as a measure of spheroid size, was calculated. Confluence of the highest dose of a reference compound was used as baseline for the LC (Low control) and the confluence of DMSO treated cells was used as 0% cytotoxicity (High Control, HC).

Absolute IC₅₀ values were calculated as percent change in confluence as follows:
LC=Low Control: cells treated with e.g., 1 μM of the cytotoxic agent staurosporin, or e.g., cells treated with a high concentration of an alternative reference compound;

HC=High Control: Mean confluence (%) (DMSO treated cells);

% Effect=100−(100*(Sample-LC)/(HC-LC)); and
GraphPad Prism (version 7.00) was used to calculate the IC₅₀. Dose-response equation was used for the plot of % Effect vs Log10 compound concentration with a variable slope and fixing the maximum to 100% and the minimum to 0%.
   2b) MEIS1 mRNA Expression Assay

MEIS1 mRNA expression upon treatment of compound was examined by Quantigene Singleplex assay (Thermo Fisher Scientific). This technology allows for direct quantification of mRNA targets using probes hybridizing to defined target sequences of interest and the signal is detected using a Multimode plate reader Envision (PerkinElmer). The MOLM-14 cell line was used for this experiment. Cells were plated in 96-well plates at 3,750 cells/well in the presence of increasing concentrations of compounds. After incubation of 48 hours with compounds, cells were lysed in lysis buffer and incubated for 45 minutes at 55° C. Cell lysates were mixed with human MEIS1 specific capture probe or human RPL28 (Ribosomal Protein L28) specific probe as a normalization control, as well as blocking probes. Cell lysates were then transferred to the custom assay hybridization plate (Thermo Fisher Scientific) and incubated for 18 to 22 hours at 55° C. Subsequently, plates were washed to remove unbound materials followed by sequential addition of preamplifiers, amplifiers, and label probe. Signals (=gene counts) were measured with a Multimode plate reader Envision. IC₅₀s were calculated by dose-response modelling using appropriate software. For all non-housekeeper genes response equal counts corrected for background and relative expression. For each sample, each test gene signal (background subtracted) was divided by the normalization gene signal (RPL28: background subtracted). Fold changes were calculated by dividing the normalized values for the treated samples by the normalized values for the DMSO treated sample. Fold changes of each target gene were used for the calculation of IC₅₀s.

The results are summarized below in Table 5.

TABLE 5
Biological data - HTRF, Proliferation and MEIS1 mRNA Expression Assays

| | HTRF- | | spheroid | OCI- | spheroid |
|---|---|---|---|---|---|
| | 30 min | MEIS1 | assay_OneTime | AML3 | assay_OneTime |
| Compound | incubation | IC₅₀ | MOLM-14 IC₅₀ | IC₅₀ | KO-52 IC₅₀ |
| Number | IC₅₀ (nM) | (μM) | (μM) | (μM) | (μM) |
| 11 | 0.11 | 0.018 | 0.021 | 0.21 | 8.38 |
| A | 0.09 | 0.02 | 0.021 | 0.091 | 6.85 |
| A3 | 0.098 | 0.017 | 0.017 | 0.12 | 7.75 |
| A1 | 0.18 | 0.017 | 0.011 | 0.08 | |

### 3) Mouse PK (In Vivo T_{1/2} and Oral Bioavailability)

In vivo pharmacokinetics (PK) were assessed in fasted male CD-1 mice (age 6-8 weeks) following a single intravenous (IV, 0.5 or 1.0 mg/kg administered at 2.5 ml/kg) or oral (PO, 5 mg/kg administered at 10 ml solution/kg) dose of test article formulated in a 20% (w:vol) HP-β-CD solution or in Pyrogen free water.

Plasma and/or whole blood samples were collected from the dorsal metatarsal vein at desired timepoints via serial capillary microsampling (approx. 0.03 mL) using EDTA as an anticoagulant. Concentrations of compound in the plasma and blood samples were analyzed using a qualified LC-MS/MS method. In silico analysis of main pharmacokinetic parameters was performed using WinNonlin (Phoenix™, version 6.1) or similar software.)

### 4) Metabolic Stability in Human/Mouse Liver Microsomes

### Experimental Procedure

The objective of this study is to measure in vitro metabolic stability of test compound(s) in human and mouse liver microsomes and provide quantitative information on the rate of metabolic turnover (i.e., determination of the apparent intrinsic clearance of test).

Test items were prepared at a stock concentration of 10 mM in DMSO. For determination of metabolic turnover, a final working solution was prepared by adding 2 μL of 10 mM DMSO stock solution for test compound or positive control compounds to 198 μL of acetonitrile (100 μM final concentration).

Incubations were performed as follows: First, liver microsomes were thawed on ice and a master solution containing liver microsomes in 100 mM PBS (phosphate-buffered saline) at pH 7.4 is prepared. Next, the liver microsomes solution was added to the incubation plates and 10 mM NADPH (Nicotinamide-adenine dinucleotide phosphate) was added (MW: 833.4 g/mol; Roche Diagnostics GmbH1, Germany. Dissolved in phosphate buffer (100 mmol/L, pH 7.4)). The mixture was mixed for 10 seconds and pre-warmed in the incubation plate at 37° C. for 10 minutes. The metabolic reaction was initiated with the addition of 5 μL of the 100 μM working solution for test compound or positive control compounds to incubation plate (final test item concentration=1 μM). The reaction final mixture should contain 1 mM NADPH, 0.5 mg/mL microsomes protein and 1 μM test compound or positive control compound in 100 mM PBS at pH 7.4. The percentage of organic solvent in incubation mixture is 1% with DMSO 0.02%.

The reaction was quenched by transferring 50 μL of the incubated mixture at selected time points into the quenching plate containing 200 μL of cold methanol. After sampling of all the timepoints the quenching plate was centrifuged at 4000 rpm for 40 minutes to precipitate protein. A total of 90 μL of the supernatant was transferred to an analysis plate and ultra-pure H₂O water is added into each well for LC/MS/MS analysis. All incubations and analysis were performed in duplicate.

### Data Analysis

All calculations were carried out using Microsoft Excel. The slope value, k, was determined by linear regression of the natural logarithm of the remaining percentage of the parent drug vs. incubation time curve. The results are summarized below in Table 6.

The in vitro half-life (in vitro t_{1/2}) was determined from the slope value:

$$in vitro {t}_{1 / 2} = - ( 0.693 / k )$$

Conversion of the in vitro t_{1/2} (in min) into the in vitro intrinsic clearance (in vitro CLᵢₙₜ, in μL/min/mg proteins) was done using the following equation:

$$in vitro {Cl}_{i n t} = ( \frac{0.693}{{t}_{\frac{1}{2}}} ) * ( \frac{volume of incubation ( μL )}{amount of proteins ( mg )} )$$

TABLE 6
Mouse PK and Metabolic Stability

| | | In vivo | | | |
|---|---|---|---|---|---|
| | | T_{1/2} | Bio- | Human LM | Mouse LM |
| Example | Formulating | (IV) | availability | Clint | Clint |
| number | agent | (h) | (PO) (%) | (μl/min/mg) | (μl/min/mg) |
| A | HP-β-CD | 6.7 | 17 | 19 | <7.5 |
| A3 | Pyrogen free | 9.0 | 34 | 19 | <7.5 |
| | water | | | | |
| 11 | HP-β-CD | NA | NA | 14 | <7.5 |

“NA” means not analyzed

### 5) Protocol for Pharmacodynamics (PD) Activity in Subcutaneous (Sc or SC) Xenografts of MOLM-14 or OCI-AML3 Cells

### Test Agents and Controls

Compound A3 was formulated in 20% hydroxypropyl-beta-cyclodextrin (HP-3-CD) and prepared to reach a total volume of 0.2 mL (10 mL/kg) per dose for a 20 g animal. Doses were adjusted by individual body weight each day. Working stocks of Compound A3 were prepared once per week for each study and stored at room temperature. Compound A3 was administered orally (PO), daily.

### Assay

The in vivo pharmacodynamics (PD) activity of compounds was evaluated in subcutaneous (SC) xenografts of MOLM-14 cells or OCI-AML3 cells. Nude NMRI mice (Crl:NMRI-Foxnlnu/−) harboring MOLM-14 or OCI-AML3 tumors were treated with 3 daily doses of vehicle or compounds. Plasma samples were collected at 23 hours after day 2 dose, 0.5 hours post final dose, and 16 hours post final dose and tumor samples were collected 16 hours post final dose. To examine the effects of compounds on the expression of multiple Menin-KMT2A target genes (e.g., MEIS1, MEF2C, FLT3) QuantiGene Plex technology (Thermo Fisher Scientific) was used. Frozen tumors were homogenized and transferred to individual lysing matrix tubes in lysis buffer and incubated for 30 minutes at 55° C. Cell lysates were mixed with target-specific capture probes, Luminex beads, and blocking probes, transferred to the custom assay hybridization plate (Thermo Fisher Scientific) and incubated for 18 to 22 hours at 54° C. Subsequently, plates were transferred to a magnetic separation plate and washed to remove unbound materials from beads followed by sequential hybridization of preamplifiers, amplifiers, and label probe and subsequent streptavidin phycoerythrin binding. Signals from the beads were measured with a Luminex FlexMap three-dimensional instrument. For all non-housekeeper genes response equal counts corrected for background and relative expression. For each sample, each test gene signal (background subtracted) was divided by the normalization gene signal (RPL19, RPL28, ATP6V1A: background subtracted). Fold changes were calculated by dividing the normalized values for the treated samples by the normalized values for the DMSO treated sample. The results are summarized below in Tables 7 and 8.

TABLE 7
Expression Level (% relative to vehicle) of Selected Genes
from MOLM-14 SC Model (mean values and standard deviations)

| Compound A3 (mg/kg) | MEIS1 | FLT3 | MEF2C |
|---|---|---|---|
| 0 | 101.30 ± 15.06 | 104.80 ± 10.07 | 103.50 ± 11.02 |
| 3 | 83.49 ± 25.48 | 78.67 ± 20.74 | 85.50 ± 22.77 |
| 10 | 62.84 ± 4.06 | 74.91 ± 8.97 | 68.04 ± 14.43 |
| 30 | 23.16 ± 2.75 | 52.61 ± 4.51 | 27.83 ± 2.17 |
| 50 | 14.40 ± 3.39 | 36.14 ± 3.50 | 18.75 ± 2.38 |
| 100 | 10.97 ± 3.21 | 35.82 ± 1.10 | 14.18 ± 1.56 |

TABLE 8
Expression Level (% relative to vehicle) of Selected Genes from
OCI-AML3 SC Model (mean values and standard deviations).

| Compound A3 (mg/kg) | MEIS1 |
|---|---|
| 0 | 100.30 ± 8.53 |
| 3 | 87.90 ± 39.75 |
| 10 | 48.81 ± 15.30 |
| 30 | 32.66 ± 3.71 |
| 50 | 23.83 ± 1.34 |
| 100 | 16.76 ± 1.92 |

Tables 7a and 8a show median values based on repeated experiments in optimized conditions with fresh tumor samples.

TABLE 7a
Expression level (% relative to vehicle) of selected genes from
MOLM-14 SC model (Median values and Standard Deviations).

| Compound A3 (mg/kg) | MEISI | FLT3 | MEF2C |
|---|---|---|---|
| 0 | 100.0 ± 13.5 | 100.0 ± 10.1 | 100.0 ± 11.0 |
| 3 | 83.7 ± 22.8 | 89.2 ± 20.7 | 87.7 ± 22.8 |
| 10 | 49.3 ± 5.9 | 79.8 ± 9.0 | 64.6 ± 14.4 |
| 30 | 14.7 ± 3.9 | 54.5 ± 4.5 | 28.8 ± 2.2 |
| 50 | 4.7 ± 1.1 | 37.6 ± 3.5 | 18.8 ± 2.4 |
| 100 | 3.3 ± 1.4 | 35.4 ± 1.1 | 13.6 ± 1.6 |

TABLE 8a
Expression level (% relative to vehicle) of selected gene from
OCI-AML3 SC model (Median values and Standard Deviations).

| Compound A3 (mg/kg) | MEISI |
|---|---|
| 0 | 100.0 ± 11.2 |
| 3 | 71.2 ± 15.1 |
| 10 | 26.5 ± 4.3 |
| 30 | 25.1 ± 11.2 |
| 50 | 8.5 ± 2.2 |
| 100 | 9.4 ± 1.2 |

### 6) Efficacy Study in MOLM-14 Subcutaneous Model

### Test Agents and Controls

Compound A3 was formulated in 20% hydroxypropyl-beta-cyclodextrin (HP-3-CD) and prepared to reach a total volume of 0.2 mL (10 mL/kg) per dose for a 20 g animal. Doses were adjusted by individual body weight each day. Working stocks of Compound A3 were prepared once per week for each study and stored at 25° C.

### Animals

Female NMRI Nude mice (MOLM-14 SC) were used when they were approximately 6 to 8 weeks of age and weighed approximately 25 g. All animals could acclimate and recover from any shipping-related stress for a minimum of 7 days prior to experimental use. Autoclaved water and irradiated food were provided ad libitum, and the animals were maintained on a 12 hour light and dark cycle. Cages, bedding, and water bottles were autoclaved before use and changed weekly. Further details are provided below in Table 9.

TABLE 9
Tissue Culture and Cell Injection Reagents

DPBS (Dulbecco's phosphate-buffered saline)
Heat-inactivated fetal bovine serum
RPMI 1640 medium
L-glutamine
Gentamycin
T175 Culture Flask
Roller Bottle

### Tumor Model and Cell Culture Method

Human AML MOLM-14 cells were cultured at 37° C., 5% CO₂ in the indicated complete culture media (RPMI 1640+10% HI-FBS+2 mM L-glutamine+50 ug/ml Gentamycin). Cells were harvested while in logarithmic growth and resuspended in cold (4° C.) Roswell Park Memorial Institute (RPMI) 1640 in serum-free medium.

Each mouse received 5×10⁶ MOLM-14 cells in 50% Matrigel in the right flank, in a total volume of 0.2 mL using a 1 cc syringe and a 27-gauge needle.

### Study Designs

Compound A3 was administered orally (PO), daily.

Day 0 is the day of tumor cell implantation and study initiation.

Mice bearing SC MOLM-14 tumors were randomized on Day 16 post-tumor implantation and assigned to treatment groups according to tumor volume (mean of ˜130 mm³; n=10/group). Treatment with vehicle or Compound A3 (at 30 and 100 mg/kg) was initiated on the same day, with daily oral dosing for 21 days. Plasma was collected at 1, 2, 4, 8, and 23 hours after the last dose (n=4-5/group/time point) for PK (pharmacokinetics) analysis.

### Animal Monitoring

SC tumor volume was measured for each animal 2 to 3 times per week or more throughout the study.

### Calculations

Tumor volume was calculated using the formula:

Tumor volume (mm³)=(D×d²/2); where ‘D’ represents the larger diameter and ‘d’ the smaller diameter of the tumor as determined by caliper measurements. Tumor volume data was graphed as the mean tumor volume ±SEM.

The % ΔTGI was defined as the difference between mean tumor burden of the treatment and control groups, calculated as % ΔTGI=([(TV_{c}TVc₀)(TVₜTVₜ₀)]/(TV_{c}TVc₀))×100 where ‘TV_{c}’ is the mean tumor burden of a given control group, ‘TVc₀’ is the mean initial tumor burden of a given control group, ‘TVₜ’ is the mean tumor burden of the treatment group, and ‘TVₜ₀’ is the mean initial tumor burden of the treatment group. % TGI was defined as the difference between.

Mean tumor volumes of the treated and control groups, calculated as:

% TGI=((TV_{c}TVₜ)/TV_{c})×100 where ‘TV_{c}’ is the mean tumor volume of the control group and ‘TVₜ’ is the mean tumor volume of the treatment group. As defined by National Cancer Institute criteria, ≥60% TGI is considered biologically significant.

The % Tumor Regression (TR), quantified to reflect the treatment-related reduction of tumor volume as compared to baseline independent of the control group, was calculated as % TR=(1-mean (TVₜi/TVₜ₀i))×100 where ‘TVₜi’ is the tumor burden of individual animals in a treatment group, and ‘TVₜ₀i’ is the initial tumor burden of the animal.

### Data Analysis

Tumor volumes were graphed using Prism software (GraphPad version 7 or 8). Statistical significance for most studies was evaluated for Compound A3-treated groups compared with HPβCD vehicle-treated controls on the last day of the study when 2/3 or more mice remained in each group. Differences between groups were considered significant when p≤0.05.

Statistical significance for animal tumor volume was calculated using the linear mixed-effects (LME) analysis in R software version 3.4.2 (using Janssen's internally developed Shiny application version 4.0), with treatment and time as fixed effects and animal as random effect. Logarithmic transformation was performed if individual longitudinal response trajectories were not linear.

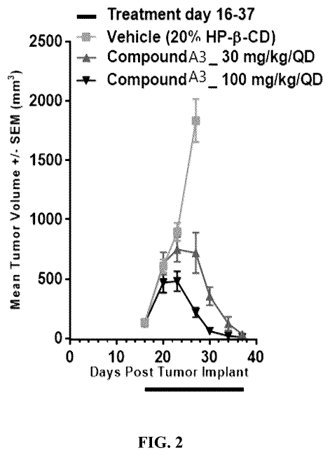

The information derived from this model was used to make pairwise treatment comparisons of tumor volumes to that of the control group or between all the treatment groups. The results are shown in FIG. 2.

7) Cardio-Electrophysiological Effects of the Testing Compounds in Synchronously Beating Human Pluripotent Stem Cell-Derived Cardiomyocytes (hSC-CMs) Using a Ca²⁺-Fluorescence Assay (CTCM Human)

### Protocol

Compounds were tested in the 96-well plates.

Compounds were tested at 0.1 μM, 0.2 μM, 0.5 μM, 1 μM, 2.5 μM and 5 μM (n=4 per dose) on Cor.4U®-Cardiomyocytes or on iCell® Cardionyocytes2.

Alternatively, compounds were tested at 0.1 μM, 0.3 μM; 1 μM, 3 μM, 10 μM and 30 μM (n=4 per dose) mostly on iCell® Cardiomyocytes2.

### Positive and Negative Controls

| | |
|---|---|
| Dofetilide | at 3 nM |
| Isoproterenol | at 100 nM |
| Nimodipine | at 100-300 nM |
| Cetirizine | at 3 μM. |

Vehicle control: Dimethylsulfoxide (DMSO). The solutions of the compound in DMSO or its solvent (final concentration of 0.1% DMSO; n=8).

### Preparation of Test Article and Controls

Tested compounds were dissolved in DMSO at 1000-fold the intended concentrations. A compound “mother-plate” was made, containing the test compounds and positive and negative controls at 1000-fold the final concentrations. At the experiment day, these stock solutions were diluted with Tyrode (Sigma), supplemented with 10 mM HEPES (Gibco), to 2-fold the intended concentration (in round bottom compound plates). Final DMSO concentration in test solutions and vehicle control was 0.1%.

### Cells

hSC-CMs (Cor.4U® Cardiomyocytes) were obtained from CDI (Ncardia, Germany). Cells are pre-plated and seeded in fibronectin-coated 96-well plates at a density suited to form a monolayer and maintained in culture in a stage incubator (37° C., 5% CO₂), according to the instructions of the cell provider.

Second line hSC derived cardiomyocyte called iCell® Cardiomyocytes2 were purchased from FUJIFILM Cellular Dynamics (USA). The experiments with test drugs are carried out 5 to 7 days after plating the cells onto the plate to have a living, beating monolayer of hiPSC-derived cardiomyocytes. The beating monolayer in 96-well-plates are normally taken from 2 Vials of frozen iCell® Cardiomyocytes2 (≈5 million cells/vial), which will be plated onto three 96-well plates (≈50K/well).

### Before Start of Experiment

At least one hour before the start of the experiments the normal cell medium was replaced with Tyrode solution with Calcium dye (see below).

Cal 520 dye (AAT Bioquest) was dissolved in 11 ml of Tyrode supplemented with 10 mM HEPES and warmed up to 37 C before adding to the cells.

35 μl cell culture medium was removed from each well and replaced with 35 μl of pre-warmed Cal 520 dye solution and cell plate was incubated for 45 min at 37° C./5% CO₂. Cells were incubated for 5 min at 37° C.

### Experiment

Spontaneous electrical activity is recorded, using Cal520™ (AAT Bioquest) calcium fluorescence-dye signalling. This dye integrates the total intracellular calcium activity over the whole well. A bottle of Ca1520 dye (50 μg, MW: 1103/mol) is dissolved with 50 μl DMSO as a stock solution of 0.9 mM. 50 μL of the stock solution of the dye was added to 10 ml Tryodes solution to have dye concentration of 4.5 μM. Subsequently, 35 μl of this dye solution was added into each well, to have a final dye concentration of 1.58 μM. The current dye protocol on this CTCM human assay was established recently (Ivan Kopljar et al, Journal of Pharmacological and toxicological methods 2018. 91: 80-86; Lu et al., Tox Sci 2019. 170 (2): 345-356).

Fluorescent signals (Ca²⁺ transient morphology) were measured using the Functional Drug Screen System (FDSS/μCell; Hamamatsu, Japan) and the recordings were subsequently analyzed off-line, using appropriate software e.g., Notocord.

The cell plate was loaded into the FDSS/μCell for a test run: Ca²⁺ transients were measured for 4 minutes to check for synchronous beating of the cardiomyocytes in each well. All 96 wells were measured simultaneously (sampling interval: 0.06 s, short exposure time: 10 ms; excitation wavelength 480 nm; emission wavelength 540 nm; FDSS/μCell warmed to 37° C.). When all showed synchronous beating, the 96-well plate was measured repeatedly for 3 times (to verify synchronous beating in all 96-well at baseline, wells that did not meet the preset criteria were excluded from the study and not treated with compound):
T=0: control period (−5 to −1 min)+compound addition, followed for 3 min.
T=30: measured from 29 to 34 min after compound addition

During the compound addition step, 100 μl of the respective double-concentrated test solutions was pipetted into each well simultaneously.

Data were analyzed off-line using appropriate software e.g., Notocord-Hem (version 4.3).

The following parameters of the Ca2+ transient morphology were measured:
beat rate (BR)
amplitude of the Ca²⁺ transient (Amp),
CTD₉₀: Ca²⁺ transient duration at 90% (time to 90% of the initial base value).

The presence of various ‘arrhythmia-like’ activities were also noted during the experimental periods. These included:
‘early afterdepolarization-like’ (EAD-like) events (defined as “an extra small peak of the transient waveform following the initial peak of the transient”),
‘ventricular tachycardia-like’ (VT-like) events (defined as a very fast beating rate) or
‘ventricular fibrillation-like’ (VF-like) events (defined as “small amplitude, fast-rate Ca²⁺ waveforms with irregularities and non-measurable transient potentials)
‘cessation of beating’ of the cells (no Ca²⁺ transients observed).
   If compound-induced changes on the calcium transient signal could not be analyzed by the software, then these signals were identified as BQL (below quality analyses level).

### Data Analysis

Data, measured from the FDSS-μCell, were copied for off-line analysis and were analyzed and uploaded in SPEC-II (our operational management system) for further analysis. The values of the variables before and after administration of the compound were collected and transferred into an Excel workbook.

All values (actual units and percentage changes from the baseline values) are expressed as median (minimum and maximum). Changes versus the corresponding baseline values (in actual units) observed in the compound group were compared with those in the solvent control group using the Wilcoxon-Mann-Whitney Test. Two-tailed tests with Bonferroni correction for multiplicity adjustment were conducted. Since there are 10 treatment groups each compared to the solvent group, alpha level of 0.05/10 (0.005) was considered to reflect a statistically significant difference from the solvent group. All statistical analysis was performed using appropriate software e.g., R software version 3.5.2.

Quality Control of the hiPSC-CMs in the plate:

Plates were rejected if they did not meet following criteria:
Stable regular beating
Amplitude >500 relative units
Beat rate between 25 and 80 beats per minute
CTD₉₀ between 300 and 800 ins.

In the present study, the hiPSC-CMs in the plates met the above criteria.

These parameters combined with incidence of arrhythmia or cessation of beating were used to calculate the potential hazard level using a weighted scoring method (based on Kopljar et al., Stem Cell Reports 2018. 11, 1365-1377). This hazard score is calculated per concentration by adding weighted points based on the Tolerance Intervals (TI) on the changes of CTD₉₀, the beat rate and amplitude (ΔΔ %) and incidence of beating stop and early afterdepolarization (EAD). Consequently, for each concentration one of four different hazard levels will be generated. This will be done after 30-min of incubated with compound. The hazard levels are:
No hazard: within the vehicle effect levels or small non-relevant changes.
Low hazard: relevant effect but potentially low risk for cardiac liabilities.
High hazard: relative high risk for cardiac liabilities.
Very high hazard: very high risk due to arrhythmic like events (EAD's).

The ‘Hazard Score’ results provide an identification for potential acute cardiac drug-induced effects at free drug equivalent (as no plasma proteins are added to the wells). Evaluation of hazard identification is conducted using a ‘scoring reference book’ called CTCM_Scoring_version 1 (Kopljar et al., Stem Cell Reports 2018. 11: 1365-1377), and levels are indicated according to the following color scheme of Table 10.

TABLE 10
Color Schemes of Hazardous Identification Legend

| | |
|---|---|
| Green | No concern |
| Yellow | Low concern |
| Red | High concern |
| Black | Very high concern due to arrhythmic events |

Ranking of a testing compound according to hazard score severity on the Ca²⁺ transient assay measured in HiPSc-CMs as listed above in different colors and in the associated table.

### Results

Using iCell® Cardionyocytes2 as Cell Line

Positive and negative controls: The positive and negative controls all had expected pharmacological effects in this assay. The results are summarized below in Tables 11 and 12.

TABLE 11
Hazard Scoring for Compound A3

| | Color @ | Color @ | Color @ | Color @ | Color @ | Color @ |
|---|---|---|---|---|---|---|
| Compound | 0.1 μM | 0.2 μM | 0.5 μM | 1 μM | 2.5 μM | 5 μM |
| A3 | Green | Green | Green | Green | Green | Green |

TABLE 12
Hazard Scoring for Compounds A1 and 11

| | Color @ | Color @ | Color @ | Color @ | Color @ | Color @ |
|---|---|---|---|---|---|---|
| Compound | 0.1 μM | 0.3 μM | 1 μM | 3 μM | 10 μM | 30 μM |
| A1 | Green | Green | Green | Green | Green | yellow |
| 11 | Green | Green | Green | Green | Green | Green |

For Compound A1: with an efficacious dose in mouse xenograft models of 30 mg/kg, CTCM human concentration vs free Cₘₐₓ would be estimated as follows:
Margin CTCM human 10 μM vs free Cmax >16 (mouse, human)
Margin CTCM human 30 μM vs free Cmax >45 (mouse, human).
   8) Effect on the Membrane Potassium Current I_{Kr} in hERG Transfected Cell Lines

Protocol 1:

### Abbreviations

| | |
|---|---|
| CHO | Chinese hamster ovary cell line |
| DMSO | Dimethylsulfoxide |
| hERG | human ether-à-go-go-related gene |
| I_{Kr} | rapidly activating delayed-rectifier K⁺ current |

### Methods

Experiments were performed using CHO cells stably expressing the hERG potassium channel. Cells were grown at 37° C. and 5% CO₂ in culture flasks in Ham's F12 Medium supplemented with 10% heat-inactivated fetal calf serum, hygromycin B (100 μg/ml) and geneticin (100 μg/ml). For use in the automated patch-clamp system QPatch (Sophion) cells were harvested to obtain cell suspension of single cells.

Solutions: The bath solution contained (in mM) 145 NaCl, 4 KCl, 10 glucose, 10 HEPES ((4-(2-hydroxyethyl)-1-piperazineethanesulfonic acid), 2 CaCl₂ and 1 MgCl₂ (pH 7.4 with NaOH). The pipette solution contained (in mM) 120 KCl, 10 EGTA (Ethylene glycol-bis(2-aninoethylether)-N,N,N′,N-tetraacetic acid), 10 HEPES, 5.374 CaCl₂ and 1.75 MgCl₂ (pH 7.2 with KOH).

Patch-clamp experiments were performed in the voltage-clamp mode and whole-cell currents were recorded with an automated patch-clamp assay utilizing the QPatch system (Sophion). Current signals were amplified and digitized, stored and analyzed by using the QPatch assay software.

The holding potential was −80 mV. The hERG current (K⁺-selective outward current) was determined as the maximal tail current at −40 mV after a 2 second depolarization to +60 mV. Pulse cycling rate was 15 s. A short pulse (90 ms) to −40 mV served as a baseline step to calculate the tail current amplitude. After establishing whole-cell configuration and a stability period, the solvent control (0.3% DMSO) was applied for 5 minutes followed by the test substance by four increasing concentrations of 3×10⁻⁷ M, 3×10⁻⁶ M, 10⁻⁵ M and 3×10⁻⁵ M. Each concentration of the test substance was applied twice. The effect of each concentration was determined after 5 min as an average current of 3 sequential voltage pulses. To determine the extent of block the residual current was compared with vehicle pre-treatment.

Concentration/response relations were calculated by non-linear least-squares fits to the individual data points. The half-maximal inhibiting concentration (IC50) was calculated by the fitting routine.

### Protocol 2:

### Cells

The compound, vehicle control and positive control were tested on hERG-transfected HEK293 cells. A human embryonic kidney cell line (HEK293) with a stable transfection of hERG (Zhou Z et al. Biophysical Journal 1998. 74, 230-241; McDonald T. V. et al, Nature 1997. 388, 289-292) was used (University of Wisconsin, Madison, USA). The cells were kept in culture in MEM (Minimum Essential Medium, Gibco) which was supplemented with (amounts indicated added to 500 ml MEM): 5 ml L-Glutamine-Penicillin-Streptomycin (Sigma), 50 ml Fetal Bovine serum (Bio-Whittaker), 5 ml Non-essential Amino Acids 100× (Gibco), 5 mil sodium pyruvate 100 mM (Gibco) and 4 ml geneticin 50 mg/ml (Gibco) using T175 flasks. The cells were incubated at 37° C. in 5% CO₂ atmosphere (in air).

### Cell Harvesting for Assay

Cells were harvested as described below using Accumax™ (Sigma) as the dissociating reagent. Cells were then resuspended in a mixture of 33% DMEM/F12 (Dulbecco's Modified Eagle Medium/Nutrient Mixture F-12—Sigma) media/67% extracellular physiological solution.

The flasks were washed twice carefully with ˜5-10 ml phosphate buffered saline (PBS) (Gibco™) containing 2 mM EDTA (Ethylenediaminetetraacetic acid) (Sigma). The cells were dissociated using ˜3 ml of Accumax™ (cell detachment solution) and incubated for ˜5 to 10 min. at 37° C. Cold external physiological solution (2-5 ml) was added and the flasks are incubated at ˜4° C. for 5-10 min. Then, the cell suspension in each flask was gently dissociated with a 5 ml pipette. The cell suspension was transferred to a low binding petri-dish (˜10 mm diameter). Each flask was washed with ˜additional 5 ml cold external physiological solution and this solution was also added to the petri-dish. The petri-dish was then incubated for another 5 to 10 minutes at ˜4° C. After another gentle dissociation of the cell suspension in the petri dish, the cells were transferred to a reservoir kept on an orbital shaker at 200 rpm at 16° C. Before experiments were performed, the cells recovered for ˜20 min.

### Compounds

A 10 mM solution of the compound was used and plated in a 384 well plate. Aliquots of the stock solutions are diluted with the recording solution using automated liquid handling (Biomek FXP; final DMSO concentration: 0.03 to 0.3%). A standard range of screening concentrations was used ranging from 1 μM to 30 μM.

A positive control (E-4031) was included within each run to evaluate the sensitivity of the assay. Table 13 summarizes the external and intracellular solutions used in the experiments. In Table 13, the composition of the intracellular and external buffer solutions is shown in [mM] and “NMDG” means N-methyl-D-glucamine.

TABLE 13
External and Intracellular Solutions

| | | Extracellular | | | | | |
|---|---|---|---|---|---|---|---|
| | | Physiological | | | | | |
| Intracellular | | Solution/Chip | | Seal Enhancer | | | |
| Solution | | Fill Solution | | Solution | | Recording Solution | |
| KCl | 10 | NaCl | 140 | NMDG | 60 | NMDG | 60 |
| KF | 110 | KCl | 4 | NaCl | 80 | NaCl | 80 |
| NaCl | 10 | Glucose | 5 | KCl | 4 | KCl | 4 |
| HEPES | 10 | HEPES | 10 | CaCl₂ | 10 | CaCl₂ | 2 |
| EGTA | 10 | CaCl₂ | 2 | MgCl₂ | 1 | MgCl₂ | 1 |
| | | MgCl₂ | 1 | Glucose | 5 | Glucose | 5 |
| | | | | HEPES | 10 | HEPES | 10 |

| | | | |
|---|---|---|---|
| pH 7.2 (KOH) | pH 7.4 (NaOH) | pH 7.4 (HCL) | pH 7.4 (HCL) |

### Study Design

The whole cell patch clamp technique on transfected cells allows the study of ion-channels with no—or limited interference from other ion-channels. The effects of the compounds on the hERG current were studied with an automated planar patch clamp system, SyncroPatch 384PE (Obergrussberger et al, Journal of Laboratory Automation 2016. 21 (6), 779-793). All cells were recorded in the whole cell mode of the patch clamp technique. The module is incorporated in a liquid handling pipetting robot system, Biomek FXP, for application of cells and compounds, vehicle control and positive control.

The different concentrations of the compounds were applied in two cumulatively increasing concentrations for the compounds (1 μM and 10 μM, and 3 μM and 30 μM, respectively). The hERG current was determined as the maximal tail current at −30 mV and percent inhibition upon compound or vehicle and positive control addition was reported.

After cells are caught onto the individual holes of the recording chips using the chip fill solution, the seal is increased with the seal enhancer solution (increased [Ca²⁺]; then the cells were washed twice with recording solution before using a pressure protocol to go into the whole cell mode.

After the whole cell mode was achieved, test pulses were given for ˜10 minutes to quantify the hERG current in control conditions. During this control period vehicle control solution (recording solution containing 0.03% DMSO) was added three times into the individual wells. While continuing the pulse protocol, cumulatively increasing concentrations of the vehicle control, compound or positive control was added. The effect of the vehicle, compound and positive control was measured after 5 minutes of drug application. Two concentrations of the compound were tested per cell.

The use of the internal and recording solutions will result in ˜10 mV liquid junction potential and the command voltage step will take this into account.

Electrophysiological measurements: The membrane current of the cells was measured at distinct membrane potentials with the patch clamp technique by means of an automated patch clamp system. The holding potential is −70 mV. The hERG current (K⁺-selective outward current) was determined as the maximal tail current at −30 mV after a 2 second depolarization to +70 mV (refs. 1, 4). Pulse cycling rate was 15 s.

### Data Analysis

The leak corrected hERG current (K⁺-selective outward current) was determined as the maximal tail current at −30 mV after a 2-second of depolarization to +70 mV measured between 2336.3 ms and 3083.6 ms. The median of three current amplitudes was taken at the end of the control period and at the end of each addition of compound, vehicle and positive control to calculate the percent inhibition.

QC parameters were set in the SyncroPatch 384PE PatchControl384 software to automatically exclude wells from the analysis if values fall outside the range. The QC criteria are dependent on the type of recording plate (chip). Typically, a 4×Chip (medium size hole) was used to record from hERG-transfected HEK293 cells. QC criteria 4-6 were set before the first addition of the compound; QC criteria 4 and 5 were also set at the end of each compound addition.

QC Criteria and acceptable ranges:
1. Board Check: −500 pA-500 pA
2. Contact seal resistance: −100 kOhm-10 MOhm
3. Junction potential offset: 0-100 mV
4. Rseal ≥100 MOhm
5. Rseries: between 1-25 MOhm
6. hERG tail current ≥0.2 nA before compound addition

Each compound was replicated on the same plate in at least 5 wells. Percent inhibition of at least 2-3 replicates per concentration will be reported as median. The results are summarized below in Tables 14 and 15 for Protocol 1 and Protocol 2, respectively.

TABLE 14
hERG IC₅₀ (μM) from Protocol 1

| Compound Number | hERG- IC₅₀ (μM) |
|---|---|
| A | >30.2 |

TABLE 15
hERG IC₅₀ (μM) from Protocol 2

| Example Number | hERG IC₅₀ μM |
|---|---|
| 11 | >30.2 |

### 9) Efficacy Study in Disseminated OCI-AML3 Model

### Test Agents and Controls

Compound A3 was formulated in 20% hydroxypropyl-beta-cyclodextrin (HP-3-CD) and prepared to reach a total volume of 0.2 mL (10 mL/kg) per dose for a 20 g animal. Doses were adjusted by individual body weight each day. Working stocks of Compound A3 were prepared once per week for each study and stored at 25° C.

### Animals

Female SCID beige mice (CB17.Cg-PrkdcscidLystbg-J/Crl/−) were used when they were approximately 6 to 8 weeks of age and weighed approximately 25 g. All animals could acclimate and recover from any shipping-related stress for a minimum of 7 days prior to experimental use. Autoclaved water and irradiated food were provided ad libitum, and the animals were maintained on a 12 hour light and dark cycle. Cages, bedding, and water bottles were autoclaved before use and changed weekly. The tissue culture and cell injection reagents are summarized below in Table 16.

TABLE 16
Tissue Culture and Cell Injection Reagents

DPBS (Dulbecco's phosphate-buffered saline)
Heat-inactivated fetal bovine serum
MEM Alpha medium
L-glutamine
Gentamycin
T175 Culture Flask
Roller Bottle

### Tumor Model and Cell Culture Method

Human AML cell line OCI-AML3 was cultured at 37° C., 5% CO₂ in the indicated complete culture media (MEM Alpha+20% HI-FBS (Heat-Inactivated Fetal Bovine Serum)+2 mM L-glutamine+50 ug/ml Gentamycin). Cells were harvested while in logarithmic growth and resuspended in cold (4° C.) MEM ((Minimum Essential Medium) Alpha in serum-free medium.

For the disseminated OCI-AML3 model, each mouse received 5×10⁵ cells via IV injection in a total volume of 0.2 mL using a 26-gauge needle.

### Study Designs

Compound A3 was administered orally (PO), daily.

Day 0 is the day of tumor cell implantation and study initiation.

In the efficacy study, mice bearing IV OCI-AML3 xenograft tumors were randomly assigned to treatment groups 3 days post-tumor cell engraftment. Treatment with vehicle or Compound A3 (at 30, 50, 100 mg/kg) was initiated on the same day, with daily dosing for 28 days.

### Animal Monitoring

Animals were monitored daily for clinical signs related to either compound toxicity or tumor burden (i.e., hind limb paralysis, lethargy, etc.).

### Calculations

For survival assessment, results were plotted as the percentage survival against days post tumor implant. Negative clinical signs and/or ≥20% body weight loss was used as a surrogate endpoint for death. Median survival was determined utilizing Kaplan-Meier survival analysis. The percent increased life span (ILS) was calculated as: ((median survival day of treated group—median survival day of control group)/median survival day of control group)×100. Animals failing to reach the surrogate endpoint due to adverse clinical signs (such as ulcerated tumors, body weight loss, etc.) or death unrelated to treatment were censored for the survival assessment. As defined by NCI criteria, ≥25% ILS is considered biologically significant. (Johnson J I et al. Br J Cancer. 2001. 84(10), 1424-1431).

### Data Analysis

Survival and body weight data were graphically represented utilizing Prism (Version 7). Statistical significance for body weights was evaluated as described above. Statistical significance was evaluated for Kaplan-Meier survival plots comparing therapeutic treatment group vs. appropriate vehicle-treated control using log-rank (Mantel-Cox) test in R software version 3.4.2. Differences between groups were considered significant when the p value was ≤0.05.

### Survival

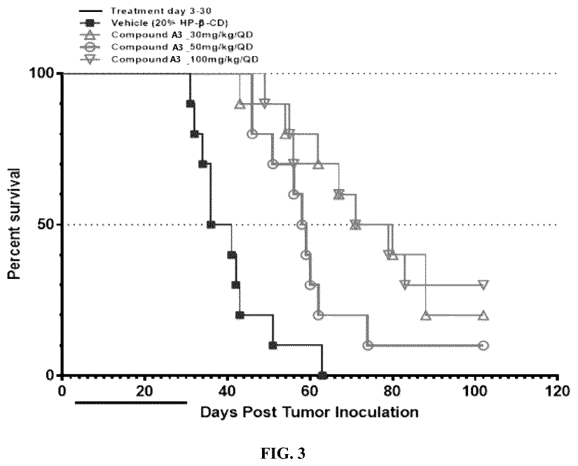

The Kaplan-Meier survival curve is shown in FIG. 3. Mice bearing established OCI-AML3 tumors were orally dosed daily with Compound A3 at 30, 50, 100 mg/kg in 20% HP-β-CD formulation for a total of 28 days (n=9-10/group). For Compound A3 treated groups, the median days of survival were reached at the following days for 30 mg/kg at day 75.5, for 50 mg/kg at day 58.5 and for 100 mg/kg at day 75 this compared to a median survival of 38.5 days for the vehicle-treated control group. Compound A3 treatment resulted in statistically significant increased lifespan of OCI-AML3 tumor-bearing mice by 96.1%, 51.9% and 94.8% (at the 30, 50 and 100 mg/kg dose levels) as compared to that of control mice, (p≤0.001). This was a biologically significant ILS as per NCI criteria threshold of ≥25% ILS (Johnson J I et al. Br J Cancer. 2001. 84(10), 1424-1431).

10) Efficacy of Menin-MLL Inhibitor in Combination with Azacitidine in OCI-AML3 (NPM1c) IV Model in NSG Mice

The disseminated OCI-AML3 model described above was also used to examine the efficacy of menin-MLL-inhibitor (formulated as described above) in combination with azacitidine (formulated in 10% dimethyl sulfoxide [DMSO]) with the following differences: (i) instead of female SCID beige, NSG (non-obese diabetic [NOD] scid gamma or NOD.Cg-Prkdc^{scid} II2rg^{tm1Wj1}/SzJ) were utilized, (ii) instead of 5×10⁵ cells, each mouse received an injection of 1×10⁶ cells, (iii) instead of 3 days, treatment with compound(s) was initiated 5 days after cell injection, and (iv) instead of a total volume of 0.2 mL (10 ml/kg) per dose, each compound was prepared to reach a volume of 8 ml/kg. Compound A1 was given in the morning, and azacitidine was given in the afternoon, approximately 6-8 hr apart. The treatment of each group using compound(s) in this efficacy study is summarized in Table 17.

TABLE 17
Treatment of OCI-AML3 tumors in mice

| | TREATMENT | | | | |
|---|---|---|---|---|---|
| GROUP | (n = 10) | DOSE | | ROUTE | SCHEDULE |
| 1 | Vehicles | 8 | mL/kg | ip/po | QD × 7/QD × 28 |
| 2 | Azacitidine | 2 | mg/kg | Ip | QD × 7 |
| 3 | Compound A1 | 30 | mg/kg | Po | QD × 28 |
| 4 | Azacitidine + | 2 | mg/kg | ip/po | QD × 7/QD × 28 |
| | Compound A1 | 30 | mg/kg | | |

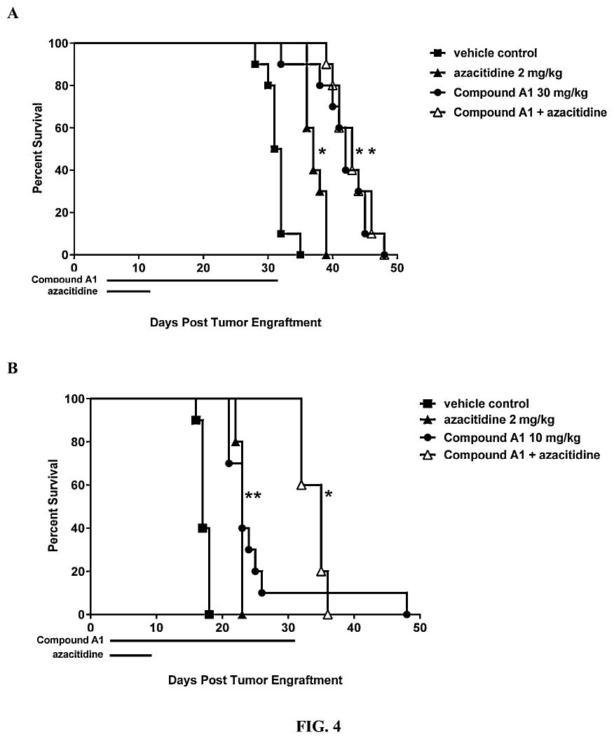

The Kaplan-Meier survival curve is shown in FIG. 4A. The impact on life span of treating mice bearing established OCI-AML3 tumors for 4 weeks is summarized in Table 18.

TABLE 18
Impact on Life Span following Treatment
of OCI-AML3-tumors in mice

| TREATMENT | % ILS |
|---|---|
| Azacitidine | *18 |
| Compound A1 | *†33 |
| Azacitidine + | *†37 |
| Compound A1 | |

*p < 0.05 as compared with vehicle control and
†azacitidine treated mice.

As reflected in Table 18, treatment of mice with Compound A1 in combination with azacitidine resulted in statistically significant increased lifespan (ILS) of OCI-AML3 tumor-bearing mice as compared to that of vehicle control treated mice or with azacitidine alone. There was no statistical difference, and therefore no antagonism, with Compound A1 in combination with azacitidine as compared to Compound A1 alone.

11) Efficacy of Menin-MLL Inhibitor in Combination with Azacitidine, in MOLM-13 (KMT2A-r) IV Model in NSG Mice

A similar approach to the disseminated OCI-AML3 model described herein for examining the efficacy of menin-MLL-inhibitor in combination with azacitidine was adopted with the MOLM-13 cells being used instead of OCI-AML3 cells.

### Tumor Model and Cell Culture Method

Human AML cell line MOLM-13 was cultured at 37° C., 5% CO2 in the indicated complete culture media (RPMI, Roswell Park Memorial Institute [RPMI] 1640+10% HI FBS+2 mM L-glutamine+50 μg/ml Gentamycin). Cells were harvested while in logarithmic growth and resuspended in cold (4° C.) serum-free RPMI 1640 medium. Each mouse received an injection of 1×10⁵ MOLM-13 cells, and all other parameters were conducted as described for experiment (10).

The treatment of each group using compound(s) in this efficacy study is summarized in Table 19.

TABLE 19
Treatment of MOLM-13-tumors in mice

| | TREATMENT | | | | |
|---|---|---|---|---|---|
| GROUP | (n = 10) | DOSE | | ROUTE | SCHEDULE |
| 1 | Vehicles | 8 | mL/kg | ip/po | QD × 7/QD × 28 |
| 2 | Azacitidine | 2 | mg/kg | Ip | QD × 7 |
| 3 | Compound A1 | 10 | mg/kg | Po | QD × 28 |
| 4 | Azacitidine + | 2 | mg/kg | ip/po | QD × 7/QD × 28 |
| | Compound A1 | 10 | mg/kg | | |

The Kaplan-Meier survival curve is shown in FIG. 4B. The impact on life span of treating mice bearing established MOLM-13 tumors for 4 weeks is summarized in Table 20.

TABLE 20
Impact on Life Span following Treatment
of MOLM-13 tumors in Mice

| TREATMENT | % ILS |
|---|---|
| Azacitidine | *35 |
| Compound A1 | *†35 |
| Azacitidine + | *†106 |
| Compound A1 | |

*p < 0.05 as compared with vehicle control and
†azacitidine treated mice.

As reflected in Table 20, treatment of mice with Compound A1 in combination with azacitidine resulted in statistically significant increased lifespan (ILS) of MOLM-13 tumor-bearing mice as compared to that of mice treated with azacitidine or Compound A1 alone.

12) Efficacy of Menin-MLL Inhibitor in Combination with Gilteritinib, in MOLM-13 (KMT2A-r) IV Model in NSG Mice

A similar approach to the disseminated OCI-AML3 model described herein for examining the efficacy of menin-MLL-inhibitor in combination with gilteritinib was adopted with the MOLM-13 cells being used instead of OCI-AML3 cells.

### Tumor Model and Cell Culture Method

Human AML cell line MOLM-13 was cultured at 37° C., 5% C₀₂ in the indicated complete culture media (RPMI, Roswell Park Memorial Institute [RPMI] 1640+10% HI FBS+2 mM L-glutamine+50 μg/ml Gentamycin). Cells were harvested while in logarithmic growth and resuspended in cold (4° C.) serum-free RPMI 1640 medium. Each mouse received an injection of 1×10⁵ MOLM-13 cells, and all other parameters were conducted as described for experiment (10 above entitled “Efficacy of Menin-MLL inhibitor in Combination with Azacitidine in OCI-AML3 (NPM1c) IV Model in NSG Mice”).

The treatment of each group using compound(s) in this efficacy study is summarized in Table 21.

TABLE 21
Treatment of MOLM-13-tumors in mice

| | TREATMENT | | | | |
|---|---|---|---|---|---|
| GROUP | (n = 10) | DOSE | | ROUTE | SCHEDULE |
| 1 | Vehicles | 8 | mL/kg | po/po | QD × 28/QD × 28 |
| 2 | Gilteritinib | 10 | mg/kg | po | QD × 28 |
| 3 | Compound A1 | 10 | mg/kg | po | QD × 28 |
| 4 | Gilteritinib + | 10 | mg/kg | po/po | QD × 28/QD × 28 |
| | Compound A1 | 10 | mg/kg | | |

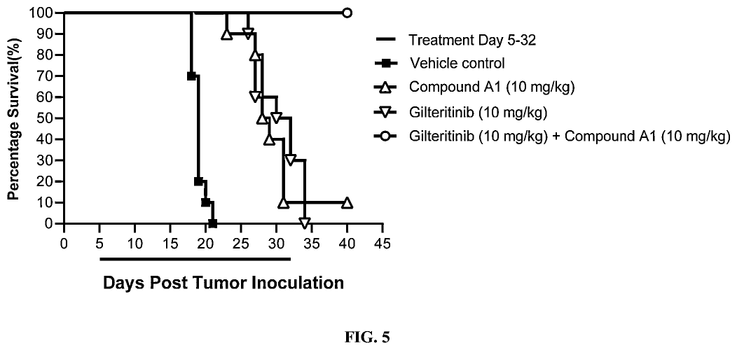

The Kaplan-Meier survival curve is shown in FIG. 5. The impact on life span of treating mice bearing established MOLM-13 tumors for 4 weeks is summarized in Table 22.

TABLE 22
Impact on Life Span following Treatment
of MOLM-13 tumors in Mice

| TREATMENT | % ILS |
|---|---|
| Gilteritinib | *63 |
| Compound A1 | *50 |
| Gilteritinib + | *†>111 |
| Compound A1 | |

*p < 0.05 as compared with vehicle control and
†gilteritinib treated mice.

As reflected in Table 22, treatment of mice with Compound A1 in combination with gilteritinib resulted in statistically significant increased lifespan (ILS) of MOLM-13 tumor-bearing mice as compared to that of mice treated with gilteritinib or Compound A1 alone.

13) In Vitro Proliferation—Menin-MLL Inhibitor in Combination with FLT3 Inhibitor (Gilteritinib or Midostaurin), DNA Intercalating Agent (Idarubicin) or Hyponethylating Agent (Decitabine)

### Cell Lines

AML cell lines MOLM-13, OCI-AML3 and MV4-11 were purchased from DSMZ. MOLM-13 were grown in RPMI medium supplemented with 10% Fetal Bovine Serum (FBS) and 1% penicillin/streptomycin. MV4-11 cells were grown in IMDM medium supplemented with 10% FBS and 1% penicillin/streptomycin. OCI-AML3 cells were cultured in 80-90% alpha-MEM (with ribo- and deoxyribonucleosides)+10-20% FBS. All cell lines were cultured at 37° C. in 5% CO2 atmosphere.

### Cell Titer Glo Assays

AML cell lines (5×10³ cells/well) were seeded in 96-well plates and grown for 6 days in serum (10%)-containing medium in the presence or absence of inhibitors at the indicated concentrations. Proliferation was analyzed by means of a Cell Titer Glo assay using the CellTiter 96 Aqueous One Solution Cell Proliferation Assay (Promega, Madison, WI, USA), according to the manufacturer's instructions. Data are mean with standard deviation from two to four independent experiments in technical triplicates.

### Synergy Calculations

R-based Biochemically Intuitive Generalized Loewe (BIGL) model implemented with a highest single agent (HSA) null model. Specifically, the BIGL methodology was applied to calculate drug-drug interactions (Van der Borght, K., Tourny, A., Bagdziunas, R. et al. BIGL: Biochemically Intuitive Generalized Loewe null model for prediction of the expected combined effect compatible with partial agonism and antagonism. Sci Rep 7, 17935 (2017); Thas, O., Tourny, A., Verbist, B., Hawinkel, S., Nazarov, M., Mutambanengwe, K., & Bijnens, L. Statistical detection of synergy: New methods and a comparative study. Pharmaceutical Statistics (2021)).

Synergy matrix results of the BIGL analysis of the cell line data with HSA as mean model calculated on the basis of the cellular metabolic activity using Cell Titer-Glo assay. Bootstrap confidence intervals are indicated. Effect sizes and their confidence intervals are shown. Notably, each data point based on the p-value and sign of the respective maxR statistic with the size of the dots reflecting the degree of synergy or antagonism corresponding to graded scale. No significant average effect if zero is included in the interval.

### Results

(A) Menin-MLL Inhibitor in Combination with FLT3 Inhibitor (Gilteritinib or Midostaurin)

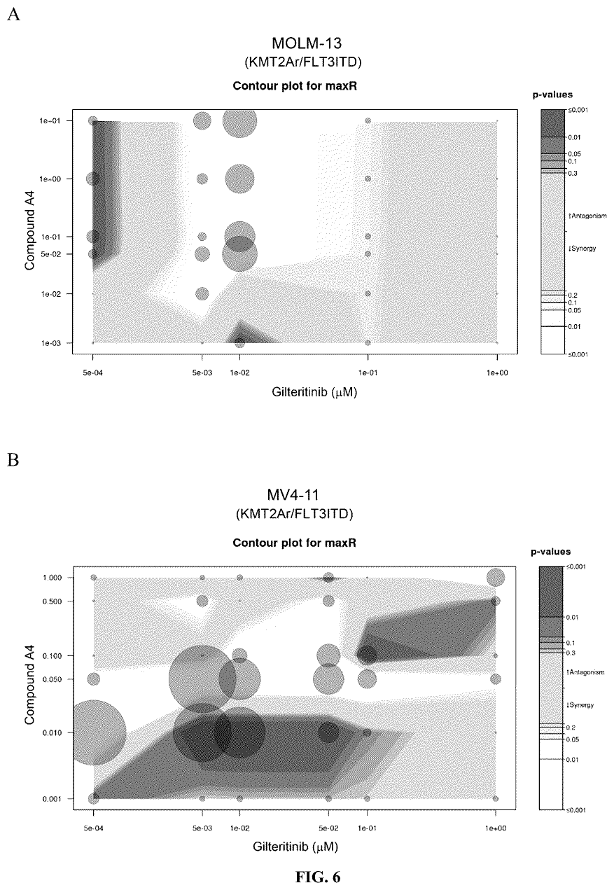

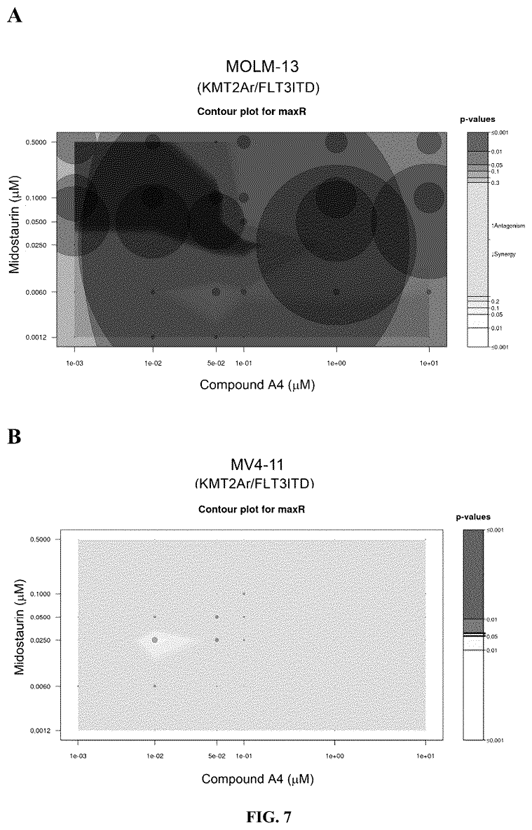

A pairwise matrix combination of gilteritinib or midostaurin with Compound A4 was evaluated in MOLM-13 (KMT2A rearranged) and MV4-11 (FLT3-ITD) AML cell lines using a 6-day CellTiter-Glo assay format. Notably, the combination of Compound A4 with either gilteritinib or midostaurin is not antagonistically cytotoxic in either of these cell lines (as reflected in FIGS. 6A and 6B plus detailed below in Tables 23A and 23B for gilteritinib; and in FIGS. 7A and 7B plus detailed below in Tables 24A and 24B for midostaurin).

Low doses of the more selective FLT3 inhibitor gilteritinib (i.e., 5 nM or 0.5 nM) in combination with Compound A4 were found to have a synergistic effect in MOLM-13 and MV4-11 cell lines, respectively, as indicated by a greater decrease in cell proliferation compared to that which would be expected from an additive response (see FIGS. 6A and 6B as well as Tables 23A and 23B).

TABLE 23A
Effect of Compound A4 in Combination with Gilteritinib on MOLM-13 Cell Proliferation.

| | MOLM-13 | | | | | |
|---|---|---|---|---|---|---|
| Compound | 0.001 | −0.017 | −0.0065 | 0.0527* | −0.0555 | 0.0005 |
| A4 (μM) | | (−0.0517, 0.0177) | (−0.0471, 0.0 [symbol] 4) | (0.0101, 0.0952) | (−0.0891, −0.0219) | (−0.0316, 0.032 [symbol] ) |
| | 0.01 | −0.0217 | −0.0802** | −0.0143 | −0.0559 | 0 |
| | | (−0.0681, 0.0247) | (−0.1202, −0.0401) | (−0.0564, 0.0278) | (−0.0 [symbol] 95, −0.0223) | (−0.0 [symbol] 21, 0.0 [symbol] 21) |
| | 0.05 | −0.0395 | −0.1 [symbol] 95** | [symbol] 0.3031** | −0.0 [symbol] 68 | −0.0004 |
| | | (−0.0813, 0.0023) | (−0.2 [symbol] 0 [symbol] , −0.1487) | (−0.3432, −0.2631) | (−0.0 [symbol] 4, −0.0232) | (−0.0325, 0.0317) |
| | 0.1 | −0.0281 | −0.157** | −0.2794** | −0.0573 | −0.0006 |
| | | (−0.064, 0.0077) | (−0.1919, −0.1222) | (−0.3133, [symbol] 0.2455) | (−0.0909, 0.0237) | (−0.0 [symbol] 27, 0.0314) |
| | 1 | −0.0177 | −0.1611** | −0.2593** | −0.0576 | −0.0008 |
| | | (−0.0538, 0.0184) | (−0.1961, −0.1261) | (−0.29 [symbol] 5, [symbol] 0.22 [symbol] ) | (−0.0912, −0.024) | (−0.0329, 0.03 [symbol] ) |
| | 10 | −0.0426* | −0.1987** | −0.284 [symbol] ** | −0.058 | −0.0009 |
| | | (−0.0792, −0.0059) | (−0.2342, −0.16 [symbol] 2) | (−0.319 [symbol] , −0.2495) | (−0.0916, 0.0244) | (−0.0 [symbol] , 0.0 [symbol] 11) |

| | | | | | |
|---|---|---|---|---|---|
| Gilteritinib (μM) | 0.0005 | 0.005 | 0.01 | 0.1 | 1 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively. [symbol]
indicates data missing or illegible when filed

TABLE 23B
Effect of Compound A4 in Combination with Gilteritinib on MV4-11 Cell Proliferation.

| | | MV4-11 | | | | |
|---|---|---|---|---|---|---|
| Compound | 0.001 | −0.0484 | −0.0103 | −0.0083 | −0.0081 | −0.0098 |
| A4 (μM) | | (−0.1364, 0.0396) | (−0.0597, 0.0392) | (−0.0351, 0.0185) | (−0.0337, 0.0175) | (−0.0353, 0.0158) |
| | 0.01 | −0.4743** | −0.0204 | −0.0174 | −0.0172 | −0.0174 |
| | | (−0.5379, −0.4106) | (−0.069, 0.0282) | (−0.0429, 0.0081) | (−0.0416, 0.0071) | (−0.042, 0.0072) |
| | 0.05 | −0.0784** | −0.0219 | −0.0184 | −0.0178 | −0.0177 |
| | | (−0.1366, −0.0202) | (−0.0705, 0.0267) | (−0.0439, 0.007) | (−0.0422, 0.0055) | (−0.0423, 0.0069) |
| | 0.1 | −0.0331 | −0.0218 | −0.0186 | −0.0178 | −0.0173 |
| | | (−0.0638, −0.0024) | (−0.0633, 0.0198) | (−0.044, 0.0068) | (−0.0422, 0.0065) | (−0.0419, 0.0072) |
| | 1 | −0.0191 | −0.0217 | −0.0183 | −0.0176 | −0.0175 |
| | | (−0.0553, 0.017) | (−0.0475, 0.0042) | (−0.0391, 0.0025) | (−0.0384, 0.0032) | (−0.0385, 0.0036) |
| | 10 | −0.0198 | −0.0204 | −0.0184 | −0.0171 | −0.0173 |
| | | (−0.0564, 0.0168) | (−0.0464, 0.0056) | (−0.0392, 0.0024) | (−0.0378, 0.0037) | (−0.0382, 0.0037) |

| | | | | | |
|---|---|---|---|---|---|
| Gilteritinib (μM) | 0.0005 | 0.005 | 0.01 | 0.1 | 1 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively.

TABLE 24A
Effect of Compound A4 in Combination with Midostaurin on MOLM-13 Cell Proliferation.

| | MOLM-13 | | | | | | |
|---|---|---|---|---|---|---|---|
| Compound | 0.001 | −0.09 | −0.15 | −0.05 | −0.0 [symbol] | 0.0009 | 0.011 |
| A4 (μM) | | (−0.77, 0. [symbol] 9) | (−0. [symbol] , 0. [symbol] 7) | (−0. [symbol] , 0.24) | (−0.154, 0.09 [symbol] ) | (−0.141 [symbol] , 0.14 [symbol] 4) | (−0.1494, 0.1715) |
| | 0.01 | 0. [symbol] 8 | −0.3 [symbol] | −0.1 | −0.02 [symbol] | 0.0005 | 0.0 [symbol] 05 |
| | | (−0.84, 0.28) | (−0.8 [symbol] 4, 0.1 [symbol] 45) | (−0. [symbol] 2, 0.11) | (−0.1545, 0.0 [symbol] ) | (−0.142, 0.1 [symbol] ) | (−0.1 [symbol] , 0.1711) |
| | 0.05 | −0.21 | −0.4 [symbol] 24* | −0.114 | −0.0 [symbol] | 0.000 [symbol] | 0.01 |
| | | (−0.73, 0. [symbol] 2) | (−0. [symbol] , −0.0 [symbol] 26) | (−0. [symbol] , 0.0 [symbol] ) | (−0. [symbol] 47, 0.0 [symbol] 5) | ( [symbol] 0.1422, 0.1429) | (−0.1 [symbol] , 0.17) |
| | 0.1 | −0.11 | −0. [symbol] | −0.11 [symbol] 7 | −0.0 [symbol] 02 | 0 | 0.0101 |
| | | (−0.65, 0.44) | (−0.7521, 0.10 [symbol] 2) | (−0.3293, 0.0 [symbol] 98) | (−0.1559, 0.0 [symbol] ) | (−0.142 [symbol] , 0.142 [symbol] ) | (−0.150 [symbol] , 0.1707) |
| | 1 | −0.05 | −0.2989 | −0.125 [symbol] | −0.0 [symbol] 11 | −0.000 [symbol] | 0.01 |
| | | ( [symbol] 0.55, 0.44) | (−0. [symbol] , 0.0 [symbol] 52) | (−0.33 [symbol] , 0.0841) | (−0.1 [symbol] , 0.0945) | (−0.1429, 0.1422) | (−0.150 [symbol] , 0.1706) |
| | 10 | −0.02 | −0.227 [symbol] | −0.1 [symbol] 29 | −0.0 [symbol] 17 | −0.0002 | 0.01 |
| | | (−0. [symbol] 2, 0.48) | (−0. [symbol] 2, 0.14 [symbol] 3) | (−0.3 [symbol] 25, 0.0766) | (−0. [symbol] 574, 0.0938) | ( [symbol] 0.1428, 0.1423) | (−0.1 [symbol] 04, 0.1705) |

| | | | | | | |
|---|---|---|---|---|---|---|
| Midostaurin (μM) | 0.0012 | 0.006 | 0.025 | 0.05 | 0.1 | 0.5 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively. [symbol]
indicates data missing or illegible when filed

TABLE 24B
Effect of Compound A4 in Combination with Midostaurin on MV4-11 Cell Proliferation.

| | MV4-11 | | | | | | |
|---|---|---|---|---|---|---|---|
| Compound | 0.001 | 0.04 | 0. [symbol] 6 | −0.12 | −0.07 | −0.0 [symbol] | 0.04 |
| A4 (μM) | | (−0.83, 0.9 [symbol] ) | (−0.72, 1.04) | (−0. [symbol] , 0.34) | (−0.38, 0.2 [symbol] ) | (−0.2 [symbol] , 0.27) | (−0.27, 0.34) |
| | 0.03 | −0.09 | −0.2 [symbol] | −0.2 [symbol] | −0.1 | −0.02 | 0.0 [symbol] |
| | | (−0.71, 0.5 [symbol] ) | (−0.77, 0.26) | (−0.58, 0.08) | (−0. [symbol] , 0. [symbol] ) | (−0.2 [symbol] , 0.24) | (−0.2 [symbol] , 0.34) |
| | 0.05 | 0.01 | −0.09 | −0.14 | −0.1 | −0.0 [symbol] | 0.03 |
| | | (−0.41, 0.43) | (−0.04, 0.25) | (−0.4, 0.12) | (−0.32, 0.11) | (−0.26, 0.21) | (−0.25, 0. [symbol] 2) |
| | 0.1 | −0.02 | −0.0 [symbol] | −0.09 | −0.09 | −0.0 [symbol] | 0.0 [symbol] |
| | | (−0.35, 0. [symbol] ) | (−0.0 [symbol] , 0.1 [symbol] ) | (−0. [symbol] , 0.15) | (−0. [symbol] , 0.11) | (−0.02 [symbol] , 0.19) | (−0.23, 0. [symbol] ) |
| | 1 | 0 | −0.03 | −0.04 | −0.04 | −0.0 [symbol] | 0.0 [symbol] |
| | | (−0.61, 0. [symbol] 1) | (−0. [symbol] 1, 0.26) | (−0. [symbol] , 0.22) | (−0.28, 0.19) | (−0.2 [symbol] , 0.19) | (−0. [symbol] , 0.27) |
| | 10 | −0.0 [symbol] | −0.0 [symbol] | −0.04 | −0.04 | −0.02 | 0.0 [symbol] |
| | | (−0.32, 0. [symbol] ) | (−0.0 [symbol] 2, 0.28) | (−0.31, 0.24) | (−0.29, 0.2 [symbol] ) | (−0.24, 0. [symbol] ) | (−0. [symbol] , 0.27) |

| | | | | | | |
|---|---|---|---|---|---|---|
| Midostaurin (μM) | 0.0012 | 0.006 | 0.025 | 0.05 | 0.1 | 0.5 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively. [symbol]
indicates data missing or illegible when filed

(B) Menin-MLL Inhibitor in Combination with Idarubicin

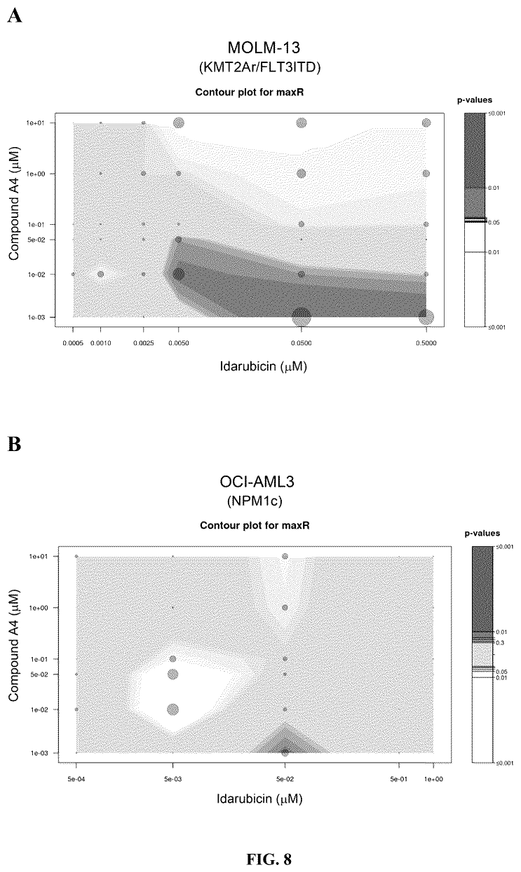

A pairwise matrix combination of idarubicin and Compound A4 was evaluated in MOLM-13 (KMT2A-AF9; FLT3-ITD) and OCI-AML3 (NPM1c AML) cells using a 6-day Cell Titer-Glo assay format. Notably, the combination of idarubicin and Compound A4 is not antagonistically cytotoxic in MOLM-13 (KMT2A-AF9; FLT3-ITD) cells as reflected in the contour plot (FIG. 8A) and detailed below in Table 25A.

TABLE 25A
Effect of Compound A4 in Combination with Idarubicin on MOLM-13 Cell Proliferation.

| | MOLM-13 | | | | | | |
|---|---|---|---|---|---|---|---|
| Compound | 0.001 | 0.01 | −0.06 | 0.0 [symbol] | −0.0 [symbol] | −0.01 | 0 |
| A4 (μM) | | (−1.17, 1.2) | (−1.14, 1.02) | (−0.86, 0. [symbol] ) | (−0.37, 0.14) | (−0.2 [symbol] , 0.24) | (−0.27, 0.28) |
| | 0.01 | −0.29 | −0.37 | −0.22 | −0.12 | −0.01 | 0 |
| | | (−0.97, 0. [symbol] ) | (−0.85, 0.1) | (−0.54, 0.09) | (−0.36, 0.1 [symbol] ) | (−0.26, 0.24) | (−0.28, 0.2 [symbol] ) |
| | 0.05 | 0.05 | −0.2 | −0.22 | −0.12 | −0.01 | 0 |
| | | (−1.1, 1.2) | (−1.0 [symbol] , 0.61) | (−0.56, 0. [symbol] ) | (−0.36, 0.1 [symbol] ) | (−0.26, 0.24) | (−0.28, 0.28) |
| | 0.1 | −0.13 | −0. [symbol] | −0.22 | −0.12 | −0.01 | 0 |
| | | (−1.04, 0.78) | (−1, 0.6) | (−0. [symbol] , 0.11) | (−0. [symbol] 6, 0.13) | (−0.26, 0.24) | (−0.28, 0.28) |
| | 1 | −0.04 | −0.2 [symbol] | −0.2 [symbol] | −0.12 | −0.01 | 0 |
| | | (−1.0 [symbol] , 0.97) | (−0.94, 0. [symbol] ) | (−0. [symbol] , 0.08) | (−0.36, 0.1 [symbol] ) | (−0.2 [symbol] , 0.24) | (−0.28, 0.28) |
| | 10 | −0.09 | −0.22 | −0.23 | −0. [symbol] 2 | −0.01 | 0.0 [symbol] |
| | | (−1.02, 0. [symbol] ) | (−0.9 [symbol] , 0.49) | (−0.54, 0.09) | (−0.36, 0. [symbol] ) | (−0.26, 0.24) | (−0.2, 0.27) |

| | | | | | | |
|---|---|---|---|---|---|---|
| Idarubicin (μM) | 0.0005 | 0.001 | 0.0025 | 0.005 | 0.05 | 0.5 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively. [symbol]
indicates data missing or illegible when filed

Likewise, the combination of idarubicin and Compound A4 is not antagonistically cytotoxic in OCI-AML3 (NPM1c AML) cells as reflected in the contour plot (FIG. 8B) and detailed below in Table 25B.

TABLE 25B
Effect of Compound A4 in Combination with Idarubicin on OCI-AML3 Cell Proliferation.

| | OCI-AML3 | | | | | | |
|---|---|---|---|---|---|---|---|
| Compound | 0.001 | 0.1007 | 0.360 [symbol] | −0.0 [symbol] 27 | 0.05 [symbol] | −0.0 [symbol] 7 | −0.0 [symbol] 1 |
| A4 (μM) | | (−1. [symbol] 384, 1.73 [symbol] 8) | (−1.4806, 2.2013) | (−1.2 [symbol] 5, 1.151) | (−0. [symbol] 76, 0. [symbol] 2 | (−0.4 [symbol] 68, 0. [symbol] 29) | (−0.4 [symbol] 81, 0. [symbol] 618) |
| | 0.01 | 0.017 | 0.0815 | −0.0 [symbol] | 0. [symbol] 474 | −0.0 [symbol] 74 | −0.0 [symbol] 87 |
| | | (−1.5 [symbol] 4, 1.5 [symbol] 4) | (−1. [symbol] , [symbol] .7226) | (−1.21 [symbol] , 1.1 [symbol] ) | (−0.5313, 0.62 [symbol] 1) | (−0.4372, 0. [symbol] 25) | (−0.4386, 0.361 [symbol] ) |
| | 0.05 | 0.1 [symbol] 06 | −0.0079 | −0.2 [symbol] 7 | 0.0217 | −0.0 [symbol] 78 | −0.0 [symbol] 89 |
| | | (−1.4135, 1.7147) | (−1.4 [symbol] 59, [symbol] ) | (−1.14 [symbol] , 0. [symbol] ) | (−0.4979, 0. [symbol] 414) | (−0. [symbol] 37 [symbol] , 0. [symbol] 2) | (−0.4389, 0.36 [symbol] ) |
| | 0.1 | 0. [symbol] 19 | 0.2051 | −0.2418 | 0.0167 | −0.0 [symbol] 1 | −0.0 [symbol] |
| | | (−1.1162, 1. [symbol] 43; | (−1. [symbol] , 1. [symbol] 62) | (−0.8807, 0.3 [symbol] 72) | (−0.4 [symbol] 4, 0.5228) | (−0.4 [symbol] 79, 0. [symbol] 617) | (−0.4391, 0. [symbol] 608) |
| | | 0.2842 | 0.2 [symbol] | −0.2579 | 0.0157 | −0.0 [symbol] | −0.0 [symbol] |
| | | (− [symbol] .0472, 1.6157) | (−1. [symbol] 486, 1.5 [symbol] ) | (−0.80 [symbol] , 0.2911) | (−0.4875, 0. [symbol] 19) | (−0.4 [symbol] 84, 0.3 [symbol] ) | (−0. [symbol] , 0.3 [symbol] ) |
| | 10 | 0.1 [symbol] 71 | 0.1199 | −0. [symbol] 011 | −0.0 [symbol] 57 | −0.0 [symbol] 8 | −0.0395 |
| | | (−1.0457, 1.3209) | (−1.0 [symbol] 4, 1. [symbol] 853) | (−0.7 [symbol] , 0.1 [symbol] 2) | (−0.4 [symbol] 47, 0.4 [symbol] ) | (−0.4 [symbol] 86, 0.351) | (−0. [symbol] 4, 0. [symbol] 04) |

| | | | | | | |
|---|---|---|---|---|---|---|
| Idarubicin (μM) | 0.0005 | 0.001 | 0.0025 | 0.005 | 0 05 | 0.5 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively. [symbol]
indicates data missing or illegible when filed

(C) Menin-MLL Inhibitor in Combination with Decitabine

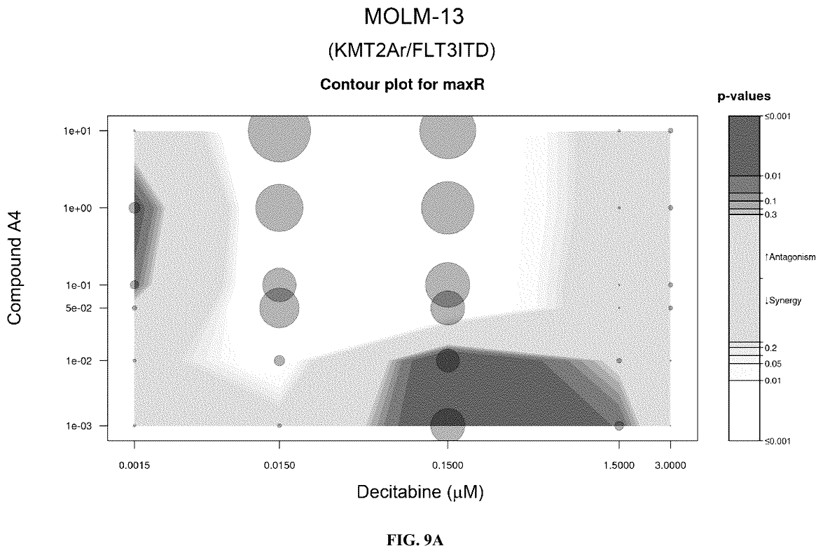

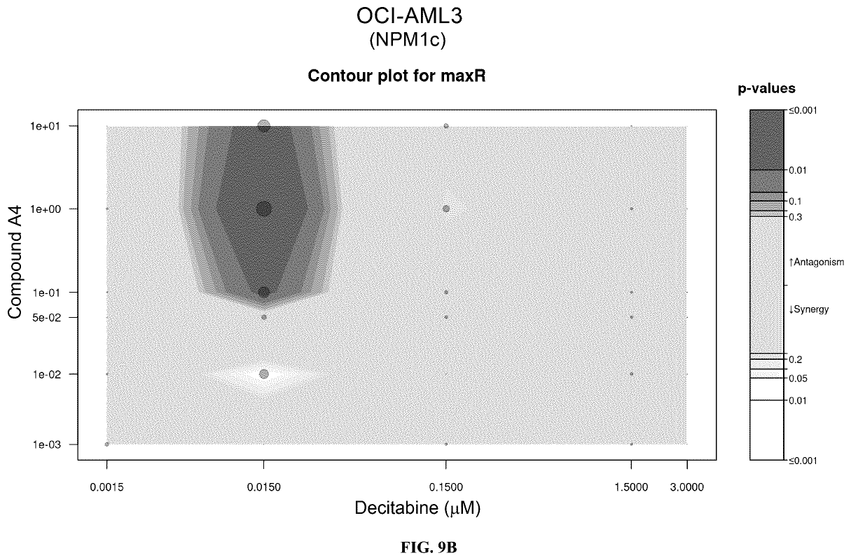

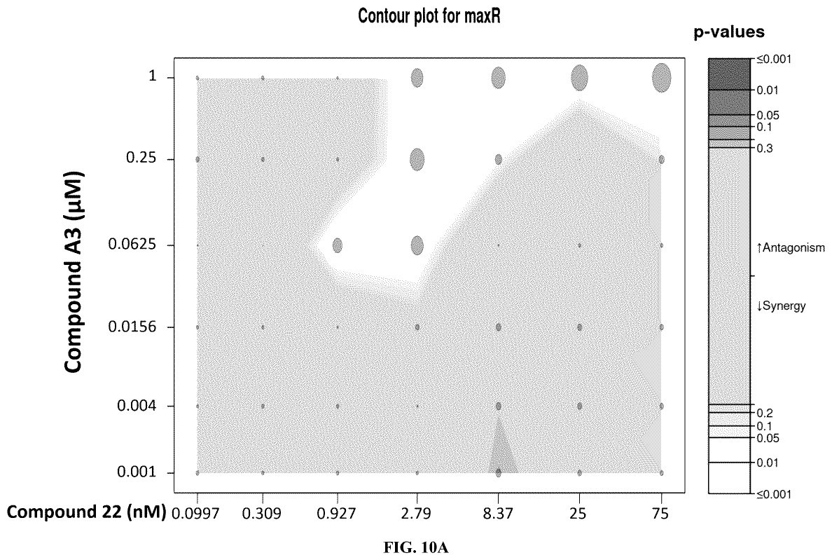

A pairwise matrix combination of decitabine and Compound A4 was evaluated in MOLM-13 (KMT2A-AF9; FLT3-ITD) and OCI-AML3 (NPM1c AML) cells using a 6-day CellTiter-Glo assay format. Notably, the combination of decitabine and Compound A4 was found to be synergistically cytotoxic in MOLM-13 (KMT2A-AF9; FLT3-ITD) cells as reflected in the contour plot (FIG. 9A) and detailed below in Table 26A.

TABLE 26A
Effect of Compound A4 in Combination with Decitabine on MOLM-13 Cell Proliferation.

| | | MOLM-13 | | | | |
|---|---|---|---|---|---|---|
| Compound | 0.001 | −0.0237 | −0.0274 | −0.0185 | −0.0029 | −0.0014 |
| A4 (μM) | | (−0.0627, 0.0153) | (−0.0651, 0.0103) | (−0.0436, 0.0066) | (−0.0152, 0.0095) | (−0.0138, 0.0109) |
| | 0.01 | −0.0156 | −0.0637** | −0.0463 | −0.0109 | −0.0059 |
| | | (−0.0565, 0.0254) | (−0.1007, −0.0267) | (−0.0706, −0.022) | (−0.0228, 0.0009) | (−0.0179, 0.0062) |
| | 0.05 | −0.0232 | −0.1574** | −0.1644** | −0.0192 | −0.0101 |
| | | (−0.0569, 0.0106) | (−0.1879, −0.1268) | (−0.1846, −0.1442) | (−0.0305, −0.008) | (−0.0219, 0.0017) |
| | 0.1 | −0.0058 | −0.1435** | −0.1783** | −0.0211 | −0.0107 |
| | | (−0.0363, 0.0248) | (−0.1703, −0.1166) | (−0.198, −0.1587) | (−0.0323, −0.01) | (−0.0224, 0.001) |
| | 1 | −0.0041 | −0.1694** | −0.1895** | −0.022 | −0.0114 |
| | | (−0.0328, 0.0245) | (−0.1932, −0.1456) | (−0.2087, −0.1703) | (−0.033, −0.0109) | (−0.0231, 0.0003) |
| | 10 | −0.0387 | −0.2** | −0.1958** | −0.0214 | −0.0336 |
| | | (−0.0665, −0.0108) | (−0.223, −0.177) | (−0.2148, −0.1768) | (−0.0825, −0.0102) | (−0.0233, 0.0001) |

| | | | | | |
|---|---|---|---|---|---|
| Decitabine (μM) | 0.0015 | 0.015 | 0.15 | 1.5 | 3 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively.

Antagonistic effects in OCI-AML3 (NPM1c AML) cells were observed at low doses of decitabine (15 nM) as reflected in the contour plot (FIG. 9B) and detailed below in Table 26B.

TABLE 26B
Effect of Compound A4 in Combination with Decitabine on OCI-AML3 Cell Proliferation.

| | | OCI-AML3 | | | | |
|---|---|---|---|---|---|---|
| Compound | 0.001 | −0.0483 | −0.0238 | −0.0156 | −0.024 | −0.0127 |
| A4 (μM) | | (−0.1014, 0.0047) | (−0.0813, 0.0338) | (−0.065, 0.0338) | (−0.0524, 0.0043) | (−0.0404, 0.0151) |
| | 0.01 | −0.0234 | −0.1175** | −0.038 | −0.0267 | −0.0128 |
| | | (−0.0831, 0.0364) | (−0.1731, −0.062) | (−0.0869, 0.0109) | (−0.055, 0.0015) | (−0.0405, 0.0149) |
| | 0.05 | 0.0136 | 0.0588 | −0.0571 | −0.0258 | −0.0136 |
| | | (−0.0364, 0.0636) | (0.0078, 0.1097) | (−0.1055, −0.0086) | (−0.0541, 0.0025) | (−0.0413, 0.0141) |
| | 0.1 | 0.0139 | 0.1279* | −0.0603 | −0.0228 | −0.0136 |
| | | (−0.0299, 0.0576) | (0.0814, 0.1745) | (−0.1086, −0.0119) | (−0.0512, 0.0056) | (−0.0413, 0.0141) |
| | 1 | 0.0141 | 0.1688* | −0.0504 | −0.0246 | −0.013 |
| | | (−0.0288, 0.057) | (0.122, 0.2156) | (−0.094, −0.0067) | (−0.053, 0.0037) | (−0.0407, 0.0147) |
| | 10 | 0.0288 | 0.1406* | −0.0324 | −0.018 | −0.0114 |
| | | (−0.0156, 0.0732) | (0.0934, 0.1877) | (−0.0767, 0.012) | (−0.0466, 0.0106) | (−0.0392, 0.0163) |

| | | | | | |
|---|---|---|---|---|---|
| Decitabine (μM) | 0.0015 | 0.015 | 0.15 | 1.5 | 3 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively.

14) In Vitro Proliferation—Menin-MLL Inhibitor in Combination with DHODH Inhibitor (Compound 22)

The effect of Compound A3 in combination with a DHODH inhibitor (Compound 22) was determined in proliferation assays using the MOLM-13 (KMT2A-r) and OCI-AML3 (NPM1c) cell lines. MOLM-13 cells were seeded at 2000 cells/well and OCI-AML3 cells were seeded at 1000 cells/well in 96-well plates and exposed to the indicated drug in combination at the specified concentration detailed in Table 27A and Table 27B. In particular, a concentration range of Compound A3 (a 6-point, 4-fold serial dilution starting at an initial concentration of 1 M in MOLM-13 or 15 μM in OCI-AML3) was combined with a concentration range of Compound 22 (a 7-point, 3-fold serial dilution starting at an initial concentration of 75 nM in MOLM13 or 250 nM in OCI-AML3). MOLM-13 and OCI-AML3 cells were incubated at 37° C., 5% CO₂ with the indicated drug combination for 8 days (Compound A3 and Compound 22) in quadruplicate.

Spheroid-like growth was monitored in real-time by non-invasive live-cell imaging using the 4× objective of the Incucyte ZOOM live cell imaging system (Essen BioScience), and acquiring images on Day 8. The percent confluence, as a measure of spheroid size, was determined using an integrated analysis tool that is the part of the Incucyte ZOOM software “IncuCyte ZOOM 2016B” (Essen BioScience). The DMSO content was normalized to 0.3% and the percent confluence from the DMSO well was used as baseline response.

Potential combination effects were evaluated at day 8 and analyzed for synergy using an R-based Biochemically Intuitive Generalized Loewe (BIGL) model implemented with a highest single agent (HSA) null model (Van der Borght 2017). Data from 2 independent experiments from MOLM-13 or 3 independent experiments from OCI-AML3 cells were pooled and analyzed.

### Results

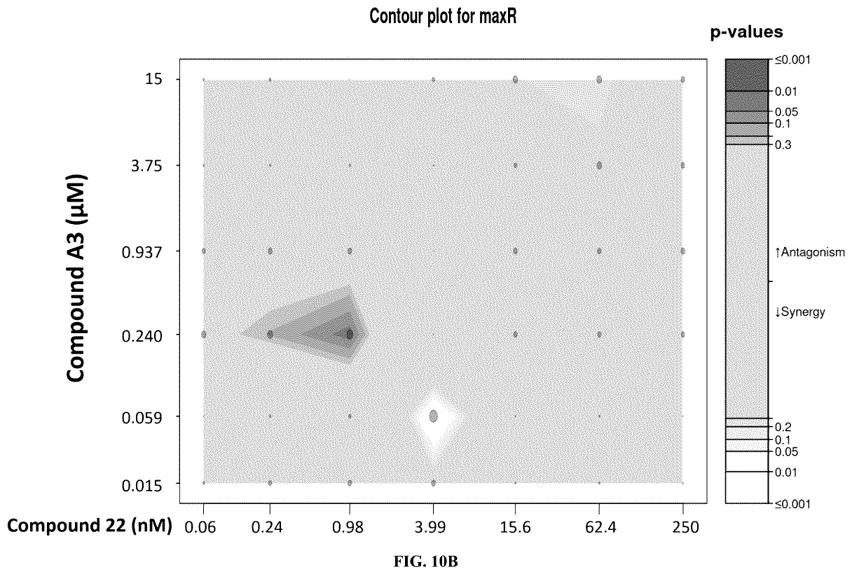

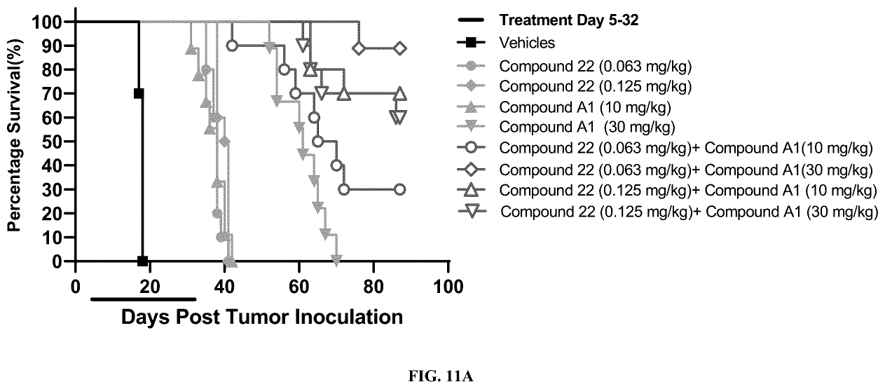

The doublet combination of Compound A3 with Compound 22 led to significantly increased inhibition of cell proliferation compared to Compound A3 or Compound 22 monotherapy in MOLM-13 cell line (see FIG. 10A). Strong synergistic effects were observed across a range of concentrations in MOLM-13 cells as reflected in the contour plot (FIG. 10A) and detailed below in Table 27A where effect size is represented as the difference between the observed outcome and the expected outcome of combination treatments with 95% confidence interval shown in parentheses.

In OCI-AML3 cells, Compound A3 and Compound 22 monotherapy mediated potent growth inhibition. No synergistic or antagonistic effects were observed for the doublet combination therapy of Compound A3 and Compound 22 as reflected in the contour plot (FIG. 10B) and detailed below in Table 27B.

TABLE 27A
Effect of Compound A3 in Combination with DHODH inhibitor (Compound 22) on MOLM-13 Cell Proliferation.

| | | | | | | | | |
|---|---|---|---|---|---|---|---|---|
| Compound | 1 | −0.02 | −0.02 | −0.08 | −0.16** | −0.12** | −0.11** | −0.11** |
| A3 (μM) | | (−0.13, 0.09) | (−0.13, 0.09) | (−0.19, 0.02) | (−0.25, −0.07) | (−0.19, −0.05) | (−0.16, −0.05) | (−0.16, −0,05) |
| | 0.25 | 0.00 | −0.01 | −0.10 | −0.22** | −0.09** | −0.04 | −0.09 |
| | | (−0.12, 0.13) | (−0.14, 0.11) | (−0.21, 0.01) | (−0.32, −0.13) | (−0.17, −0.02) | (−0.11, 0.03) | (−0.15, 0.03) |
| | 0.0625 | −0.01 | −0.04 | −0.21** | −0.24** | −0.03 | −0.02 | −0.02 |
| | | (−0.15, 0.14) | (−0.18, 0.11) | (−0.33, −0.10) | (−0.34, −0.13) | (−0.12, 0.06) | (−0.09, 0.05) | (−0.09, 0.06) |
| | 0.0156 | 0.07 | 0.06 | −0.04 | −0.13 | 0.02 | 0.01 | −0.00 |
| | | (−0.07, 0.20) | (−0.08, 0.20) | (−0.17, 0.09) | (−0.25, −0.01) | (−0.07, 0.12) | (−0.07, 0.08) | (−0.08, 0.08) |
| | 0.004 | 0.05 | 0.08 | 0.07 | −0.04 | 0.03 | 0.01 | −0.00 |
| | | (−0.07, 0.20) | (−0.06, 0.22) | (−0.07, 0.21) | (−0.17, 0.09) | (−0.07, 0.12) | (−0.07, 0.10) | (−0.08, 0,08) |
| | 0.001 | 0.09 | 0.08 | 0.07 | −0.01 | 0.03 | −0.00 | −0.01 |
| | | (−0.05, 0.23) | (−0.06, 0.22) | (−0.07, 0.21) | (−0.15, 0.12) | (−0.06, 0.13) | (−0.08, 0.08) | (−0.09, 0.07) |

| | | | | | | | |
|---|---|---|---|---|---|---|---|
| Compound 22 (nM) | 0.0997 | 0.309 | 0.927 | 2.79 | 8.37 | 25 | 75 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively.

TABLE 27B
Effect of Compound A3 in Combination with DHODH inhibitor (Compound 22) on OCI-AML3 Cell Proliferation.

| | | | | | | | | |
|---|---|---|---|---|---|---|---|---|
| Compound | 15 | −0.03 | −0.04 | −0.01 | −0.05 | −0.07 | −0.09 | −0.08 |
| A3 (μM) | | (−0.08, 0.02) | (−0.08, 0.01) | (−0.06, 0.04) | (−0.09, 0.00) | (−0.12, −0.03) | (−0.13, −0.04) | (−0.12, −0.03) |
| | 3.75 | 0.00 | 0.01 | 0.01 | −0.02 | −0.05 | −0.08 | −0.07 |
| | | (−0.04, 0.05) | (−0.04, 0.05) | (−0.04, 0.05) | (−0.07, 0.03) | (−0.10, −0.01) | (−0.13, −0.04) | (−0.12, −0.03) |
| | 0.937 | 0.03 | 0.04 | 0.04 | −0.01 | −0.06 | −0.07 | −0.08 |
| | | (−0.02, 0.08) | (−0.01, 0.09) | (−0.01, 0.09) | (−0.05, 0.04) | (−0.10, −0.02) | (−0.11, −0.03) | (−0.12, −0.03) |
| | 0.240 | 0.06 | 0.07 | 0.08* | 0.00 | −0.06 | −0.07 | −0.07 |
| | | (0.00, 0.11) | (0.01, 0.12) | (0.03, 0.13) | (−0.05, 0.05) | (−0.10, −0.01) | (−0.11, −0.02) | (−0.12, −0.03) |
| | 0.059 | −0.00 | 0.03 | 0.04 | −0.10** | −0.02 | −0.04 | −0.03 |
| | | (−0.07, 0.06) | (−0.04, 0.09) | (−0.03, 0.10) | (−0.17, −0.04) | (−0.08, 0.03) | (−0.09, 0.01) | (−0.08, 0.02) |
| | 0.015 | 0.03 | 0.05 | 0.06 | −0.08 | 0.00 | −0.01 | −0.01 |
| | | (−0.03, 0.10) | (−0.01, 0.12) | (−0.00, 0.13) | (−0.14, −0.01) | (−0.05, 0.05) | (−0.05, 0.04) | (−0.06, 0.04) |

| | | | | | | | |
|---|---|---|---|---|---|---|---|
| Compound 22 (nM) | 0.06 | 0.24 | 0.98 | 3.99 | 15.6 | 62.4 | 250 |

Note that absence of any asterisk indicates an additive effect, antagonism is indicated by “*” and synergy is indicated by “**”. Antagonistic and synergistic effects are derived from the significant calls based on the maxR test and numbers associated with same interpreted as antagonistic or synergistic, respectively.

15) In Vivo Efficacy of Menin-MLL Inhibitor in Combination with DHODH Inhibitor in Disseminated Models of Human Acute Myeloid Leukemia (MOLM-13 and OCI-AML)
15A) Efficacy of Menin-MLL Inhibitor in Combination with DHODH Inhibitor in MOLM-13 (KMT2A-r) IV Model in NSG Mice

### Study Designs

Menin-MLL inhibitor, Compound A1, was administered orally (PO) and was formulated in 20% beta-hydroxypropyl-cyclodextrin (HP-3-CD). DHODH inhibitor, Compound 22, was administered orally (PO) and was formulated in 0.5% methylcellulose.
Day 0 is the day of tumor cell implantation and study initiation.
In the MOLM-13 efficacy study, mice bearing IV MOLM-13 xenograft tumors were randomly assigned to treatment groups 5 days post-tumor cell engraftment.
Treatment with vehicle, Compound A1 (10 mg/kg or 30 mg/kg), Compound 22 (0.063 mg/kg or 0.125 mg/kg) or the combination Compound 22 (0.063 mg/kg or 0.125 mg/kg)+Compound A1 (10 mg/kg, PM) was initiated on day 5, with daily dosing for 28 days. In the combination cohort mice were dosed with Compound 22 in the AM with an 8 hr spaced dose of Compound A1. The treatment of each group using compound(s) in this efficacy study is summarized in Table 28.

TABLE 28
Treatment of MOLM-13-tumors in mice

| | TREATMENT | | | | |
|---|---|---|---|---|---|
| GROUP | (n = 10) | DOSE | | ROUTE | SCHEDULE |
| 1 | Vehicles | 8 | mL/kg | po/po | QD × 28/ |
| | | | | | QD × 28 |
| 2 | Compound A1 | 10 | mg/kg | po | QD × 28 |
| 3 | Compound A1 | 30 | mg/kg | po | QD × 28 |
| 4 | Compound 22 | 0.063 | mg/kg | po | QD × 28 |
| 5 | Compound 22 | 0.125 | mg/kg | po | QD × 28 |
| 6 | Compound 22 + | 0.063 | mg/kg | po | QD × 28 |
| | Compound A1 | 10 | mg/kg | po | QD × 28 |
| 7 | Compound 22 + | 0.063 | mg/kg | po | QD × 28 |
| | Compound A1 | 30 | mg/kg | po | QD × 28 |
| 8 | Compound 22 + | 0.125 | mg/kg | po | QD × 28 |
| | Compound A1 | 10 | mg/kg | po | QD × 28 |
| 9 | Compound 22 + | 0.125 | mg/kg | po | QD × 28 |
| | Compound A1 | 30 | mg/kg | po | QD × 28 |

TABLE 29
Impact on Median survival and Life Span following
Treatment of MOLM-13 tumors in Mice

| TREATMENT | Median Survival | % ILS |
|---|---|---|
| Vehicles | 18 | |
| Compound 22 (0.063 mg/kg) | 38 | *111 |
| Compound 22 (0.0125 mg/kg) | 40.5 | *125 |
| Compound A1 (10 mg/kg) | 38 | *111 |
| Compound A1 (30 mg/kg) | 61 | *239 |
| Compound 22 (0.063 mg/kg) + | 67.5 | *275 |
| Compound A1 (10 mg/kg) | | |
| Compound 22 (0.063 mg/kg) + | NA | *†>383 |
| Compound A1 (30 mg/kg) | | |
| Compound 22 (0.125 mg/kg) + | NA | *†>383 |
| Compound A1 (10 mg/kg) | | |
| Compound 22 (0.12 5mg/kg) + | NA | *†>383 |
| Compound A1 (30 mg/kg) | | |

*p < 0.05 as compared with vehicle control and
†DHODH inhibitor treated mice;
NA = median survival not reached

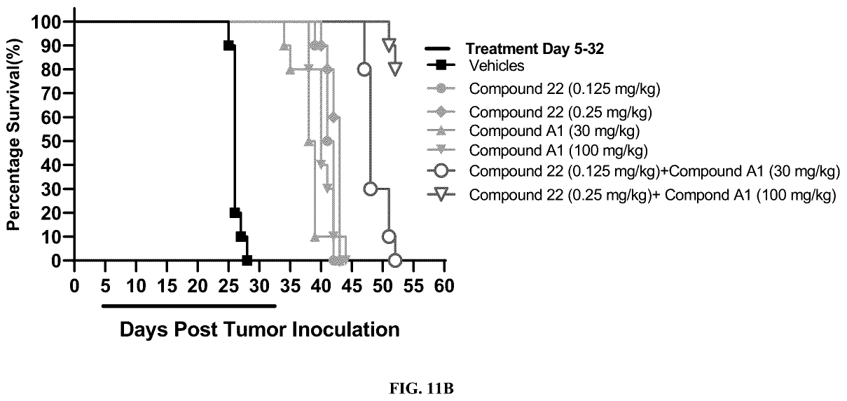

The Kaplan Meier survival curve is shown in FIG. 11A and median survival and % ILS are presented Table 29. In terms of % ILS, the treatment of mice with the DHODH inhibitor, Compound 22, in combination with Compound A1 resulted in statistically significant increased life span of MOLM-13 tumor bearing mice as compared to that of mice treated with vehicles. When 0.063 mg/kg of Compound 22 was combined with 30 mg/kg of Compound A1, or when 0.125 mg/kg of Compound 22 was combined with either 10 or 30 mg/kg of Compound A1, significant extension of lifespan was observed that was greater than additive and was statistically significant as compared to either Compound 22 or Compound A1 as a monotherapy.

15B) Efficacy of Menin-MLL Inhibitor in Combination with DHODH Inhibitor in OCI-AML3 (NPM1c) IV Model in NSG Mice
In the OCI-AML3 efficacy study, mice bearing IV OCI-AML3 xenograft tumors were randomly assigned to treatment groups 5 days post-tumor cell engraftment. Treatment with vehicle (as described above for MOLM-13), Compound A1 (30 mg/kg or 100 mg/kg), Compound 22 (0.125 mg/kg or 0.25 mg/kg) or the combination Compound 22 (0.125 mg/kg or 0.25 mg/kg)+Compound A1 (30 mg/kg or 100 mg/kg, PM) was initiated on day 5, with daily dosing for 28 days. In the combination cohorts, mice were dosed with Compound 22 in the AM with an 8 hr spaced dose of Compound A1. Higher doses of Compound A1 or Compound 22 were used compared to the MOLM-13 disseminated study because of the differential sensitivity of the tumors to the monotherapy treatment. The treatment of each group using compound(s) in this efficacy study is summarized in Table 30.

TABLE 30
Treatment of OCI-AML3-tumors in mice

| | TREATMENT | | | | |
|---|---|---|---|---|---|
| GROUP | (n=10) | DOSE | | ROUTE | SCHEDULE |
| 1 | Vehicles | 8 | mL/kg | po/po | QD × 28/ |
| | | | | | QD × 28 |
| 2 | Compound A1 | 30 | mg/kg | po | QD × 28 |
| 3 | Compound A1 | 100 | mg/kg | po | QD × 28 |
| 4 | Compound 22 | 0.125 | mg/kg | po | QD × 28 |
| 5 | Compound 22 | 0.25 | mg/kg | po | QD × 28 |
| 6 | Compound 22 + | 0.125 | mg/kg | po | QD × 28 |
| | Compound A1 | 30 | mg/kg | po | QD × 28 |
| 7 | Compound 22 + | 0.25 | mg/kg | po | QD × 28 |
| | Compound A1 | 100 | mg/kg | po | QD × 28 |

TABLE 31
Impact on Median survival and Life Span following
Treatment of OCI-AML3 tumors in Mice

| TREATMENT | Median Survival | % ILS |
|---|---|---|
| Vehicles | 26 | |
| Compound 22 (0.125 mg/kg) | 41.5 | *60 |
| Compound 22 (0.25 mg/kg) | 43 | *65 |
| Compound A1 (30 mg/kg) | 38.5 | *48 |
| Compound A1 (100 mg/kg) | 40 | *54 |
| Compound 22 (0.125 mg/kg) + | 48 | *†85 |
| Compound A1 (30 mg/kg) | | |
| Compound 22 (0.25 mg/kg) + | NA | *†100 |
| Compound A1 (100 mg/kg) | | |

*p < 0.05 as compared with vehicle control and
†DHODH inhibitor treated mice;
NA = median survival not reached

The Kaplan Meier survival curve is shown in FIG. 11B and median survival and % ILS are presented Table 31. For mice treated with any of the combination of Compound 22+Compound A1, median survival were reached with a greater number of days than that of vehicle treated or compared with each compound in monotherapy as reflected in the Table 31. In terms of % ILS, the treatment of mice with the DHODH inhibitor, Compound 22, in combination with Compound A1 resulted in statistically significant increased life span of OCI-AML3 tumor bearing mice as compared to that of mice treated with vehicles or either Compound 22 or Compound A1 as a monotherapy.

1. A combination comprising:
a therapeutically effective amount of a menin-mixed-lineage leukemia 1 (MLL) inhibitor of Formula (I), or a tautomer or a stereoisomeric form thereof, or a pharmaceutically acceptable salt or a solvate thereof; and
a therapeutically effective amount of at least one other therapeutic agent, wherein the at least one other therapeutic agent is a hypomethylating agent, cytidine deaminase inhibitor, a DNA intercalating agent, a pyrimidine analog, a purine analog, a kinase inhibitor, a CD20 inhibitor, an isocitrate dehydrogenase inhibitor, an immunomodulatory agent or a dihydroorotate dehydrogenase inhibitor;
wherein the menin-MLL inhibitor of Formula (I) has the structure: 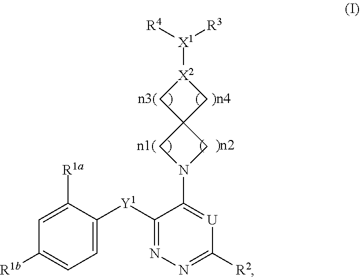
wherein
R^{1a} represents —C(═O)—NR^{xa}R^{xb}; Het; or 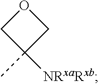
Het represents a 5- or 6-membered monocyclic aromatic ring containing one, two or three nitrogen atoms and optionally a carbonyl moiety; wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}—, —O— or —NR^{5c}—;
R² is selected from the group consisting of hydrogen, halo, C₁₋₄alkyl, —O—C₁₋₄alkyl, and
—NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{1a}R^{1b}, —C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl; wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl; and
R^{9a}, R^{9b}, R^{10a}, R^{10b}, R^{10c}, R¹¹, R^{12a}, and R^{12b} are each independently selected from the group consisting of hydrogen and C₁₋₆alkyl.

2. The combination according to claim 1, wherein the menin-MLL inhibitor of Formula (I) is Compound A: 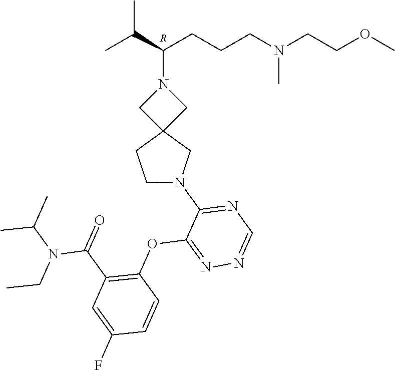
or a pharmaceutically acceptable salt or solvate thereof.

3. The combination according to claim 1, wherein the menin-MLL inhibitor of Formula (I) is Compound A4-a: 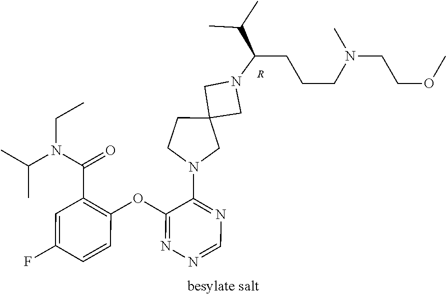
or a solvate thereof.

4. The combination according to claim 1, wherein the at least one other therapeutic agent is a hypomethylating agent.

5. The combination according to claim 4, wherein the hypomethylating agent is azacitidine or a pharmaceutically acceptable salt or solvate thereof.

6. The combination according to claim 1, wherein the at least one other therapeutic agent is a dihydroorotate dehydrogenase inhibitor.

7. The combination according to claim 6, wherein the dihydroorotate dehydrogenase inhibitor is a compound having the structure of Formula (Z): 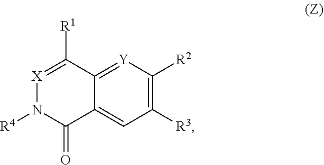
(Z), wherein
X is CH or N;
Y is CH or N;
R¹ is selected from the group consisting of C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₂₋₆haloalkenyl; N(CH₃)₂; C₃₋₆cycloalkyl; C₃₋₆cycloalkyl substituted with C₁₋₆alkyl; and phenyl;
R² is 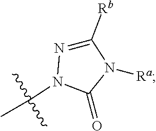
wherein
R^{a} is selected from the group consisting of C₁₋₆alkyl, C₁₋₆haloalkyl, and C₃₋₆cycloalkyl;
R^{b} is C₁₋₆alkyl or C₁₋₆alkyl substituted with a member selected from the group consisting of: OH, halo, CN, OC₁₋₆alkyl, OC₁₋₆haloalkyl and OC₃₋₆cycloalkyl;
R³ is selected from the group consisting of: H, halo, CH₃ and OCH₃;
R⁴ is selected from the group consisting of:
C₁₋₆alkyl; C₁₋₆alkyl substituted with one or two OCH₃; C₃₋₆cycloalkyl; C₃₋₆cycloalkyl substituted with CH₃, or OCH₃; CH₂—C₃₋₆cycloalkyl; and 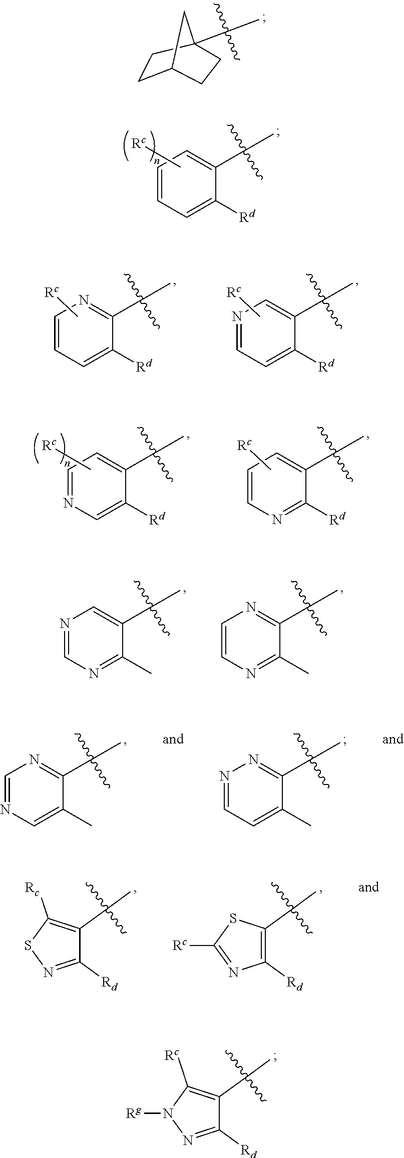
wherein
each R^{c} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; NO₂; OH; O—CH₂CH₂OH; and OC₁₋₆alkyl;
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; CN; and OC₁₋₆alkyl;
R^{g} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
or a pharmaceutically acceptable salt, isotope, N-oxide, solvate, or stereoisomer thereof;
or a compound selected from:
2-(2-Chloro-6-fluorophenyl)-6-(4-ethyl-3-(hydroxymethyl)-5-oxo-4,5-dihydro-1H-1,2,4-triazol-1-yl)isoquinolin-1(2H)-one;
6-(4-Ethyl-3-(hydroxymethyl)-5-oxo-4,5-dihydro-1H-1,2,4-triazol-1-yl)-2-(2-fluoro-4-nitrophenyl)-4-iodoisoquinolin-1(2H)-one;
2-(2-Chloro-6-fluorophenyl)-7-(4-ethyl-3-(hydroxymethyl)-5-oxo-4,5-dihydro-1H-1,2,4-triazol-1-yl)-6-fluoro-4-(prop-1-en-2-yl)phthalazin-1(2H)-one;
2-(2-Chloro-6-fluorophenyl)-7-(4-ethyl-3-(hydroxymethyl)-5-oxo-4,5-dihydro-1H-1,2,4-triazol-1-yl)-6-methoxy-4-(prop-1-en-2-yl)phthalazin-1(2H)-one;
or a pharmaceutically acceptable salt, N-oxide, solvate, or stereoisomer thereof.

8. The combination according to claim 7, wherein the dihydroorotate dehydrogenase inhibitor is 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one or a pharmaceutically acceptable salt, solvate, stereoisomer, isotopic variant, or N-oxide thereof.

9. A pharmaceutical composition comprising a combination as claimed in claim 1 and a pharmaceutically acceptable carrier.

10. A combination as claimed in claim 1 for use as a medicament.

11. A combination as claimed in claim 1 for use in the prevention or treatment of a hematopoietic disorder.

12. The combination or pharmaceutical composition for use according to claim 11 wherein the hematopoietic disorder is a nucleophosmin 1 (NPM1)-mutated leukemia or MLL-rearranged leukemia.

13. The combination or pharmaceutical composition for use according to claim 11 wherein the hematopoietic disorder is acute myeloid leukemia (AML) or acute lymphoblastic leukemia (ALL).

14. A method for treating a subject who has been diagnosed with cancer comprising administering to the subject:
a therapeutically effective amount of a menin-mixed lineage leukemia 1 (MLL) inhibitor of Formula (I), or a tautomer or a stereoisomeric form thereof, or a pharmaceutically acceptable salt or a solvate thereof; and
a therapeutically effective amount of at least one other therapeutic agent, wherein the at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor;
wherein the menin-MLL inhibitor of Formula (I) has the structure: 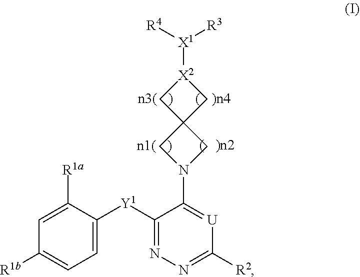
wherein
R^{1a} represents —C(═O)—NR^{xa}R^{xb}; Het; or 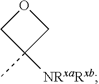
Het represents a 5- or 6-membered monocyclic aromatic ring containing one, two or three nitrogen atoms and optionally a carbonyl moiety; wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}—, —O— or —NR^{5c}—;
R² is selected from the group consisting of hydrogen, halo, C₁₋₄alkyl, —O—C₁₋₄alkyl, and
—NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}, —C₁₋₆alkyl-C(═O)—NR^{9a}R^{9b}, —C₁₋₆alkyl-OH, or
—C₁₋₆alkyl-NR¹¹—C(═O)—O—C₁₋₄alkyl-O—C(═O)—C₁₋₄alkyl; wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen;
C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl; and
R^{9a}, R^{9b}, R^{10a}, R^{10b}, R^{10c}, R¹¹, R^{12a}, and R^{12b} are each independently selected from the group consisting of hydrogen and C₁₋₆alkyl.

15. The method according to claim 14, wherein the menin-MLL inhibitor of Formula (I) is Compound A: 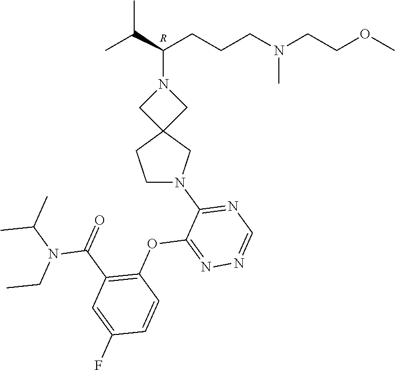
or a pharmaceutically acceptable salt or solvate thereof.

16. The method according to claim 14, wherein the menin-MLL inhibitor of Formula (I) is Compound A4-a: 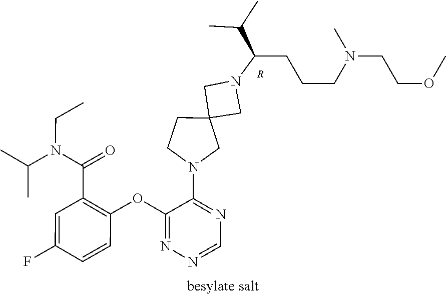
or a solvate thereof.

17. The method according to claim 14, wherein the at least one other therapeutic agent is a hypomethylating agent.

18. The method according to claim 17, wherein the hypomethylating agent is azacitidine or a pharmaceutically acceptable salt or solvate thereof.

19. The method according to claim 14, wherein the at least one other therapeutic agent is a dihydroorotate dehydrogenase inhibitor.

20. The method according to claim 19, wherein the dihydroorotate dehydrogenase inhibitor is a compound having the structure of Formula (Z): 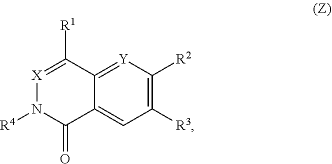
wherein
X is CH or N;
Y is CH or N;
R¹ is selected from the group consisting of C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₂₋₆haloalkenyl; N(CH₃)₂; C₃₋₆cycloalkyl; C₃₋₆cycloalkyl substituted with C₁₋₆alkyl; and phenyl;
R² is 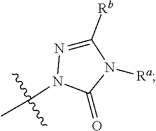
wherein
R^{a} is selected from the group consisting of C₁₋₆alkyl, C₁₋₆haloalkyl, and C₃₋₆cycloalkyl;
R^{b} is C₁₋₆alkyl or C₁₋₆alkyl substituted with a member selected from the group consisting of: OH, halo, CN, OC₁₋₆alkyl, OC₁₋₆haloalkyl and OC₃₋₆cycloalkyl;
R³ is selected from the group consisting of H, halo, CH₃ and OCH₃;
R⁴ is selected from the group consisting of:
C₁₋₆alkyl; C₁₋₆alkyl substituted with one or two OCH₃; C₃₋₆cycloalkyl; C₃₋₆cycloalkyl substituted with CH₃, or OCH₃; CH₂—C₃₋₆cycloalkyl; and; 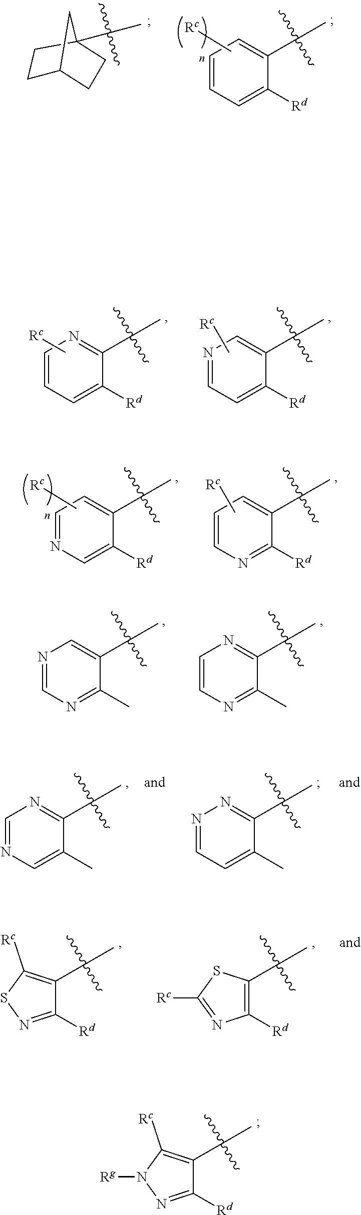
wherein
each R^{c} is independently selected from the group consisting of H; halo; C₁₋₆alkyl; C₁₋₆alkyl substituted with a member selected from the group consisting of OH, OCH₃, SCH₃, and OCF₃; C₁₋₆haloalkyl; C₁₋₆haloalkyl substituted with a member selected from the group consisting of OH, and OCH₃; NO₂; OH; O—CH₂CH₂H; and OC₁₋₆alkyl;
R^{d} is selected from the group consisting of H; halo; C₁₋₆alkyl; C₁₋₆alkyl substituted with a member selected from the group consisting of OH, OCH₃, SCH₃, and OCF₃; C₁₋₆haloalkyl; C₁₋₆haloalkyl substituted with a member selected from the group consisting of: OH, and OCH₃; CN; and OC₁₋₆alkyl;
R^{g} is selected from the group consisting of H; C₁₋₆alkyl; C₁₋₆alkyl substituted with a member selected from the group consisting of OH, OCH₃, SCH₃, and OCF₃; C₁₋₆haloalkyl; and C₁₋₆haloalkyl substituted with a member selected from the group consisting of: OH, and OCH₃; and
n is 1, or 2;
or a pharmaceutically acceptable salt, isotope, N-oxide, solvate, or stereoisomer thereof;
or a compound selected from:
2-(2-Chloro-6-fluorophenyl)-6-(4-ethyl-3-(hydroxymethyl)-5-oxo-4,5-dihydro-1H-1,2,4-triazol-1-yl)isoquinolin-1(2H)-one;
6-(4-Ethyl-3-(hydroxymethyl)-5-oxo-4,5-dihydro-1H-1,2,4-triazol-1-yl)-2-(2-fluoro-4-nitrophenyl)-4-iodoisoquinolin-1(2H)-one;
2-(2-Chloro-6-fluorophenyl)-7-(4-ethyl-3-(hydroxymethyl)-5-oxo-4,5-dihydro-1H-1,2,4-triazol-1-yl)-6-fluoro-4-(prop-1-en-2-yl)phthalazin-1(2H)-one;
2-(2-Chloro-6-fluorophenyl)-7-(4-ethyl-3-(hydroxymethyl)-5-oxo-4,5-dihydro-1H-1,2,4-triazol-1-yl)-6-methoxy-4-(prop-1-en-2-yl)phthalazin-1(2H)-one;
or a pharmaceutically acceptable salt, N-oxide, solvate, or stereoisomer thereof.

21. The method according to claim 19, wherein the dihydroorotate dehydrogenase inhibitor is 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one or a pharmaceutically acceptable salt, solvate, stereoisomer, isotopic variant, or N-oxide thereof.

22. The method according to claim 14, wherein the menin-MLL inhibitor is Compound A or a pharmaceutically acceptable salt or solvate thereof, and at least one other therapeutic agent is a dihydroorotate dehydrogenase inhibitor.

23. The method according to claim 14, wherein the cancer is a hematopoietic disorder.

24. The method according to claim 23, wherein the hematopoietic disorder is a nucleophosmin 1 (NPM1)-mutated leukemia or MLL-rearranged leukemia.

25. The method according to claim 23, wherein the hematopoietic disorder is acute myeloid leukemia (AML) or acute lymphoblastic leukemia (ALL).

26. A pharmaceutical composition as claimed in claim 9 for use as a medicament.

27. A pharmaceutical composition as claimed in claim 9 for use in the prevention or treatment of a hematopoietic disorder.
